(19) United States
(12) Patent Application Publication
(10) Pub. No.: US 2023/0340528 A1
(43) Pub. Date: Oct. 26, 2023

(54) METHODS OF TREATING CARDIAC DISORDERS AND CONGESTIVE HEART FAILURE AND ADMINISTERING AAV VECTORS

(71) Applicants: Anna TRETIAKOVA, Research Triangle Park, NC (US); Michael W. O’CALLAGHAN, Research Triangle Park, CA (US); Michael L. ROBERTS, Midlothian (GB); Jorge Omar YANEZ-CUNA, Midlothian (GB); Juan Manuel IGLESIAS GONZALEZ, Midlothian (GB); Antonia EVRIPIOTI, Midlothian (GB); Sinclair COOPER, Midlothian (GB); Ferzin SETHNA, Research Triangle Park, NC (US); Roger HAJJAR, Research Triangle Park, NC (US); ASKLEPIOS BIOPHARMACEUTICAL INC., Research Triangle Park, NC (US); SYNPROMICD LTD, Midlothian (GB)

(72) Inventors: Michael W. O’CALLAGHAN, Research Triangle Park, NC (US); Michael L. ROBERTS, Midlothian (GB); Jorg Omar YANEZ-CUNA, Midlothian (GB); Juan Manuel IGLESIAS GONZALEZ, Midlothian (GB); Antonia EVRIPIOTI, Midlothian (GB); Sinclair COOPER, Midlothian (GB); Ferzin SETHNA, Research Triangle Park, NC (US); Roger HAJJAR, Research Triangle Park, NC (US); Anna TRETIAKOVA, Research Triangle Park, NC (US)

(73) Assignee: ASKLEPIOS BIOPHARMACEUTICAL INC., Research Triangle Park, NC (US)

(21) Appl. No.: 18/019,421

(22) PCT Filed: Aug. 5, 2021
(86) PCT No.: PCT/US2021/044650

§ 371 Date: Feb. 2, 2023

Related U.S. Application Data
(60) Provisional application No. 63/214,119, filed on Jun. 23, 2021.
(60) Provisional application No. 63/061,342, filed on Aug. 5, 2020.

(51) Int. Cl. C12N 15/86; A61P 9/04

(52) U.S. Cl. CPC C12N 15/86; A61P 9/04; C12N 2750/14143; C12N 2830/008

(57) ABSTRACT

The present invention relates to methods of administration of rAAV vectors in a single administration method comprising a series of sub-administrations of sub-doses of rAAV vectors. The present invention also relates to rAAV vectors comprising cardiac-specific promoters, cardiac-cell specific promoters, multi-cell cardiac specific promoters, and elements thereof. The invention also relates to rAAV vectors, pharmaceutical compositions and uses thereof in methods for treating cardiovascular disease, heart diseases and heart failure in subjects in need thereof.

### CROSS REFERENCE TO RELATED APPLICATIONS

This application is a 35 U.S.C. § 371 National Phase Entry Application of international patent application No. PCT/US2021/044650 filed Aug. 5, 2021, which designated the U.S., which claims benefit under 35 U.S.C. § 119(e) of U.S. Provisional Application Nos 63/061,342 filed Aug. 5, 2020; and 63/214,119 filed Jun. 23, 2021, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Aug. 4, 2021, is named 046192-097980WOPT _SL.txt and is 1,059,646 bytes in size.

### FIELD OF THE INVENTION

The technology herein relates to AAV vectors and regulatory nucleic acid sequences, in particular cardiac-specific promoters, muscle cell specific promoters, and elements thereof for the treatment of cardiac disorders, heart failure, including chronic heart failure (CHF). The technology herein also relates to expression methods of administering AAV vectors for the treatment of cardiac disorders. The technology herein also relates to constructs, vectors, virions, pharmaceutical compositions and cells comprising such promoters for decreasing phosphatase activity to improve β-adrenergic responsiveness, and to methods of their use.

### BACKGROUND OF THE INVENTION

The following discussion is provided to aid the reader in understanding the disclosure and does not constitute any admission as to the contents or relevance of the prior art.

Heart failure-defined by the ACC/AHA as the complex clinical syndrome that results from any structural or functional impairment of ventricular filling or ejection of blood-remains one of the most common, costly, and debilitating diseases in the United States. Based on National Health and Nutrition Examination Survey data from 2011 to 2014, an estimated 6.5 million US adults have it, with projections of more than 8 million by 2030. More than 960,000 new cases are thought to occur annually, with a lifetime risk of developing it of roughly 20% to 45%. Heart failure (HF), also called congestive heart failure (CHF) is therefore a disease of epidemic portions in the United States.

Heart failure is a disorder in which the contractility of the heart muscle decreases, and the heart loses its ability to pump blood efficiently. Heart failure is almost always a chronic, long-term condition, and consumes an inordinate amount of medical intervention and human resource dollars. In particular, the consequences of heart failure to the rest of the body organs can be devastating both in terms of the overall reduction in productive life of the patient, and the expense of treatment. The condition may affect the right side, the left side, or both sides of the heart. As the pumping action of the heart is compromised, blood begins backing up into other areas of the body. Many organs and organ systems begin to suffer cumulative damage from lack of oxygen and nutrients.

In slightly over one-half of affected individuals, function of the heart is reduced, as demonstrated by a decrease in ejection fraction (heart failure with reduced ejection fraction; HFrEF), and the left ventricle is dilated. New drugs that target pathways critical to progression of HF, along with implantable cardiac defibrillators and resynchronization devices, have been introduced over the past 3 decades. However, both the morbidity and mortality associated with HFrEF remains at unacceptable levels, with as many as 50% of affected individuals dying within 5 years of diagnosis. This has led investigators to evaluate the role of gene therapy in mitigating or curing HFrEF by increasing the amount of a specific protein in the heart

Protein kinases and their phospho-protein substrates are important in the heart’s pumping action and have been well characterized, however, the protein phosphatases that reverse the increased cardiac contractility are also important. Stemming from a common gene family, the major Ser/Thr phosphatases (type 1, type 2A and type 2B (calcineurin), are highly homologous proteins (40-50%) (Cohen, P., 1990 Phosphoprotein Res; 24:230-5) that play critical roles in the control of cardiac contractility and hypertrophy. Overexpression of the catalytic subunit of protein phosphatase 2A has been shown to decrease cardiac function and lead to a pathologic cardiac hypertrophy (Brewis, N. et al., 2000 Am J Physiol Heart Circ Physiol; 279:H1307-18; Gergs, U. et al., 2004 J Biol Chem.). Furthermore, calcineurin, a calcium dependent phosphatase, induces hypertrophy by its regulation of the NFAT transcription factor activity. 5 Interestingly, inhibition of this phosphatase blocks cardiac hypertrophy in vivo and in vitro (Brewis, N. et al., 2000; Molkentin, J. D., 1998 Cell; 93:215-28).

In human and experimental heart failure, the activity of the type 1 phosphatase (PP1) associated with the sarcoplasmic reticulum (SR) is significantly increased, suggesting that this may be a contributing factor to depressed function, dilated cardiomyopathy and premature death (Huang, B. et al., 1999 Circ Res; 85:848-55; Sande, J. B., et al., 2002 Cardiovasc Res; 53:382-91; Boknik, P. et al., 2000 Naunyn Schmiedebergs Arch Pharmacol; 362:222-31; Gupta, R. C. et al., 1997 Circulation; 96 (Suppl 1):1-361; Neumann, J. 1997 J Mol Cell Cardiol; 29:265-72; Carr, A. N. et al., 2002, Mol Cell Biol; 22:4124-35).

It has been previously established that Protein Kinase C alpha (“PKC-α”) activity is increased in the pathological state of heart failure. Inhibiting the activity of Protein Phosphatase-1 (“PP-1”) results in enhanced cardiac contractility (Pathak, A., et al. 2005 Circ Res 15:756′-66).

There is a need for an effective treatment of, and also, the prevention of heart failure in subjects. Gene therapy has been useful for the treatment of a variety of diseases and disorders. However, it is important that both the delivery of the gene therapy is optimal, as well as the expression of the gene or nucleic acid to a particular tissue and/or cell type.

Gene therapy has the potential to not only cure genetic disorders, but to also facilitate the long-term non-invasive treatment of acquired and degenerative diseases using a virus. One gene therapy vector is adeno-associated virus (AAV). AAV itself is a non-pathogenic-dependent parvovirus that needs helper viruses for efficient replication. AAV has been utilized as a virus vector for gene therapy because of its safety and simplicity. AAV has a broad host and cell type tropism capable of transducing both dividing and non-dividing cells.

Following extensive study of the internal mechanisms of gene regulation within the body, research focus has recently shifted to regulation of gene expression by introducing exogenous nucleic acid sequences into cells. This is done conventionally in research and bioprocessing, wherein the nucleic acid sequence of a desired expression product operably liked to a promoter is introduced into a production cell line, often in the form of a vector.

In the field of gene therapy, this has been of particular interest for genetic disorders such as single gene disorders (or Mendelian disorders) which are caused by the presence of a faulty gene into the cells of a patient. In gene therapy, controlling the expression of the exogenous nucleic acid which has been introduced into the cells is of paramount importance for the health and safety of the patients. The level of an expression product not only needs to be within a therapeutic window but also the expression needs to be within a required tissue or even a specific region within the required tissue for the treatment to be effective. Additionally, expression may need to be restricted to a specific cell type or a multiple cell types in order to avoid side effects. Expression outside the therapeutic window (i.e. lower or higher) or expression outside the therapeutic region, or even outside the specific cell or combination of cells, may not be useful therapeutically or even be deleterious.

Therefore, there is a need for promoters driving expression in the heart, in particular in the smooth muscle or cardiac muscle of the heart, particularly in specific cell types such as cardiomyocytes and the like. Expression in the heart includes expression in cardiac cells, e.g., cardiomyocytes, as well as smooth muscle cells in the heart.

Therefore, there is a need for effective treatment of heart failure, including congestive heart failure (CHF), and other heart diseases and disorders. There is also a need for effective administration of viral vectors for treatment of diseases. One or more aspects of the present invention are intended to address one or more of the above-mentioned problems.

### SUMMARY OF THE INVENTION

The technology described herein relates generally to gene therapy constructs, compositions and methods of administration, for the treatment of cardiovascular conditions, heart disease and heart failure. Also disclosed herein are methods for administration AAV vectors, as well as compositions and methods comprising AAV vectors for the treatment of heart disorders and diseases, including heart failure and congestive heart failure (CHF). In some aspects, AAV vectors for the treatment of heart disorders and diseases include, e.g., an AAV vector encoding a phosphatase inhibitor, for example, for expression of the phosphatase inhibitor in heart cells for the treatment of cardiac disorders, e.g., heart failure. Decreasing phosphatase activity can improve β-adrenergic responsiveness.

In particular, aspects of the present invention are directed to novel methods of administration and novel rAAV compositions for the treatment of subjects with heart failure, including methods of administration comprising administering to a subject with a classification of heart failure a dose of a rAAV where upon at least 12-months after the administration the classification of the heart failure is improved by at least one, or at least two stages or classification levels. In some embodiments, the methods of administration disclosed herein can be used in combination with other agents, including but not limited to use of immunomodulators and/or vasodilators, as well as rAAV vectors comprising a codon optimized nucleic acid sequence to encode I-1c (a constitutively active truncated inhibitor-1 that inhibits protein phosphatase 1 activity), and/or rAAV vectors comprising novel cardiac-specific muscle promoters. Moreover, the inventors have demonstrated different ways to treat subjects with heart failure, including subjects with non-ischemic cardiomyopathy and ischemic cardiomyopathy that have the ability to significantly improve the subjects’ categorization in a classification system used to assess heart failure. A range of classification systems to categorize the extent of a subjects’ of heart failure can be used and are well known in the art, and includes but are not limited to American Heart Association (AHA), the American College of Cardiology (ACC), Minnesota LIVING WITH HEART FAILURE® Questionnaire (MLHFQ), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA), or modified assessments or combinations or merged assessments thereof.

For exemplary purposes only and without wishing to be limited to theory, the methods of treating a subject with heart failure as disclosed herein, or administration methods as disclosed herein have been demonstrated to improve a subject’s classification of heart failure, within at 12-months post-administration of a rAAV disclosed herein, from, e.g., a category IV to a category III or less than category III, or for e.g., from a category III to a category II or less than category II, according to a heart failure classification system as disclosed herein. In some embodiments, a system equivalent to the NYHA or the AHA or the ACC classifications are used, or any other comparative heart failure classification system known to a person of ordinary skill in the art.

Other aspects of the technology described herein relates to novel rAAV vectors encoding a I-1c protein operatively linked to a cardiac specific promoter, or a cardiac muscle specific promoter, or a skeletal muscle promoter specifically active in the cardiac muscle as disclosed herein. In some embodiments, the rAAV vector encoding a I-Ic protein comprises a codon optimized nucleic acid sequence encoding I-1c, e.g., selected from any of SEQ ID NO: 385-412, or a nucleic acid sequence that has at least 85% sequence identity thereto. In some embodiments, the rAAV vector encoding a I1c protein comprise a codon optimized nucleic acid sequence encoding I1c e.g. selected from any of SEQ ID NOS: 385-412, or, a nucleic acid sequence that has at least 85% sequence identity thereto, is operatively linked with CMV promoter, or, a cardiac specific promoter, or a cardiac muscle specific promoter, or a skeletal muscle specific active in cardiac muscle as disclosed herein. In some embodiments, the rAAV vector comprising codon optimized nucleic acid sequence encoding I1c, e.g. selected from any of SEQ ID NOS: 385-412, or a nucleic acid sequence that has 85% sequence identity thereto, further comprise reverse poly A or, ds RNA termination element. In some embodiments, the rAAV vector encoding a I-Ic protein for use in the methods and compositions as disclosed herein comprises a nucleic acid sequence selected from any of SEQ ID NO: 413-440, or a nucleic acid sequence that has at least 85% sequence identity thereto.

Also provided herein is close ended linear duplex DNA (or, also referred to as closed linear DNA herein) comprising a nucleic acid sequence of any of SEQ ID NO: 357-384. In some embodiments, the rAAV vector lacking bacterial sequences and encoding a I-Ic protein for use in the methods and compositions as disclosed herein is manufactured using a close ended linear duplex DNA of SEQ ID NO: 357-384.

One aspect of the technology described herein relates to a method to administer a rAAV vector, where the method is a single administration to the subject, where the single total dose administration comprises at least 2, or 3, or 4, or 5 or more sub-doses within the single administration. That is, stated differently, in some embodiments, the method comprises administering a rAAV vector to the subject in a single administration, where the single total dose administration comprises the administration of rAAV from least 2, or 3, or 4, or 5 or more vials, in which the total rAAV dose us administered from each vial over a time period between 1-5 minutes, or more than 5 minutes. In some embodiments, the rAAV vector is selected from the group consisting of AAV2, AAV6, AAV8, AAV9, AAV2i8, rh10, AAV2.5 and AAV2G9. In certain embodiments, the rAAV vector is AAV2i8 (also referred to as BNP116). In yet another embodiment, the rAAV vector comprises a VP1, a VP2, and/or VP3 capsid protein from a serotype selected from the group of AAV serotypes listed in table 11, which also lists the capsid protein sequences known in the art.

In some embodiments, the methods relate to administration of a rAAV vector to the heart of a subject, e.g., a human subject. In some embodiments, the rAAV vector comprises a cardiac-specific promoter, for example, an exemplary cardiac-specific promoter selected from any of these disclosed in Table 2A herein, or a functional variant or functional fragment thereof, or any cardiac specific promoter (CSP) selected from Tables 2 and 3 herein. In some embodiments, the rAAV vector is administered according to the disclosed methods for the treatment of cardiovascular conditions, heart failure or a heart disease or disorder. In some embodiments, the rAAV vector administered according to the methods disclosed herein is a rAAV vector comprising a nucleic acid encoding a therapeutic agent for treatment of heart failure, wherein the nucleic acid is operatively linked to a cardiac-specific promoter as disclosed in Table 2A herein, or a functional variant or functional fragment thereof, or any CSP selected from Tables 2 and 3 herein.

In some embodiments, the technology described herein relates to a method to co-administer a rAAV vector with an immune modulator, as disclosed herein.

Another aspect of the technology described herein relates to gene therapy constructs, methods and composition, for the treatment of heart failure. More particularly, the technology relates to adeno-associated (AAV) virions configured for delivering an inhibitor of protein phosphate 1 (PP1) to a subject, and more particularly for delivering a PP1 inhibitor for expression in the heart of a subject.

Accordingly, in one aspect, this disclosure features a method that includes administering, into heart cells, e.g., cardiomyocytes, a rAAV vector expressing an agent that modulates phosphatase activity, e.g., type 1 phosphatase activity, in the cells. The heart cells can be in vitro or in vivo. For example, the heart cells can be in a heart of a subject. The method can be used to treat a subject, e.g., a subject having a cardiac disorder, e.g., heart failure. Typically, the subject is a mammal, e.g., a human or non-human mammal. Type 1 phosphatases include, but are not limited to, PP1cα, PP1cβ, PP1cδ and PP1cγ.

In one embodiment, the agent is a nucleic acid that comprises a sequence encoding a protein that inhibits phosphatase activity, e.g., type 1 phosphatase activity. The rAAV vector can be administered in an amount effective to decrease phosphatase activity and/or increase β-adrenergic responsiveness in the treated cells.

In some embodiments, the rAAV vector expresses a nucleic acid that increases expression of an endogenous nucleic acid that encodes a protein that inhibits phosphatase activity. For example, the nucleic acid can include a sequence that encodes a transcription factor, e.g., an engineered transcription factor such as a chimeric zinc finger protein. In still another example, the nucleic acid is a regulatory sequence that integrates in or near the endogenous nucleic acid that encodes a protein that inhibits phosphatase activity, e.g., in or near a gene encoding phosphatase inhibitor-1 (“I-1”).

In still another embodiment, the rAAV vector expresses a nucleic acid that can provide a nucleic acid modulator of gene expression. For example, the nucleic acid can be a nucleic acid that can express such a nucleic acid modulator, e.g., a dsRNA (e.g., siRNA), an anti-sense RNA, or a ribozyme.

In one embodiment, the rAAV vector disclosed herein comprises, in its genome: 5′ and 3′ AAV inverted terminal repeats (ITR) sequences, and located between the 5′ and 3′ ITRs, a heterologous nucleic acid sequence encoding a protein phosphate 1 (PP1) inhibitor, wherein the heterologous nucleic acid is operatively linked to a cardiac-specific promoter (CSP). In some embodiments, the PP1 inhibitor is Inhibitor-1 (I-1) or a functional variant thereof. In some embodiments, the cardiac-specific promoter is a synthetic cardiac specific promoter selected from any promoter listed in Table 2A herein, or a functional variant or functional fragment thereof, or any CSP selected from Tables 2 and 3 herein.

In one embodiment, the rAAV is administered by an injection, e.g., a direct injection into the heart, e.g., a direct injection into the left ventricle surface. In some embodiments, the rAAV is administered into a lumen of the circulatory system, e.g., into a chamber or the lumen of the heart or a blood vessel of the heart of a subject. For example, the pericardium can be opened and the rAAV can be injected into the heart, e.g., using a syringe and a catheter. The rAAV can be administered into the lumen of the aorta, e.g., the aortic root, introduced into the coronary ostia or introduced into the lumen of the heart. The rAAV can be administered into a coronary artery. It is also possible to restrict blood flow to increase resident time in the blood vessel, e.g., in the coronary artery, e.g., using an antegrade or retrograde blockade. In some embodiments, the rAAV is administered using syringe fitted with injection pump or, infusion pump. In some embodiments, the rAAV is administered using manually controlled syringe.

In one embodiment, the rAAV vector as disclosed herein is introduced by a percutaneous injection, e.g., retrograde from the femoral artery retrograde to the coronary arteries. In still another embodiment, the rAAV vector as disclosed herein is introduced, e.g., using a stent. For example, the rAAV vector as disclosed herein is coated on the stent and the stent is inserted into a blood vessel, such as a coronary artery, peripheral blood vessel, or cerebral artery.

In one embodiment, introducing the rAAV vector as disclosed herein includes restricting blood flow through coronary vessels, e.g., partially or completely, introducing the viral delivery system into the lumen of the coronary artery, and allowing the heart to pump, while the coronary vein outflow of blood is restricted. Restricting blood flow through coronary vessels can be performed, e.g., by inflation of at least one, two, or three angioplasty balloons. Restricting blood flow through coronary vessels can last, e.g., for at least one, two, three, or four minutes. Introduction of the viral particle into the coronary artery can be performed, e.g., by an antegrade injection through the lumen of an angioplasty balloon. The restricted coronary vessels can be: the left anterior descending artery (LAD), the distal circumflex artery (LCX), the great coronary vein (GCV), the middle cardiac vein (MCV), or the anterior interventricular vein (AIV). Introduction of the viral particle can be performed after ischemic preconditioning of the coronary vessels, e.g., by restricting blood flow by e.g., inflating at least one, two, or three angioplasty balloons. Ischemic preconditioning of the coronary vessels can last for at least one, two, three, or four minutes.

In one embodiment, introducing the rAAV vector as disclosed herein includes restricting the aortic flow of blood out of the heart, e.g., partially or completely, introducing the viral delivery system into the lumen of the circulatory system, and allowing the heart to pump, e.g., against a closed system (isovolumically), while the aortic outflow of blood is restricted. Restricting the aortic flow of blood out of the heart can be performed by redirecting blood flow to the coronary arteries, e.g., to the pulmonary artery. Restricting the aortic flow of blood can be accomplished by clamping, e.g., clamping a pulmonary artery. Introducing the viral particle can be performed e.g., with the use of a catheter or e.g., by direct injection. Introducing the viral particle can be performed by a delivery into the aortic root.

A cardiac specific promoter can be expressed in other cells. However, it has a higher degree of expression in the cardiac cells such as cardiomyocytes in the heart, as well as non-cardiomyocyte cells or located in the heart. For example, a cardiac-specific promoter expresses a gene at least 25%, or at least 35%, or at least 45%, or at least 55%, or at least 65%, or at least 75%, or at least 80%, or at least 85%, or at least 90%, or at least 95%, or any integer between 25%-95% higher in cells located in the heart, including cardiomyocyte and non-cardiomyocyte cells located in the heart as compared to cells located outside the heart.

Functional variants are defined herein below. Suitably, the synthetic cardiac-specific promoter may comprise a sequence which is at least 60%, 65%, 70%, 75%, 80%, 90%, 95%, 96%, 97%, 98% or 99% identical to any one of SEQ ID NOs: 3-64. Suitably the synthetic cardiac-specific promoter may comprise a sequence which is at least 60%, 65%, 70%, 75%, 80%, 90%, 95%, 96%, 97%, 98% or 99% identical to any one of SEQ ID NO: 3-64, operably linked to a sequence which is at least 60%, 65%, 70%, 75%, 80%, 90%, 95%, 96%, 97%, 98% or 99% identical to any one of SEQ ID NOs: 3-32.

In one embodiment, there is provided a synthetic cardiac-specific promoter comprising or consisting of at least one of the following cis-regulatory elements (CREs) disclosed herein.

One aspect of the technology disclosed herein relates to a method of treating a patient having a heart failure, comprising: administering into heart cells of the patient, at least one total dose of a rAAV vector comprising a nucleic acid sequence encoding a phosphatase inhibitor protein that inhibits phosphatase activity, wherein, at least one dose of the rAAV is selected from a total dose-range of about 10¹³vg to about 10¹⁵vg, and wherein, six months post-administration NT-proBNP level in serum of the patient is below 900 pg/ml.

Another aspect of the technology described herein relates to a method of treating a patient having a heart failure, comprising: administering into heart cells of the patient, at least one total dose of a rAAV vector comprising (i) a nucleic acid sequence encoding a phosphatase inhibitor protein that inhibits phosphatase activity, (ii) a synthetic promoter, operatively linked to the phosphatase inhibitor (I-1) protein.

Another aspect of the technology described herein relates to a method of treating a patient having a cardiovascular condition or a heart disease, comprising: administering into heart cells of the patient, at least one total dose of a rAAV vector comprising a therapeutic nucleic acid operatively linked to a cardiac-specific promoter selected from Table 2A or a variant thereof, or a muscle-specific promoter active in cardiac and skeletal muscle selected from Table 5A or a variant thereof, wherein the therapeutic nucleic acid is RNA or DNA, and wherein the therapeutic nucleic acid expresses a therapeutic protein selected from Table 18A or 18B.

Another aspect of the technology described herein relates to a method of treating a patient having congestive heart failure, comprising: administering to a patient, at least one dose of a rAAV vector, wherein the rAAV vector is AAV2i8 and comprises a nucleic acid encoding phosphatase inhibitor 1 (I-1) operatively linked to a promoter selected from: a CMV promoter, a cardiac-specific promoter selected from Table 2A or a variant thereof, or a muscle-specific promoter active in cardiac and skeletal muscle selected from Table 5A or a variant thereof.

In all aspect of the methods and compositions as disclosed herein, in some embodiments, the rAAV vector comprises a nucleic acid sequence encoding a polypeptide comprising amino acids 1-65 of SEQ ID NO: 2, wherein threonine at position 35 of SEQ ID NO: 1 is replaced with aspartic acid (T35D). In some embodiments, the nucleic acid sequence is a codon optimized nucleic acid sequence selected from any of SEQ ID NO: 385-412.

Also provided are closed linear DNA constructs comprising a nucleic acid sequence of any of SEQ ID NO: 385-412. The closed linear DNA can be used in methods for making rAAV that lack bacterial DNA sequences. Thus, also provided herein are pharmaceutical compositions for treatment of heart failure that comprise rAAV encoding constitutively active I-1c, wherein the rAAV compositions lack bacterial nucleic acid sequences.

In all aspect of the methods and compositions as disclosed herein, in some embodiments, the rAAV vector further comprises CMV promoter or a synthetic promoter, operatively linked to the phosphatase inhibitor protein. In some embodiments, the synthetic promoter is a cardiac-specific promoter selected from Table 2A or a variant thereof, or a muscle-specific promoter active in cardiac and skeletal muscle selected from Table 5A or a variant thereof. In some embodiments, the synthetic promoter causes expression of the therapeutic nucleic acid or phosphatase inhibitor protein preferentially in smooth muscle cells. In some embodiments, the synthetic promoter causes expression of the therapeutic nucleic acid or phosphatase inhibitor protein preferentially in cardiac cells. In some embodiments, the expression of the therapeutic nucleic acid or phosphatase inhibitor protein by the cardiac- or muscle-specific promoter is equivalent to, or greater than, the expression caused by CMV promoter.

In all aspect of the methods and compositions as disclosed herein, in some embodiments, the total dose is administered over a period of time of about 20 minutes to about 30 minute. In some embodiments, the total dose is performed in sub-doses, wherein each sub-dose is administered over a period of time of 1-5 minutes, for example, the administration of the total dose is performed in five sub-doses, each sub-dose is administered over a period of time of 1-5 minutes, where, for example, the five sub-doses are administered over a period of about 20 minutes to about 30 minutes.

In all aspect of the methods and compositions as disclosed herein, in some embodiments, the rAAV is selected from the group consisting of AAV2, AAV6, AAV8, AAV9, AAV2i8, rh10, AAV2.5 and AAV2G9. In some embodiments, the rAAV is AAV2i8 or AAV9. In some embodiments, the rAAV is selected from any AAV serotypes disclosed in Table 11.

In all aspect of the methods and compositions as disclosed herein, in some embodiments, the at least one total dose of the rAAV is 10¹³ vg, 3×10¹³ vg, 10¹⁴ vg, 3×10¹⁴ vg, or, 10¹⁵ vg. In some embodiments, the at least one total dose of the rAAV is selected from a dose-range of about 10¹³vg to about 10¹⁵vg.

In all aspect of the methods and compositions as disclosed herein, in some embodiments, at least about six-months post administration of the rAAV dose, NT-proBNP level in serum of the patient is measured and is below 900 pg/ml.

In all aspect of the methods and compositions as disclosed herein, in some embodiments, the method further comprises administering an immune modulator. In some embodiments, the administration further comprises nitroprusside or nitroglycerin. In all aspect of the methods and compositions as disclosed herein, the administration is into the lumen of the coronary artery of the heart of the patient or systemic administration.

Also provided herein is a close ended linear duplex DNA (also referred to as closed linear DNA in the application) of any of SEQ ID NO: 357-384 that is used to generate rAAV of the present invention.

In all aspects of the methods and compositions as disclosed herein, the methods are useful to treat heart failure, including congestive heart failure (CHF), wherein the HF or CHF is selected from any of: left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

In all aspects of the methods and compositions as disclosed herein, the methods are useful to treat cardiomyopathy.

In all aspects of the methods and compositions as disclosed herein, the methods are useful to treat non-ischemic cardiomyopathy, or non-ischemic heart failure.

In all aspects of the methods and compositions as disclosed herein, the methods are useful to treat ischemic cardiomyopathy, or non-ischemic heart failure.

One aspect of the technology described herein relates to a method of treating a patient having a heart failure, comprising: (i) administering into heart cells of the patient having a classification of congestive heart failure (CHF), at least one total dose of a rAAV vector comprising a nucleic acid sequence encoding a phosphatase inhibitor (I-1) protein that inhibits phosphatase activity, where at least one dose of the rAAV is selected from a total dose-range of about 10¹³vg to about 10¹⁵vg, and where. at least twelve months post-administration, there is an improvement in the classification of congestive heart failure.

In all aspects of the methods disclosed herein, the classification of heart failure is based upon a classification system used by the American Heart Association (AHA), the American College of Cardiology (ACC), Minnesota LIVING WITH HEART FAILURE® Questionnaire (MLHFQ), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA), or an equivalent classification system thereof. In some embodiments, there is an improvement in the classification of at least one level, or at least two levels, after 12 months after administration of the rAAV. In some embodiments, there is an improvement in the classification of at least one level, or two levels within six months after administration of the rAAV.

In all aspects of the methods disclosed herein, the classification of heart failure is NYHA and the level of classification is selected from the group consisting of: Class I, Class II, Class III, and Class IV. In some embodiments, the classification system is the American College of Cardiology/American Heart Association (ACC/AHA) complementary staging system and the level of classification is selected from the group consisting of: Stages A, Stage B, Stage C, Stage D. In some embodiments, the classification system is KCCQ and the level of classification is a KQQC overall summary score range selected from the group consisting of: KCCQ fair to excellent scores of 50 to 100, very poor to fair scores of 0 to 49, good to excellent scores of 75 to 100, and very poor to good scores of 0 to 74.

Another aspect of the technology described herein relates to a method of treating a patient having cardiomyopathy, comprising administering into heart cells of the patient at least one total dose of a rAAV vector comprising a nucleic acid sequence encoding a phosphatase inhibitor (I-1) protein that inhibits phosphatase activity, where at least one dose of the rAAV is selected from a total dose-range of about 10¹³vg to about 10¹⁵vg, and where, at least twelve months post-administration, there is an improvement in the at least one parameter from a baseline level in the patient, where the at least one parameter is selected from the group consisting essentially of: (i) ejection fraction (EF), (ii) end systolic volume (ESV), (iii) cardiac contractility, selected from ejection fraction (EF) and fractional shortening (FS); (iv) cardiac volumes selected from any of: end diastolic volume (DV) and end systolic volume (ESV), (v) functional criteria, selected from any of: a 6-minute walk test (6MWT), exercise and VO2max; (vi) BNP level, Pro-BNP level, (vii) biomarker level, wherein the biomarker level is selected from the group of: troponin, serum creatinine, cystatin-C, or hepatic transaminases, (viii) Patient-reported outcomes (PROs), such as reduced symptoms, health-related quality of life (HRQOL), or patient perceived health status, and (ix)decrease in any of: mortality risk due to heart failure, reduced hospitalization due to heart failure symptoms, or therapeutic intervention for treatment of heart failure.

In all aspects of the methods disclosed herein, there is an improvement of at least 2, or at least 3, or at least 4, or at least 5 parameters at least 12 months after administration. In some embodiments, there is an improvement of at least 2, or at least 3, or at least 4, or at least 5 parameters at least 6 months after administration. In some embodiments, the improvement is selected from any of: (a) at least a 5% or more increase in ejection fraction from baseline, (b) at least a 10% decrease, or at least a 20 ml decrease in end systolic volume from baseline, (c) at least a 50-meter increase in 6-minute walk test from baseline, (d) at least a 40% decrease in BNP levels (pg/ml) in the blood from baseline, (e) at least a 35% decrease in pro-BNP levels (pg/ml) in the blood from baseline, (f) at least a 10% reduction in a biomarker selected from: troponin, serum creatinine, cystatin-C, or hepatic transaminases from a baseline level of the same biomarker, (g) at least a 1.5 ml/kg/min increase in myocardial oxygen consumption (MVO2) from baseline, or, (h) a discharge from hospital due to improved HF symptoms, or a reduced intervention selected from a decrease in the use of any of: inotropes, vasodilators, diuretcis due to improved HF symptoms in the subject.

In all aspects of the methods and compositions disclosed herein, the rAAV vector further comprises CMV promoter or a synthetic promoter, operatively linked to the phosphatase inhibitor protein. In all aspects of the methods and compositons disclosed herein, a total dose or rAAV is administered as any of the following administration methods: (a) over a period of time of about 20 minutes to about 30 minutes, (b) administered in a series of sub-doses, wherein each sub-dose is administered over a period of time of about 1 minute to about 5 minutes or (c) administered in a series of five sub-doses, each sub-dose is administered over a period of time of about 1 minute to about5 minutes, and wherein the five sub-doses are administered over a period of about 20 minutes to about 30 minutes.

In all aspects of the methods and compositons disclosed herein, the rAAV vector comprises a capsid that detargets the liver - that is, the rAAV preferentially targets tissues other than the liver. In some embodiments of all aspects described herein, the rAAV can preferentially target muscle cells, including but not limited to, cardiac muscle and cardiomyocytes. In all aspects of the methods and compositions disclosed herein, the rAAV is selected from the group consisting of: AAV1, AAV2, AAV6, AAV8, AAV9, AAV2i8, rh10, AAV2.5 and AAV2G9, or any rAAV selected from Table 11. In some embodiments, the rAAV vector is AAV2i8.

In all aspects of the methods and compositions disclosed herein, the rAAV vector is administered in at least one total dose of the rAAV is 10¹³ vg, 3×10¹³ vg, 10¹⁴ vg, 3×10¹⁴ vg, or, 10¹⁵ vg.

In all aspects of the methods and compositions as disclosed herein for the treatment of cardiomyopathy, the rAAV vector encodes a protein selected from any of the proteins listed in Table 18A or 18B. In all aspects of the methods and compositions as disclosed herein for the treatment of cardiomyopathy, the rAAV vector comprises a nucleic acid sequence encoding a phosphatase inhibitor (I-1) protein, for example a constitutively active protein (I-1c). In all aspects of the methods and compositions disclosed herein, the I-1c is selected from any of: (a) a polypeptide comprises at least amino acid residues 1-54 of SEQ ID NO: 1, wherein SEQ ID NO: 1 is truncated at the C-terminus at amino acid 70, 67, 66, 65, or 61, or 54, and where the there is an aspartic acid at position 35 (T35D), (b) a polypeptide comprising amino acids 1-54 of SEQ ID NO:1 or a functional fragment thereof, wherein the functional fragment has at least 85% sequence identity to amino acid residues 1-54 of SEQ ID NO: 1, or truncated at the C-terminus at amino acid 70, 67, 66, 65, or 61, or 54, and where the there is an aspartic acid at position 35 (T35D), or (c) a polypeptide selected from any of: SEQ ID NOS: 507 or 527-532 or a functional equivalent thereof having at least 85% sequence identity to amino acid residues of any of SEQ ID NOS: 507 or 527-532.

In all aspects of the methods and compositions disclosed herein, the rAAV genome comprises nucleic acid sequence selected from the group consisting of: SEQ ID NO: 413-441. In some embodiments, the nucleic acid sequence encoding the I-1 polypeptide is selected from: (a) a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), or (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

In all aspects of the methods and compositions disclosed herein, the nucleic acid sequence encoding the I-1 protein is a codon optimized nucleic acid sequence, for example, but not limited to, a nucleic acid sequence encoding the I-1 protein is selected from any of SEQ ID NO: 385-412, or a nucleic acid sequence having at least 80% sequence identity to SEQ ID NO: 385-412.

In all aspects of the technology described herein, the methods and compositions disclosed herein can be used to treat a subject with cardiomyopathy, wherein the subject with cardiomyopathy has non-ischemic heart failure and/or non-ischemic cardiomyopathy, including but not limited to, acquired cardiomyopathy, cardiomyopathy acquired as a result of an infection, or toxin, etc., or a congenital cardiomyopathy or a genetic disorder with a cardiac manifestation. In some embodiments, a subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

In all aspects of the technology described herein, the methods and compositions disclosed herein can be used to treat a subject with cardiomyopathy, wherein the subject with cardiomyopathy has an ischemic cardiomyopathy.

In all aspects of the technology described herein, the methods and compositions disclosed herein can be used to treat a subject with cardiomyopathy, where the subject with cardiomyopathy has heart failure. In such an embodiment, the subject with heart failure has a classification that is equivalent to class III or above in the New York Heart Association (NYHA) classification system. In some embodiments, the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

In all aspects of the technology described herein, the methods and compositions disclosed herein can be used to treat a subject with cardiomyopathy, where the subject with cardiomyopathy has reduced ejection fraction (rEF or HFrEF), or, preserved ejection fraction (HFpEF).

In some embodiments in the methods to treat a subject with cardiomyopathy and where the subject has heart failure, at least twelve months post-administration of the rAAV, there is an improvement of at least one class in a classification of heart failure from a baseline level, wherein the classification of heart failure is assessed by at least one of the following: (a) a classification system used by the American Heart Association (AHA), the American College of Cardiology (ACC), the 2016 European Society of Cardiology guidelines (ESCG), the Japanese heart failure Society (JHFS) guidelines, The Japanese Circulation Society (JCS) Guidelines, or, the New York Heart Association (NYHA); or equivalent thereof, or (b) a health-related quality of life (HRQL) questionnaire selected from the group consisting from any of: Minnesota LIVING WITH HEART FAILURE® Questionnaire (MLHFQ), or Kansas City Cardiomyopathy questionnaire (KCCQ), Chronic Heart Failure Questionnaire (CHFQ), Quality of Life Questionnaire for Severe Heart Failure (QLQ-SHF), Left Ventricular Dysfunction (LVD-36) questionnaire, and the Left Ventricular Disease Questionnaire (LVDQ).

In all aspects of the methods disclosed herein, an improvement of a classification of HF is an improvement of at least 2, or at least 3, or at least 4, or at least 5 parameters at least 12 months after administration. In some embodiments, there is an improvement of at least 2, or at least 3, or at least 4, or at least 5 parameters at least 6 months after administration. In some embodiments, there is an improvement in the classification of at least one level, or at least two levels, within six months after administration of the rAAV. In some embodiments, there is an improvement of at least a 10 point decrease in quality of life MLWHFQ or KCCQ from the baseline level.

In all aspects of the methods and compositions disclosed herein, the subject is administered a vasodilator concurrent with and/or, before, and/or, after the administration of the at least one total dose of a rAAV vector. In all aspects of the methods and compositions disclosed herein, the subject is administered an immune modulator concurrent with, or before, or after the administration of the at least one total dose of a rAAV vector.

Another aspect of the technology described herein relates to a pharmaceutical composition comprising an AAV vector that comprises a codon optimized I-Ic nucleic sequence selected from any of SEQ ID NO: 385-412, or nucleic acid sequence having at least 80% sequence identity to SEQ ID NOS: 385-412. In some embodiments, the codon optimized nucleic acid sequence is operably linked to a CMV promoter or a synthetic promoter, for example a cardiac-specific promoter selected from any of Table 2A, or a muscle-specific promoter active in cardiac and skeletal muscle, e.g., a promoter selected from Table 5A or 13A, or a variant thereof. In some embodiments, the pharmaceutical composition comprises a nucleic acid sequence selected from the group consisting of: SEQ ID NO: 41-42, or a nucleic acid sequence having at least 80% sequence identity to SEQ ID NOS: 385-412. In some embodiments, the pharmaceutical composition can be used in a method to treat a subject with cardiomyopathy, including non-ischemic cardiomyopathy or ischemic cardiomyopathy, as disclosed herein. In some embodiments, the pharmaceutical composition can be used in a method to treat a subject with heart failure as disclosed herein.

Another aspect of the technology described herein relates to an adeno-associated virus (AAV) vector comprising a nucleic acid sequence encoding a Phosphatase inhibitor (I-1) polypeptide operably linked to a promoter selected from any of: (a) a cardiac-specific promoter selected from Table 2A or a variant thereof, or (b) a muscle-specific promoter active in cardiac and skeletal muscle, or a variant thereof, or (c) any promoter when a cardiac tissue specific enhancer is present. In some embodiments of the AAV vector, the muscle-specific promoter active in cardiac and skeletal muscle is selected from Table 5A or Table 13A or a variant thereof.

In all aspects of the rAAV composition and methods of using to treat a subject with cardiomyopathy and/or heart failure as disclosed herein, the AAV is selected from the group consisting of adeno-associated virus-1 (AAV1), AAV2, AAV3, AAV4, AAV5, AAV6, AAV7, AAV8, AAV9 and AAV2i8. In some embodiments, the AAV comprises a capsid that detargets the liver - that is, the capsid targets the rAAV to a tissue other than the liver in vivo. In some embodiments, the rAAV vector is AAV2i8.

In all aspects of the rAAV compositions and methods as disclosed herein, the phosphatase inhibitor (I-1) polypeptide is a constitutively active protein (I-1c), for example, where the I-1c is selected from any of: (a) a polypeptide comprises at least amino acid residues 1-65 of SEQ ID NO: 1 or a functional equivalent thereof; (b) a polypeptide comprising at least amino acids 1-54 of SEQ ID NO: 1, wherein the polypeptide is truncated at a C-terminus at amino acid selected from residue 70, 67, 66, 65, or 61, or 54, and where the there is an aspartic acid at position 35 (T35D), (c) a polypeptide comprising amino acids 1-65 of SEQ ID NO:1 or a functional equivalent thereof having at least 85% sequence identity to amino acid residues 1-65 of SEQ ID NO: 1, or, (d) a polypeptide selected from any of: SEQ ID NOS: 507 or 527-532 or a functional equivalent thereof having at least 85% sequence identity to amino acid residues of any of SEQ ID NOS: 507 or 527-532. In some embodiments of all aspects disclosed herein, the AAV vector comprises a nucleic acid sequence encoding a I-1 polypeptide is selected from: (a) a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

In some embodiments of all aspects disclosed herein, the rAAV encodes a I-1c polypeptide selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D), or a conservative amino acid of aspartate.

In some embodiments of all aspects disclosed herein, the nucleic acid sequence encoding the I-1 polypeptide is a codon optimized nucleic acid sequence, for example, where the codon optimized nucleic acid sequence has reduced CpG content or reduced CpG islands as compared to the wild-type reference sequence of a SEQ ID NO: 1, or a fragment thereof. In some embodiments of all aspects disclosed herein, the nucleic acid sequence encoding the I-1 polypeptide is a codon optimized nucleic acid sequence selected from any of: SEQ ID NO: 385-412 or a nucleic acid sequence at least 80%, or at least 85%, or at least 90% or at least 95% or at least 98% sequence identity to SEQ ID NO: 385-412.

In some embodiments of all aspects disclosed herein, the rAAV vector can comprise at least one ITR located 5′ of the nucleic acid sequence encoding a Phosphatase inhibitor (I-1) polypeptide operably linked to the cardiac-specific promoter or muscle-specific promoter. In some embodiments of all aspects disclosed herein, the rAAV vector can comprise at least two ITRs flanking the nucleic acid sequence encoding a Phosphatase inhibitor (I-1) polypeptide operably linked to the cardiac-specific promoter or muscle-specific promoter. Any ITR sequence known to a person of ordinary skill in the art can be used, and includes, but is not limited to an ITR sequences selected from any one or more of: SEQ ID NO: 70-78, or a nucleic acid sequence having at least 85%, or at least 90% or at least 95% or at least 98% sequence identity to SEQ ID NOS: 70-78.

In some embodiments of all aspects of the compositions and methods as disclosed herein, the AAV vector comprises a reverse poly A sequence or double stranded RNA termination element, wherein the reverse polyA sequence or double stranded termination element are located 3′ of the nucleic acid sequence encoding a Phosphatase inhibitor (I-1) polypeptide. In some embodiments, the reverse poly A sequence, or double stranded RNA termination element is located between 3′ of the nucleic acid sequence encoding a Phosphatase inhibitor (I-1) polypeptide and 5′ of the right ITR. In some embodiments of all aspects of the compositions and methods as disclosed herein, the AAV vector further comprising a polyA sequence selected from any of SV40 polyA (SEQ ID NO: 334), HGH poly A (SEQ ID NO: 66), SEQ ID NO: 284-287, SEQ ID NO 331-335, or a nucleic acid sequence at least 85%, or at least 90% or at least 95% or at least 98% sequence identity to SEQ ID NOS: 334, 66, 284-287 or 331-335, wherein the polyA sequence is located 3′ of the nucleic acid sequence encoding a Phosphatase inhibitor (I-1) polypeptide.

In some embodiments of all aspects of the compositions and methods as disclosed herein, the AAV vector can further comprise a nucleic acid sequence encoding at least one immune modulator and/or a vasodilator, as disclosed herein. In some embodiments of all aspects of the compositions and methods as disclosed herein, the rAAV vector can be present in a composition or solution, where the solution further comprises an immune modulator. In some embodiments of all aspects of the compositions and methods as disclosed herein, the rAAV vector can be present in a composition or solution, where the solution further comprises a vasodilator.

Another aspect of the technology described herein relates to a pharmaceutical composition comprising: (i)adeno-associated virus (AAV) vector comprising a nucleic acid sequence encoding a Phosphatase inhibitor (I-1) polypeptide operably linked to any one of: (a) a cardiac-specific promoter selected from Table 2A or a variant thereof, (b) a muscle-specific promoter active in cardiac and skeletal muscle, or (c) any promoter when a cardiac tissue specific enhancer is present, or a variant thereof; and a pharmaceutically acceptable carrier. In some embodiments of all aspects of the compositions and methods as disclosed herein, the muscle-specific promoter active in cardiac and skeletal muscle is selected from Table 5A or Table 13A or a variant thereof, e.g., a variant comprising a nucleic acid sequence having at least at least 85%, or at least 90% or at least 95% or at least 98% sequence identity to a promoter listed in Table 5A or Table 13A.

In some embodiments of all aspects of the compositions and methods as disclosed herein, the AAV is selected from the group consisting of adeno-associated virus-1 (AAV1), AAV2, AAV3, AAV4, AAV5, AAV6, AAV7, AAV8, AAV9 and AAV2i8. In some embodiments of all aspects of the compositions and methods as disclosed herein, the AAV comprises a capsid that detargets the liver, as disclosed herein. In some embodiments of all aspects of the compositions and methods as disclosed herein, the AAV is AAV2i8. In some embodiments, the pharmaceutical composition comprises a AAV which comprises a nucleic acid selected from the group consisting of SEQ ID NO: 413-440, or a nucleic acid sequence at least 80%, or at least 85%, or at least 90% or at least 95% or at least 98% sequence identity sequence to a sequence selected from SEQ ID NO: 413-440, wherein the nucleic acids set forth in SEQ ID NO: 413-440 comprise a CMV promoter of SEQ ID NO: 330, wherein the CMV promoter of SEQ ID NO: 330 is replaced by any of: (a) a cardiac-specific promoter selected from Table 2A or a variant thereof (e.g., a variant comprising a nucleic acid sequence having at least at least 85%, or at least 90% or at least 95% or at least 98% sequence identity to a promoter listed in Table 2A), (b) a muscle-specific promoter active in cardiac and skeletal muscle, or (c) any promoter when a cardiac tissue specific enhancer is present, or a variant thereof, where the muscle-specific promoter active in cardiac and skeletal muscle is selected from Table 5A or Table 13A or a variant thereof, e.g., a variant comprising a nucleic acid sequence having at least at least 85%, or at least 90% or at least 95% or at least 98% sequence identity to a promoter listed in Table 5A or Table 13A.

In some embodiments, the pharmaceutical composition comprises a vasodilator. In some embodiments, the pharmaceutical composition comprises an immune modulator.

In some embodiments of all aspects disclosed herein, the pharmaceutical composition comprises AAV vector comprises a nucleic acid sequence encoding a I-1 polypeptide is selected from: (a) a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

In some embodiments of all aspects disclosed herein, the pharmaceutical composition comprises a rAAV which encodes a I-1c polypeptide selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D), or a conservative amino acid of aspartate.

In some embodiments of all aspects disclosed herein, the pharmaceutical composition comprises a nucleic acid sequence encoding the I-1 polypeptide which is a codon optimized nucleic acid sequence, for example, where the codon optimized nucleic acid sequence has reduced CpG content or reduced CpG islands as compared to the wild-type reference sequence of a SEQ ID NO: 1, or a fragment thereof. In some embodiments of all aspects disclosed herein, the nucleic acid sequence encoding the I-1 polypeptide is a codon optimized nucleic acid sequence selected from any of: SEQ ID NO: 385-412 or a nucleic acid sequence at least 80%, or at least 85%, or at least 90% or at least 95% or at least 98% sequence identity to SEQ ID NO: 385-412.

Another aspect of the technology described herein relates to the use of an AAV vector as disclosed herein, for the manufacturer of a pharmaceutical composition for the treatment of a subject having cardiomyopathy. In some embodiments of all aspects of the compositions and methods as disclosed herein, the subject with cardiomyopathy to be treated has non-ischemic heart failure and/or non-ischemic cardiomyopathy. In some embodiments of all aspects of the compositions and methods as disclosed herein, the subject with cardiomyopathy has a congenital cardiomyopathy or a genetic disorder with a cardiac manifestation, for example, a genetic disorder with a cardiac manifestation as disclosed herein, which includes, but is not limited to a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

In some embodiments of all aspects of the compositions and methods as disclosed herein, the rAAV as disclosed herein can be used to treat a subject with cardiomyopathy who has an ischemic cardiomyopathy. In some embodiments of all aspects of the compositions and methods as disclosed herein, the rAAV as disclosed herein can be used to treat a subject with cardiomyopathy who has heart failure, for example, where the subject with heart failure has a classification of heart failure based upon a classification system used by the American Heart Association (AHA), the American College of Cardiology (ACC) or the New York Heart Association (NYHA), or an equivalent classification thereof. In some embodiments of all aspects of the compositions and methods as disclosed herein, the rAAV as disclosed herein can be used to treat a subject with heart failure, e.g., where the subject with heart failure has a classification of a class III or above class III in the New York Heart Association (NYHA) classification system.

Another aspect of the technology described herein relates to the use of AAV vector as disclosed herein, for the manufacturer of a pharmaceutical composition for the treatment of a subject having a condition or disease associated with heart failure. In some embodiments, the subject has a classification of congestive heart failure (CHF) or heart failure (HF), for example, but not limited to a classification is based upon a classification system used by the American Heart Association (AHA), the American College of Cardiology (ACC) or the New York Heart Association (NYHA), or an equivalent classification system as disclosed herein. In some embodiments, the subject has non-ischemic heart failure or non-ischemic cardiomyopathy. In some embodiments, the subject has ischemic heart failure or ischemic cardiomyopathy. In some embodiments of all aspects of the compositions and methods as disclosed herein, the subject being treated according to the methods and compositions as disclosed herein has a reduced ejection fraction (rEF or HFrEF).

Another aspect of the technology described herein relates to a cell comprising a AAV vector as disclosed herein. In some embodiments, the cell is a cardiac cell or a muscle cell, and in some embodiments, the cell is in cell culture (i.e., in vitro), and in some embodiments, the cell is present in a subject (e.g., in vivo).

Another aspect of the technology described herein relates to the use of a AAV vector as disclosed herein, or a pharmaceutical formulation as disclosed herein, or a cell as disclosed herein, for use in the treatment of a subject having cardiomyopathy.

In some embodiments of all aspects of the compositions and methods as disclosed herein, the delivery of the rAAV vector disclosed herein (e.g., expressing an agent that modulates phosphatase activity, e.g., type 1 phosphatase activity) is cells which are non-mitotic, (e.g., cardiomyocytes). Accordingly, transgene expression can persist for the life of the cell, after at least 1 dose.

Another aspect of the technology described herein relates to the use of a AAV vector as disclosed herein, or a pharmaceutical formulation as disclosed herein, or a cell as disclosed herein, for use in the treatment of a patient having heart failure. In some embodiments of all aspects of the compositions and methods as disclosed herein, the AAV vector for use according to the methods disclosed herein is administered to a subject who has a classification of congestive heart failure (CHF), e.g., where the classification is based upon a classification system used by the American Heart Association (AHA), the American College of Cardiology (ACC) or the New York Heart Association (NYHA), or an equivalent classification system thereof.

In some embodiments of all aspects of the compositions and methods as disclosed herein, the subject has non-ischemic heart failure or non-ischemic cardiomyopathy. In some embodiments of all aspects of the compositions and methods as disclosed herein, the subject has ischemic heart failure or ischemic cardiomyopathy. In some embodiments of all aspects of the compositions and methods as disclosed herein, the subject has reduced ejection fraction (rEF or HFrEF). In some embodiments of all aspects of the compositions and methods as disclosed herein, the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension. In some embodiments of all aspects of the compositions and methods as disclosed herein, the subject with heart failure has any one of: ischemia, arrhythmia, myocardial infarction, abnormal heart contractibility, or abnormal Ca2+ metabolism.

In some embodiments of all aspects of the compositions and methods as disclosed herein, the subject has one or more of: (a) non-ischemic heart failure; (b) non-ischemic cardiomyopathy, (c) a classification of congestive heart failure (CHF) is based upon a classification system used by the American Heart Association (AH), the American College of Cardiology (ACC) or the New York Heart Association (NYHA) or an classification using an equivalent classification system; or (d) a reduced ejection fraction (rEF or HFrEF).

Another aspects of the technology disclosed herein relates to a method of expressing a phosphatase inhibitor (I-1) polypeptide in a subject with cardiomyopathy, the method comprising introducing into the subject with cardiomyopathy, at least one dose of the AAV vector according to the methods as disclosed herein, where the subject with cardiomyopathy has a classification of heart failure, and where the at least one dose of the rAAV is selected from a total dose-range of about 10¹³vg to about 10¹⁵vg, and where at least twelve months post-administration there is an improvement in the classification of heart failure. In some embodiments, the classification of heart failure is based upon a classification system used by the American Heart Association (AH), the American College of Cardiology (ACC) or the New York Heart Association (NYHA), or an equivalent classification system. In some embodiments of all aspects of the methods as disclosed herein, there is an improvement of classification of at least one level, or at least two levels, at least 12 months after administration of the rAAV, or in some embodiments, an improvement of classification of at least one level, or at least two levels, at least 6 months after administration of the rAAV. In some embodiments, at least twelve months post-administration there is an improvement of at least 1, or 2 levels in the classifications in any one or more of: the American Heart Association (AH), the American College of Cardiology (ACC), or the New York Heart Association (NYHA) classification systems, or equivalent HF classification systems.

In some embodiments of all aspects of the methods as disclosed herein, the rAVV is administered with an immune modulator concurrent with, or before, or after the administration of the at least one dose of a rAAV vector. In some embodiments of all aspects of the methods as disclosed herein, the rAVV is administered with a vasodilator concurrent with, and/or before, and/or after the administration of the at least one dose of a rAAV vector.

In some embodiments of all aspects of the methods as disclosed herein, administration of the rAAV is into the lumen of the coronary artery of the heart of the patient. In some embodiments where administration of the rAAV is for treatment of a subject with ischemic cardiomyopathy, administration is directly into the muscle of the heart, e.g., into the ischemic cardiac muscle or MI.

In some embodiments of all aspects of the methods as disclosed herein, administration of at least one dose of the rAAV is a total dose-range of about 10¹³vg to about 10¹⁵vg., and can be administered in one dose or 2 to 5 sub-doses. In some embodiments of all aspects of the methods as disclosed herein, the total dose is administered as any of the following administration methods: (a) over a period of time of about 20 minutes to about 30 minutes, (b) administered in a series of sub-doses, wherein each sub-dose is administered over a period of time of about 1 minute to about5 minutes, and (c) administered in a series of five sub-doses, each sub-dose is administered over a period of time of about 1 minute to about 5 minutes, and wherein the five sub-doses are administered over a period of about 20 minutes to about 30 minutes.

Another aspect of the technology described herein relates to a method of treating a patient having a heart failure, comprising: administering into heart cells of the patient, at least one total dose of a rAAV vector comprising a nucleic acid sequence encoding a phosphatase inhibitor protein that inhibits phosphatase activity, wherein, at least one total dose of the rAAV is selected from a dose-range of about 10¹³vg to about 10¹⁵vg, wherein, the total dose is administered over a period of time of about 20 minutes to about 30 minutes, wherein, the administration of the total dose is performed in sub-doses, wherein each sub-dose is administered over a period of time of 1-5 minutes. In some embodiments, the heart failure is selected from any one or more of: ischemia, arrhythmia, myocardial infarction, abnormal heart contractibility, or abnormal Ca2+ metabolism.

In some embodiments, the rAAV vector further comprises CMV promoter or a synthetic promoter, operatively linked to the phosphatase inhibitor protein. In some embodiments, the synthetic promoter is a cardiac-specific promoter selected from Table 2A or a variant thereof, or a muscle-specific promoter active in cardiac and skeletal muscle selected from Table 5A or a variant thereof. In some embodiments, the synthetic promoter causes expression of the therapeutic nucleic acid or phosphatase inhibitor protein preferentially in smooth muscle cells. In some embodiments, the synthetic promoter causes expression of the therapeutic nucleic acid or phosphatase inhibitor protein preferentially in cardiac cells. In some embodiments, the expression of the therapeutic nucleic acid or phosphatase inhibitor protein by the cardiac- or muscle-specific promoter is equivalent to, or greater than, the expression caused by CMV promoter.

In some embodiments, in the method for a method of treating a patient having a heart failure, the rAAV is selected from the group consisting of AAV2, AAV6, AAV8, AAV9, AAV2i8, rh10, AAV2.5 and AAV2G9. In some embodiments, the rAAV is AAV2i8 or AAV9. In some embodiments, the rAAV is selected from any AAV serotypes disclosed in Table 11.

In some embodiments, in the method for a method of treating a patient having a heart failure, the administration of the total dose is performed in five sub-doses, each sub-dose is administered over a period of time of 1-5 minutes. In some embodiments, the five sub-doses are administered over a period of about 20 minutes to about 30 minutes.

In some embodiments, in the method for a method of treating a patient having a heart failure, the at least one total dose of the rAAV is 10¹³ vg, 3×10¹³ vg, 10¹⁴ vg, 3×10¹⁴ vg, or, 10¹⁵ vg. In some embodiments, at least one sub-dose of the rAAV is 10¹³ vg, 3×10¹³ vg, 10¹⁴ vg, 3×10¹⁴vg, or, 10¹⁵ vg. In some embodiments, at least one dose is a total dose-range of about 10¹³vg to about 10¹⁵vg., administered in 2 to 5 sub-doses. In some embodiments, in the method for a method of treating a patient having a heart failure, the administration is into the lumen of the coronary artery of the heart of the patient.

In all aspect of the methods and compositions as disclosed herein, in some embodiments, the phosphatase inhibitor I-1 comprises amino acids 1-65 of SEQ ID NO: 1 or a functional fragment thereof, wherein the threonine at position 35 of SEQ ID NO: 1 is replaced with an aspartate acid (T35D). In all aspect of the methods and compositions as disclosed herein, in some embodiments, the nucleic acid encoding phosphatase inhibitor encodes a constitutively active fragment of I-1 (I-1c) comprising a fragment of SEQ ID NO: 1, wherein the fragment is selected from: amino acids 1-54 of SEQ ID NO: 1, 1-61 of SEQ ID NO: 1, 1-65 of SEQ ID NO: 1, 1-66 of SEQ ID NO: 1, 1-67 of SEQ ID NO: 1 or 1-77 of SEQ ID NO: 1, or a functional variant thereof, wherein the threonine at position 35 of SEQ ID NO: 1 is replaced with an aspartate acid (T35D).

Another aspect of the technology described herein relates to an adeno-associated virus (AAV) vector comprising a nucleic acid sequence encoding a polypeptide comprising at least amino acids 1-54 of SEQ ID NO: 1, wherein threonine at amino acid 35 of SEQ ID NO: 1 is replaced with an aspartic acid, and wherein said nucleic acid sequence is operably linked to a promoter selected from any of: a CMV promoter, a cardiac-specific promoter selected from Table 2A or a variant thereof, or a muscle-specific promoter active in cardiac and skeletal muscle selected from Table 5A or a variant thereof. In some embodiments, the polypeptide is selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D). In some embodiments, the rAAV is selected from the group consisting of AAV2, AAV6, AAV8, AAV9, AAV2i8, rh10, AAV2.5 and AAV2G9. In some embodiments, the rAAV is AAV2i8 or AAV9. In some embodiments, the rAAV is selected from any AAV serotypes disclosed in Table 11.

Another aspect of the technology described herein relates to a pharmaceutical composition comprising (i) adeno-associated virus (AAV) vector comprising a nucleic acid sequence encoding a polypeptide comprising at least amino acids 1-54 of SEQ ID NO: 1, wherein threonine at amino acid 35 of SEQ ID NO: 1 is replaced with an aspartic acid (T35D), and wherein said nucleic acid sequence is operably linked to a cardiac-specific promoter selected from Table 2A or a variant thereof, or a muscle-specific promoter active in cardiac and skeletal muscle selected from Table 5A or a variant thereof, and (ii) a pharmaceutically acceptable carrier.

In some embodiments, the rAAV vector of the pharmaceutical composition comprises a nucleic acid sequence encoding a polypeptide selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D). In some embodiments, the rAAV in the pharmaceutical composition is selected from the group consisting of AAV2, AAV6, AAV8, AAV9, AAV2i8, rh10, AAV2.5 and AAV2G9. In some embodiments, the rAAV is AAV2i8 or AAV9. In some embodiments, the rAAV is selected from any AAV serotypes disclosed in Table 11.

In all aspect of the methods and compositions as disclosed herein, the method can be performed more than once. For example, the patient can be administered the rAAV vector at a first time point, and for example, after about 3 months, or after about 6 months, or after about 12-months, or after about 2 years, or after about 3 years, the patient can be administered the rAAV vector according to the methods disclosed herein a second time. In some embodiments, the subject or patient can be administered the rAAV vector according to the methods as disclosed herein multiple times, e.g., at least 2, or at least 3, or at least 4, or at least 5 or at least 6 or more than 6 times according to the methods disclosed herein.

All other aspects of the technology are described below.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings, in which:

FIG. 1 shows in vivo expression of the luciferase gene in heart tissue (cardiac muscle) from the muscle specific promoter SP0067, as compared to other control promoters (CBA and CK8b intron), or a saline control.

FIG. 2 shows in vivo expression of luciferase gene in Tibialis Anterion (TA) muscle from the muscle specific promoter SP0067, as compared to other control promoters (CBA and CK8b intron), or a saline control.

FIG. 3 shows the in vivo expression of luciferase gene from synthetic cardiac-specific promoter SP0067 in diaphragm (diaph), quadriceps (Quad), tibialis anterior (TA), heart, intestine and liver. SP0067 is active in vivo in heart muscle but not active in skeletal muscles (diaphragm (diaph), quadriceps (Quad), tibialis anterior (TA)). SP0067 also has some in vivo activity in liver.

FIGS. 4A-4B shows the average in vitro expression of synthetic cardiac-specific promoters in human cardiac and skeletal muscle cells (H9C2 or H2K cells). FIG. 4A shows expression of a marker gene in a pAAV-SYNP vector operatively linked to SP0067, SP0424 or SP0425 synthetic promoters in H2K mouse skeletal muscle cells, where H2K cells have been differentiated into skeletal muscle myotubes, where the data is normalised to the activity of the known promoter CBA. A relative activity of 1 is equal to the activity of CBA. The error bar is standard deviation. FIG. 4B shows the average expression of a marker gene in a pAAV-SYNP vector operatively linked to SP0067, SP0424, SP0425, SP0429, SP0430, SP0344, SP0433, SP0435, SP0436 synthetic promoters in H9C2 rat cardiomyocyte cells, where H9C2 cells have been differentiated into cardiac muscle (heart) myotubes, and normalised to the activity of the known promoter CBA. A relative activity of 1 is equal to the activity of CBA. The error bar is standard deviation. The error is standard deviation of at least three replicate experiments.

FIGS. 5A-5F shows in vivo activity of synthetic muscle specific promoters that are active in skeletal and cardiac muscle. FIG. 5A shows the in vivo activity of synthetic muscle specific promoters, the control promoters CBA and CK8 as well as saline negative control in the heart. FIG. 5B shows the in vivo activity of synthetic muscle specific promoters, the control promoters CBA CK8 as well as saline negative control in the diaphragm. FIG. 5C shows the in vivo activity of synthetic muscle specific promoters, the control promoters CBA and CK8 as well as saline negative control in the quadriceps. FIG. 5D shows the in vivo activity of synthetic muscle specific promoters, the control promoters CBA and CK8 as well as saline negative control in the intestine. FIG. 5E shows the in vivo activity of synthetic muscle specific promoters, the control promoters CBA and CK8 as well as saline negative control in the tibialis anterior. FIG. 5F shows the in vivo activity of synthetic muscle specific promoters, the control promoters CBA and CK8 as well as saline negative control in the liver.

FIGS. 6A-6K shows in vivo activity of exemplary synthetic muscle specific promoters SP0173, SP0270, SP0268, SP0320, SP0279, SP0134, SP0057, SP0229, SP0067, SP0310 and SP0267 that are active in cardiac and skeletal muscle. FIG. 6A shows the in vivo activity of synthetic muscle specific promoter SP0173 in the diaphragm, heart, intestine, liver, quadriceps (quad) and tibialis anterior (TA). FIG. 6B shows the in vivo activity of synthetic muscle specific promoter SP0270 in the diaphragm, heart, intestine, liver, quadriceps (quad) and tibialis anterior (TA). FIG. 6C shows the in vivo activity of synthetic muscle specific promoter SP0268 in the diaphragm, heart, intestine, liver, quadriceps (quad) and tibialis anterior (TA). FIG. 6D shows the in vivo activity of synthetic muscle specific promoter SP0320 in the diaphragm, heart, intestine, liver, quadriceps (quad) and tibialis anterior (TA). FIG. 6E shows the in vivo activity of synthetic muscle specific promoter SP0279 in the diaphragm, heart, intestine, liver, quadriceps (quad) and tibialis anterior (TA). FIG. 6F shows the in vivo activity of synthetic muscle specific promoter SP0134 in the diaphragm, heart, intestine, liver, quadriceps (quad) and tibialis anterior (TA). FIG. 6G shows the in vivo activity of synthetic muscle specific promoter SP0057 in the diaphragm, heart, intestine, liver, quadriceps (quad) and tibialis anterior (TA). FIG. 6H shows the in vivo activity of synthetic muscle specific promoter SP0229 in the diaphragm, heart, intestine, liver, quadriceps (quad) and tibialis anterior (TA). FIG. 6I shows the in vivo activity of synthetic muscle specific promoter SP0067 in the diaphragm, heart, intestine, liver, quadriceps (quad) and tibialis anterior (TA). FIG. 6J shows the in vivo activity of synthetic muscle specific promoter SP0310 in the diaphragm, heart, intestine, liver, quadriceps (quad) and tibialis anterior (TA). FIG. 6K shows the in vivo activity of synthetic muscle specific promoter SP0267 in the diaphragm, heart, intestine, liver, quadriceps (quad) and tibialis anterior (TA).

FIGS. 7A-7P shows in vivo activity of exemplary synthetic cardiac muscle specific promoters SP0067, SP0451, SP0452, SP0430, SP0450, SP0429, SP0424, SP0435, SP0436, SP0433, SP0449, SP0344, SP0475 that are active in cardiac muscle, in selected tissues (liver, heart, tibialis anterior (TA), quadriceps (Quad), soleus, and diaphragm (Diaph), as compared to the muscle specific promoters CK8, or cardiac specific promoters control 1 or control 2. FIG. 7A shows the in vivo activity of the exemplary comparison liver promoter CK8 in the liver, heart, tibialis anterior (TA), quadriceps (Quad), soleus, and diaphragm (Diaph), showing liver expression at 1×10⁵ RLU/mg and heart expression at 1×10⁶⁻⁷ RLU/mg. FIG. 7B shows the in vivo activity of the cardiac muscle promoter SP0067 in the liver, heart, tibialis anterior (TA), quadriceps (Quad), soleus, and diaphragm (Diaph), showing specific expression in the heart at 1×10⁵ RLU/mg and lower expression in skeletal or smooth muscle tissues, and also lower expression in liver at 1×10⁴ RLU/mg. FIG. 7C shows the in vivo activity of the cardiac muscle promoter SP0344 in the liver, heart, tibialis anterior (TA), quadriceps (Quad), soleus, and diaphragm (Diaph), showing specific expression in the heart at about 1×10⁷ RLU/mg and lower expression in skeletal or smooth muscle tissues. FIG. 7D shows the in vivo activity of the cardiac muscle promoter SP0424 in the liver, heart, tibialis anterior (TA), quadriceps (Quad), soleus, and diaphragm (Diaph), showing very specific expression in the heart at 1×10⁷ to 1×10⁸ RLU/mg and lower expression at about 1×10⁴ RLU/mg in the liver and skeletal or smooth muscle tissues. FIG. 7E shows the in vivo activity of the cardiac muscle promoter SP0429 in the liver, heart, tibialis anterior (TA), quadriceps (Quad), soleus, and diaphragm (Diaph), showing very specific expression in the heart at 1×10⁸ RLU/mg and lower expression at about 1×10⁴ to about 1×10⁵ RLU/mg in the liver and skeletal or smooth muscle tissues. FIG. 7F shows the in vivo activity of the cardiac muscle promoter SP0430 in the liver, heart, tibialis anterior (TA), quadriceps (Quad), soleus, and diaphragm (Diaph), showing very specific expression in the heart at 1×10⁸ RLU/mg and lower expression at about 1×10⁴ to about 1×10⁵ RLU/mg in the liver and skeletal or smooth muscle tissues. FIG. 7G shows the in vivo activity of the cardiac muscle promoter SP0433 in the liver, heart, tibialis anterior (TA), quadriceps (Quad), soleus, and diaphragm (Diaph), showing specific expression in the heart at 1×10⁷ RLU/mg and lower expression in skeletal or smooth muscle tissues and in the liver. FIG. 7H shows the in vivo activity of the positive control cardiac muscle promoter control 1 in the liver, heart, tibialis anterior (TA), quadriceps (Quad), soleus, and diaphragm (Diaph), showing expression in the heart at 1×10⁷ RLU/mg and lower expression in skeletal or smooth muscle tissues, and in the liver tissue. FIG. 7I shows the in vivo activity of the cardiac muscle promoter SP0435 in the liver, heart, tibialis anterior (TA), quadriceps (Quad), soleus, and diaphragm (Diaph), showing very specific expression in the heart at between 1×10⁷ and 1×10⁸ RLU/mg and lower expression at about 1×10⁴ RLU/mg in the liver and skeletal or smooth muscle tissues. FIG. 7J shows the in vivo activity of the cardiac muscle promoter SP0436 in the liver, heart, tibialis anterior (TA), quadriceps (Quad), soleus, and diaphragm (Diaph), showing expression in the heart at 1×10⁷ RLU/mg and expression levels below about 1×10⁴ and 1×10⁶ RLU/mg in the liver and skeletal or smooth muscle tissues. FIG. 7K shows the in vivo activity of the positive control cardiac muscle promoter control 2 in the liver, heart, tibialis anterior (TA), quadriceps (Quad), soleus, and diaphragm (Diaph), showing very specific expression in the heart at 1.5×10⁶ RLU/mg and expression levels below about 1×x10⁴ in skeletal or smooth muscle tissues. FIG. 7L shows the in vivo activity of the cardiac muscle promoter SP0449 in the liver, heart, tibialis anterior (TA), quadriceps (Quad), soleus, and diaphragm (Diaph), showing expression in the heart at 1×10⁷ RLU/mg and expression levels below about 1.5×10⁵ RLU/mg in the liver and skeletal or smooth muscle tissues. FIG. 7M shows the in vivo activity of the cardiac muscle promoter SP0450 in the liver, heart, tibialis anterior (TA), quadriceps (Quad), soleus, and diaphragm (Diaph), showing very specific expression in the heart at 1.0×10⁸ RLU/mg and expression levels below about 1×10⁵ in the liver and skeletal or smooth muscle tissues. FIG. 7N shows the in vivo activity of the cardiac muscle promoter SP0451 in the liver, heart, tibialis anterior (TA), quadriceps (Quad), soleus, and diaphragm (Diaph), showing specific expression in the heart at above 1.0×10⁸ RLU/mg and expression levels below about 1.5×10⁵ in skeletal or smooth muscle tissues, and expression in liver is even below at about 1×10⁴ RLU/mg. FIG. 7O shows the in vivo activity of the cardiac muscle promoter SP0452 in the liver, heart, tibialis anterior (TA), quadriceps (Quad), soleus, and diaphragm (Diaph), showing specific expression in the heart at above 1.0×10⁸ RLU/mg and expression levels below about 1.5×10⁵ in skeletal or smooth muscle tissues, and expression in liver at about 1.5×10⁴ RLU/mg. FIG. 7P shows the in vivo activity of the cardiac muscle promoter SP0475 in the liver, heart, tibialis anterior (TA), quadriceps (Quad), soleus, and diaphragm (Diaph), showing expression in the heart at above 1×10⁵ RLU/mg and expression levels below about 1.5×10⁴ RLU/mg in the liver and skeletal or smooth muscle tissues.

FIG. 8 shows the in vitro activity of synthetic muscle specific promoter SP0521 and SP4169 in the muscle cell line H9C2 as compared to CBA and CK8 control promoters. This figure shows the average activity, normalized to the CBA promoter, of synthetic short muscle-specific promoters SP0521 and SP4769 in H9C2 cell line differentiated into heart myotubes. The error bar is standard deviation from triplicate experiments.

### DETAILED DESCRIPTION

Disclosed herein are methods for administration AAV vectors, as well as methods and compositions comprising AAV vectors for the improved treatment of cardiovascular diseases and disorders, heart disorders and diseases, including cardiomyopathy, heart failure and congestive heart failure (CHF). In some embodiments, the heart failure is non-ischemic heart failure or the subject has non-ischemic cardiomyopathy. In some embodiments, the heart failure is ischemic heart failure or the subject has ischemic cardiomyopathy. Non-ischemic heart failure includes genetic based and nutritionally caused failures.

In particular, aspects of the present invention are directed to novel methods of administration and novel rAAV compositions for the treatment of subjects with heart failure, including methods of administration comprising administering to a subject with a classification of heart failure a dose of a rAAV where upon at least 12-months after the administration the classification of the heart failure is improved by at least one, or at least two stages or classification levels. In some embodiments, the methods of administration disclosed herein can be used in combination with other agents, including but not limited to use of immunomodulators and/or vasodilators, as well as rAAV vectors comprising a codon optimized nucleic acid sequence to encode I-1c, and/or rAAV vectors comprising novel cardiac-specific muscle promoters. Moreover, the inventors have demonstrated different ways to treat subjects with heart failure, including subjects with non-ischemic cardiomyopathy and ischemic cardiomyopathy that have the ability to significantly improve the subjects’ categorization in a classification system used to assess heart failure. A range of classification systems to categorize the extent of a subjects’ of heart failure can be used and are well known in the art, and includes but are not limited to American Heart Association (AHA), the American College of Cardiology (ACC), Minnesota LIVING WITH HEART FAILURE® Questionnaire (MLHFQ or, MLWHF), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA), or equivalent or modified assessments or combinations or merged assessments thereof.

For exemplary purposes only and without wishing to be limited to theory, the methods of treating a subject with heart failure as disclosed herein, or administration methods as disclosed herein have been demonstrated to improve a subject’s classification of heart failure, within at 12-months post-administration of a rAAV disclosed herein, from, e.g., a category IV to a category III or less than category III, or for e.g., from a category III to a category II or less than category II, according to a heart failure classification system as disclosed herein. In some embodiments, the NYHA or the AHA or the ACC classifications are used, or any other comparative heart failure classification system known to a person of ordinary skill in the art.

In particular, the one aspect of the technology described herein generally relates to a method to administer a recombinant AAV (rAAV) vector, where the method is a single administration to the subject, where the single administration comprises at least 2, or 3, or 4, or 5 or more doses within the single administration. That is, stated differently, in some embodiments, the method comprises administering a rAAV vector to the subject in a single administration constituting multiple doses, e.g., using a bolus to administer a discrete amount over a specific time period in small sub-doses within that time period. The single administration can also comprise the administration of rAAV from least 2, or 3, or 4, or 5 or more vials or, syringes, in which the delivery of the rAAV from each vial or, each syringe between 1-5 minutes, or more than 5 minutes. Without limiting to any theory, an exemplary syringe can be a simple reciprocating pump consisting of a plunger (though in modem syringes, it is actually a piston) that fits tightly within a cylindrical tube called a barrel. The plunger is linearly pulled and pushed along the inside of the tube, allowing the syringe to take in and expel liquid or gas through a discharge orifice at the front (open) end of the tube. The open end of the syringe is fitted with a hypodermic needle, a nozzle or, tubing to direct the flow into and out of the barrel. In some embodiments, the methods relate to administration of a rAAV vector to the heart. In some embodiments, the rAAV vector comprises a cardiac-specific promoter, for example, an exemplary cardiac-specific promoter disclosed in Tables 1-3 herein. In some embodiments, the rAAV vector is administered according to the disclosed methods for the treatment of cardiovascular conditions, heart failure or a heart disease or disorder. In some embodiments, the rAAV vector administered according to the methods disclosed herein is a rAAV vector comprises a nucleic acid encoding a therapeutic agent for treatment of heart failure, where the nucleic acid is operatively linked to a cardiac-specific promoter as disclosed in Tables 1-3. In some embodiments, the promoter can be a regulatable promoter, e.g., an inducible promoter or a repressible promoter, or a promoter with zinc-fingers, TALONS, etc., as known in the art.

In some embodiments, the technology described herein relates to a method where administration of a single doses of a viral vector in sub-doses are intended as disclosed herein, and the viral vector is co-administered with an immune modulator, as disclosed herein.

Another aspect of the disclosure described herein relates to recombinant AAV (rAAV) vectors and constructs for rAAV genomes for gene therapy for delivering an inhibitor of protein phosphatase 1 (PP1) to a subject. In particular, the technology described herein relates in general to a rAAV vector, or a rAAV genome for producing an inhibitor of PP1, e.g., a I-1 polypeptide, or functional fragment or variant thereof, that is expressed in the heart, for example, human cardiac and skeletal muscle cells. For example, the technology relates to a rAAV vector for expressing a transgene in the heart, e.g., cardiac and smooth muscle cells.

In particular, in some embodiments, the inhibitor of PP1 is expressed under the control of cardiac-specific promoters (CSP) in a recombinant rAAV vector.

One aspect of the technology described herein relates to a rAAV vector that comprises a nucleotide sequence containing inverted terminal repeats (ITRs), a promoter, a heterologous gene, a polyA tail and potentially other regulator elements for use to treat a cardiovascular condition, heart disorder or heart disease, such as heart failure, and further, for the treatment of heart failure, wherein the heterologous gene is an inhibitor of PP1 and wherein the rAAV PP1 inhibitor can be administered to a patient in a therapeutically effective dose that is delivered to the appropriate tissue and/or organ for expression of the heterologous gene and treatment of the disease.

One aspect of the technology described herein relates to a rAAV vector that comprises in its genome the following in a 5′ to 3′ direction: 5′- and 3′-AAV inverted terminal repeats (ITR) sequences, and located between the 5′ and 3′ ITRs, a heterologous nucleic acid sequence encoding an inhibitor of protein phosphatase 1 (PP1), wherein the heterologous nucleic acid is operatively linked to a cardiac-specific promoter (CSP), for example, a cardiac specific promoter disclosed in Table 2A herein, or a functional variant thereof.

In some embodiments, the a rAAV vector described herein is from any serotype. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations). In some embodiments, the virion can be rational haploid, or a chimeric or any mutant, such as capsids can be tailored for increased update at a desired location, e.g., the heart. Other capsids can include capsids from any of the known AAV serotypes, including AAV1, AAV2, AAV3, AAV4, AAV5, AAV6, AAV7, AAV8, AAV9, AAV10, etc. In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

### I. Methods of Treatment of Subjects With a Cardiovascular Condition, Heart Disease and Heart Failure, and CHF

### A. Method of Treatment of Heart Disorders and Heart Disease

### Subjects Amenable

Heart failure (HF) is a complex syndrome caused by the heart not functioning properly. Different types of heart failure are classified by specific characteristics, including symptoms and limitations of function. Heart failure can have identifiable or unknown causes. The diagnosis of heart failure, according to established guidelines, is based on criteria which include the presence of symptoms and signs, evidence of reduced cardiac function on diagnostic tests, and/or a favourable response to treatment.

There are multiple classifications of heart failure, but the two main ones are the New York Heart Association (NYHA) and the American College of Cardiology/American Heart Association (ACC/AHA) which have complementary classification systems. The severity of the heart failure is dependent on the subjects’ symptoms and level of heart function.

One aspect of the technology disclosed herein is a method of treating a subject with heart failure with a rAAV, or where upon after 12-months or earlier after administration there is an improvement in at least one level of the subject’s classification of heart failure. For exemplary purposes, a subject can be assessed by a physician as going from, e.g., a category IV to a category III or less than category III, or for e.g., from a category III to a category II or less than category II, in one or more heart failure classification system, as disclosed herein. In some embodiments, the NYHA or the AHA or the ACC classifications are used, or any other comparative heart failure classification system known to a person of ordinary skill in the art.

### B. Heart Failure Classification Systems

Heart failure classification systems are well known in the art, and includes but are not limited to American Heart Association (AHA), the American College of Cardiology (ACC), Minnesota LIVING WITH HEART FAILURE® Questionnaire (MLHFQ), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA), or modified assessments or combinations or merged assessments thereof.

### (I) New York Heart Association (NYHA):

One heart failure classification system useful in the methods disclosed herein is the NYHA classification system. The NYHA (New York Heart Association) classifies HF into classes I, II, III, and IV based on symptom severity. Yancy CW, et al., Circulation 2013;128:e240-e327; Adapted from Dolgin M, Association NYH, Fox AC, Gorlin R, Levin RI, New York Heart Association. Criteria Committee. Nomenclature and criteria for diagnosis of diseases of the heart and great vessels. 9th ed. Boston, MA: Lippincott Williams and Wilkins; Mar. 1, 1994; Original source: Criteria Committee, New York Heart Association, Inc. Diseases of the Heart and Blood Vessels. Nomenclature and Criteria for diagnosis, 6th edition Boston, Little, Brown and Co. 1964, p 114.

Physicians can classify a subject’s heart failure according to the severity of their self-reported symptoms. The classification system used most often is the New York Heart Association (NYHA) Functional Classification. Four levels of clinical classification are used to classify people according to symptoms and limitations experienced during physical activity. Symptom severity is compared to normal breathing, shortness of breath, and/or angina (chest pain or discomfort). Classification of heart failure based on function during physical activity, often called exertion, is often an important indicator of prognosis. There are 4 classes or stages in the NYHA which are as follows:

Class I (Mild): No limitation of physical activity. ordinary physical activity does not cause undue fatigue, palpitation, dyspnea (shortness of breath).

Class II (Mild): Slight limitation of physical activity. Comfortable at rest. Ordinary physical activity results in fatigue, palpitation, dyspnea.

Class III (Moderate): Marked limitation of physical activity. Comfortable at rest. Less than ordinary activity causes fatigue, palpitation, or dyspnea.

Class IV (Severe): Symptoms of heart failure occur even at rest. Unable to carry on any physical activity without discomfort. If any physical activity is undertaken, discomfort increases.

Class I and II are typically considered mild heart failure, while class III and IV are considered more severe or advanced heart failure. A person can move back and forth between these classes as they are based on symptoms. When a patient has a heart failure exacerbation, they will have more symptoms and likely be a higher class, but when their symptoms are better controlled, they will be a lower class.

Table 1A: New York Heart Association (NYHA) Classification classes/stages:

| | |
|---|---|
| NYHA Class | Symptoms |
| I | No limitation of physical activity. Ordinary physical activity does not cause undue fatigue, palpitation, dyspnea (shortness of breath). |
| II | Slight limitation of physical activity. Comfortable at rest. Ordinary physical activity results in fatigue, palpitation, dyspnea. |
| III | Marked limitation of physical activity. Comfortable at rest. Less than ordinary activity causes fatigue, palpitation, or dyspnea. |
| IV | Unable to carry on any physical activity without discomfort. Symptoms of heart failure at rest. If any physical activity is undertaken, discomfort increases. |

In certain embodiments, NYHA class III patient receiving rAAV administration as disclosed herein is improved to NYHA class II patient after 1 month, or, 2 months, or, 3 months, or, 4 months, or 5 months, or, 6 months, or, 7 months, or, 8 months, or, 9 months or, 12 months or, more months of post administration of rAAV dose. In other embodiments, NYHA class II patient receiving rAAV administration as disclosed herein is improved to NYHA class I patient after 1 month, or, 2 months, or, 3 months, or, 4 months, or 5 months, or, 6 months, or, 7 months, or, 8 months, or, 9 months or, 12 months or, more months of post administration of rAAV dose. In some embodiments, NYHA class III patient receiving rAAV administration as disclosed herein is improved to NYHA class I patient after 1 month, or, 2 months, or, 3 months, or, 4 months, or 5 months, or, 6 months, or, 7 months, or, 8 months, or, 9 months or, 12 months or, more months of post administration of rAAV dose. In some embodiments, NYHA class IV patient receiving rAAV administration as disclosed herein is improved to NYHA class III patient or, NYHA class II patient after 1 month, or, 2 months, or, 3 months, or, 4 months, or 5 months, or, 6 months, or, 7 months, or, 8 months, or, 9 months or, 12 months or, more months of post administration of rAAV dose.

### (Ii) ACC/AHA Classification:

One heart failure classification system useful in the methods disclosed herein is the ACC/AHA classification system. The American College of Cardiology (ACC) and the American Heart Association (AHA) worked together to create another classification system that complements the NYHA approach. It considers people who do not yet have HF but are at high risk for developing it.

The ACC/AHA classifies heart failure on the disease progression, and classifies HF in stages A, B, C, D according to presence of HF symptoms and signs and cardiac structural changes. See, e.g., Yancy CW, et al., Circulation 2013;128: e240-e327, and Hunt SA, et al., Circulation 2001;104:2996-3007.

The American College of Cardiology/American Heart Association (ACC/AHA) staging system defines four stages:
Stage A: High risk of heart failure but no structural heart disease or symptoms of heart failure (pre-heart failure)
Stage B: Structural heart disease but no symptoms of heart failure (pre-heart failure)
Stage C: Structural heart disease and symptoms of heart failure
Stage D: Refractory heart failure requiring specialized interventions

Table 1B: AHA/ACC 2013 - Staging System of the heart

| | | |
|---|---|---|
| Stage | Description | Examples |
| A | Presence of heart failure risk factors but no heart disease and no symptoms. Encompasses “pre heart | CAD (coronary artery disease), diabetes, hypertension, metabolic syndrome, |
| | failure” where intervention with management can overt progression to symptoms. | obesity, using cardiotoxins or alcohol, family history of cardiomyopathy, cerebrovascular accident (CVA), personal history of rheumatic fever. |
| B | People with structural heart disease but there are no signs or symptoms of heart failure (structural changes in heart before symptoms occur) | Left ventricular hypertrophy (LVH) or reduced left ventricular ejection fraction (LVEF), asymptomatic valvular heart disease, previous MI |
| | NYHA class I | |
| C | People with structural heart disease with prior or current symptoms of heart failure (symptoms have or are occurring) | Known structural heart disease with dyspnea (shortness of breath), fatigue, inability to exercise or reduced exercise tolerance |
| | NYHA class II and III | |
| D | People with advanced heart failure and severe symptoms or continued heart failure with severe symptoms difficult to manage with standard treatment or the patient requiring aggressive medical therapy. | Marked symptoms of rest despite maximal medical therapy, and recurrent hospitalizations |
| | NYHA class IV | |

The stages denote the level of risk for developing heart failure on through the development of advanced heart failure. The stages are progressive and correlated to treatment plans. As heart failure worsens, the condition advances to the next stage. There is no reverting back through the stages. With treatment, progression through the stages may be delayed. Diagnostic considerations include evaluating when heart failure starts, where it develops, how it impairs function, and whether or not it can be effectively managed with treatment.

In certain embodiments, ACC/AHA Stage D patient receiving rAAV administration as disclosed herein is improved to ACC/AHA Stage C patient after 1 month, or, 2 months, or, 3 months, or, 4 months, or 5 months, or, 6 months, or, 7 months, or, 8 months, or, 9 months or, 12 months or, more months of post administration of rAAV dose. In other embodiments, ACC/AHA Stage C patient receiving rAAV administration as disclosed herein is improved to ACC/AHA Stage B patient after 1 month, or, 2 months, or, 3 months, or, 4 months, or 5 months, or, 6 months, or, 7 months, or, 8 months, or, 9 months or, 12 months or, more months of post administration of rAAV dose. In some embodiments, ACC/AHA Stage D patient receiving rAAV administration as disclosed herein is improved to ACC/AHA Stage C or B patient or, ACC/AHA Stage A patient after 1 month, or, 2 months, or, 3 months, or, 4 months, or 5 months, or, 6 months, or, 7 months, or, 8 months, or, 9 months or, 12 months or, more months of post administration of rAAV dose. In some embodiments, ACC/AHA Stage C patient receiving rAAV administration as disclosed herein is improved to ACC/AHA Stage B or stage A patient after 1 month, or, 2 months, or, 3 months, or, 4 months, or 5 months, or, 6 months, or, 7 months, or, 8 months, or, 9 months or, 12 months or, more months of post administration of rAAV dose.

Table 1C: Comparison of the NYHA classification and the ACC/AHA guidelines (obtained from Haselhuhn et al., Cleveland Clinic Journal of Medicine February 2019, 86 (2) 123-139.)

| NYHA class I | NYHA class II | NYHA class III | NYHA class IV |
|---|---|---|---|
| No physical limitations | Slight limitation of physical activity | Marked limitation of physical activity | Symptoms at rest |
| Stage A | Stage B | Stage C | Stage D |
| Patients at risk for heart failure | Structural disease | Structural disease | End-stage disease |
| | No heart failure symptoms | Heart failure symptoms | |

In some embodiments, the ACCF/AHA guidelines for the management of heart failure can also be used, which are developed in collaboration with the American Academy of Family Physicians, American College of Chest Physicians, and the International Society for Heart and Lung Transplant, disclosed in Yancy, Clyde W., et al. “2017 ACC/AHA/HFSA focused update of the 2013 ACCF/AHA guideline for the management of heart failure: a report of the American College of Cardiology/American Heart Association Task Force on Clinical Practice Guidelines and the Heart Failure Society of America.” Journal of the American College of Cardiology 70.6 (2017): 776-803, which is incorporated herein in its entirety by reference. The 2017 focused update of the 2013 ACC/AHA guideline on heart failure contains important recommendations on prevention, novel biomarker uses, heart failure with preserved ejection fraction (HFpEF), and comorbidities such as hypertension, iron deficiency, and sleep-disordered breathing. Potential implications for management of acute decompensated heart failure will also be explored (see, e.g., Haselhuhn et al., Cleveland Clinic Journal of Medicine February 2019, 86 (2) 123-139.

### (III) 2016 European Society of Cardiology Guidelines (ESCG),

The 2016 European Society of Cardiology guidelines is disclosed in Piepoli et al., Eur Heart J. 2016 Aug 1;37(29):2315-2381, which is incorporated herein by reference, and is encompassed for use in the methods to treat HF disclosed herein, as an alternative to, or to complement the NYHA or ACC/AHA guidelines.

### (IV) Japanese Heart Failure Society (JHFS) Guidelines,

The Japanese Circulation Society (JCS) Guidelines are disclosed in Yamamoto K, et al., Japanese Heart Failure Society 2018 Scientific Statement on Nutritional Assessment and Management in Heart Failure Patients. Circ J. 2020 Jul 22;84(8):1408-1444, and is encompassed for use in the methods to treat HF disclosed herein, as an alternative to, or to complement the NYHA or ACC/AHA guidelines.

### (V) Kansas City Cardiomyopathy Questionnaire (KCCQ) Scores

KCCQ scores are scaled from 0 to 100 and frequently summarized in 25-point ranges, where scores represent health status as follows: 0 to 24: very poor to poor; 25 to 49: poor to fair; 50 to 74: fair to good; and 75 to 100: good to excellent as described in Spertus JA et al., JACC, Volume 76, Issue 20, 17 Nov. 2020, Pages 2379-2390. Because the most common means of quantifying health status in clinical practice and trials is the NYHA functional class, it is valuable to appreciate what KCCQ scores correlate to which NYHA functional class. About 85% of patients with scores of 0 to 24 are NYHA functional class III/IV; 60% of patients with scores of 25 to 49 are NYHA functional class III; one-half of patients with scores of 50 to 75 are NYHA functional class III and one-half are NYHA functional class II; and of those with scores over 75, over 80% are NYHA functional class I or II.

### (VI) Other Heart Failure Classification Systems:

Ejection Fraction: Classifies HF in HFrEF, HFmrEF, HFpEF based on left ventricular ejection fraction Ponikowski P, et al., Eur J Heart Fail 2016;18:891-975.

Aetiology: Classifies HF in stages specific aetiology of HF, e.g. ischaemic/non-ischaemic, valvular, hypertensive, infiltrative cardiomyopathy such as cardiac amyloidosis, peripartum cardiomyopathy, viral myocarditis, chemotherapy-induced cardiomyopathy, as disclosed in Yancy CW, et al., Circulation 2013;128:e240-e327 and Ponikowski P, et al., Eur J Heart Fail 2016; 18:891-975.

MOGES: classifies HF in levels of a morpho-functional phenotype (M), organ(s) involvement (O), genetic inheritance pattern (G), etiological annotation (E) including genetic defect or underlying disease/substrate, and the functional status (S). See, e.g., Arbustini E, et al., The MOGE(S) classification of cardiomyopathy for clinicians. J Am Coll Cardiol 2014;64:304-318.

INTERMACS (profiles of advanced HF): Classifies HF as Profiles 1 through 7 according to symptoms, functional capacity, haemodynamic stability for patients who are considered for advanced HF therapies. See, e.g., Stevenson LW, et al., INTERMACS profiles of advanced heart failure: the current picture. J Heart Lung Transplant 2009;28:535-541.

### (VII) Quality of Life (QOF) Questionnaires:

Assessment of heart failure can be categorized or classified using a variety of self-assessed questionnaires, e.g., selected from any of: Minnesota LIVING WITH HEART FAILURE® Questionnaire (MLHFQ, also known as MLWHF, LHFQ, MQOL)), (Rector et al.,); Chronic Heart failure Questionnaire (CHFQ, CHQ) (Guyatt et al., 1989); Quality of Life Questionnaire for Severe Heart Failure (QLQ-SHF or QQL-SHF) (Wiklund et al., 1987), Kansas city cardiomyopathy Questionnaire (KCCQ) (Spertus et al, 1999), and Left Ventricular Dysfunction Questionnaire-36 (LVD-36) (O′Learly et al., 1998), each of which are reviewed in Garin, Olatz, et al. “Disease-specific health-related quality of life questionnaires for heart failure: a systematic review with meta-analyses.” Quality of Life Research 18.1 (2009): 71-85, and Garin, O., et al. Assessing health-related quality of life in patients with heart failure: a systematic, standardized comparison of available measures. Heart Fail Rev 19, 359-367 (2014), each of which are incorporated herein in their entirety by reference.

In some embodiments, a clinically meaningful change in a Quality of Life (QOL) questionnaire is a 10 point decrease, or greater than 10 point decrease in the QOF Questionnaire score measured at least 6-months, or at least 12-months after administration of the rAAV according to the methods as disclosed herein, as compared to the QOF Questionnaire score prior to administration of the rAAV to the subject. In some embodiments, a clinically meaningful change in a Quality of Life (QOL) questionnaire score is a 10-point decrease, or a 11-point decrease, or a 12-point decrease, or a 13-point decrease, or a 14-point decrease, or a 15-point decrease, or a 16-point decrease, or a 17-point decrease, or a 18-point decrease, or a 19-point decrease, or a 20-point decrease, or a 21 point decrease, or 22 point decrease, or 23 point decrease, or 24 point decrease, or 25 point decrease, or a greater than 25-point decrease in a QOL Questionaire score e.g., in KCCQ score or, in MLWHF score measured at least 3 months, or, at least 6-months, or at least 12-months after administration of the rAAV according to the methods as disclosed herein, as compared to the ESV prior to administration of the rAAV to the subject.

### (VI) Measurements for Evaluation of Treatment:

In some embodiments, the methods to treat HF as disclosed herein improvement in the at least one parameter from a baseline level in the patient, where the at least one parameter is selected from the group consisting essentially of: (i) ejection fraction (EF or, interchangeably used as Left ventricular ejection fraction or, LVEF), (ii) end systolic volume (ESV), (iii) cardiac contractility, selected from ejection fraction (EF) and fractional shortening (FS), (iv) cardiac volumes selected from any of: end diastolic volume (DV) and end systolic volume (ESV), (iv) functional criteria, selected from any of: a 6-minute walk test (6MWT), exercise and VO2max (also referred to as pVO2max or myocardial oxygen consumption (MVO2) (measured in ml/kg/min); (v) BNP level, (vi) Pro-BNP level, (vii) biomarker level, wherein the biomarker level is selected from the group of: troponin, serum creatinine, cystatin-C, or hepatic transaminases, (viii) patient-reported outcomes (PROs), such as reduced symptoms, health-related quality of life (HRQOL), or patient perceived health status, and (ix) a decrease in any of: mortality risk due to heart failure, reduced hospitalization due to heart failure symptoms, or therapeutic intervention for treatment of HF.

In some embodiments, a clinically meaningful change in End Systolic Volume (ESV) is a 10% decrease, or greater than 10% decrease in ESV measured at least 1 month or, at least 3 months, or, at least 6-months, or at least 12-months after administration of the rAAV according to the methods as disclosed herein, as compared to the ESV prior to administration of the rAAV to the subject. In some embodiments, a clinically meaningful change in ESV is a 10% decrease, or about 11%, or about 12%, or about 13%, or about 14%, or about 15%, or greater than a 15% decrease in ESV measured at least 3 months, or at least 6-months, or at least 12-months after administration of the rAAV according to the methods as disclosed herein, as compared to the ESV prior to administration of the rAAV to the subject. In some embodiments, a clinically meaningful change in End Systolic Volume (ESV) is a 20 ml decrease, or greater than 20 ml decrease in ESV measured at least 1 month, or, at least 3 months, or, at least 6-months, or at least 12-months after administration of the rAAV according to the methods as disclosed herein, as compared to the ESV prior to administration of the rAAV to the subject.

### (A) BNP Level and NT-Pro-BNP Level

In some embodiments, the method of treatment as disclosed herein can be assessed by measuring biomarkers, such as, e.g., circulating natriuretic peptide (either BNP or NT-proBNP) levels in the serum obtained from the subject after a pre-defined period of time, such as, for example, at least about 1, or 2, or 3, or 4, or 5, or 6, or 9, or, 12, or more months after administration. In some embodiments, the method of treatment or administration as disclosed herein can be assessed by measuring BNP and/or NT-proBNP levels in the serum before administration, and after a period of about 4-6 months, or after 6-months after administration, or after more than 6-months post-administration.

Methods to measure NT-proBNP levels are well known in the art. Acute heart failure (AHF) is unlikely if the level of BNP in the serum from the subject is equal or less than 100 pg/ml or if the NT-proBNP in the serum from the subject is equal or less than 300 pg/ml. A diagnosis of HF is likely where the level of BNP in the serum from the subject is >400 pg/ml or if the NT-proBNP in the serum from the subject is >450 pg/ml for subjects less than 50 years of age, or > 900 pg/ml for subjects between 50-75 years of age, or > 1800 pg/ml for subjects >75 years of age.

Accordingly, in some embodiments, a BNP level of equal or less than 400 pg/ml in the serum of a treated patient at least 6 months after administration indicates effective treatment.

In some embodiments, a NT-proBNP level of equal or less than 450 pg/ml in the serum of a treated patient of less than 50 years of age at least 6 months after administration indicates effective treatment. In some embodiments, a NT-proBNP level of equal or less than 900 pg/ml in the serum of a treated patient of 50-75 years of age at least 6 months after administration indicates effective treatment. In some embodiments, a NT-proBNP level of equal or less than 1700 pg/ml in the serum of a treated patient of ≥75 years of age at least 6 months after administration indicates effective treatment.

In some embodiments, a clinically meaningful change in NT-pro-BNP (pg/ml) is a 35% decrease, or greater than 35% decrease in the level of NT-pro-BNP (pg/ml) measured at least 1 month, at least 3 months, or, at least 6-months, or at least 12-months after administration of the rAAV according to the methods as disclosed herein, as compared to the level of level of NT-pro-BNP (pg/ml) measured prior to administration of the rAAV to the subject. In some embodiments, a clinically meaningful change in NT-pro-BNP (pg/ml) is a 35% decrease, or about a 36%, or about a 37%, or about a 38%, or about a 39%, or about a 40%, or about a 40-45% or about a 46-50% decrease or a greater than a 50% decrease in the level of NT-pro-BNP (pg/ml) measured at least 1 month or, at least 3 months, or, at least 6-months, or at least 12-months after administration of the rAAV according to the methods as disclosed herein, as compared to the level of of NT-pro-BNP (pg/ml) measured prior to administration of the rAAV to the subject.

In some embodiments, a decrease of about 10%, or about 15%, or about 20%, or about 25%, or about 30% or >30% in BNP levels or NT-proBNP levels in the serum of a subject at least 3- months, or at least about 6-months after administration of the rAAV of as compared to levels of BNP or NT-proBNP levels pre-administration is indicative of effective treatment. In some embodiments, a decrease of about 0.1-fold, 0.2-fold, 0.3-fold, 0.4-fold, 0.5-fold, 0.6-fold, 0.7-fold, 0.8-fold, 0.9-fold, 1.0-fold, 1.1-fold, 1.2-fold, 1.3-fold, 1.4-fold, 1.5-fold, 1.6-fold, 1.7-fold, 1.8-fold, 1.9-fold, or 2.0-fold, or greater of 2.0-fold in the serum level of BNP or NT-proBNP in a subject at least 3- months, or at least about 6-months after administration of the rAAV of as compared to levels of BNP or NT-proBNP levels pre-administration is indicative of effective treatment.

In some embodiments, an effective treatment is considered if the serum level of BNP in a subject (of any age) is less than 400 pg/ml, or about 350 pg/ml, or about 300 pg/ml, or about 250 pg/ml, or about 200 pg/ml, or about 150 pg/ml, or about 100 pg/ml, or less than 100 pg/ml at least 3- months, or at least about 6-months after administration of the rAAV of as compared to levels of BNP levels pre-administration.

In some embodiments, an effective treatment is considered if the serum level of NT-proBNP in a subject aged less than 50 years of age is less than 450 pg/ml, or about 400 pg/ml, or about 350 pg/ml, or about 300 pg/ml, or about 250 pg/ml, or about 200 pg/ml, or about 150 pg/ml, or about 100 pg/ml, or less than 100 pg/ml at least 3- months, or at least about 6-months after administration of the rAAV of as compared to levels of NT-proBNP levels pre-administration. In some embodiments, the age of the patient being treated is less than 50 years of age, or, between 50-75 years of age, or, ≥75 years of age.

In some embodiments, an effective treatment is considered if the serum level of NT-proBNP in a subject aged between 50-75 years of age is less than 900 pg/ml, or about 850 pg/ml, or about 800 pg/ml, or about 750 pg/ml, or about 700 pg/ml, or about 650 pg/ml, or about 600 pg/ml, or about 550 pg/ml, or about 500 pg/ml, or about 450 pg/ml, or less than 450 pg/ml at least 3- months, or at least about 6-months after administration of the rAAV of as compared to levels of NT-proBNP levels pre-administration. In some embodiments, the age of the patient being treated is less than 50 years of age, or, between 50-75 years of age, or, ≥75 years of age.

In some embodiments, an effective treatment is considered if the serum level of NT-proBNP in a subject aged ≥75 years of age is less than about 1800 pg/ml, or about 1700 pg/ml, or about 1600 pg/ml, or about 1500 pg/ml, or about 1400 pg/ml, or about 1300 pg/ml, or about 1200 pg/ml, or about 1100 pg/ml, or about 1000 pg/ml, or about 900 pg/ml, or about 800 pg/ml, or about 700 pg/ml, or about 600 pg/ml, or about 500 pg/ml, or about 450 pg/ml or less than 450 pg/ml at least 3- months, or at least about 6-months after administration of the rAAV of as compared to levels of NT-proBNP levels pre-administration. In some embodiments, the age of the patient being treated is less than 50 years of age, or, between 50-75 years of age, or, ≥75 years of age.

In some embodiments, a clinically meaningful change in BNP (pg/ml) is a 40% decrease, or greater than 40% decrease in the level of BNP (pg/ml) measured at least 6-months, or at least 12-months after administration of the rAAV according to the methods as disclosed herein, as compared to the level of BNP (pg/ml) measured prior to administration of the rAAV to the subject. In some embodiments, a clinically meaningful change in BNP (pg/ml) is a 40% decrease, or about a 41%, or about a 42%, or about a 43%, or about a 44%, or about a 45%, or about a 45-50% or about a 51-55% decrease, or a 56-60% decrease or a greater than a 60% decrease in the level of BNP (pg/ml) measured at least 1 month or, at least 3 months, or at least 6-months, or at least 12-months after administration of the rAAV according to the methods as disclosed herein, as compared to the level of level of BMP (pg/ml) measured prior to administration of the rAAV to the subject.

### (B) Ejection Fraction (EF) (also Interchangeably Used With Left Ventricle Ejection Fraction (LVEF))

In some embodiments, cardiopulmonary exercise testing can be assessed using a modified Bruce protocol according to methods known in the art. An observed and change from baseline in Echocardiographic assessment in Left Ventricular Ejection Fraction (LVEF) can also be used to assess the treatment, where the LVEF can be assessed at the following timepoints: before administration, 18-24 hours post administration, after 4 weeks, at about 6 months, and at about 12 months-post administration.

In some embodiments, a clinically meaningful change in Ejection Fraction (EF) is a 5% increase, or greater than 5% increase in ejection fraction measured at least 1 month, or, at least 3 months, at least 6-months, or at least 12-months after administration of the rAAV according to the methods as disclosed herein, as compared to the ejection fraction (EF) prior to administration of the rAAV to the subject. In some embodiments, a clinically meaningful change in Ejection Fraction (EF) is a 5% increase, or about 6%, or about 7%, or about 8%, or about 9%, or about 10%, or greater than a 10% increase in ejection fraction measured at least 1 month, or, at least 3 months, at least 6-months, or at least 12-months after administration of the rAAV according to the methods as disclosed herein, as compared to the ejection fraction (EF) prior to administration of the rAAV to the subject.

In some embodiments, the improved function may be an improvement of any amount as compared to the cardiac function of a matched control subject receiving vehicle only. For example, the improvement (i.e., increase) in LVEF at least 3- or at least 6-months after administration of a rAAV vector according to the methods disclosed herein be about 10%, 20%, 30%, 40%, 50%, 60%, 70%, 80%, 90%, 100%, 150%, 200% or more than about 200% as compared to the LVEF measured at or before the rAAV administration.

In another example, the improvement in E/A ratio after treatment may be about 10%, 20%, 30%, 40%, 50%, 60%, 70%, 80%, 90%, 100%, 150%, 200% or more than about 200% as compared to the E/A ratio measured at or before the rAAV administration. In yet another example, the improvement in left atrial volume (LAV) may be about 10%, 20%, 30%, 40%, 50%, 60%, 70%, 80%, 90%, 100%, 150%, 200% or more than about 200% as compared to the level of LAV measured at or before the rAAV administration.

In some embodiments, if the LVEF is preserved ≥50%, or at least 55%, or at least 60% or more than 60% as compared to the level of LVEF measured, at and/or before administration of the rAAV according to the methods as disclosed herein, then the rAAV vector and the administration methods is considered an effective treatment (e.g., see Dokainish, Glob Cardiol Sci Pract. 2015; 2015: 3). Typically, a normal LVEF ranges from 50% to 75%, and a LVEF of below 53% for women and 52% for men is considered low, and a subject with a LVEF of 49% or less, or about 45%, or about 40%, or about 36%, or between 36-49% is indicative of heart failure. In some embodiments, an increase in LVEF measured in the subject by at least 1%, or about 2%, or about 3%, or about 4%, or about 5%, or about 6%, or about 7%, or about 8%, or about 9%, or about 10%, or about 15%, or about 20% or about 35%, or about 40%, or about 45%, or about 50% or more than 50% as compared to the LVEF measured at, or before administration of a rAAV according to the methods as disclosed herein is indicative of an effective treatment. For example, in an illustrative embodiment, if the subject has a LVEF of 36% measured at, or before administration of the rAAV vector according to the methods as disclosed herein, an increase in LVEF by at least 1% to 37% LVEF, or about 2% to 38% LVEF, or about 3% to 39% LVEF, or about 4% to 40% LVEF, or about 5% to 41% LVEF, or about 6% to 42% LVEF, or about 7% to 43% LVEF, or about 8% to 44% LVEF, or about 9% to 45% LVEF, or about 10% to 46% LVEF, or about 15% to 51% LVEF, or about 20% to 56% LVEF or about 25% to 61% LVEF, as measured at least 3- months, or at least about 6-months after administration of the rAAV is indicative of an effective treatment.

### (C) End Systolic Volume (ESV)/left Ventricle End Systolic Dimension (LVESD) and End Diastolic Volumes (DV)/ Left Ventricle End Diastolic Dimension (LVEDD)

In one embodiment, the methods also include administration of a rAAV vector as disclosed herein, according to the methods as disclosed herein, to attenuate cardiac remodeling. Cardiac remodeling may be measured by any method known in the art, including the methods, such as, e.g., echocardiography. As an example, left ventricle chamber size may be used as a measure for cardiac remodeling. In evaluating attenuation of cardiac remodeling, an attenuation of the increase in size of the left ventricle may be an attenuation of any amount as compared with the left ventricle size before administration of a rAAV vector as disclosed herein, according to the methods as disclosed herein.

In some embodiments, a clinically meaningful change in End Systolic Volume (ESV) is a 10% decrease, or greater than 10% decrease in ESV measured at least 1 month, or, at least 3 months, at least 6-months, or at least 12-months after administration of the rAAV according to the methods as disclosed herein, as compared to the ESV prior to administration of the rAAV to the subject. In some embodiments, a clinically meaningful change in ESV is a 10% decrease, or about 11%, or about 12%, or about 13%, or about 14%, or about 15%, or greater than a 15% decrease in ESV measured at least 1 month, or, at least 3 months, at least 6-months, or at least 12-months after administration of the rAAV according to the methods as disclosed herein, as compared to the ESV prior to administration of the rAAV to the subject.

In some embodiments, a clinically meaningful change in End Systolic Volume (ESV) is a 20 ml decrease, or greater than 20 ml decrease in ESV measured at least 1 month, or, at least 3 months, at least 6-months, or at least 12-months after administration of the rAAV according to the methods as disclosed herein, as compared to the ESV prior to administration of the rAAV to the subject. In some embodiments, a clinically meaningful change in ESV is a 20 ml decrease, or about 22 ml, or about 23 ml or about 24 ml, or about 25 ml, or about 26 ml, or about 27 ml, or about 28 ml, or about 29 ml, or about 30 ml, or greater than a 30 ml decrease in ESV measured at least 1 month, or, at least 3 months, at least 6-months, or at least 12-months after administration of the rAAV according to the methods as disclosed herein, as compared to the ESV prior to administration of the rAAV to the subject.

In some embodiments, an attenuation of the increase in size of the left ventricle may be an attenuation of any amount as compared with the left ventricle size of a matched control subject receiving vehicle only. Left ventricle chamber size may be measured, for example, by assaying left ventricle end diastolic dimension (LVEDD) or left ventricle end systolic dimension (LVESD). In an example, the change in LVEDD at least 3- or at least 6-months after administration with a rAAV vector as disclosed herein, according to the methods as disclosed herein may be 10%, 20%, 30%, 40%, 50%, 60%, 70%, 80%, 90%, 100%, 150%, 200% or more than about 200% as compared to the levels measured at, or before administration of the rAAV. In another example, the change in LVESD at least 3- or at least 6-months after administration with a rAAV vector as disclosed herein, according to the methods as disclosed herein may be 10%, 20%, 30%, 40%, 50%, 60%, 70%, 80%, 90%, 100%, 150%, 200% or more than about 200% as compared to the LVESD level measured at, or before administration of the rAAV.

### (d)VO2max (Also Referred to as pVO2max or Myocardial Oxygen Consumption (MVO₂)

In one embodiment, the methods also include administration of a rAAV vector as disclosed herein, according to the methods as disclosed herein, to improve exercise capacity in a subject having congestive heart failure. The improvement in exercise capacity may be measured by any method known in the art. For example, the improvement in exercise capacity may be measured by assaying peak VO₂ uptake or exercise capacity to peak lactate ratio. Peak oxygen uptake during exercise may be measured, for example, by indirect calorimetry. In an example, the change in exercise capacity to peak lactate ratio measured at least 3- or at least 6-months after administration with a rAAV vector as disclosed herein, according to the methods as disclosed herein may be 10%, 20%, 30%, 40%, 50%, 60%, 70%, 80%, 90%, 100%, 150%, 200% or more than about 200% as compared to the peak lactate ratio measured at, or before administration of the rAAV.

In some embodiments, a clinically meaningful change in myocardial oxygen consumption (MVO₂) is a 1.5 ml/kg/min increase, or greater than 1.5 ml/kg/min increase in MVO₂ measured at least 1 month, or, at least 3 months, at least 6-months, or at least 12-months after administration of the rAAV according to the methods as disclosed herein, as compared to the MVO₂ measured prior to administration of the rAAV to the subject. In some embodiments, a clinically meaningful change in MVO₂ is a 1.5 ml/kg/min increase, 1.6 ml/kg/min, or about a 1.7 ml/kg/min, or about 1.8 ml/kg/min, or about 1.9 ml/kg/min, or about 2.0 ml/kg/min, or about 2.1 ml/kg/min, or greater than a 2.1 ml/kg/min increase in MVO₂ measured at least 1 month, or, at least 3 months, at least 6-months, or at least 12-months after administration of the rAAV according to the methods as disclosed herein, as compared to the MVO₂ measured prior to administration of the rAAV to the subject.

In some embodiments, the method of treatment as disclosed herein can be assessed by measuring a peak oxygen update (VO₂), measured at least about 3, or 4, or 5, or 6 months or between 6-10 months, or 12 months after administration of the rAAV vector. In some embodiments, a modest pVO2 is indicative of an effect treatment - that is, a modest increase in peak VO₂ over 3 months was associated with a more favourable outcome. Accordingly, monitoring the change in peak VO₂ for treated subjects can be used to assess prognosis and therapeutic effect of the rAAV vector and method of administration. (Swank et al., Circulation: Heart Failure, Volume 5, Issue 5, September 2012, Pages 579-585). Every 6% increase in peak VO₂, adjusted for other significant predictors, was associated with a 5% lower risk of the primary end point (hazard ratio=0.95; CI=0.93-0.98; P<0.001) of all-cause mortality and all cause hospitalization; a 4% lower risk of the secondary end point of time to cardiovascular mortality or cardiovascular hospitalization (hazard ratio=0.96; CI=0.94-0.99; P<0.001); an 8% lower risk of cardiovascular mortality or heart failure hospitalization (hazard ratio=0.92; CI=0.88-0.96; P<0.001); and a 7% lower all-cause mortality (hazard ratio=0.93; CI=0.90-0.97; P<0.001). In some embodiments, post-administration peak VO₂ is increased from baseline peak VO₂ by about 2%, or, about 3%, or, about 4%, or, about 5%, or, about 6%, or, about 7%, or, about 8%, or, about 9%, or, about 10%, or, about 11%, or, about 12%, or, about 13%, or, about 14%, or, about 15%, or, about 16%, or, about 17%, or, about 18%, or, about 19%, or, about 20%, or, about 21%, or, about 22%, or, about 23%, or, about 24%, or, about 25%, or, about 26%, or, about 27%, or, about 28%, or, about 29%, or, about 30%. In some embodiments, post-administration peak VO₂ is increased from baseline peak VO₂ by at least 2%. In some embodiments, post-administration peak VO₂ is increased from baseline peak VO₂ by at least 5%. In other embodiments, post-administration peak VO₂ is increased from baseline peak VO₂ by at least 10%. In yet another embodiment, post-administration peak VO₂ is increased from baseline peak VO₂ by at least 20%. In certain embodiments, post-administration peak VO₂ is increased from baseline peak VO₂ by at least 30%. In some embodiments, post-administration peak VO₂ is increased from baseline peak VO₂ by about 1.1 fold, or, about 1.15 fold, or, about 1.2 fold, or, about 1.25 fold, or, about 1.3 fold, or, about 1.35 fold, or, about 1.4 fold, or, about 1.45 fold, or, about 1.5 fold, or, about 1.55 fold, or, about 1.6 fold, or, about 1.65 fold, or, about 1.7 fold, or, about 1.75 fold, or, about 1.8 fold, or, about 1.85 fold, or, about 1.9 fold, or, about 1.95 fold, or, about 2 fold, or, about 2.5 fold, or, about 3 fold, or, about 3.5 fold, or, about 4 fold, or, about 4.5 fold, or, about 5 fold, or, about 5.5 fold, or, about 6 fold, or, about 6.5 fold, or, about 7 fold, or, about 7.5 fold, or, about 8 fold, or, about 8.5 fold, or, about 9 fold, or, about 9.5 fold, or, about 10 fold. In certain embodiments, post-administration peak VO₂ is increased from baseline peak VO₂ by at least 1.1 fold, or, at least 1.2 fold, or, at least 1.5 fold, or, at least 2 fold, or, at least 2.5 fold, or, at least 3 fold, or, at least 3.5 fold, or, at least 4 fold, or, at least 4.5 fold, or, at least 5 fold, or, at least 6 fold, or, at least 6.5 fold, or, at least 7 fold, or, at least 8 fold, or, at least 9 fold, or, at least 10 fold, or, at least 11 fold, or, at least 12 fold, or, at least 13 fold, or, at least 14 fold, or, at least 15 fold, or, at least 16 fold, or, at least 17 fold, or, at least 18 fold, or, at least 19 fold, or, at least 20 fold, or, at least 22 fold, or, at least 25 fold, or, at least 30 fold, or, at least 35 fold, or, at least 40 fold, or, at least 45 fold or, at least 50 fold.

In evaluating improved cardiac function associated with congestive heart failure, the improved function may be an improvement of any amount as compared with the cardiac functioning prior to administration of a rAAV vector as disclosed herein, according to the methods as disclosed herein.

### (E) 6 Minute Walk Test (6MWT)

In some embodiments, the method of treatment as disclosed herein can be assessed by measuring secondary outcome measures, e.g., assessing the subject in a 6-minute walk distance test (referred to as a 6-minute walk test (6MWT) at the following timepoints: before administration, 18-24 hours post administration, after 4 weeks, at about 6 months, and at about 12 months-post administration. In certain embodiments, the 6-minute walk test (6MWT) can be performed at any of the above time points is improved from the baseline at least by 15 m, or, at least by 20 m, or at least by 25 m, or, at least by 30 m, or at least by 40 m, or, at least by 50 m, or, at least by 55 m, or, at least by 60 m, or, at least by 65 m, or, at least by 70 m, or, at least by 75 m, or, at least by 80 m, or, at least by 85 m, or, at least by 90 m, or, at least by 100 m, or, at least by 120 m, or, at least by 150 m, or, at least by 170 m, or at least by 180 m, or at least by 200 m, or more.

In some embodiments, a clinically meaningful change in the 6MWT is a 50 meters increase, or greater than 50 meter increase in the distance walked in the 6MWT measured at least 1 month, or, at least 3 months, at least 6-months, or at least 12-months after administration of the rAAV according to the methods as disclosed herein, as compared to the distance walked in the 6MWT prior to administration of the rAAV to the subject. In some embodiments, a clinically meaningful change in the 6MWT is a 50 meter increase, or an increase of about 60 m, or about 70 m, or about 80 m, or about 90 m, or about 100 m, or about 110 m, or about 120 m, or about 130 m, or about 140 m, or about 150 m, or an increase of greater than 150 meters in the distance walked in the 6MWT measured at least 1 month, or, at least 3 months, at least 6-months, or at least 12-months after administration of the rAAV according to the methods as disclosed herein, as compared to the distance walked in the 6MWT prior to administration of the rAAV to the subject.

Such a 6MWT can be conducted according to the methods as disclosed in Giannitsi et al., Ther Adv Cardiovas Disorders, 2019, 2019; 13: 1753944719870084, which is incorporated herein in its entirety by reference.

### (ƒ) Cardiac Contractibility, Including Fractional Shortening (FS) and Ejection Fraction (EF)

In one embodiment, the methods also include administration of a rAAV vector as disclosed herein, according to the methods as disclosed herein, to improve cardiac contractility. Improving cardiac contractility may include any increase in the number of cardiac myocytes available for contraction, the ability of cardiac myocytes to contract, or both. In order to evaluate the improvement of cardiac contractility, any mode of assessment may be used. For example, clinical observation, such as an increase in cardiac output or a decrease in cardiac rate or both, may lead to a determination of increased cardiac contractility. Alternatively, in vivo an increased contractility of the heart may be assessed by a determination of an increased fractional shortening of the left ventricle. Fractional shortening of the left ventricle may be observed by any available means such as echocardiograph.

In evaluating increased cardiac contractility, the increase in fractional shortening of the left ventricle may be an increase of any amount as compared with the fractional shortening before administration of a rAAV vector as disclosed herein, according to the methods as disclosed herein. For example, the increase in shortening measured at least 3- or at least 6-months after administration with a rAAV vector according to the methods as disclosed herein can about 10%, 20%, 30%, 40%, 50%, 60%, 70%, 80%, 90%, 100%, 150%, 200% or more than about 200% as compared to the levels of shortening measured at, or before the administration of rAAV. In a further aspect, prophylactic and therapeutic methods are provided. Treatment on an acute or chronic basis is contemplated. In addition, treatment on an acute basis may be extended to chronic treatment, if so indicated. In one aspect is provided a method for the treatment or prevention of a condition associated with congestive heart failure in a subject in need thereof. The method generally comprises administering to the subject an amount of rAAV vector as disclosed herein, according to the methods as disclosed herein, effective to prevent or ameliorate congestive heart failure, wherein the condition associated with congestive heart failure is thereby improved.

In some embodiments, the rAAV vector expresses an I-1 protein or a functional variant thereof as disclosed herein in the heart of the subject in an amount effective to increase cardiac contractility and reduce morphological deterioration associated with cardiac remodeling in the subject with existing heart failure. In some embodiments, the increase in contractility is determined by increased myocyte shortening (myocyte length change), rates of myocyte cell shortening (dL/dt) and relengthening (-dL/dt), a lower time constant for relaxation (tau(.tau.)), and accelerated calcium signal decay.

In yet another embodiment, the methods further comprise the identification of a subject in need of treatment. Any effective criteria may be used to determine that a subject may benefit from administration of a rAAV vector as disclosed herein, according to the methods as disclosed herein. Methods for the diagnosis of heart disease and diabetes, for example, as well as procedures for the identification of individuals at risk for development of these conditions, are well known to those in the art. Such procedures may include clinical tests, physical examination, personal interviews and assessment of family history.

### (G) Biomarkers:

Biomarkers from blood can help detect the presence of HF, determine its severity, assess risk of future events, and guide the efficacy of the treatment of a rAAV according to the methods as disclosed herein. While BNP and Pro-BNP are commonly assessed biomarkers in classification of HF, other biomarkers can be used to further assess prognoses and effectiveness of a HF therapy.

In some embodiments, a panel of biomarkers can be used to assess efficacy of treatment, as disclosed in U.S. Pat. 8,450,069, which is incorporated herein in its entirety by reference, including measuring levels of cTnI and/or BNP in combination with one or more and vascular inflammation markers, e.g., IL-6, TNFα, IL-17a.

A number of biomarkers associated with HF are well recognized, and measuring their concentrations in circulation can be a convenient and non-invasive approach to provide important information about disease severity and helps in the detection, diagnosis, prognosis, and management of HF. These include natriuretic peptides, soluble suppressor of tumorgenicity 2, highly sensitive troponin, galectin-3, midregional proadrenomedullin, cystatin-C, interleukin-6, procalcitonin, and others. Accordingly, in some embodiments, a biomarker is selected from the group of: troponin, serum creatinine, cystatin-C, or hepatic transaminases. Measurement of these biomarkers are disclosed in Chow, et al. “Role of biomarkers for the prevention, assessment, and management of heart failure: a scientific statement from the American Heart Association.” Circulation 135.22 (2017): e1054-e1091, which is incorporated herein in its entirety..

In some embodiment, the biomarker is a miRNA. Some MicroRNAs biomarkers have been used and as they are stable in the circulation, selected miRNAs can be used as potential biomarkers in coronary artery disease, myocardial infarction, hypertension, diabetes mellitus, viral myocarditis, and HF. For instance, seven miRNAs were validated to be enriched in plasma of HF patients (miR-423-5p, miR-18b*, miR-129-5p, HS_202.1, miR-622, miR-654-3p, and miR-1254), among which miR-423-5p was most strongly related to the clinical diagnosis of HF. The circulating levels of miR-423-5p were related to disease severity as shown by an inverse correlation with ejection fraction and higher levels of miR-423-5p in patients with a higher New York Heart Association (NYHA) classification. MiR-423-5p was also correlated to the levels of the current clinically used biomarker N-terminal pro-brain natriuretic peptide (NT-proBNP). (see, e.g., Goren Y, Kushnir M, Zafrir B, Tabak S, Lewis BS, Amir O. Serum levels of microRNAs in patients with heart failure. Eur J Heart Fail 14: 147-154, 2012 and Tijsen AJ, et al., Circulating microRNAs as diagnostic biomarkers for cardiovascular diseases. Am J Physiol Heart Circ Physiol. 2012; 303:H1085-H1095).

Three other circulating miRNAs are linked to the diagnosis of HF. The endothelium-specific miR-126 was found to be negatively correlated with age, BNP, and NYHA class in 10 patients with and 17 asymptomatic controls (Fukushima Y, et al., Assessment of plasma miRNAs in congestive heart failure. Circ J 75: 336-340, 2011.8). Corsten et al. (Corsten MF, et al., Circulating MicroRNA-208b and MicroRNA-499 reflect myocardial damage in cardiovascular disease. Circ Cardiovasc Genet 3: 499-506, 2010.10) found both miR-499 and miR-122 to be enriched in the plasma of patients with acute HF compared with healthy controls, of which miR-499 is probably myocardium derived.

One of the characteristics of an ideal biomarker is changing levels as disease severity changes in response to therapy. miR-499-5p and miR-423-5p have been demonstrated to show the effect of therapy in a in a rat model of HF (Montgomery RL, et al., Therapeutic inhibition of miR-208a improves cardiac function and survival during heart failure. Circulation 124: 1537-1547, 2011).

In addition, miR-1, miR-133a, miR-133b, and miR-499-5p were elevated in patients with MI, whereas miR-122 and miR-375 were reduced (see, e.g., Tijsen AJ, et al., Circulating microRNAs as diagnostic biomarkers for cardiovascular diseases. Am J Physiol Heart Circ Physiol. 2012; 303:H1085-H1095).

In some embodiments, the protection against morphological deterioration associated with cardiac remodeling is determined by measuring the heart-to-body weight ratio, infarct size, and the presence of cardiac fibrosis, wherein protection is present if expression of said human phosphatase inhibitor-1 (I-1) or variant thereof results in a reduced heart-to-body weight ratio, a decreased infarct size, or reduced cardiac fibrosis as compared to a control.

In some embodiments, a subject treated with a rAAV vector and according to the administration methods as disclosed herein can be evaluated by assessing the effect of the treatment on a parameter related to cardiac function or cardiac cellular function, e.g., contractility. For example, SR Ca2+ ATPase activity or intracellular Ca2+ concentration can be measured, using the methods described above. Furthermore, force generation by hearts or heart tissue can be measured using methods described in Strauss et al., Am. J. Physiol., 262:1437-45, 1992.

In some embodiments, a subject treated with a rAAV vector and according to the administration methods as disclosed herein can also be evaluated by its effect on a subject, e.g., according to parameters that one skilled in the art of treatment would recognize as relevant for the particular treatment. For example, in treating heart failure, exemplary parameters may relate to cardiac and/or pulmonary function. Cardiac parameters include pulse, EKG signals, lumen loss, heart rate, heart contractility, ventricular function, e.g., left ventricular end-diastolic pressure (LVEDP), left ventricular systolic pressure (LVSP), Ca2+ metabolism, e.g., intracellular Ca2+ concentration or peak or resting Ca2+, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, PI3 kinase, AKT kinase, sodium-calcium exchanger, calcium channel (L and T), CK-MB, calsequestrin or calreticulin. The evaluation can include performing angiography (e.g., quantitative angiography) and/or intravascular ultrasound (IVUS), e.g., before, after, or during the treatment. In certain embodiments, echocardiographic assessments of LVEF, LVEVD, LVEDVI, VLESV, LVEVI, SpI, GLS or, degree of mitral regurgitation or, any combination of these or, all of these are performed at 4 weeks after, or, 6 weeks after, or, 8 weeks after, or, 12 weeks after, or, 3 months after, or, 6 months after, or, 9 months after, or, 12 months or, more months after administering the rAAV vector as described herein. In some embodiments, assessment of all-cause mortality and/or, Heart failure related hospitalization indicates the safety and/or, efficacy of the rAAV treatment as described herein. In some embodiments, survival, cardiac transplantation, or, left ventricular assist device (LAVD) implantation or, any combination of these are assessed in the monitoring of the rAAV administered patients.

In some embodiments of a method according to the invention, the rAAV vector is introduced in an amount effective to result in a condition selected from the group consisting of myocyte shortening, lowering of the time constant for relaxation, and accelerating calcium signal decay, and combinations thereof. In yet another embodiment, the rAAV vector is introduced in an amount effective to improve the end-systolic pressure dimension relationship or combinations thereof.

In some embodiments, the methods of administration and treatment as disclosed herein comprise expressing a therapeutic amount of the inhibitor of PP1 (I-1, or I-1c, or variant thereof) in the heart tissue of said subject. Suitably, expressing a therapeutic amount of the inhibitor of PP1 in the heart tissue reduces the symptoms of heart failure or a heart disorder of a subject. Suitably, expressing a therapeutic amount of the inhibitor of PP1 in the heart tissue may attenuate cardiac remodelling, improve exercise capacity, or improve cardiac contractility. Suitably, expressing a therapeutic amount of the inhibitor of PP1 in the heart tissue may result in myocyte shortening, lowering of the time constant for relaxation, and accelerating calcium signal decay, improving the end-systolic pressure dimension relationship and combinations thereof.

In still another embodiment, before, or after administration of the rAAV vector as disclosed herein, or both, the method further comprises evaluating a parameter of heart function in the subject. The parameter of heart function may, without limitation, be one or more of: heart rate, cardiac metabolism, heart contractility, ventricular function, Ca2+ metabolism, and sarcoplasmic reticulum Ca2+ ATPase activity.

### C. Exemplary Subjects to Be Treated With the rAAV Vector Disclosed Herein

As disclosed herein, the methods of administration and rAAV vectors disclosed herein can be used for treating a cardiovascular condition or heart disease, wherein the rAAV vector as disclosed herein is targeted to the heart of a patient whereby the nucleic acid sequence, e.g., inhibitor of PP1, or other therapeutic agents (e.g., an angiogenic protein or protein from Table 18A-18B) is expressed in the myocardium, thus ameliorating cardiac dysfunction by improving blood flow and/or improving cardiac contractile function. Improved heart function ultimately leads to the reduction or disappearance of one or more symptoms of heart disease or heart failure and prolonged life beyond the expected mortality.

Similarly, In some embodiments, the methods of administration and rAAV vectors disclosed herein can be used for the treatment of peripheral vascular disease, wherein the rAAV vector as disclosed herein is targeted to the heart of a patient whereby the nucleic acid sequence, e.g., inhibitor of PP1, or other therapeutic agents (e.g., an angiogenic protein) is targeted to the affected tissue, for example ischemic skeletal muscle, whereby expression of the therapeutic protein, e.g., inhibitor of PP1 or angiogenic protein ameliorates and/or cures symptoms of the peripheral vascular disease, for example by increasing blood flow to the affected (e.g., ischemic) region of the tissue and/or, in muscle, by improving contractile function of the affected muscle.

Thus, in some aspects, the technology described herein relates to methods of administration and rAAV vectors disclosed herein in a method for treating a cardiovascular condition or heart disease in a patient having myocardial ischemia, comprising administering the rAAV vector according to the administration methods disclosed herein to the myocardium of the patient by intracoronary injection, preferably by injecting the rAAV vector directly into one or both coronary arteries (or grafts), whereby the expression of the transgene (e.g., inhibitor of PP1 and/or angiogenetic protein) is expressed and blood flow and/or contractile function are improved. By way of illustration, a rAAV vector is delivered to the heart where the protein or peptide is produced to a therapeutically significant degree in the myocardium continuously for sustained periods, angiogenesis can be promoted in the affected region of the myocardium.

### Heart Failure:

Subjects amenable to treatment with the rAAV vectors as disclosed herein, and methods of administration as disclosed herein include, but are not limited to, a subject having heart failure, including congestive heart failure (CHF), who has a condition selected from the group consisting of ischemia, arrhythmia, myocardial infarction (MI), abnormal heart contractility, and abnormal Ca2+ metabolism, and combinations thereof, in addition to heart failure. In yet another embodiment, the subject is human. Among such patients suffering from congestive heart failure are those exhibiting dilated cardiomyopathy and those who have exhibited severe myocardial infarctions, typically associated with severe or occlusive coronary artery disease. In certain embodiments, the subjects, to be treated, have non-ischemic cardiomyopathy.

The definition of heart failure from the American College of Cardiology/American Heart Association (ACC/AHA), HFA/ESC, and JHFS guidelines classify heart failure as a clinical syndrome, and each definition is as follows:

### (I) Acute Heart Failure (AHF)

Acute Heart failure (AHF) refers to rapid onset or worsening of symptoms and/or signs of HF. It is a life-threatening medical condition requiring urgent evaluation and treatment, typically leading to urgent hospital admission. AHF may present as a first occurrence (de novo) or, more frequently, as a consequence of acute decompensation of chronic HF, and may be caused by primary cardiac dysfunction or precipitated by extrinsic factors, often in patients with chronic HF. Acute myocardial dysfunction (ischaemic, inflammatory or toxic), acute valve insufficiency or pericardial tamponade are among the most frequent acute primary cardiac causes of AHF. Decompensation of chronic HF can occur without known precipitant factors, but more often with one or more factors, such as infection, uncontrolled hypertension, rhythm disturbances or non-adherence with drugs/diet

People with acute heart failure (AHF) have no previous signs and symptoms of heart failure. AHF can present with rapid swelling and fluid retention characterized by sudden weight gain. Coughing, wheezing, and shortness of breath, as well as an irregular heartbeat, could be symptoms of acute heart failure. In some cases, it is related to pre-existing cardiomyopathy. AHF often requires unexpected hospital admission. It can also be associated with a poor prognosis and a high risk of readmission and death post-discharge. Treatment options include medication, surgery, and implanted medical devices, as well as recommended lifestyle modifications.

In most cases, patients with AHF present with either preserved (90-140 mmHg) or elevated (.140 mmHg; hypertensive AHF) systolic blood pressure (SBP). Only 5 -8% of all patients present with low SBP (i.e.,90 mmHg; hypotensive AHF), which is associated with poor prognosis, particularly when hypoperfusion is also present.

### (II) Chronic Heart Failure (Also Used Interchangeably With Congestive Heart Failure)

Heart failure (HF) is a complex syndrome with many possible causes that result from impaired ability of the left ventricle to either fill with blood during the diastolic phase of the cardiac cycle or eject blood during the systolic phase of the cardiac cycle. The affected heart is consequently increasingly less able to pump a sufficient blood volume to meet the oxygen demands of the body.

Heart failure is a common chronic condition, which predominantly affects the elderly. Prevalence is 0.8-2% in the general population but 10-20% among those aged >70 years. With an ever-aging population, prevalence is increasing. In the US, HF currently affects 5.8 million; one estimate predicts that this will rise to more than 8 million by 2030.

Chronic heart failure (CHF) describes the heart’s inability to pump enough blood through the body and provide a sufficient supply of oxygen - that is, CHF is the inability of the heart to pump the required quantity of the blood to meet the demands of the body. This is caused by a weaker than a normal heart. When the body receives a poor inflow of the blood from the heart, several tissues and organs begin to function below their potential, making it impossible for an individual to possess the needed energy to indulge in regular activities. When the heart cannot fill properly or pump blood forward, it causes fluid to build up into the tissues of the body, resulting in congestion or swelling. The typical symptoms of CHF are shortness of breath and fatigue, however, some people with CHF only experience fatigue and decreased activity tolerance. Those who experience congestion may have swelling in the ankles and legs, abdomen, or lungs. Shortness of breath and pulmonary edema can lead to respiratory distress if not treated promptly. Heart failure can also affect the kidneys’ ability to process and eliminate sodium and water. This may result in even more fluid retention and subsequent swelling. In many cases, the improper heart functioning leads to death, as the heart fails to receive oxygen and blood to the heart muscle.

The heart failure can occur to the left side or the right side.

Left Side Cardiac Failure: Left side cardiac failure occurs when the failure occurs due to improper functioning of the left ventricle. In such cases, the left ventricle fails in pumping with necessary force to pump the blood from the heart, as needed by the body. Due to this, the left chamber accumulates with blood that eventually leads to pulmonary edema and pulmonary hypertension. The causes of the same include hypertension, ischemic heart diseases, aortic valve diseases, and primary myocardial diseases.

Right Side Heart Failure: The primary reason for the failure of right side ventricle is because of left side heart failure. However, if the occurrence of the failure of the right side is due to pathology in the lungs, then it is coronary pulmonale. CHF is most common in men and risk factors include age, high blood pressure, being overweight and the presence of metabolic disorders like diabetes. CHF is as its name describes - it is a long-term condition that can get worse over time. Prior to the current invention, it generally cannot be cured but it can be medically managed.

Ejection fraction: The ejection fraction describes the pumping ability of the heart; a muscle that contracts and relaxes with every beat. The EF measures the percentage of blood pumped out of the heart each time it contracts. With every beat, the heart pumps blood throughout the body. When the pumping ability of the heart is impaired, the ejection fraction measurements decline. The normal range for an EF is 55% to 70%. In some embodiments, the methods as disclosed herein are used for a subject who has a heart failure with a reduced EF (HFrEF).

### Diastolic Heart Failure:

Diastolic HF is also known as heart failure with preserved ejection fraction (HFpEF), and develops when the left ventricle becomes rigid or stiff and cannot relax during diastole, the time between beats. This prevents the heart from properly refilling with blood. Representing about half of all HF cases, diastolic heart failure is most common in older people and in women. It is often present when there are other underlying medical conditions (comorbidities) that can contribute to the development of HF. HFpEF denotes a preserved ejection fraction because although the muscle cannot relax as well as it should, the left ventricle is still pumping normally.

### Systolic Heart Failure:

Systolic HF is also known as heart failure with reduced ejection fraction (HFrEF), and develops when the left ventricle does not contract normally. This means the heart no longer pumps with enough force to squeeze enough blood into circulation. Conditions like high blood pressure, arrhythmias, coronary artery disease, and abuse of alcohol and drugs can contribute to the development of heart failure. HFrEF progresses as the left ventricle, the lower-left chamber of the heart, gets larger and works harder to squeeze pump the right amount of oxygen-rich blood out to fuel the body.

### Decompensated Heart Failure:

Decompensated heart failure (DHF) occurs when patients who have known HF develop worsening signs and symptoms of congestion. This is also called fluid overload, as the body has more fluid than it can get rid of. Patients may have weight gain, worsening dyspnea, swelling or edema in their legs or abdomen, nausea, and are short of breath while lying down. Decompensated heart failure can also cause fatigue, making you feel more tired when doing vigorous or everyday activities. This can interfere with carrying out household activities or any strenuous tasks at work. These patients may need to be admitted to the hospital for treatment.

There are a range of causes for heart failure. They broadly include non-ischemic heart failure and ischemic heart failure. Non-ischemic heart failure can be genetically based, caused by an illness (Kawasaki disease), cardiomyopathy, nutritionally based, such as alcohol induced heart failure, or virus infections (and possibly by Covid-19). It can also be caused by ischemia.

There are several diseases and conditions that can lead a subject to heart failure, including, e.g., hypertension, renal insufficiency, damaged heart tissue (myocardial infarction, including antecedent myocardial infarction (MI), coronary artery disease/ischemia, abnormal heart valves or chambers (congenital or acquired), Pulmonary embolus (PE) and hypertension, heart arrhythmias, cardiomyopathy, diabetes mellitus, age-related deterioration, substance abuse, toxins, Obstructive sleep apnea, and Infections (myocarditis, endocarditis).

Heart imaging (echocardiography) allows measurement of the left ventricular ejection fraction (LVEF). This is the% of the total blood volume in the left ventricle that is ejected during systole and is normally around 50-70%. HF due to impaired ventricular ejection is associated with reduced ejection fraction (rEF), i.e. less than 50% and is referred to as HFrEF or systolic heart failure. HF due to impaired ventricle filling is associated with preserved EF (i.e. >55%) and is referred to as HFpEF or diastolic heart failure. Heart imaging allows detection of left ventricular dysfunction (either systolic or diastolic) before symptoms of heart failure occur. In some embodiments, the patients administered with rAAV as disclosed herein, have 40% LVEF, or 35% LVEF, or 30% LVEF, or 25% LVEF, or 20% LVEF, or 15% LVEF, or 10% LVEF or, 5% LVEF or less LVEF. In some embodiments, the LVEF is measured by Transthoracic echocardiography (TTE).

Previous myocardial infarction (MI) and chronic hypertension (CH) are the two most common causes of HF. Diabetes is associated with increased risk of HF, independent of previous MI or chronic hypertension.

Cardinal symptoms of HF include: breathlessness after only mild exertion (dyspnea); exercise intolerance, fatigue; and eventually ankle swelling/pain due to local fluid (edema) accumulation. HF is a progressively debilitating condition. The New York Heart Association (NYHA) Functional Classification is widely used to classify the severity of HF to one of four classes based on the extent to which physical activity is limited. NYHA Class I is essentially asymptomatic HF, and NYHA Class IV is applied to patients with most severe HF who are “unable to carry on any physical activity without discomfort”. These Class IV HF patients “experience symptoms (breathlessness, fatigue, etc.) at rest.” In some embodiments, the patients administered with rAAV as disclosed herein, have either NYHA class III or, NYHA class IV heart failure.

Typically, patients have periods of chronic stable HF, punctuated by acute exacerbation, called acute (decompensated) heart failure (AHF) when symptoms and hemodynamic condition worsen significantly, requiring emergency admission to hospital. AHF, which may occur in those who have not yet been diagnosed with HF, is associated with high mortality. Around 12-15% of patients hospitalized for AHF die within 12 weeks and 30% die within 12 month of admission.

In some embodiments, the rAAV vectors and methods of administration as disclosed herein is used in a method to treat chronic non-ischemic cardiomyopathy or ischemic cardiomyopathy. In some embodiments, the subject has chronic non-ischemic cardiomyopathy. In some embodiments, subject with chronic ischemic cardiomyopathy are not amenable to treatment. In some embodiments, the subject amenable to treatment if a rAAV vector disclosed herein, administered according to the methods as disclosed herein has LVEF (left ventricle end-diastolic volume) ≤ 30% by transthoracic echocardiography (TTE) within 6 months prior to enrolment. In some embodiments, the subject has an LVEF as follows >120 ventricular volume/EDVI (ml) (severe LV enlargement), or between 100-120 ventricular volume/EDVI (ml) (moderate LV enlargement), or between 84-99 ventricular volume/EDVI (ml) (mild LV enlargement). In some embodiments, if a subject has a ventricular volume/EDVI (ml) of equal or less than 84 (normal LV) the subject is not amenable to treatment with a rAAV vector as disclosed herein as administered according to the disclosed methods.

### (III) Cardiomyopathies

Cardiomyopathy and Congestive Heart Failure (CHF) are extremely common conditions that are responsible for millions of death around the globe. Cardiomyopathy belongs to the heterogeneous group of diseases that cause either mechanical or electrical dysfunction that exhibits inappropriate dilatation. The occurrence is due to several factors, including genetic factors. They are part of multi-system disorder or confined to the heart alone that leads to cardiovascular death. Congestive heart failure, is the inability of the heart to pump the required quantity of the blood to meet the demands of the body. Cardiomyopathy and heart failure or, congestive heart failure are closely integrated with each other; cardiomyopathy is the pathology of heart muscle wheras heart failure is the syndrome that happens when there is cardiomyopathy.

There are Three Types of Cardiomyopathy: (1) Dilated Cardiomyopathy which occurs due to progressive cardiac dilatation with concomitant hypertrophy. Causes include genetic mutations, childbirth, iron overload, myocarditis, and alcohol abuse. (2) Hypertrophic Cardiomyopathy, which occurs due to genes, myocardial hypertrophy, and improper functioning of the left ventricular myocardium. They lead to abnormal diastolic filling and obstruct the intermittent ventricular outflow. (3) Restrictive Cardiomyopathy which is least common and appears due to decrease in ventricular compliance, which results in an impaired ventricular filling. The causes include radiation fibrosis, amyloidosis, metastatic tumors, and sarcoidosis.

In all aspects disclosed herein, the methods encompass treating a subject with heart failure. In some embodiments, the subject has non-ischemic cardiomyopathy. In other embodiments, the subject has ischemic cardiomyopathy.

The term “cardiomyopathy” refers to “cardio” (heart), “myo” (muscle), “pathy” (disease of). A dilated cardiomyopathy results in left ventricular chamber enlargement, systolic dysfunction and clinical manifestations of congestive heart failure.

In some embodiments, the subject with a non-ischemic cardiomyopathy has a dilated cardiomyopathy, where there is dilation and impaired contraction of one or both ventricles.

In some embodiments, a subject has non-ischemic cardiomyopathy due to toxins, e.g., alcohol, drugs (e.g., anthracyclines). In some embodiments, a subject has non-ischemic cardiomyopathy due to an infiltrative agent, e.g., sarcoid, iron overload (haemochromatosis or excess blood transfusions). In some embodiments, a subject has non-ischemic cardiomyopathy due to dietary issues, e.g., beri-beri (thiamine deficiency.

In some embodiments, a subject has non-ischemic cardiomyopathy due to a current or prior infection, such as, but not limited to, viral infection, e.g., Chagas’ disease, HI, coxsackie or Lyme disease, coronaviruses (MERS-CoV (causes MERS), SARS-CoV (causes SARS), SARS-CoV2 (causes COVID-19), and human coronaviruses 229E, NL63, OC43 and HKU1. In some embodiments, a subject with non-ischemic cardiomyopathy has long-Covid or a long term complication or symptom due to a covid infection.

In some embodiments, a subject has non-ischemic cardiomyopathy due to a hereditary condition, e.g., familial DCM, muscular dystrophies (Duchenne, myotonia, mitochondrial). In some embodiments of the methods to treat a subject heart failure as disclosed herein, subject has non-ischemic cardiomyopathy due a genetic disorder with a cardiac manifestation, selected from the group comprising: 22q11.2 deletion syndrome, Abdominal aortic aneurysm, Aberrant subclavian artery, Adult polyglucosan body disease, Alpha-mannosidosis, Alström syndrome, Andersen-Tawil syndrome, Aneurysm of sinus of Valsalva, Arrhythmogenic right ventricular cardiomyopathy, Arterial tortuosity syndrome, Ehlers-Danlos syndrome, Atrial myxoma, familial, Atrial septal defect ostium primum, Atrial septal defect sinus venosus, Baroreflex failure, Barth syndrome, Becker muscular dystrophy, Bidirectional tachycardia, Blue rubber bleb nevus syndrome, Brachydactyly long thumb type, Broken heart syndrome, Brugada syndrome, Brugada syndrome 3, Brugada syndrome 4, Budd-Chiari syndrome, Buerger disease, Cardiac hydatid cysts with intracavitary expansion, Cardiac rupture, Cardiac-Valvular Ehlers-Danlos syndrome, Cardioencephalomyopathy, Cardiofaciocutaneous syndrome, Cardiomyopathy cataract hip spine disease, Cardiomyopathy dilated with woolly hair and keratoderma, Carney complex, Carnitine-acylcarnitine translocase deficiency, Catecholaminergic polymorphic ventricular tachycardia, Chaotic atrial tachycardia, CHARGE syndrome, Chromosome 1p36 deletion syndrome, COG1-CDG (CDG-IIg), COG7-CDG (CDG-IIe), Combined oxidative phosphorylation deficiency 16, Congenital generalized lipodystrophy type 4, Congenital heart block, Congenitally corrected transposition of the great arteries, Cor triatriatum dexter, Cor triatriatum sinister, Costello syndrome, Cystic medial necrosis of aorta, Danon disease, DCMA syndrome, Diffuse cutaneous systemic sclerosis, Dilated cardiomyopathy, Dilated cardiomyopathy with hypergonadotropic hypogonadism,DOLK-CDG (CDG-Im), DPM3-CDG (CDG-Io), Duchenne muscular dystrophy (DMD), Ebstein’s anomaly, Ellis Yale Winter syndrome, Ellis-Van Creveld syndrome, Eosinophilic granulomatosis with polyangiitis, Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibrocartilaginous embolism, Fibromuscular dysplasia, Friedreich ataxia, Fucosidosis, Gaucher disease, Gaucher disease type 1, Glutaric acidemia type II, Glycogen storage disease type 2, Glycogen storage disease type 3, Glycogen storage disease type 4, Heart-hand syndrome, Slovenian type, Heart-hand syndrome, Spanish type, HEC syndrome, His bundle tachycardia, Holt-Oram syndrome, Human HOXA1 Syndromes, Hurler syndrome, Hurler-Scheie syndrome, Hypereosinophilic syndrome, Hypoplastic left heart syndrome, Infantile histiocytoid cardiomyopathy, Intracranial arteriovenous malformation, Isobutyryl-CoA dehydrogenase deficiency, Ivemark syndrome, Jervell Lange-Nielsen syndrome, Kallikrein hypertension, Kawasaki disease, Kearns-Sayre syndrome, LCHAD deficiency, Leber hereditary optic neuropathy, Left ventricular noncompaction, LEOPARD syndrome, Limb-girdle muscular dystrophy type 1B, Limb-girdle muscular dystrophy type 2E, Limb-girdle muscular dystrophy type 2F, Limb-girdle muscular dystrophy type 2M - See Limb-girdle muscular dystrophy, Limb-girdle muscular dystrophy, type 2C, Limb-girdle muscular dystrophy, type 2D, Limited cutaneous systemic sclerosis, Limited systemic sclerosis, Loeys-Dietz syndrome type 2, Loeys-Dietz syndrome type 4, Long QT syndrome 1, Lymphedema and cerebral arteriovenous anomaly, Lymphocytic vasculitis, Mannosidosis, beta A, lysosomal, McLeod neuroacanthocytosis syndrome, Medulloblastoma, MGAT2-CDG (CDG-IIa), Microcephaly-cardiomyopathy; Mitochondrial encephalomyopathy lactic acidosis and stroke-like episodes, Mitochondrial trifunctional protein deficiency, Mitral atresia, Mitral valve prolapse, familial, autosomal dominant, Musculocontractural Ehlers-Danlos syndrome, Myoclonic epilepsy with ragged red fibers, Myotonic dystrophy type 1, Nathalie syndrome, Naxos disease, Neonatal stroke, Neurofibromatosis-Noonan syndrome, Noonan syndrome, Noonan syndromes 1 -, 2-, 3-, 4-, 5- and 6, Noonan-like syndrome with loose anagen hair, Ostium secundum atrial septal defect, Paroxysmal ventricular fibrillation, Patent ductus arteriosus, Patent ductus venosus, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block type 1A, Progressive familial heart block type 1B, Progressive familial heart block type 2, Pseudohypoaldosteronism type 2, Pseudoxanthoma elasticum, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis,Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency, White forelock with malformations, and Williams syndrome.

### (Iv) Cardiovascular Condition and Cardiovascular Diseases in General:

In some embodiments, the rAAV vectors and methods of administration as disclosed herein are also provided for use in peripheral vascular diseases such as peripheral arterial occlusive disease (PAOD). As described and illustrated herein, these methods are thus useful for treating a cardiovascular condition, heart disease, peripheral vascular disease and similar disorders.

In some embodiments, the rAAV vectors and methods of administration as disclosed herein are useful in methods to treat dilated cardiomyopathy (DCM), a type of heart failure that is typically diagnosed by the finding of a dilated, hypocontractile left and/or right ventricle. DCM can occur in the absence of other characteristic forms of cardiac disease such as coronary occlusion or a history of myocardial infarction. DCM is associated with poor ventricular function and symptoms of heart failure. In these patients, chamber dilation and wall thinning generally results in a high left ventricular wall tension. Many patients exhibit symptoms even under mild exertion or at rest, and are thus characterized as exhibiting severe, i.e. “Type-III” or “Type-IV”, heart failure, respectively (see, e.g., NYHA classification of heart failure). As noted above, many patients with coronary artery disease may progress to exhibiting dilated cardiomyopathy, often as a result of one or more heart attacks (myocardial infarctions).

In some embodiments, the rAAV vectors and methods of administration as disclosed herein are useful in a method to prevent, inhibit, slow the progression, or at least lessen deleterious left ventricular remodeling (a.k.a., deleterious remodeling, for short), which refers to chamber dilation after myocardial infarction that can progress to severe heart failure. Even if ventricular remodeling has already initiated, it is still desirable to promote an increase in blood flow, as this can still be effective to offset ventricular dysfunction. Similarly, promotion of angiogenesis can be useful, since the development of a microvascular bed can also be effective to offset ventricular dysfunction. Further, such rAAV vectors and methods of administration as disclosed herein can also have other enhancing effects. In a patient who has suffered a myocardial infarction, deleterious ventricular remodeling is prevented if the patient lacks chamber dilation and if symptoms of heart failure do not develop. Deleterious ventricular remodeling is alleviated if there is any observable or measurable reduction in an existing symptom of the heart failure. For example, the patient may show less breathlessness and improved exercise tolerance. Methods of assessing improvement in heart function and reduction of symptoms are essentially analogous to those described above for DCM. Prevention or alleviation of deleterious ventricular remodeling as a result of improved collateral blood flow and ventricular function and/or other mechanisms is expected to be achieved within weeks after in vivo angiogenic gene transfer in the patient using methods as described herein.

In another example, the rAAV vectors and methods of administration as disclosed herein transfer of a transgene encoding an inhibitor of PP1, an angiogenic protein or a therapeutic protein selected from Table 18A-18B, is used to treat conditions associated with congestive heart failure (CHF).

In one embodiment, the disease may be cardiovascular condition or heart disease and disorders. In one embodiment, the disease may be heart failure such as congestive heart failure. In one embodiment, the subject may have non-ischemic cardiomyopathy.

In some embodiments, the disease may be selected from congestive heart failure, coronary artery disease, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease.

In one embodiment, the disease may be selected from arrhythmia, abnormal heart contractility, non-ischemic cardiomyopathy, peripheral arterial occlusive disease, and abnormal Ca2+ metabolism, and combinations thereof. In some embodiments, the disease may be selected from the group of: congestive heart failure, cardiomyopathy, vascular disease, acquired heart disease, congenital heart disease, atherosclerosis, dysfunctional conduction systems, dysfunctional coronary arteries, pulmonary heart hypertension.

In some embodiments, the muscular disease is a vascular disease. Vascular disease may be coronary artery disease, peripheral arterial disease, cerebrovascular disease, renal artery stenosis or aortic aneurysm. In some embodiments, the muscular disease may be cardiomyopathy. The cardiomyopathy may be hypertensive heart disease, heart failure (such as congestive heart failure), pulmonary heart disease, cardiac dysrhythmias, inflammatory heart disease (such as endocarditis, inflammatory cardiomegaly, myocarditis), valvular heart disease, congenital heart disease and rheumatic heart disease.

In some embodiments, the cardiomyopathy is hypertrophic cardiomyopathy, arrhythmogenic right ventricular dysplasia, dilated cardiomyopathy, restrictive cardiomyopathy, left ventricular noncompaction, Takotsubo cardiomyopathy, myocarditis and eosinophilic myocarditis. Preferably, the hypertrophic cardiomyopathy is CMH1 (Gene: MYH7), CMH2 (Gene: TNNT2), CMH3 (Gene: TPM1), CMH4 (Gene: MYBPC3), CMH5, CMH6 (Gene: PRKAG2), CMH7 (Gene: TNNI3), CMH8 (Gene: MYL3), CMH9 (Gene: TTN), CMH10 (Gene: MYL2), CMH11 (Gene: ACTC1), or CMH12 (Gene: CSRP3). Preferably, the arrhythmogenic right ventricular dysplasia is ARVD1 (Gene: TGFB3), ARVD2 (Gene: RYR2), ARVD3, ARVD4, ARVD5 (Gene: TMEM43), ARVD6, ARVD7 (Gene: DES), ARVD8 (Gene: DSP), ARVD9 (Gene: PKP2), ARVD10 (Gene: DSG2), ARVD11 (Gene: DSC2), and/or ARVD12 (Gene: JUP).

In another example, the rAAV vectors and methods of administration as disclosed herein can be used for the treatment of any of: congestive heart failure, non-ischemic cardiomyopathy, vascular disease, acquired heart disease, congenital heart disease, atherosclerosis, dysfunctional conduction systems, dysfunctional coronary arteries, pulmonary heart hypertension. In some embodiments, the disease is selected from the group consisting of congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease.

Heart failure, also called congestive heart failure (CHF), is a disorder in which the contractility of the heart muscle decreases, and the heart loses its ability to pump blood efficiently. It is estimated to affect over 10 million Americans, alone. Heart failure is almost always a chronic, long-term condition, and consumes an inordinate amount of medical intervention and human resource dollars. In particular, the consequences of heart failure to the rest of the body organs can be devastating both in terms of the overall reduction in productive life of the patient, and the expense of treatment. The condition may affect the right side, the left side, or both sides of the heart. As the pumping action of the heart is compromised, blood begins backing up into other areas of the body. Many organs and organ systems begin to suffer cumulative damage from lack of oxygen and nutrients.

There may be many underlying causes, and heart failure becomes more common with advancing age. Problematically, some patients with heart failure have no obviously noticeable symptoms, permitting serious peripheral conditions to manifest without the benefit of early intervention to ward off or abate the rate of serious organ damage.

### (IV) Ischemic Cardiomyopathy

In some embodiments, the rAAV vectors and methods of administration as disclosed herein is used in a method for substantially reducing myocardial ischemia.

In one example, the rAAV vectors and methods of administration as disclosed herein is used in a method for substantially reducing myocardial ischemia.

In one embodiment, the disease may be selected from ischemia, myocardial infarction (MI), ischemic cardiomyopathy and combinations thereof. In some embodiments, the disease may be selected from the group of: infarction, tissue ischemia, cardiac ischemia, atherosclerosis or CAD.

Myocardial ischemia is an aspect of heart dysfunction that occurs when the heart muscle (the myocardium) does not receive adequate blood supply and is thus deprived of necessary levels of oxygen and nutrients. Myocardial ischemia may result in a variety of heart diseases including, for example, angina, heart attack and/or congestive heart failure. The most common cause of myocardial ischemia is atherosclerosis (also referred to as coronary artery disease or “CAD”), which causes blockages in the coronary arteries, blood vessels that provide blood flow to the heart muscle. Present treatments for myocardial ischemia include pharmacological therapies, coronary artery bypass surgery and percutaneous revascularization using techniques such as balloon angioplasty. Standard pharmacological therapy is predicated on strategies that involve either increasing blood supply to the heart muscle or decreasing the demand of the heart muscle for oxygen and nutrients. For example, increased blood supply to the myocardium can be achieved by agents such as calcium channel blockers or nitroglycerin. These agents are thought to increase the diameter of diseased arteries by causing relaxation of the smooth muscle in the arterial walls. Decreased demand of the heart muscle for oxygen and nutrients can be accomplished either by agents that decrease the hemodynamic load on the heart, such as arterial vasodilators, or those that decrease the contractile response of the heart to a given hemodynamic load, such as beta-adrenergic receptor antagonists. Surgical treatment of ischemic heart disease is generally based on the bypass of diseased arterial segments with strategically placed bypass grafts (usually saphenous vein or internal mammary artery grafts). Percutaneous revascularization is generally based on the use of catheters to reduce the narrowing in diseased coronary arteries. In some embodiments, the patients to be treated with rAAV as disclosed herein, are co-administered with nitroglycerin or, nitroprusside.

Many patients with heart disease, including many of those whose severe myocardial ischemia resulted in a heart attack, are diagnosed as having congestive heart failure. Congestive heart failure is defined as abnormal heart function resulting in inadequate cardiac output to meet metabolic needs (Braunwald, E. (ed), In: Heart Disease, W. B. Saunders, Philadelphia, page 426, 1988). An estimated 5 million people in the United States suffer from congestive heart failure. Once symptoms of CHF are moderately severe, the prognosis is worse than most cancers in that only half of such patients are expected to survive for more than 2 years (Braunwald, E. (ed), In: Heart Disease, W. B. Saunders, Philadelphia, page 471-485, 1988). Medical therapy can initially attenuate the symptoms of CHF (e.g., edema, exercise intolerance and breathlessness), and in some cases prolong life. However, the prognosis for this disease, even with medical treatment, remains grim, and the incidence of CHF has been increasing (see, e.g., Baughman, K., Cardiology Clinics 13: 27-34, 1995). Symptoms of CHF include breathlessness, fatigue, weakness, leg swelling and exercise intolerance. On physical examination, patients with heart failure tend to have elevations in heart and respiratory rates, rates (an indication of fluid in the lungs), edema, jugular venous distension, and, in general, enlarged hearts. The most common cause of CHF is atherosclerosis which, as discussed above, causes blockages in the coronary arteries that supply blood to the heart muscle. Thus, congestive heart failure is most commonly associated with coronary artery disease that is so severe in scope or abruptness that it results in the development of chronic or acute heart failure. In such patients, extensive and/or abrupt occlusion of one or more coronary arteries precludes adequate blood flow to the myocardium, resulting in severe ischemia and, in some cases, myocardial infarction or death of heart muscle. The consequent myocardial necrosis tends to be followed by progressive chronic heart failure or an acute low output state - both of which are associated with high mortality.

Subjects amenable to treatment by the methods as disclosed herein can be identified by any method to diagnose myocardial infarction (commonly referred to as a heart attack). Methods of diagnosing these conditions are well known by persons of ordinary skill in the art. By way of non-limiting example, myocardial infarction can be diagnosed by (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

Most patients with congestive heart failure tend to develop enlarged, poorly contracting hearts, a condition referred to as “dilated cardiomyopathy” (or DCM, as used herein). DCM is a condition of the heart typically diagnosed by the finding of a dilated, hypocontractile left and/or right ventricle. Again, in the majority of cases, the congestive heart failure associated with a dilated heart is the result of coronary artery disease, often so severe that it has caused one or more myocardial infarcts. In a significant minority of cases, however, DCM can occur in the absence of characteristics of coronary artery disease (e.g., atherosclerosis). In a number of cases in which the dilated cardiomyopathy is not associated with CAD, the cause of DCM is known or suspected. Examples include familial cardiomyopathy (such as that associated with progressive muscular dystrophy, myotonic muscular dystrophy, Freidrich’s ataxia, and hereditary dilated cardiomyopathy), infections resulting in myocardial inflammation (such as infections by various viruses, bacteria and other parasites), noninfectious inflammations (such as those due to autoimmune diseases, peripartum cardiomyopathy, hypersensitivity reactions or transplantation rejections), metabolic disturbances causing myocarditis (including nutritional, endocrinologic and electrolyte abnormalities) and exposure to toxic agents causing myocarditis (including alcohol, as well as certain chemotherapeutic drugs and catecholamines). In the majority of non-CAD DCM cases, however, the cause of disease remains unknown and the condition is thus referred to as “idiopathic dilated cardiomyopathy” (or “IDCM”). Despite the potential differences in underlying causation, most patients with severe CHF have enlarged, thin-walled hearts (i.e., DCM) and most of those patients exhibit myocardial ischemia (even though some of them may not have apparent atherosclerosis). Furthermore, patients with DCM can experience angina pectoris even though they may not have severe coronary artery disease.

Further complicating the physiological conditions associated with CHF are various natural adaptations that tend to occur in patients with dysfunctional hearts. Although these natural responses can initially improve heart function, they often result in other problems that can exacerbate the disease, confound treatment, and have adverse effects on survival. There are three such adaptive responses commonly observed in CHF patients: (i) volume retention induced by changes in sodium reabsorption, which expands plasma volume and initially improves cardiac output; (ii) cardiac enlargement (from dilation and hypertrophy) which can increase stroke volume while maintaining a relatively normal wall tension; and (iii) increased norepinephrine release from adrenergic nerve terminals impinging on the heart which, by interacting with cardiac beta-adrenergic receptors, tends to increase heart rate and force of contraction, thereby increasing cardiac output. However, each of these three natural adaptations tends ultimately to fail for various reasons. In particular, fluid retention tends to result in edema and retained fluid in the lungs that impairs breathing. Heart enlargement can lead to deleterious left ventricular remodeling with subsequent severe dilation and increased wall tension, thus exacerbating CHF. Finally, long-term exposure of the heart to norepinephrine tends to make the heart unresponsive to adrenergic stimulation and is linked with poor prognosis.

Diseases of the peripheral vasculature, like heart disease, often result from restricted blood flow to the tissue (e.g. skeletal muscle) which (like cardiac disease) becomes ischemic, particularly when metabolic needs increase (such as with exercise). Thus, atherosclerosis present in a peripheral vessel may cause ischemia in the tissue supplied by the affected vessel. This problem, known as peripheral arterial occlusive disease (PAOD), most frequently affects in the lower limbs of patients. As with other forms of cardiovascular disease, this condition or at least some of its symptoms, may be treated by using drugs, such as aspirin or other agents that reduce blood viscosity, or by surgical intervention, such as arterial grafting, surgical removal of fatty plaque deposits or by endovascular treatments, such as angioplasty. While symptoms may be improved, the effectiveness of such treatments is typically inadequate, for reasons similar to those referred to above.

### II. Methods of Administration

One aspect of the technology described herein relates to a method to administer a rAAV vector, where the method is a single administration of a total dose of a rAAV to the subject, where the single administration comprises delivery of a total dose of rAAV that is divided into at least 2, or 3, or 4, or 5 or more sub-doses within the single administration. That is - stated differently, in some embodiments, the method comprises administering a bolus of rAAV vector to the subject in a single administration, where the single administration of the bolus comprises the administration of rAAV from least 2, or 3, or 4, or 5 doses, and in some embodiments, the doses can be from 2, 3, 4, 5, or 6 or more vials or syringes, where the delivery of the rAAV from each vial or syringe takes between 1-5 minutes, or more than 5 minutes. In other aspects of the technology describe herein relates to a method to administer a rAAV vector, where the method comprises at least one administration or, more than one administration of rAAV to the subject. In some aspects of the embodiment, the method to administer rAAV vector comprises two administrations, three administrations, four administrations, or, five administrations of rAAV to the subject, where each administration comprises delivery of a total dose of rAAV that is divided into at least 2, or, 3, or, 4, or, 5 or, more sub doses.

In some embodiments, the method to administer AAV vectors is a single injection that comprises within the single injection, discrete pulses of delivery of the AAV vector. That is, in a single injection administration, the rAAV delivery is divided into a number of temporally spaced sub-administrations. For example, a single administration can be a total amount (or total dose, also referred to as “TD”) of rAAV that is divided into at least 2, or at least 3, or at least 4, or at least 5 or more sub-doses (“SD”), where each sub-dose is administered in a sub-administration, where each sub-administration is temporally spaced by a pre-defined period of time, e.g., at least 1, or at least 2, or at least 3, or at least 4, or at least 5, or at least 6, or at least 7, or at least 8, or at least 9, or at least 10 or more than 10 minutes between each sub-administration of each sub-dose. For example, a single administration of a total dose of rAAV can include a series of pulses of sub-doses, and each sub-dose is injected in a sub-administration (i.e., pulses of a single administration).

Without being limited to theory, an exemplary method of administration comprises administration of a single administration of a total dose (TD) of rAAV vector is between about 10¹³vg to about 10¹⁵vg, which can be divided into at least 2, or at least 3, or at least 4, or at least 5 or more sub-doses (SD), wherein the sub-doses are administered to the subject spaced at least 5 seconds, or at least 10 seconds, or at least 20 seconds, or at least 30 seconds, or at least 40 seconds, or at least 50 seconds or at least 1 minute, or at least 2, or at least 3, or at least 4, or at least 5, or at least 6, or at least 7, or at least 8, or at least 9, or at least 10 or more than 10 minutes apart, wherein the administration of all the sub-doses for the total rAAV dose takes between about 10 minutes to about 30 minutes, or between about 10 minutes to about 20 minutes, or between about 15 minutes to about 25 minutes, or between about 15 minutes to about 30 minutes, or, between about 25 minutes to about 30 minutes, or, between about 20 minutes to about 40 minutes, or between about 40 minutes to about 60 minutes, or more than 60 minutes, and wherein each subdose is administered over a period of time of 1 minute, or about 2 minutes, or, about 3 minutes or, about 4 minutes, or about 5 minutes, or about 6 minutes, or about 7 minutes or, about 8 minutes or about 9 minutes, or about 10 minutes or longer. In some embodiments, wherein the total rAAV dose administration is performed for about 10 minutes, or, about 15 minutes, or about 20 minutes, or about 25 minutes or about 30 minutes or about 35 minutes or, about 40 minutes or, about 45 minutes, or about 50 minutes, or about 60 minutes or longer. In certain embodiments, the total rAAV dose administration is performed for about 20 minutes to about 30 minutes. In certain aspects of the embodiments, the rAAV is selected from the group consisting of AAV2, AAV6, AAV8, AAV9, AAV2i8, rh10, AAV2.5 and AAV2G9. In some aspects of the embodiments, the rAAV administration is performed for one to five minutes in each of total five subdoses, e.g. five syringes, wherein each subdose has 8 ml, or, 9 ml, or, 10 ml, or, 12 ml, or, 15 ml, or, 20 ml, or 25 ml or more volume of diluent. In certain aspects of the embodiments, the total volume of rAAV administration is 20 ml, or 25 ml, or 30 ml, or, 35 ml, or 40 ml, or 45 ml, or 50 ml, or 60 ml, or 70 ml, or 80 ml, or 90 ml, or 100 ml or, more. Without limiting to any theory, the diluent can be saline, or different ratios of saline-blood mixture. In some aspects of the embodiments, the rAAV administered comprises a nucleic acid encoding phosphatase inhibitor protein e.g., I-1 or a variant thereof such as I-1c, and a promoter selected from CMV promoter or a synthetic promoter selected from Table 18A or 18B or a variant thereof. In all aspects of the embodiments, the rAAV comprises self-complimentary (sc) genome.

In some embodiments, where a single administration of a total dose (TD) of rAAV vector is divided into at least 2, or at least 3, or at least 4, or at least 5 or more sub-doses (SD), each sub-dose can be administered or injected into the subject over a pre-defined time period, e.g., at least 1, or at least 2, or at least 3, or at least 4, or at least 5, or at least 6, or at least 7, or at least 8, or at least 9, or at least 10 or more than 10 minutes, and wherein there is an interval of at least 1, or at least 2, or at least 3, or at least 4, or at least 5, or at least 6, or at least 7, or at least 8, or at least 9, or at least 10 or more than 10 minutes between administration of each sub-dose. In some embodiments, each sub-dose is administered over a period of 1-5 minutes. In some embodiments, the time interval between the sub-doses can be consistent, e.g., same time in-between each sub-dose, or can vary. For example, in an exemplary administration method where the total dose (TD) of a rAAV vector is divided into 5 sub-doses (sd1, sd2, sd3, sd4, sd5), the interval between administration of sd1 and sd2 can be, e.g., at least 2 minutes, and the interval between administration of sd3 after sd2 can be, e.g., 5 minutes.

In some embodiments, the sub-doses of rAAV is by a bolus or in separate vials or separate syringes.

In some embodiments, the single administration of the rAAV vector is co-administered with an additional agent, or therapeutic agent. In some embodiments, the additional agent is an immune modulator as disclosed herein. In some embodiments, the additional agent is administered before, or after, or both (before and after) the single injection of the complete rAAV dose. In some embodiments, the additional agent (e.g., immune modulator) is administered to the subject in the intervals between sub-doses of the rAAV, that is - for example, in an exemplary administration method where the total dose (TD) of a rAAV vector is divided into 5 sub-doses (sd1, sd2, sd3, sd4, sd5), the additional agent, e.g., immune modulator can be administered between any one or more of: between sd1 and sd2, between sd2 and sd3, between sd3 and sd4, between sd4 and sd5. In some embodiments, the additional agent, e.g., immune modulator is present in the sub-doses of rAAV.

In some embodiments, the total-dose of the rAAV is selected from any of: about 10¹¹ vg, about 3×10¹¹ vg, about 5×10¹¹ vg, about 10¹² vg, about 3×10¹² vg, about 5×10¹² vg, about 10¹³ vg, about 3×10¹³ vg, about 10¹⁴ vg, 3×10¹⁴ vg, or, about 10¹⁵ vg, or more than about 10¹⁵ vg. In some embodiments, total-dose of the rAAV is between about 10¹³ vg to about 10¹⁵ vg. In some embodiments, at least one, or at least two or at least three or more total doses of rAAV is administered. In some embodiments, each subdose of rAAV is between about 10¹¹ vg to about 10¹⁵ vg. In certain embodiments, each subdose of rAAV is between about 10¹³ vg to about 10¹⁵ vg. In one embodiment, each subdose of the rAAV is selected from any of about 10¹¹ vg, about 3×10¹¹ vg, about 5× 10¹¹ vg, about 10¹² vg, about 3×10¹² vg, about 5×10¹² vg, about 10¹³ vg, about 3×10¹³ vg, about 10¹⁴ vg, 3×10¹⁴ vg, or, about 10¹⁵ vg, or more than about 10¹⁵ vg.

In some embodiments, the rAAV administration is performed for one to five minutes in each of total five subdoses e.g., in each of total five syringes, wherein each subdose has 10 ml volume e.g., saline. In certain embodiments, the rAAV administration is performed for one to five minutes in each of total five syringes, wherein each syringe has 8 ml, or, 9 ml, or, 10 ml, or, 12 ml, or, 15 ml, or, 20 ml, or 25 ml or more volume of diluent. The diluent is saline or, different ratios of saline-blood mixture. In certain embodiments, the total volume of rAAV administration is 20 ml, or 25 ml, or 30 ml, or, 35 ml, or 40 ml, or 45 ml, or 50 ml, or 60 ml, or 70 ml, or 80 ml, or 90 ml, or 100 ml or, more. In some embodiments, the rAAV administration is performed with 1 syringe, or 2 syringes, or 3 syringes, or 4 syringes, or 5 syringes, or 6 syringes, or 7 syringes, or 8 syringes or more syringes. The rAAV subdose in each syringe is administered over a period of time of 1 minute, or 2 minutes, or 3 minutes, or 4 minutes, or 5 minutes, or 6 minutes, or 7 minutes, or 8 minutes, or 9 minutes, or 10 minutes or, longer. In some embodiments, the method comprises administering the rAAV vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections. In one embodiment, the gene therapy vector may be delivered by injection into the cardiac tissue.

In some embodiments, administration of AAV vector or virion comprising the synthetic cardiac-specific promoter or expression cassette according to this invention is intravascular. Suitably, the AAV vector or virion comprising the synthetic cardiac-specific promoter or expression cassette according to this invention may be administered in the veins of the dorsal hand or the veins of the anterior forearm. Suitable veins in the anterior forearm are the cephalic, median or basilic veins. This is because this administration route is generally safe for the patient.

In some embodiments, the rAAV vector is directly injected into heart tissue. U.S. Ser. No. 10/914,829 describes a protocol for direct injection. Direct injection or application of a viral vector into the myocardium can restrict expression of the transferred genes to the heart (Gutzman et al, 1993, Cric. Res. 73: 1202-7; French et al., 1994, Circulation. 90:2414-24).

In some embodiments, the rAAV vector is introduced into the lumen of one or more coronary arteries. Passage of blood out of the coronary arteries can be restricted. The preparation comprising rAAV vectors can be delivered antegrade and allowed to reside in the arteries for between one to five minutes, e.g., between one to three minutes. Non-viral vehicles may be delivered by similar methods.

In some embodiments, the rAAV vector can be administered to a subject by standard methods. For example, the agent can be administered by any of a number of different routes including intravenous (systemic), intradermal, subcutaneous, oral (e.g., inhalation or ingestion), transdermal (topical), transmucosal or by catheter or, by syringes, or, by a combination of catheter and syringe. In one embodiment, the agent is administered by injection, e.g., intra-arterially, intramuscularly, or intravenously.

In some embodiments, flow of blood through coronary vessels of the heart of the subject is restricted, and the rAAV vector as disclosed herein is introduced into the lumen of a coronary artery in the subject. In yet another embodiment, the heart is pumping while coronary vein outflow is restricted. In yet another embodiment, flow of blood through the coronary vessels is completely restricted. The restricted coronary vessels may comprise, without limitation: the left anterior descending artery (LAD), the distal circumflex artery (LCX), the great coronary vein (GCV), the middle cardiac vein (MCV), or the anterior interventricular vein (AIV). In yet another embodiment, the introduction of the rAAV vector as disclosed herein occurs after ischemic preconditioning of the coronary vessels. In still another embodiment, the and the rAAV vector as disclosed herein is injected into the heart of the subject while aortic flow of blood out of the heart is restricted, thereby allowing the nucleic acid molecule to flow into the heart.

In some embodiments of a method according to the invention, the administering or the rAAV vector as disclosed herein comprises the steps of: restricting aortic flow of blood out of the heart, such that blood flow is re-directed to coronary arteries; injecting the nucleic acid molecule into the lumen of the heart, aorta, or coronary ostia to provide the nucleic acid molecule to a coronary artery; pumping the heart while the aortic flow of blood out of the heart is restricted; and reestablishing the aortic flow of blood. In yet another embodiment, the rAAV vector as disclosed herein is injected into the heart with a catheter. In still another embodiment, the rAAV vector as disclosed herein is directly injected into a muscle of the heart.

In some embodiments, a rAAV vector as disclosed herein can be injected into an affected vessel, e.g., an artery, or an organ, e.g., the heart. In one method of treatment embodiment, flow of blood through coronary vessels of a heart is restricted and a rAAV vector as disclosed herein is introduced into the lumen of a coronary artery. In a specific embodiment, the heart is permitted to pump while coronary vein outflow is restricted. In another specific embodiment, a rAAV vector as disclosed herein is injected into the heart while restricting aortic flow of blood out of the heart, thereby allowing the viral delivery system to flow in to and be delivered to the heart. In other embodiments, the flow of blood through the coronary vessels is completely restricted, and in specific such embodiments, the restricted coronary vessels comprise: the left anterior descending artery (LAD, the distal circumflex artery (LCX), the great coronary vein (GCV), the middle cardiac vein (MCV), or the anterior interventricular vein (AIV). In certain embodiments, the introduction of a rAAV vector as disclosed herein occurs after ischemic preconditioning of the coronary vessels.

In some embodiments, a rAAV vector as disclosed herein injected into the heart by a method comprising the steps of: restricting aortic flow of blood out of the heart, such that blood flow is re-directed to coronary arteries; injecting the vector into lumen of the heart, aorta or coronary ostia such that the vector flows into the coronary arteries; permitting the heart to pump while the aortic flow of blood out of the heart is restricted; and reestablishing the aortic flow of blood. In a more specific embodiment, a rAAV vector as disclosed herein is injected into the heart with a catheter, and in an even more specific embodiment, a rAAV vector as disclosed herein is directly injected into a muscle of the heart.

In some embodiments, the method of delivery comprises restricting blood flow to one or more of the great coronary vein (GCV), the middle cardiac vein (MCV), or the anterior interventricular vein (AIV). In some embodiments, the rAAV vector as disclosed herein is introduced into the lumen of the coronary artery after ischemic preconditioning of the left anterior descending artery (LAD) and/or the distal circumflex artery (LCX). In some embodiments, the rAAV vector as disclosed herein is introduced into the lumen of the coronary artery with a catheter, e.g., the distal circumflex artery (LCX), or a coronary vessel, e.g., a left anterior descending artery (LAD). In some embodiments, the coronary artery is the left anterior descending artery (LAD) or the distal circumflex artery (LCX).

In some embodiments, the AAV vector or virion disclosed herein may be administered concurrently or sequentially with one or more additional therapeutic agents or with one or more saturating agents designed to prevent clearance of the vectors by the reticular endothelial system, e.g., can be administered with one or more immune modulators as disclosed herein.

In some embodiments, where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg. In some embodiments, the amount of the viral vector is between 1×10¹¹ and 1×10¹⁶ plaque forming units (pfu).

In general, the subject in need thereof will be a mammal, and preferably a primate, more preferably a human. Typically, the subject in need thereof will display symptoms characteristic of a cardiovascular condition, e.g., heart disease or heart failure. The method typically comprises ameliorating the symptoms displayed by the subject in need thereof, by expressing the therapeutic amount of the therapeutic product.

The present invention also provides a method of gene therapy of a subject, preferably a human, in need thereof, the method comprising: administering to the subject (suitably introducing into the heart of the subject) a synthetic cardiac-specific expression cassette, vector, virion or pharmaceutical composition of the present invention, which comprises a gene encoding an inhibitor of the PP1, or an angiogenic protein or peptide or any protein disclosed in Tables 4A-4B herein.

The method suitably comprises expressing a therapeutic amount of the inhibitor of PP1 in the heart tissue of said subject. Various conditions and diseases that can be treated are discussed herein. Genes encoding suitable therapeutic products are discussed herein and include but not limited to those disclosed in Tables 4A-4B.

Gene therapy protocols for therapeutic gene expression in target cells in vitro and in vivo, are well-known in the art and will not be discussed in detail here. Briefly, they include intravenous or intraarterial administration (e.g. intra-corotid artery, intra-hepatic artery, intra-hepatic vein), intracerebroventricular, intracranial administration, intramuscular injection, interstitial injection, instillation in airways, application to endothelium and intra-hepatic parenchyme, of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices and the like. Gene transfer into mammalian heart cells can been performed using both ex vivo and in vivo procedures. The ex vivo approach typically requires harvesting of heart cells (e.g., cardiomyocytes), in vitro transduction with suitable expression vectors, followed by reintroduction of the transduced cardiomyocytes into the heart. This approach is generally less preferred due to the difficulty and danger of harvesting and reintroducing cardiomyocytes in the heart. In vivo gene transfer has been achieved by injecting DNA or viral vectors directly into the heart, e.g. by intracranial injection, or by intravenous or intraarterial injection of viral vectors.

In one embodiment, the gene therapy vector may be administered to a subject (e.g., to the heart of a subject) in a therapeutically effective amount to reduce the symptoms of heart failure or a heart disorder of a subject (e.g., determined using a known evaluation method).

### Repeat Administration

In some aspects of the present invention, the use of cardiac specific synthetic promoters or, skeletal muscle specific synthetic promoters active in cardiac cells as listed in Tables 2A, 5A, or 13A provide activity in cardiac cells along with liver detargeting effect.

In some embodiments, the invention provides a method for repeat dosing comprising two administrations, wherein, the repeat dosing comprises one administration of a liver detargeting AAV virion with activity in cardiac cells, and other administration of any other AAV virion that is not used in prior administration, wherein, in one administration, the AAV virion comprises a nucleic acid encoding phosphatase inhibitor (I-1), wherein the nucleic acid operatively linked to a promoter selected from the group of CMV, CK7, myosin, CBA, CK8.intron, JET promoter, or the like, wherein, in the other administration, the AAV virion comprises a nucleic acid encoding phosphatase inhibitor, wherein the nucleic acid operatively linked to a cardiac specific promoter selected from the Table 2A or, variant thereof, or a muscle-specific promoter active in cardiac and skeletal muscle selected from Table 5A or a variant thereof, or a shortened muscle-specific promoter active in cardiac and skeletal muscle selected from Table 13A or a variant thereof, and wherein, in one administration, the AAV virion comprises an AAV capsid that is different than prior administration. In some embodiments, the I-1 comprises amino acids 1-65 of SEQ ID NO: 1 or a functional fragment thereof, wherein the threonine at position 35 of SEQ ID NO: 1 is replaced with an aspartate acid (T35D). In some embodiments, the nucleic acid encoding phosphatase inhibitor encodes a constitutively active fragment of l-1 (I-1c) comprising a fragment of SEQ ID NO: 1, wherein the fragment is selected from: amino acids 1-54 of SEQ ID NO: 1, 1-61 of SEQ ID NO: 1, 1-65 of SEQ ID NO: 1, 1 -66 of SEQ ID NO: 1, l-67 of SEQ ID NO: 1 or 1 -77 of SEQ ID NO: 1, or a functional variant thereof, wherein the threonine at position 35 of SEQ ID NO: 1 is replaced with an aspartate acid (T35D). In some embodiments, the nucleic acid sequence encoding a polypeptide comprises at least amino acids 1-54 of SEQ ID NO: 1, wherein threonine at amino acid 35 of SEQ ID NO: 1 is replaced with an aspartic acid.

Without being construed to any limitation, an example of a method of repeat dosing (administration) comprises a first and a second administration, wherein in the first administration,, AAV2i8 (or, BNP 116) vector comprising nucleic acid encoding phosphatase inhibitor (I-1) polypeptide as described herein is used to administer subjects having congestive heart failure, and wherein the nucleic acid is operatively linked with CMV promoter; in the second administration, recombinant AAV2/9 vector comprising nucleic acid encoding phosphatase inhibitor polypeptide (I-1) as described herein is used to administer said subjects, and wherein the nucleic acid is operatively linked with synthetic promoters selected from the group of SP0173, SP0320, SP0279, SP0134, SP0057, SP0229, SP0067, SP0310, SP0311, SP0267, or a variant thereof.

In some embodiments, rAAV vector comprising cardiac specific promoter selected from the Table 2A or, variant thereof, or a muscle-specific promoter active in cardiac and skeletal muscle selected from Table 5A or a variant thereof or a shortened muscle-specific promoter active in cardiac and skeletal muscle selected from Table 13A, or a variant thereof allow efficacious repeat administration of rAAV vector to treat heart disease (e.g., congestive heart failure) using a rAAV virion with any AAV capsid that is different than the prior administrations, and wherein the rAAV comprises a nucleic acid encoding phosphatase inhibitor (I-1) that is operatively linked to said promoter. In some embodiments, the rAAV comprising cardiac specific promoter selected from the Table 2A or, variant thereof, or a muscle-specific promoter active in cardiac and skeletal muscle selected from Table 5A or a variant thereof, or a shortened muscle-specific promoter active in cardiac and skeletal muscle selected from Table 13A, or a variant thereof allow efficacious repeat administration of rAAV vector to treat heart disease (e.g., congestive heart failure) using a rAAV virion with any AAV capsid that is different than the prior administrations, wherein the rAAV comprises a nucleic acid encoding phosphatase inhibitor (I-1) that is operatively linked to said promoter, and wherein the rAAV is from about 1×10¹¹ vg/ml to about 1×10¹³ vg/ml. In some embodiments, the rAAV comprising cardiac specific promoter selected from the Table 2A or, variant thereof, or a muscle-specific promoter active in cardiac and skeletal muscle selected from Table 5A or a variant thereof, or a shortened muscle-specific promoter active in cardiac and skeletal muscle selected from Table 13A, or a variant thereof allow efficacious repeat administration of rAAV vector to treat heart disease (e.g., congestive heart failure) using a rAAV virion with any AAV capsid that is different than the prior administrations, wherein the rAAV comprises a nucleic acid encoding phosphatase inhibitor (I-1) that is operatively linked to said promoter, and wherein at least one total dose of rAAV is from about 10¹¹ vg to about 10¹⁵ vg. In some embodiments, at least one total dose of rAAV is from about 10¹¹ vg to about 10¹⁴ vg. In some embodiments, at least one total dose of rAAV is 10¹² vg, or, 10¹³ vg, or, 3×10¹³ vg, or, 10¹⁴ vg, or, 3×10¹⁴ vg. For example, SP0173, SP0320, SP0279, SP0134, SP0057, SP0229, SP0067, SP0310, SP0311, or, SP0267, allow efficacious repeat administration of rAAV vector to treat heart disease (e.g., congestive heart failure) using a rAAV virion with any AAV capsid that is different than prior administration, i.e. an AAV capsid with a different immune profile.

### (I) Immune Modulators:

In some embodiments, the methods and compositions for treating heart failure, as described herein, further comprises administering an immune modulator. In some embodiments, the immune modulator can be administered at the time of rAAV vector administration, before rAAV vector administration or, after the rAAV vector administration.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in US 7,666,582, US 8,133,483, US 20180037962, US 20180023070, US 20170209550, US 8,889,128, WO 2010057626, US 9,707,279, US 8,323,908, US 20190345533, US 20190262434, and, WO 2020016318, each of which are incorporated in their entirety by reference.

In some embodiments, the immune modulator is Proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in US 10,028,993, US 9,592,247, and, US 8,809,282, each of which are incorporated in their entirety by reference.

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in US 10,071,114, US 20160067228, US 20160074531, US 20160074532, US 20190076458, US 10,046,064, are incorporated in their entirety. In other aspects of the embodiment, the immune modulator is synthetic nanocarriers comprising an immunosuppressant. Non limiting examples of references of immunosuppresants, immunosuppressants coupled to synthetic nanocarriers, synthetic nanocarriers comprising rapamycin, and/or, toloregenic synthetic nanocarriers, their doses, administration and use as described in US20150320728, US 20180193482, US 20190142974, US 20150328333, US20160243253, US 10,039,822, US 20190076522, US 20160022650, US 10,441,651, US 10,420,835, US 20150320870, US 2014035636, US 10,434,088, US 10,335,395, US 20200069659, US 10,357,483, US 20140335186, US 10,668,053, US 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila’s QS21 stimulon. In another further embodiment, the immunomodulator or adjuvant is poly-ICLC

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS¾A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B- cell costimulation pathways, such as agents that interfere with the coupling of T-cells and B-cells via the CTLA4 and B7 pathways, as disclosed in U.S. Pat. Pub. No 2002/0182211. In one embodiment, an immunosuppressive agent is cyclosporine A. Other examples include myophenylate mofetil, rapamicin, and anti- thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. Pat. Application No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

### (II) Vasodilators

In some embodiments, the methods and compositions for treating heart failure, as described herein, further comprises administering a vasodilator. In some embodiments, the vasodilator can be administered at the time of rAAV vector administration (i.e., concurrent with, or substantially concurrent with the rAAV administration), before rAAV vector administration or, after the rAAV vector administration. Vasodilators can assist delivery of the rAAV vector by enlarging (dilating) blood vessels. In some embodiments, the vasodilator is administered at least about 1 minute prior, at least about 5 minutes prior, at least about 10 minutes prior, at least about 15 minutes prior, at least about 20 minutes prior, at least about 25 minutes prior, at least about 30 minutes prior, at least about 35 minutes prior, at least about 40 minutes prior, or more than 40 minutes prior the rAAV vector administration.

In some embodiments, the rAAV vector can be administered with at least one vasodilator selected from any of: Isosorbide dinitrate (ISORDIL®), Nesiritide (NATRECOR®), Hydralazine (APRESOLINE®), Nitrate drugs, Minoxidil, 4CAPTOPRIL™, Nitrovasodilators (nitroglycerin, isosorbide mononitrate, isosorbide dinitrate, and sodium nitroprusside, serelaxin, Endothelin antagonists (e.g., endothelin-1 (ET-1) antagonists e.g, tezosentan); other natriuretic peptides (e.g., Ularitide, CD-NP), Relaxin, Cenderitide, Clevidipine, TRV120027, Cinaciguat, BAY 1021189, BAY 28-2667 (N) and heme-independent soluble G protein activator), 1021189 CXL-1020, adenosine blockers, inhaled pulmonary vasodilators (iPVD) (e.g., iNO or inhaled epoprostenol); vasodilating inotropes (milrinone, dobutamine), [Pyr1]apelin-13 (see, e.g, El Mathari, B et al. “Apelin improves cardiac function mainly through peripheral vasodilation in a mouse model of dilated cardiomyopathy.” Peptides 142 (2021): 170568.) Vasodilators are well known to the skilled artisan and are encompassed for use in the methods and compositions as disclosed herein. Some vasodilators useful in the methods as disclosed herein are disclosed in: Holt et al., Vasodilator Therapies in the Treatment of Acute Heart Failure. Curr Heart Fail Rep. 2019 Feb;16(1):32-37, Travessa AM, Menezes Falcão L. Vasodilators in acute heart failure - evidence based on new studies. Eur J Intern Med. 2018 May; 51:1-10, et al., Levy et al., “Vasodilators in acute heart failure: review of the latest studies.” Current emergency and hospital medicine reports 2.2 (2014): 126-132; Levy, et al., 2014; Vasodilators in acute heart failure: review of the latest studies. Current emergency and hospital medicine reports, 2(2), 126-132; Kumaret al., “New drugs you are going to read about: serelaxin, ularitide, TRV027.” Current emergency and hospital medicine reports 3.2 (2015): 66-73.; Ibrahim N.E., et al., (2021) Diagnosis and Management of Acute Heart Failure. In: Gaggin H.K., Januzzi Jr. J.L. (eds) MGH Cardiology Board Review. Springer, Cham., each of which are incorporated herein in their entirety by reference.

In some embodiments, rAAVs can be administered with vasoactive agents or, vasculature permeability agents along with vasodilators. In some embodiments, rAAVs can be administered with only vasoactive agents or, vasculature permeability agents. In some embodiments, vasoactive agents and vasodilators are co administered at different times. Exemplary vasoactive agents or, vasculature permeability agents can be without limitation, histamine, histamine agonist, vascular endothelial growth factor protein (VEGF protein), serotonin, bradykinin, platelet activating factor (PAF), prostaglandin E1 (PGE1), zona occludens toxin (ZOT), interleukin 2, bradykinin, other plasmakinins as described in International Publication no. WO1999040945A3; U.S. Pat. no. 6,855,701 all of which are incorporated herein by reference in their entirety.

### III. Exemplary rAAV for Administration and rAAV Genome Elements

### A. Agent That Modulates Protein Phosphate Activity.

Increases in protein phosphorylation and enhanced cardiac function are reversed by protein phosphatases in an efficient and highly regulated process. Two main classes of serine/threonine phosphatases, referred to as phosphatase types 1 and 2 regulate cardiac muscle contractile performance (Neumann, J. et al., 1997 J Mol Cell Cardiol; 29(1): 265-72). Protein phosphatase 1 (“PP1”) accounts for a significant amount of the cardiac enzymatic activity, and has been implicated as the key class of regulatory phosphatase enzymes. PP1 is largely associated with the membrane fraction as well as glycogen particles and is important in glycogenolysis and glycogen synthesis. It is anchored to these locales by large, non-catalytic targeting subunits, which serve to enhance substrate availability and specificity. Furthermore, this enzyme is regulated by two heat and acid stable proteins, phosphatase inhibitors-1 and -2. Phosphatase Inhibitor-1 (“I-1”) is the main physiological modulator and is an effective inhibitor when phosphorylated on threonine-35 by PKA (Endo, S. et al., 1996 Biochemistry; 35(16): 5220-8). Inhibition of PP1, removes its opposition to the actions of PKA protein phosphorylation, leading to amplification of the β-agonist responses in the heart (Ahmad, Z. J. 1989 Biol Chem; 264:3859-63; Gupta, R. C. et al., 1996 Circulation; (Suppl 1):1-361).

This fine-tuning regulation of cardiac regulatory protein phosphorylation by protein kinases and phosphatases becomes even more important in heart failure, since decreases in cAMP levels by desensitization of β-receptors (Koch, Lefkowitz et al. 2000) would be expected to lead to inactivation of PKA, while the levels and activity of protein phosphatase 1 are increased.

That is phosphatase activity is increased in heart failure. Reducing phosphatase activity (e.g., phosphatase 1 activity) in cardiomyocytes can relieve one or more symptoms of associated with heart failure. Reduced phosphatase activity is associated with attenuated β-adrenergic responsiveness. Accordingly, expression of a phosphatase inhibitor in heart cells can be used to treat cardiac disorders, e.g., heart failure. Decreasing phosphatase activity can improve β-adrenergic responsiveness.

Accordingly, one aspect of the disclosure is a method of treating a subject having heart failure comprising administering a rAAV vector expressing an inhibitor the phosphorylation activity of PKC-α.

In one embodiment, phosphatase activity can be decreased by inhibiting type 1 phosphatases (PP1). Type 1 phosphatases include, but are not limited to PP1cα, PP1cβ, PP1cδ and PP1cγ. See Sasaki et. al. (1990) Jpn J Cancer Res. 81: 1272-1280, the contents of which are incorporated herein by reference. The phosphatase inhibitor-1 (or “I-1”) protein is an endogenous inhibitor of type 1 phosphatase. Increasing I-1 levels or activity can restore β-adrenergic responsiveness in failing human cardiomyocytes.

In specific embodiments, the rAAV vector comprises a nucleic acid encoding a constitutively active I-1 protein. One such rAAV vector comprises a nucleic acid encoding I-1T35D, which comprises a truncation of the I-1 cDNA to encode for the first 65 amino acids and introduction of nucleotide changes to replace the PKA phosphorylation site (GGT: Thr35) with aspartic acid (GTC: Asp35), resulting in a constitutively active inhibitor. In some embodiments, the rAAV vector comprises a nucleic acid construct encoding a constitutively active I-1 protein, where threonine 35 is replaced with glutamic acid instead of aspartic acid. These substitutions can also be made in a full length inhibitor molecule. Failing human cardiomyocytes expressing I-1T35D exhibit normal contractile function under basal conditions and their beta adrenergic function is restored to normal. Thus, delivery of inhibitor-1 completely restores function and reverses remodeling in the setting of pre-existing heart failure.

The rAAV vector as disclosed herein can comprise nucleic acid sequences encoding other phosphatase inhibitors and other variants of I-1. For examples, in some embodiments a rAAV vector as disclosed herein can comprise a nucleic acid encoding any one or more of the inhibitors selected from: phosphatase inhibitor 2 (PP2); okadaic acid or caliculin; and nippl which is an endogenous nuclear inhibitor of protein phosphatase 1. In one embodiment, the rAAV vector as disclosed herein comprises a nucleic acid encoding a phosphatase inhibitor is specific for protein phosphatase 1 (PP1). Examples of proteins that modulate cardiac activity include, but are not limited to: a protein that modulates phosphatase activity (e.g., a phosphatase type 1 inhibitor, e.g., I-1) or a sacroplasmic reticulum Ca2+ ATPase (SERCA), e.g., SERCA1 (e.g., 1a or 1b), SERCA2 (e.g., 2a or 2b), or SERCA3.

In some embodiments, a method of treatment according to an embodiment of the technology described herein comprises introduction into the heart cells of the subject, a rAAV vector comprising a nucleic acid sequence encoding a mutant form of phosphatase inhibitor-1 protein, wherein the mutant form comprises at least one amino acid at a position that is a PKC-α phosphorylation site in the wild type, wherein the at least one amino acid is constitutively unphosphorylated or mimics an unphosphorylated state in the mutant form.

### (I) Phosphatase Inhibitor Protein-1 (I-1) and Constitutively Active I-1 (I-1c) as Inhibitors of Protein Phosphatase 1 (PP1)

Phosphatase Inhibitor Protein-I (I-1) is a key regulator of cardiac contractility. I-1 is also known as Type-1 phosphtatase (PP1 or PP-1) is known to regulate cardiac contractility by inhibiting the activity of Protein Phosphatase-1 (“PP-1”). I-1′s ability to inhibit PP-1 is further known to be regulated by phosphorylation. When threonine 35 of I-1 is phosphorylated by Protein Kinase A (PKA), PP-1 activity is inhibited, cardiac contractility is enhanced (Pathak, A., et al. 2005 Circ Res 15:756′-66).

In one embodiment, phosphatase activity can be decreased by inhibiting type 1 phosphatases (PP1). Type 1 phosphatases include, but are not limited to PP1cα, PP1cβ, PP1cδ and PP1cγ. See Sasaki et. al. (1990) Jpn J Cancer Res. 81: 1272-1280, the contents of which are incorporated herein by reference. The phosphatase inhibitor-1 (or “I-1”) protein is an endogenous inhibitor of type 1 phosphatase. Increasing I-1 levels or activity can restore β-adrenergic responsiveness in failing human cardiomyocytes.

In specific embodiments, a constitutively active I-1 protein can be administered. One such construct exemplified herein (I-1T35D) entails truncation of the I-1 cDNA to encode for the first 65 amino acids and introduction of nucleotide changes to replace the PKA phosphorylation site (GGT: Thr35) with aspartic acid (GTC: Asp35), resulting in a constitutively active inhibitor. Another way to make a constitutively active inhibitor is to substitute threonine 35 with glutamic acid instead of aspartic acid. These substitutions can also be made in a full length inhibitor molecule. Failing human cardiomyocytes expressing I-1T35D exhibit normal contractile function under basal conditions and their beta adrenergic function is restored to normal. Thus, delivery of inhibitor-1 completely restores function and reverses remodeling in the setting of pre-existing heart failure.

The nucleic acid encoding I-1 is shown as follows:

agtgtccccg gagccgcgag ctgggagcgc tgtgccggga gccgggagcc gagcgcgccg 60
ggctggggcc ggggccggag cggagcggag agggagcgcg cccgccccag ccccgagtcc 120
cgccgccttc cctcccgccg cagcgcgggc ccaccggccg ccgccccagc catggagcaa 180
gacaacagcc cccaaaagat ccagttcacg gtcccgctgc tggagccgca ccttgacccc 240
gaggcggcgg agcagattcg gaggcgccgc cccacccctg ccaccctcgt gctgaccagt 300
gaccagtcat ccccagagat agatgaagac cggatcccca acccacatct caagtccact 360
ttggcaatgt ctccacggca acggaagaag atgacaagga tcacacccac aatgaaagag 420
ctccagatga tggttgaaca tcacctgggg caacagcagc aaggagagga acctgagggg 480
gccgctgaga gcacaggaac ccaggagtcc cgcccacctg ggatcccaga cacagaagtg 540
gagtcaaggc tgggcacctc tgggacagca aaaaaaactg cagaatgcat ccctaaaact 600
cacgaaagag gcagtaagga acccagcaca aaagaaccct caacccatat accaccactg 660
gattccaagg gagccaactc ggtctgagag aggaggaggt atcttgggat caagactgca 720
gtttgggaat gcatggacac cggatttgtt tcttattcct tcacttttgg ggaaaatctc 780
ttgtttttaa aaagtgataa atttggtgtt aggtccttgg cactttcctt cttttccaac 840
tgggagaatc ctttctccct gccttcttgc cctgccctct ctgtagcccc caccctcctg 900
ccaagctgcc tctgggaagg aagaaacagg agctaggcag aagccttgag cagggaagag 960
ttcttccctt agccctgact ttacttgctg tgggaagaga gatgagggtc agataggtgg 1020
gaggactaac ttccagggtg ccaagaagga agaaaagccc caggttctct tttcttattg 1080
aggaacgatc cgaccacctc acaggcctgc cctgcagctg gaagactcgg cgctctaagg 1140
cctgtgccgt gtccagctgt gactgtgcgg tgggctccat ctgctggaca aaaggggaac 1200
tgcaccatgg cacttggccc atgggaaaga gggtgtggtg gtgtgccaat acctcctcgc 1260
ctgccctcca agccccagct gccttccttt tggattccca agcttcagga tgtgttccct 1320
cttccagctg tgggaccgct gtcccttatt tcaacccgtt agcaacaatg gatagagaac 1380
acagtggcta ttaatgaaga ggcccatgct ggagactgga agggttccct tgtcctagac 1440
attgaggggc ccagataaga ccaaaaccaa gcataagaga agaaactgtc tcagatctca 1500
cggccaggcc tctctcctgc tgctgttttt gattttccca ggtagtggga gagaggaaag 1560
gagggaaggc aagattcttt ccccctccct gctgaagcat gtggtacaga ggcaagagca 1620
gagcctgaga agcgtcaggt cccacttctg ccatgcagct actatgagcc ctcggggcct 1680
cctcctgggc ctcagcttgc ccagatacat acctaaatat atatatatat atatgaggga 1740
gaacgcctca cccagatttt atcatgctgg aaagagtgta tgtatgtgaa gatgcttggt 1800
caacttgtac ccagtgaaca cacaaaaaaa aaaaaaaaa 1839
(SEQ ID NO: 2)

In some embodiments of the methods and compositions as disclosed herein, the rAAV vector comprises a nucleic acid sequence encoding a I-1 or I-1c protein of amino acids SEQ ID NO: 1 ora modified variant of SEQ ID NO: 1. In some embodiments, the rAAV vector comprises a nucleic acid sequence of SEQ ID NO: 2, or a fragment thereof, wherein the fragment of SEQ ID NO: 2 encodes amino acids 1-65 of SEQ ID NO: 1, or a fragment from amino acid 1 to C-terminal amino acid 70, 67, 66, 65, or 61, or 54 of SEQ ID NO: 1, where threonine at position 35 of SEQ ID NO: 1 is replaced with an aspartic acid (T35D). In some embodiments of the methods and compositions as disclosed herein, the rAAV vector comprises a nucleic acid sequence encoding a I-1 or I-1c protein which is a codon optimized nucleic acid sequence, for enhanced expression in vivo and/or to reduce CpG islands, and/or to reduce the innate immune response. Exemplary codon optimized I-1 or I-1c nucleic sequences encompassed for use in the methods and rAAV compositions as disclosed herein can be used, or a nucleic acid sequence having at least 60%, or 70%, or 80%, 85% or 90% or 95%, or 98%, or 99% sequence identity to SEQ ID NO: 2 or a portion thereof, or SEQ ID NOS: 385-412, which is codon optimized according to methods known in the art, or a nucleic acid sequence that has at least 81%, 82%, 83%, 84%, 85%, 86%, 87%, 88%, 89%, 90%, 91%, 92%, 93%, 94%, 95%, 96%, 97%, 98%, or 99% sequence identity to any of SEQ ID NO: 2 or 385-412.

In addition, in some embodiments, the I-1 or I-1c nucleic acid sequences encompassed for use in the methods and rAAV compositions as disclosed herein are further modified with at least one or more of the following modifications: (i) removal of at least one, or two or in some embodiments, all alternative reading frames, (ii) removal of one or more CpGs islands, (iii) modification of the Kozak sequence, (iv) modification of a translational terminator sequence, and (v) removal of a spacer between promoter and Kozak sequence.

In some embodiments of the methods and compositions as disclosed herein, the human I-1 protein expressed by the AAV is encoded by a codon optimized nucleic acid sequence, for example, a sequence selected from any of SEQ ID NO: 385-412. In some embodiments of the methods and compositions as disclosed herein, the I-1 protein expressed by the rAAV vector is encoded by a nucleic acid sequence having at least 60%, or 70%, or 80%, 85% or 90% or 95%, or 98%, or 99% sequence identity to SEQ ID NOS: 385-412. In some embodiments, such a nucleic acid sequence having at least 60%, or 70%, or 80%, 85% or 90% or 95%, or 98%, or 99% sequence identity to SEQ ID NOS: 385-412 can be assessed using in vitro assays as disclosed in the Examples herein, or using cardiomyocytes from failing hearts (e.g., tissues from failing left ventricles (LV)) where PP1 activity can be assayed with 32P-labeled rabbit glycogen phosphorylase as the substrate. To maintain the phosphorylation status of I-1, PP2A (okadaic acid, 4 nM) and calcineurin phosphatase (EDTA, 0.5 mM) inhibitors can be included in preparations of tissue extracts and in the enzyme reactions), as disclosed in Carr, et al. “Type 1 phosphatase, a negative regulator of cardiac function.” Molecular and cellular biology 22.12 (2002): 4124-4135; which is incorporated herein in its entirety by reference.

In some embodiments, such a nucleic acid sequence having at least 60%, or 70%, or 80%, 85% or 90% or 95%, or 98%, or 99% sequence identity to SEQ ID NOS: 385-412 can also be assessed using in vivo and in vitro assays as disclosed in the Examples herein and disclosed in Patkak et al, e.g., by assessing in vivo cardiac function by non-invasive echocardiography and echocardiographic assessment, and in vitro contractility was examined using the Langendorff perfusion system. Cardiac catheritization and pressure-volume loop measurements in the murine heart can also be performed, as disclosed in Patjak, et al. “Enhancement of cardiac function and suppression of heart failure progression by inhibition of protein phosphatase 1.” Circulation research 96.7 (2005): 756-766, which is incorporated herein in its entirety by reference.

The amino acid sequence of I-1 is as follows:

MEQDNSPQKIQFTVPLLEPH LDPEAAEQIRRRRPT PATLV LTSDQSSPEIDEDRIPNPHL
KSTLAMSPRQRKKMTRITPT MKELQMMVEHHLGQQQQGEE PEGAAESTGTQESRPPGIPD
TEVESRLGTSGTAKKTAECI PKTHERGSKEPSTKEPSTHI PPLDSKGANSV* (SEQ ID NO: 1)

To determine the long-term in vivo effects of decreased protein phosphatase 1 activity, we expressed a constitutively active, truncated inhibitor-1 (I1c) in a cardiomyocyte restricted manner. This form of inhibitor-1 was chosen because it specifically inhibits protein phosphatase 1, albeit at higher concentration than the native phosphorylated inhibitor.

Accordingly, in some embodiments, the rAAV vector disclosed herein comprises a nucleic acid sequence encoding a polypeptide comprising amino acids 1-65 of SEQ ID NO: 1, wherein threonine at position 35 of SEQ ID NO: 1 is replaced with an aspartic acid (T35D); and wherein said nucleic acid sequence is operably linked to a cardiac-specific promoter as disclosed in Table 2A, 3 or 4 herein. In some embodiments, the I-1 protein or a functional variant thereof is expressed in the heart of the subject in an amount effective to increase cardiac contractility and reduce morphological deterioration associated with cardiac remodeling in the subject with existing heart failure.

In some embodiments, the rAAV vector disclosed herein comprises a nucleic acid sequence encoding a constitutively active fragment of I-1 (I-1c), wherein I-1c is a polypeptide comprising amino acids of SEQ ID NO: 1, wherein SEQ ID NO: 1 is truncated at the C-terminus at amino acid 70, 67, 66, 65, or 61, or 54, and where the there is an aspartic acid at position 35 (T35D).

The I-1 polypeptide is described in U.S. Pats. 9,114,148, which is incorporated herein in its entirety by reference. In some embodiments, the I-1 polypeptide can comprise a secretory signal (SS). In some embodiments, one of ordinary skill in the art can appreciate particular positions of I-1 or I-1c which a secretory signal peptide (SS) can be fused. Accordingly, in one aspect the invention relates to a I-1 protein, beginning at amino acid 1 and terminating at amino acid 70, 67, 66, 65, or 61, or 54 of human I-1 of SEQ ID NO: 1, or a modified I-1 protein of SEQ ID NO: 1, where there is an aspartic acid at position 35 (T35D) of SEQ ID NO: 1.

In some embodiments of the methods and compositions as disclosed herein, the human I-1 protein expressed by the AAV comprises amino acids of SEQ ID NO: 1, or fragments or variants thereof, for example a I-1 protein beginning at residue 70, 67, 66, 65, or 61, or 54 of SEQ ID NO: 1. In some embodiments of the methods and compositions as disclosed herein, the I-1 protein expressed by the rAAV vector comprises amino acids beginning at any one or 70, 67, 66, 65, or 61, or 54 of SEQ ID NO: 1, or a protein at least 60%, or 70%, or 80%, 85% or 90% or 95%, or 98%, or 99% identical to SEQ ID NO: 1, beginning at amino acids 70, 67, 66, 65, or 61, or 54. In some embodiments of the methods and compositions as disclosed herein, the I-1 protein expressed by the rAAV comprises amino acids beginning at residue 70, 67, 66, 65, or 61, or 54 of SEQ ID NO: 1, or a protein at least 60%, or 70%, or 80%, 85% or 90% or 95%, or 98%, or 99% identical thereto. In some embodiments, the I-1 protein expressed by the rAAV vector comprises amino acids of beginning at residue 70, 67, 66, 65, or 61, or 54 of any of SEQ ID NO: 1, where there is an aspartic acid at position 35 (T35D) of SEQ ID NO: 1, or a protein at least 60%, or 70%, or 80%, 85% or 90% or 95%, or 98%, or 99% identical thereto.

### (II) Evaluation of Different rAAV Vector Constructs and I-1c

In some embodiments, the effect of a rAAV vector as disclosed herein on phosphatase enzymatic activity can be evaluated in vitro. For example, protein phosphatase 1 activity can be assayed as described (Endo, S., et al. (1996) Biochemistry 35, 5220-5228) in a 30-µl reaction mixture containing 50 mM Tris.HCl (pH 7.4), 1 mM DTT, 0.5 mM MnCl2, 10 µM [32P]phosphorylase a, and 0.5 µg/ml PP1. The reaction is initiated by the addition of 1 µl of PP 1 to 20 µl of assay mixture containing the rest of the assay components. After 20 min at 30° C. the reaction is terminated by adding 10 µl of 50% trichloroacetic acid to the assay mixture. The assay mixture is then cooled on ice and centrifuged. A 20 µl aliquot from the supernatant was spotted onto filter paper and placed in a scintillation counter to determine the amount of released [32P]Pi. [32P]Phosphorylase a used for PP1 assays was prepared at 30° C. for 30 min as described. [32P]Phosphorylase a was dialyzed in 50 mM Tris.HCl, pH 7.4, 1 mM EDTA, 1 mM DTT and stored frozen at -80° C. until used (see also Huang et al. Proc Natl Acad Sci USA. 2000 May 23; 97(11):5824-9).

The efficacy of a rAAV vector as disclosed herein expressing an inhibitor of PP1, e.g., I-1, I-1c or a variant thereof can be assessed by generating dose response curves from data obtained using various concentrations of the test compounds. Moreover, a control assay can also be performed to provide a baseline for comparison. In the control assay, the heart cell is incubated in the absence of a test compounds.

Table 17 shows, without being construed to any limitation exemplary nucleic acid sequences encoding I1c genes (SEQ ID NO: 385-412), where the rAAV vector can comprise a nucleic acid sequence selected from any of SEQ ID NOS 413-440, which comprise the I-1c nucleic acid and other components and flanked within left and right ITR sequences, or a nucleic acid sequence that has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 413-440. Exemplary close ended linear duplex useful herein can comprises a nucleic acid sequence selected from of any of SEQ ID NO: 357-384, or a nucleic acid that has at least 81%, 82%, 83%, 84%, 85%, 86%, 87%, 88%, 89%, 90%, 91%, 92%, 93%, 94%, 95%, 96%, 97%, 98%, or 99% identity thereto.

| | | |
|---|---|---|
| ITR-ITR + backbone | I-1c | Left ITR-right ITR |
| SEQ ID NO: 357 | SEQ ID NO: 385 | SEQ ID NO: 413 |
| SEQ ID NO: 358 | SEQ ID NO: 386 | SEQ ID NO: 414 |
| SEQ ID NO: 359 | SEQ ID NO: 387 | SEQ ID NO: 415 |
| SEQ ID NO: 360 | SEQ ID NO: 388 | SEQ ID NO: 416 |
| SEQ ID NO: 361 | SEQ ID NO: 389 | SEQ ID NO: 417 |
| SEQ ID NO: 362 | SEQ ID NO: 390 | SEQ ID NO: 418 |
| SEQ ID NO: 363 | SEQ ID NO: 391 | SEQ ID NO: 419 |
| SEQ ID NO: 364 | SEQ ID NO: 392 | SEQ ID NO: 420 |
| SEQ ID NO: 365 | SEQ ID NO: 393 | SEQ ID NO: 421 |
| SEQ ID NO: 366 | SEQ ID NO: 394 | SEQ ID NO: 422 |
| SEQ ID NO: 367 | SEQ ID NO: 395 | SEQ ID NO: 423 |
| SEQ ID NO: 368 | SEQ ID NO: 396 | SEQ ID NO: 424 |
| SEQ ID NO: 369 | SEQ ID NO: 397 | SEQ ID NO: 425 |
| SEQ ID NO: 370 | SEQ ID NO: 398 | SEQ ID NO: 426 |
| SEQ ID NO: 371 | SEQ ID NO: 399 | SEQ ID NO: 427 |
| SEQ ID NO: 372 | SEQ ID NO: 400 | SEQ ID NO: 428 |
| SEQ ID NO: 373 | SEQ ID NO: 401 | SEQ ID NO: 429 |
| SEQ ID NO: 374 | SEQ ID NO: 402 | SEQ ID NO: 430 |
| SEQ ID NO: 375 | SEQ ID NO: 403 | SEQ ID NO: 431 |
| SEQ ID NO: 376 | SEQ ID NO: 404 | SEQ ID NO: 432 |
| SEQ ID NO: 377 | SEQ ID NO: 405 | SEQ ID NO: 433 |
| SEQ ID NO: 378 | SEQ ID NO: 406 | SEQ ID NO: 434 |
| SEQ ID NO: 379 | SEQ ID NO: 407 | SEQ ID NO: 435 |
| SEQ ID NO: 380 | SEQ ID NO: 408 | SEQ ID NO: 436 |
| SEQ ID NO: 381 | SEQ ID NO: 409 | SEQ ID NO: 437 |
| SEQ ID NO: 382 | SEQ ID NO: 410 | SEQ ID NO: 438 |
| SEQ ID NO: 383 | SEQ ID NO: 411 | SEQ ID NO: 439 |
| SEQ ID NO: 384 | SEQ ID NO: 412 | SEQ ID NO: 440 |

### Animal Models

Important prerequisites for successful studies of cardiovascular gene therapy are (1) constitution of an animal model that is applicable to clinical cardiovascular disease that can provide useful data regarding mechanisms for increased blood flow and/or contractile function, and (2) accurate evaluation of the effects of gene transfer. Thus, in some embodiments, a porcine models can be used. The pig is a particularly suitable model for studying a cardiovascular condition, including heart diseases of humans because of its relevance to human physiology. The pig heart closely resembles the human heart in the following ways. The pig has a native coronary circulation very similar to that of humans, including the relative lack of native coronary collateral vessels. Secondly, the size of the pig heart, as a percentage of total body weight, is similar to that of the human heart. Additionally, the pig is a large animal model, therefore allowing more accurate extrapolation of various parameters such as effective vector dosages, toxicity, etc. In contrast, the hearts of animals such as dogs and members of the murine family have a lot of endogenous collateral vessels. Additionally, relative to total body weight, the size of the dog heart is twice that of the human heart.

An exemplary porcine model is a myocardial ischemia porcine model, which mimics clinical coronary artery disease, is described in U.S. Application 2003/0148968, which is incorporated herein in its entirety by reference. Based on published studies, those skilled in the art will appreciate that the results in a pig model are expected to be predictive of results in humans.

Another animal model, described in Example 1 of U.S. Application 2003/0148968 can also be used, which induces dilated cardiomyopathy such as that observed in clinical congestive heart failure.

Thus, these models can be used to determine whether the methods of administration of a rAAV vector as disclosed herein, and/or the rAAV vector encoding an inhibitor of PP1 (e.g., I-1, I-1c or a variant thereof) and/or another therapeutic protein (e.g., angiogenic protein or peptide) is effective to alleviate at least one cardiac dysfunctions associated with these conditions. These models are particularly useful in providing some of the parameters by which to assess the effectiveness of in vivo gene therapy for the treatment of congestive heart failure and ventricular remodeling.

### (Iii) Other Agents to Be Expressed by rAAV Vectors for the Treatment of Heart Failure

In some embodiments, the rAAV vector as disclosed herein can express other phosphatase inhibitors and other variants of I-1. Examples of such other inhibitors include phosphatase inhibitor 2; okadaic acid or caliculin; and nippl which is an endogenous nuclear inhibitor of protein phosphatase 1. In one embodiment, the phosphatase inhibitor is specific for protein phosphatase 1.

In some embodiments, the rAAV vector as disclosed herein can express other therapeutic agents to treat heart failure, e.g., adenylyl cyclase 6 (AC6, also referred to as adnenylyl cyclase VI), S100A1, β-adrenergic receptor kinase-ct (βARKct), sarco/endoplasmic reticulum (SR) Ca -ATPase (SERCA2a), IL-18, VEGF, VEGF activators, urocortins, and B-cell lymphoma 2 (Bcl2)-associated anthanogene-3 (BAG3).

Table 18A: Exemplary genes to be encoded by a rAAV vector comprising a synthetic cardiac-specific promoter as disclosed herein.

| Protein | mRNA sequence ID | SEQ ID NO: | Amino acids of Transcript Variants |
|---|---|---|---|
| VEGF (VEGF-A) | NM_001025366 | 450 | See, e.g., SEQ ID NOS: 95-99 and 140-151 of U.S. Pat. 10/086,043 |
| activin A | NM_001105 | 451 | See, e.g., SEQ ID NO: 152 of U.S. Pat. 10/086,043 |
| activin B | NM_002193.2 | 452 | N/A |
| insulin-like growth factor (IGF1) | NM_000618 | 453 | See, e.g., SEQ ID NOS: 153-155 of U.S. Pat. 10/086,043 |
| bone morphogenic protein | NM_006132 | 454 | See, e.g., SEQ ID NOS: 156-157 of U.S. Pat. 10/086,043 |
| fibroblast growth factor | NM_000800 | 455 | See, e.g., SEQ ID NOS: 158-162 of U.S. Pat. 10/086,043 |
| platelet-derived growth factor | NM_002607 | 456 | See, e.g., SEQ ID NO: 163 of U.S. Pat. 10/086,043 |
| insulin, leukemia inhibitory factor (LIF) | NM_002309 | 457 | N/A |
| epidermal growth factor (EGF) | NM_001178130 | 458 | See, e.g., SEQ ID NOS: 164-165 of U.S. Pat. 10/086,043 |
| TGFalpha | NM_001099691 | 459 | See, e.g., SEQ ID NO: 166 of U.S. Pat. 10/086,043 |
| TDGF1 | NM_003212 | 460 | See, e.g., SEQ ID NO: 167 of U.S. Pat. 10/086,043 |
| vWF | NM_000552 | 461 | N/A |
| GATA-4 | NM_002052 | 462 | N/A |
| Nkx2.5 | NM_001166175 | 463 | See, e.g., SEQ ID NOS: 168-169 of U.S. Pat. 10/086,043 |
| Mef2-C | NM_002397 | 464 | See, e.g., SEQ ID NOS: 170-174 of U.S. Pat. 10/086,043 |
| LGMD-2B; limb girdle dystrophy 2B (Dysferlin) | NM_003494 | 465 | See, e.g., SEQ ID NOS: 175-187 of U.S. Pat. 10/086,043 |
| Dystrophin (DMD) | NM_004006 | 466 | See, e.g., SEQ ID NOS: 188-204 of U.S. Pat. 10/086,043 |
| Emerin (EMD) | NM_0001 17 | 467 | N/A |
| Lamin A/C | NM_170707 | 468 | See, e.g., SEQ ID NOS: 205-206 of U.S. Pat. 10/086,043 |
| alpha-1 anti-trypsin (A I AT) | NM_001002235 | 469 | See, e.g., SEQ ID NOS: 207-216 of U.S. Pat. 10/086,043 |
| CFTR | NM_000492 | 470 | N/A |
| ANG | NM_001097577 | 471 | See, e.g., SEQ ID NO: 217 of U.S. Pat. 10/086,043 |
| Presenilin (PSEN2) | NM_000447 | 472 | See, e.g., SEQ ID NO: 218 of U.S. Pat. 10/086,043 |
| Isl1 | NM_002202 | 473 | N/A |
| SERCA 1a (ATP2A1) | NM_004320 | 474 | See, e.g., SEQ ID NO: 219 of U.S. Pat. 10/086,043 |
| SERCA2a (ATP2A2) | NM_001681 | 475 | See, e.g., SEQ ID NO: 220 of U.S. Pat. 10/086,043 |
| Phospholamban (PLN) | NM_002667 | 476 | N/A |
| βARK (ADRBK1, GRK2, BARK1) | NM_001619 | 477 | N/A |
| beta-adrenergic receptor (ADRB1) | NM_000684 | 478 | N/A |
| Akt (AKT1) | NM_005163 | 479 | See, e.g., SEQ ID NOS: 221-222 of U.S. Pat. 10/086,043 |
| adenylyl cyclase VI (ADCY6) | NM_020983 | 480 | 223 |
| FGF 1 | NM_000800 | 481 | See, e.g., SEQ ID NOS: 241-245 of U.S. Pat. 10/086,043 |
| FGF 2 (FGFB) | NM_002006 | 482 | N/A |
| FGF 4 | NM_002007 | 483 | N/A |
| FGF 5 | NM_004464 | 484 | See, e.g., SEQ ID NO: 246 of U.S. Pat. 10/086,043 |
| HGF | NM_000601 | 485 | See, e.g., SEQ ID NOS: 247-250 of U.S. Pat. 10/086,043 |
| Ang1 (ANGPT1; angiopoietin1) | NM_001146 | 486 | See, e.g., SEQ ID NOS: 251, 353-354 of U.S. Pat. 10/086,043 |
| MGP1 (Matrix Gla Protein) | NM_000900 | 487 | See, e.g., SEQ ID NO: 252 of U.S. Pat. 10/086,043 |
| OMG (oligodendrocyte myelin glycoprotein) | NM_002544 | 488 | N/A |
| G-CSF (CSF3; Colony Stimulating Factor 3) | NM_172220 | 489 | See, e.g., SEQ ID NO: 253-255 of U.S. Pat. 10/086,043 |
| PDGF (PDGFA) | NM_002607 | 490 | See, e.g., SEQ ID NO: 256 of U.S. Pat. 10/086,043 |
| IGF1 | NM_000618 | 491 | See, e.g., SEQ ID NO: 257-259 of U.S. Pat. 10/086,043 |
| IGF2 | NM_000612 | 492 | See, e.g., SEQ ID NOs: 260-261 of U.S. Pat. 10/086,043 |
| EGR1 | NM_001964 | 493 | N/A |
| Prax1 (benzodiazapine receptor associated protein 1; BZRAP1; PRAX-1, KIAA0612) | NM_004758 | 494 | See, e.g., SEQ ID NO: 262 of U.S. Pat. 10/086,043 |
| Neuregulin 1 (NRG1, GGF) | NM_001159995 | 495 | See, e.g., SEQ ID NOS: 263-278 of U.S. Pat. 10/086,043 |
| ErbB4 (ERBB4) | NM_001042599 | 496 | See, e.g., SEQ ID NO: 279 of U.S. Pat. 10/086,043 |
| Periostin (POSTN) | NM_006475 | 497 | 280-282 of U.S. Pat. 10/086,043 |
| HAND1 | NM_004821 | 498 | N/A |
| E2F4 | NM_001950 | 499 | N/A |
| Skp2 | NM_005983 | 500 | See, e.g., SEQ ID NOS: 283-284 of U.S. Pat. 10/086,043 |
| Akt1 | NM_005163 | 501 | See, e.g., SEQ ID NOS: 285-286 of U.S. Pat. 10/086,043 |
| BAG3 | NM_004281 | 502 | |
| I-1c | I-1c (T35D) | 507 | |
| truncated I-1c (aal-54;T35D) | I-1c (1-54)(T35D) | 527 | |
| truncated I-1c (aal-61;T35D) | I-1c (1-61)(T35D) | 528 | |
| truncated I-1c (aal-66;T35D) | I-1c (1-66)(T35D) | 529 | |
| truncated I-1c (aal-67;T35D) | I-1c (1-67)(T35D) | 530 | |
| truncated I-1c (aal-70;T35D) | I-1c (1-77)(T35D) | 531 | |
| truncated I-1c (aal-77;T35D) | I-1c (1-77)(T35D) | 532 | |
| β-adrenergic receptor kinase-carboxyl terminus (βARKct), | NM_001619 | 503 | Last 194 aa of c-terminus of βARK 1 (NM_001619) |
| S100A1 | NM_006271 | 504 | |
| IL-18 binding protein (IL-18BP) | SEQ ID NO: 1 in U.S. Pat. 7,799,541 | 505 | As disclosed in WO 99/09063 and US9,566,313, US20140112915 |
| Urocortin (UCN) | NM_003353 | 506 | |

Table 18B: Exemplary therapeutic agents to be expressed by the rAAV vectors for the treatment of arrhythmia or myopathy

| Protein | mRNA sequence ID | SEQ ID NO: | Transcript Variants SEQ ID NO |
|---|---|---|---|
| HERG (LQT2, short QT - Ikr, Kv11.1; KCNH2) | NM_000238 | 508 | 2 See, e.g., SEQ ID NOS: 87-289 of U.S. Pat. 10/086,043 |
| KCNQ1 (LQT - Iks; KCNA9) | NM_000218 | 509 | N/A |
| SCN5A (LQT3, Brugada) | NM_198056 | 510 | See, e.g., SEQ ID NOS: 290-294 of U.S. Pat. 10/086,043 |
| ANK2 (Ankyrin B; LQT4) | NM_001148 | 511 | See, e.g., SEQ ID NOS: 295-296 of U.S. Pat. 10/086,043 |
| KCNE1 (LQT5; MinK, ISK, JLNS2) | NM_000219 | 512 | See, e.g., SEQ ID NOS: 297-299 of U.S. Pat. 10/086,043 |
| KCNE2 (LQT6; MiRP1) | NM_172201 | 513 | N/A |
| KCNJ2 (LQT7, short QT; Kir2.1, IRK1) | NM_000891 | 514 | N/A |
| CACNA1c (LQT8, Brugada; Cav1.2, CACH2, CACN2, TS) | NM_000719 | 515 | See, e.g., SEQ ID NOS: 300-321 of U.S. Pat. 10/086,043 |
| SCN4B (LQT10) | NM_001142348 | 516 | See, e.g., SEQ ID NOS: 322-323 of U.S. Pat. 10/086,043 |
| SERCA (CPVT; APT2A1) | NM_004320 | 517 | See, e.g., SEQ ID NO: 324 of U.S. Pat. 10/086,043 |
| KCNQ2 (short QT; Kv7.2, ENB1, BFNC, KCNA11, HNSPC) | NM_172109 | 518 | See, e.g., SEQ ID NOS: 325-328 of U.S. Pat. 10/086,043 |
| SCN1B (Brugada) | NM_001037 | 519 | See, e.g., SEQ ID NO: 329 of U.S. Pat. 10/086,043 |
| KCNE3 (Brugada; MiRP2, HOKPP) | NM_005472 | 520 | N/A |
| Nebulin (NEB) (Nemaline myopathy) | NM_004543 | 521 | See, e.g., SEQ ID NOS: 330-331 of U.S. Pat. 10/086,043 |
| MTM1 (myotubularin 1) (X-linked myotubular myopathy) | NM_000252 | 522 | N/A |
| Lamin A/C (LMNA; LMN1; PRO1; LMNL1) | NM_170707 | 523 | See, e.g., SEQ ID NOS: 332-333 of U.S. Pat. 10/086,043 |
| Emerin (EMD) | NM_000117 | 524 | N/A |
| Desmin (DES; CMD1I, CSM1, CSM2) | NM_001927 | 525 | N/A |
| Delta-sarcoglycan (SGCD; DAGD, LGMD2F, CMD1L) | NM_000337 | 526 | See, e.g., SEQ ID NOS: 334-335 of U.S. Pat. 10/086,043 |

In some embodiments, the rAAV vector encodes a nucleic acid sequence as disclosed in Table 1, 2, 3, 4, 5, 6 or 7 of U.S. Pat. 10/086,043, which is incorporated in its entirety by reference.

In some embodiments, the rAAV vector for use in the methods and compositions as disclosed herein comprises a nucleic acid sequence that encodes a protein disclosed in Table 18A or variant thereof, where the nucleic acid sequence is selected from any of: SEQ ID NOS: 1, 450-507, or 527-532 or a variant thereof having at least 80%, or at least 85%, or at least 90%, or at least 95% or at least 98% sequence identity to any of SEQ ID NOS: 1, 450-507, or 527-532. In some embodiments, the rAAV vector for use in the methods and compositions as disclosed herein comprises a nucleic acid sequence that is codon optimized that encodes for a protein or variant of a protein selected from any in Table 18A, where the codon optimized nucleic acid sequence is a codon optimized nucleic acid sequence selected from any of selected from any of: SEQ ID NOS: 1, 450-507, or 527-532 and has at least 60%, or at least 70% or at least 80%, or at least 85%, or at least 90%, or at least 95% or at least 98% sequence identity to SEQ ID NOS: SEQ ID NOS: 1, 450-507, or 527-532. In some embodiments, rAAV vector comprises a nucleic acid sequence encoding any of SEQ ID NOS: 1, 450-507, or 527-532, and is codon optimized to have at least 50%, or 60% or 70% or 75%, 80%, 85%, 90%, 95% reduced CpG site content relative to the CpG site content of the nucleic acid sequence of SEQ ID NOS: 1, 450-507, or 527-532.

In some embodiments, the rAAV vector for use in the methods and compositions as disclosed herein comprises a nucleic acid sequence that encodes a protein disclosed in Table 18B or variant thereof, where the nucleic acid sequence is selected from any of: SEQ ID NOS: 508-526 or a variant thereof having at least 80%, or at least 85%, or at least 90%, or at least 95% or at least 98% sequence identity to any of SEQ ID NOS: 508-526. In some embodiments, the rAAV vector for use in the methods and compositions as disclosed herein comprises a nucleic acid sequence that is codon optimized that encodes for a protein or variant of a protein selected from any in Table 18B, where the codon optimized nucleic acid sequence is a codon optimized nucleic acid sequence selected from any of selected from any of: SEQ ID NOS: 508-526 and has at least 60%, or at least 70% or at least 80%, or at least 85%, or at least 90%, or at least 95% or at least 98% sequence identity to SEQ ID NOS: SEQ ID NOS: 508-526. In some embodiments, rAAV vector comprises a nucleic acid sequence encoding any of SEQ ID NOS: 508-526 and is codon optimized to have at least 50%, or 60% or 70% or 75%, 80%, 85%, 90%, 95% reduced CpG site content relative to the CpG site content of the nucleic acid sequence of SEQ ID NOS: 508-526.

In some embodiments, the rAAV vector encodes a nucleic acid for increasing angiogenesis, e.g., an angiogenic protein, as defined herein. Angiogenesis refers generally to the development and differentiation of blood vessels. A number of proteins, typically referred to as “angiogenic proteins,” are known to promote angiogenesis. Such angiogenic proteins include members of the fibroblast growth factor (FGF) family, the vascular endothelial growth factor (VEGF) family, the platelet-derived growth factor (PDGF) family, the insulin-like growth factor (IGF) family, and others. For example, the FGF and VEGF family members have been recognized as regulators of angiogenesis during growth and development. The angiogenic activity of the FGF and VEGF families has been examined. For example, it has been shown that acidic FGF (“aFGF”) protein, within a collagen-coated matrix, when placed in the peritoneal cavity of adult rats, resulted in a well vascularized and normally perfused structure (Thompson et al., Proc. Natl. Acad. Sci. USA, 86: 7928-7932, 1989). Injection of basic FGF (“bFGF”) protein into adult canine coronary arteries during coronary occlusion reportedly led to decreased myocardial dysfunction, smaller myocardial infarctions, and increased vascularity in the bed at risk (Yanagisawa-Miwa et al., Science, 257: 1401-1403, 1992). Similar results have been reported in animal models of myocardial ischemia using bFGF protein (Harada et al., J. Clin. Invest., 94: 623-630, 1994; Unger et al., Am. J. Physiol., 266: H1588-H-1595, 1994). An increase in collateral blood flow was shown in dogs treated with VEGF protein (Banai et al. Circulation 89: 2183-2189, 1994).

In some embodiments, a rAVV vector as disclosed herein comprising a cardiac-specific promoter encodes one or more angiogenic proteins or peptide, such as, for example, FGF-5, FGF-4, aFGF, bFGF and/or a VEGF, or variants thereof. Suitable angiogenic proteins or peptides are exemplified by members of the family of fibroblast growth factors (FGF), vascular endothelial growth factors (VEGF), platelet-derived growth factors (PDGF), insulin-like growth factors (IGF), and others. Members of the FGF family include, but are not limited to, aFGF (FGF-1), bFGF (FGF-2), FGF-4 (also known as “hst/KS3”), FGF-5, FGF-6. In some embodiments, the rAAV vector disclosed herein encodes a secreted angiogenic protein, such as, FGF-4, FGF-5, or FGF-6 since these proteins contain functional secretory signal sequences and are readily secreted from cells. Many if not most human VEGF proteins (including but not limited to VEGF-121 and VEGF-165) also are readily secreted and diffusible after secretion. VEGF has been shown to be expressed by cardiac myocytes in response to ischemia in vitro and in vivo; it is a regulator of angiogenesis under physiological conditions as well as during the adaptive response to pathological states (Banai et al. Circulation 89:2183-2189, 1994). The VEGF family, includes, but is not limited to, members of the VEGF-A sub-family (e.g. VEGF-121, VEGF-145, VEGF-165, VEGF-189 and VEGF-206), as well as members of the VEGF-B sub-family (e.g. VEGF-167 and VEGF-186) and the VEGF-C sub-family. PDGF includes, e.g., PDGF A and PDGF B, and IGF includes, for example, IGF-1. Other angiogenic proteins or peptides are known in the art and new ones are regularly identified. The nucleotide sequences of genes encoding these and other proteins, and the corresponding amino acid sequences are likewise known in the art (see, e.g., the GENBANK sequence database).

Angiogenic proteins and peptides include peptide precursors that are post-translationally processed into active peptides and “derivatives” and “functional equivalents” of angiogenic proteins or peptides. Derivatives of an angiogenic protein or peptide are peptides having similar amino acid sequence and retaining, to some extent, one or more activities of the related angiogenic protein or peptide. As is well known to those of skill in the art, useful derivatives generally have substantial sequence similarity (at the amino acid level) in regions or domains of the protein associated with the angiogenic activity. Similarly, those of skill in the art will readily appreciate that by “functional equivalent” is meant a protein or peptide that has an activity that can substitute for one or more activities of a particular angiogenic protein or peptide. Preferred functional equivalents retain all of the activities of a particular angiogenic protein or peptide; however, the functional equivalent may have an activity that, when measured quantitatively, is stronger or weaker than the wild-type peptide or protein.

For details on the FGF family, see, e.g., Burgess, Ann. N.Y. Acad. Sci. 638: 89-97, 1991; Burgess et al. Annu. Rev. Biochem. 58: 575-606, 1989; Muhlhauser et al., Hum. Gene Ther. 6: 1457-1465, 1995; Zhan et al., Mol. Cell. Biol., 8: 3487, 1988; Seddon et al., Ann. N.Y. Acad. Sci. 638: 98-108, 1991. For human hst/KS3 (i.e. FGF-4), see Taira et al. Proc. Natl. Acad. Sci. USA 84: 2980-2984, 1987. For human VEGF-A protein, see e.g., Tischer et al. J. Biol. Chem. 206: 11947-11954, 1991, and references therein; Muhlhauser et al., Circ. Res. 77: 1077-1086, 1995; and Neufeld et al., WO 98/10071 (Mar. 12, 1998). Other variants of known angiogenic proteins have likewise been described; for example variants of VEGF proteins and VEGF related proteins, see e.g., Baird et al., WO 99/40197, (Aug. 12, 1999); and Bohlen et al., WO 98/49300, (Nov. 5, 1998). Combinations of angiogenic proteins and gene delivery vectors encoding such combinations are described in Gao et al. U.S. Ser. No. 09/607,766, filed Jun. 30, 2000, entitled “Dual Recombinant Gene Therapy Compositions and Methods of Use”, hereby incorporated by reference in its entirety. As is also appreciated by those of skill in the art, angiogenic proteins can promote angiogenesis by enhancing the expression, stability or functionality of other angiogenic proteins. Examples of such angiogenic proteins or peptides include, e.g., regulatory factors that are induced in response to hypoxia (e.g. the hypoxia-inducible factors such as Hif-1, Hif-2 and the like; see, e.g., Wang et al., Proc. Natl. Acad. Sci. USA 90(9): 4304-8, 1993; Forsythe et al., Mol. Cell. Biol. 16(9): 4604-13, 1996; Semenza et al., Kidney Int., 51(2): 553-5, 1997; and O’Rourke et al., Oncol. Res., 9(6-7): 327-32, 1997; as well as other regulatory factors, such as, for example, those that are induced by physiological conditions associated with cardiovascular disease, such as inflammation (e.g., inducible nitric oxide synthase (iNOS), as well as the constitutive counterpart, CNOS; see e.g., Yoshizumi et al., Circ. Res., 73(1): 205-9, 1993; Chartrain et al., J. Biol. Chem., 269(9): 6765-72, 1994; Papapetropoulos et al., Am. J. Pathol., 150(5): 1835-44, 1997; and Palmer, et al., Am. J. Physiol., 274(2 Pt 1): L212-9, 1998). Additional examples of such angiogenic proteins include certain insulin-like growth factors (e.g., IGF-1) and angiopoietins (Angs), which have been reported to promote and/or stimulate expression and/or activity of other angiogenic proteins such as VEGF (see e.g. Goad, et al, Endocrinology, 137(6):2262-68 (1996); Warren, et al., J. Bio. Chem., 271(46):29483-88 (1996); Punglia, et al, Diabetes, 46(10):1619-26 (1997); and Asahara, et al., Circ. Res., 83(3):233-40 (1998) and Bermont et al. Int. J. Cancer 85: 117-123, 2000). Similarly, hepatocyte growth factor (also referred to as Scatter factor), which has been reported to induce blood vessel formation in vivo (see, e.g., Grant et al. Proc. Natl. Acad. Sci. USA 90: 1937-1941, 1993) has also been reported to increase expression of VEGF (see, e.g., Wojta et al., Lab Invest. 79:427-438, 1999). Additional examples of angiogenic polypeptides include natural and synthetic regulatory peptides (angiogenic polypeptide regulators) that act as promoters of endogenous angiogenic genes. Native angiogenic polypeptide regulators can be derived from inducers of endogenous angiogenic genes. Hif, as described above, is one illustrative example of such an angiogenic gene which has been reported to promote angiogenesis by inducing expression of other angiogenic genes. Synthetic angiogenic polypeptide regulators can be designed, for example, by preparing multi-finger zinc-binding proteins that specifically bind to sequences upstream of the coding regions of endogenous angiogenic genes and which can be used to induce the expression of such endogenous genes. Studies of numerous genes has led to the development of “rules” for the design of such zinc-finger DNA binding proteins (see, e.g., Rhodes and Klug, Scientific American, February 1993, pp 56-65; Choo and Klug, Proc. Natl. Acad. Sci. USA, 91(23): 11163-7, 1994; Rebar and Pabo, Science, 263(5147): 671-3, 1994; Choo et al., J. Mol. Biol., 273(3): 525-32, 1997; Pomerantz et al., Science 267: 93-96, 1995; and Liu et al., Proc. Natl. Acad. Sci. USA, 94: 5525-5530, 1997. As will be appreciated by those of skill in the art, numerous additional genes encoding proteins or peptides having the capacity to directly or indirectly promote angiogenesis are regularly identified and new genes will be identified based on similarities to known angiogenic protein or peptide encoding genes or to the discovered capability of such genes to encode proteins or peptides that promote angiogenesis. Sequence information for such genes and encoded polypeptides is readily obtainable from sequence databases such as GenBank or EMBL. Polynucleotides encoding these proteins can also be obtained from gene libraries, e.g., by using PCR or hybridization techniques routine in the art.

In some embodiments, the protein can be an inhibitor of a cytokine such as an IL-18 inhibitor. For example, the GSK antibody SGK-1070806 antibody, a null Cas9 to IL-18, a siRNA, miRNA, or an IL-18 binding protein (IL-18BP). IL-18BP is a soluble protein having a high affinity for IL-18 (Novick et al., 1999; as disclosed in WO 99/09063).

IL-18BP is not the extracellular domain of one of the known IL18 receptors, but a secreted, naturally circulating protein. It belongs to a novel family of secreted proteins, further including several Poxvirus-encoded proteins (Novick et al., 1999). Urinary as well as recombinant IL-18BP specifically bind IL-18 with a high affinity and modulate the biological affinity of IL-18. IL-18 binds with high affinity and signals through the IL-18 receptor (IL-18R), a heteromeric complex of alpha and beta chains encoded by the genes IL18R1 and IL18RAP, respectively (Torigoe K et al (1997) J Biol Chem; 272(41):25737-42). The bioactivity of IL-18 is negatively regulated by the IL18BP, a naturally occurring and highly specific inhibitor. This soluble protein forms a complex with free IL-18 preventing its interaction with the IL-18 receptor, thus neutralizing and inhibiting its biological activity (Dinarello C A (2000) Ann Rheum Dis; 59 Suppl 1:i17-20). IL-18BP is a constitutively secreted protein with high affinity binding to IL-18. Alternate mRNA splicing variants of IL-18BP result in four isoforms. The prominent ‘a’ isoform is present in the serum of healthy humans at 20-fold molar excess compared with IL-18 (Dinarello and Kaplanski (2005) Expert Rev Clin Immunol, 1(4), 619-632).

The IL-18BP gene was localized to the human chromosome 1 lq13, and no exon coding for a transmembrane domain was found in an 8.3 kb genomic sequence. Four splice variants or isoforms of IL-18BP generated by alternative mRNA splicing have been found in humans so far. They were designated IL-18BP a, b, c and d, all sharing the same N-terminus and differing in the C-terminus (Novick et al, 1999). These isoforms vary in their ability to bind IL-18. Of the four, hIL-18BP isoforms a and c are known to have a neutralizing capacity for IL-18. Human IL-18BP isoform binds to murine IL-18. The rAAV encodes IL-18 inhibitor as disclosed in WO2015032932 or US20140112915, or IL-18BP, as disclosed in WO1999009063 which is incorporated herein in its entirety.

In some embodiments, the rAAV vector can encode a beta-adrenergic signaling protein (beta-ASPs) (including beta-adrenergic receptors (beta-ARs), G-protein receptor kinase inhibitors (GRK inhibitors) and adenylylcyclases (ACs)) to enhance cardiac function as described and illustrated in detail in U.S. Pat. Application Ser. No. 08/924,757, filed Sep., 1997 (based on U.S. No. 60/048,933 filed Jun. 16, 1997 and U.S. Pat. No. 08/708,661 filed Sep., 1996), as well as PCT/US97/15610 filed Sep., 1997, and U.S. continuing case Ser. No. 09/008,097, filed Jan. 16, 1998, and U.S. continuing case Ser. No. 09/472,667, filed Dec. 27, 1999, each of which is incorporated by reference herein.

In some embodiments, rAAV vectors comprising cardiac-specific promoters as disclosed herein can be assessed using a myocardial infarction (MI) model as disclosed in Angeli et al., Comparative Medicine, 2009, 59(3), 272-279.

### B. Cardiac Specific Promoters (CSP)

In some embodiments, the rAAV vector comprises a nucleic acid encoding the therapeutic agent, e.g., inhibitor of PP1 or other agent, operatively linked to a cardiac-specific promoter. Exemplary cardiac specific promoters are disclosed in Table 2A, 2B, 3 and 4 herein. In some embodiments, the cardiac specific promoter is a synthetic cardiac specific promoter.

In some embodiments, to achieve appropriate levels of expression of the transgene, e.g., inhibitor of PP1 (e.g., I-1 or I-lc), the rAAV genotype comprises a cardiac specific promoter (CSP). A CSP enables expression of the operatively linked gene in the heart tissue, and can in some embodiments, be an inducible CSP. In an embodiment, a CSP is located upstream 5′ and is operatively linked to the heterologous nucleic acid sequence encoding the transgene, e.g., inhibitor of PP1 (e.g., I-1 or I-1c). Exemplary cardiac-specific promoters are disclosed herein, e.g., any selected from Tables 1 herein, or functional variants thereof. In some embodiments of the compositions and methods disclosed herein, a cardiac-specific promoter includes a cardiac-specific cis-regulatory element (CRE), a synthetic cardiac-specific cis-regulatory module (CRM) or a synthetic cardiac-specific promoter as disclosed in Tables 1-3.

In some embodiments, to achieve appropriate levels of expression of a PP1 inhibitor (e.g., I-1 or I-1c expression), the rAAV genotype comprises a cardiac specific promoter (CSP). A CSP enables expression of the operatively linked gene in the heart, and can in some embodiments, be and inducible CSP. In an embodiment, a CSP is located upstream 5′ and is operatively linked to the heterologous nucleic acid sequence encoding the PP1 inhibitor protein. Exemplary CSP are disclosed herein, and include for example, the CSP listed in Table 2A herein or functional variants thereof. In some embodiments of the compositions and methods disclosed herein, a cardiac-specific promoter includes a cardiac-specific cis-regulatory element (CRE), a synthetic cardiac-specific cis-regulatory module (CRM) or a synthetic cardiac-specific promoter that comprises elements of minimal cardiac-specific promoters or cardiac-specific proximal promoters.

### I. Cardiac-Specific Promoters

Table 2A shows nucleic acid sequences of exemplary cardiac-specific promoters for use in the methods and composition as disclosed herein.

TABLE 2A

| NAME | LENGTH | NAME | LENGTH |
|---|---|---|---|
| SP0067 (SEQ ID NO: 3) | 443 | SP0480 (SEQ ID NO: 22) | 496 |
| SP0075 (SEQ ID NO: 4) | 433 | SP0481 (SEQ ID NO: 23) | 456 |
| SP0424 (SEQ ID NO: 5) | 765 | SP0482 (SEQ ID NO: 24) | 517 |
| SP0425 (SEQ ID NO: 6) | 631 | SP0483 (SEQ ID NO: 25) | 453 |
| SP0429 (SEQ ID NO: 7) | 806 | SP0484 (SEQ ID NO: 26) | 348 |
| SP0430 (SEQ ID NO: 8) | 842 | SP0485 (SEQ ID NO: 27) | 442 |
| SP0344 (SEQ ID NO: 9) | 460 | SP0488 (SEQ ID NO: 28) | 463 |
| SP0433 (SEQ ID NO: 10) | 554 | SP0487 (SEQ ID NO: 29) | 337 |
| SP0435 (SEQ ID NO: 11) | 609 | SP0488 (SEQ ID NO: 30) | 544 |
| SP0436 (SEQ ID NO: 12) | 632 | SP0489 (SEQ ID NO: 31) | 553 |
| SP0449 (SEQ ID NO: 13) | 782 | SP0490 (SEQ ID NO: 32) | 497 |
| SP0450 (SEQ ID NO: 14) | 823 | SP0491 (SEQ ID NO: 33) | 478 |
| SP0451 (SEQ ID NO: 15) | 859 | SP0492 (SEQ ID NO: 34) | 378 |
| SP0452 (SEQ ID NO: 16) | 851 | SP0493(SEQ ID NO: 35) | 359 |
| SP0475 (SEQ ID NO: 17) | 647 | SP0494 (SEQ ID NO: 36) | 545 |
| SP0476 (SEQ ID NO: 18) | 501 | SP0495 (SEQ ID NO: 37) | 567 |
| SP0477 (SEQ ID NO: 19) | 484 | SP0496 (SEQ ID NO: 38) | 548 |
| SP0478 (SEQ ID NO: 20) | 465 | SP0448 (SEQ ID NO: 288) | 763 |
| SP0479 (SEQ ID NO: 21) | 456 | SP0434 (SEQ ID NO: 289) | 420 |

Aspects of the technology relate to a rAAV vector comprising a synthetic cardiac-specific promoter as disclosed in Table 2A. In some embodiments, the rAAV vector comprises a synthetic cardiac-specific promoter disclosed in Table 2A, which operatively linked to a nucleic acid encoding an inhibitor of PP1 as disclosed herein, or a gene as disclosed in Table 18A or 18B disclosed herein.

In some embodiments, the synthetic cardiac-specific promoter disclosed herein can comprise one or more cis-regulatory elements (CREs) and/or a minimal promoter or proximal promoters, and/or a regulatory element (RE) such as a 5′ UTR or intron, or RE that functions as both a 5′UTR and intron (e.g., CMV-IE), which are disclosed herein.

Table 2B: CRE and minimal/proximal promoters of the embodiments of cardiac-specific promoters of Table 2A.

| | | | | |
|---|---|---|---|---|
| Promoter | CRE | CRE | Minimal or proximal promoter | 5′Intron/UTR (if present) |
| SP0067 (SEQ ID NO: 3) | | CRE0033 (SEQ ID NO: 41) | SKM_18 (SEQ ID NO: 55) | |
| SP0075 (SEQ ID NO” SEQ ID NO: 4) | | CRE0033 (SEQ ID NO: 41) | SKM_20 (SEQ ID NO: 56) | |
| SP0424 (SEQ ID NO: 5) | | CRE0004 (SEQ ID NO: 39) | CRE0082 (SEQ ID NO: 57) | |
| SP0425 (SEQ ID NO: 6) | | CRE0028 (SEQ ID NO: 40) | CRE0082 (SEQ ID NO: 57) | |
| SP0429 (SEQ ID NO: 7) | | CRE0095 (SEQ ID NO: 44) | CRE0082 (SEQ ID NO: 57) | |
| SP0430 (SEQ ID NO: 8) | | CRE0096 (SEQ ID NO: 45) | CRE0082 (SEQ ID NO: 57) | |
| SP0344 (SEQ ID NO: 9) | | CRE0033 (SEQ ID NO: 41) | CRE0038 (SEQ ID NO: 64) | |
| SP0433 (SEQ ID NO: 10) | CRE0033 (SEQ ID NO: 41) | CRE0071.3 (SEQ ID NO:43) | CRE0070 (SEQ ID NO:42) | |
| SP0435 (SEQ ID NO: 11) | | CRE0033 (SEQ ID NO: 41) | CRE0082 (SEQ ID NO: 57) | |
| SP0436 (SEQ ID NO: 12) | CRE0033 (SEQ ID NO: 41) | CRE0033 (SEQ ID NO: 41) | SKM_18 (SEQ ID NO: 55) | |
| SP0449 (SEQ ID NO: 13) | CRE0004 (SEQ ID NO:39) | CRE0033 (SEQ ID NO: 41) | SKM_18 (SEQ ID NO: 55) | |
| SP0450 (SEQ ID NO: 14) | CRE0095 (SEQ ID NO: 44) | CRE0033 (SEQ ID NO: 41) | SKM_18 (SEQ ID NO: 55) | |
| SP0451 (SEQ ID NO: 15) | CRE0096 (SEQ ID NO: 45) | CRE0033 (SEQ ID NO: 41) | SKM_18 (SEQ ID NO: 55) | |
| SP0452 (SEQ ID NO: 16) | CRE0082 (SEQ ID NO:57) | CRE0033 (SEQ ID NO: 41) | SKM_18 (SEQ ID NO: 55) | |
| SP0475 (SEQ ID NO: 17) | | CRE0033 (SEQ ID NO: 41) | SKM_18 (SEQ ID NO: 55) | CMV-IE 5′UTR and intron (A SEQ ID NO: 65) |
| SP0476 (SEQ ID NO: 18) | | CRE0105 (SEQ ID NO: 46) | SKM_18 (SEQ ID NO: 55) | |
| SP0477 (SEQ ID NO: 19) | | CRE0106 (SEQ ID NO: 47) | SKM_18 (SEQ ID NO: 55) | |
| SP0478 (SEQ ID NO: 20) | | CRE0107 ((SEQ ID NO: 48) | SKM_18 (SEQ ID NO: 55) | |
| SP0479 (SEQ ID NO: 21) | | CRE0108 (SEQ ID NO:49) | SKM_18 (SEQ ID NO: 55) | |
| SP0480 (SEQ ID NO: 22) | | CRE0109 (SEQ ID NO:50) | SKM_18 (SEQ ID NO: 55) | |
| SP0481 (SEQ ID NO: 23) | | CRE0033 (SEQ ID NO: 41) | CRE0110 (SEQ ID NO: 59) | |
| SP0482 (SEQ ID NO: 24) | | CRE01 1 1 ((SEQ ID NO: 51) | SKM_18 (SEQ ID NO:55) | |
| SP0483 (SEQ ID NO: 25) | | CRE0033 (SEQ ID NO: 41) | CRE0112 (SEQ ID NO: 60) | |
| SP0484 (SEQ ID NO: 26) | | CRE0033 (SEQ ID NO: 41) | CRE0113 (SEQ ID NO: 61) | |
| SP0485 (SEQ ID NO: 27) | | CRE0114 (AU) | SKM_18 (SEQ ID NO: 55) | |
| SP0486 (SEQ ID NO: 28) | | CRE0033 (SEQ ID NO: 41) | CRE0115 (SEQ ID NO: 62) | |
| SP0487 (SEQ ID NO: 29) | | CRE0033 (SEQ ID NO: 41) | CRE0116 (SEQ ID NO: 63) | |
| SP0488 (SEQ ID NO: 30) | | CRE0117 (SEQ ID NO: 53) | SKM_18 (SEQ ID NO: 55) | |
| SP0489 (SEQ ID NO: 31) | | CRE0033 (SEQ ID NO: 41) | CRE0104 (SEQ ID NO: 58) | |
| SP0490 (SEQ ID NO: 32) | | CRE0106 (SEQ ID NO:47) | CRE0110 (SEQ ID NO: 59) | |
| SP0491 (SEQ ID NO: 33) | | CRE0107 (SEQ ID NO: 48) | CRE0110 (SEQ ID NO: 59) | |
| SP0492 (SEQ ID NO: 34) | | CRE0106 (SEQ ID NO: 47) | CRE0116 (SEQ ID NO: 63) | |
| SP0493 (SEQ ID NO: 35) | | CRE0107 (SEQ ID NO: 48) | CRE0116 (SEQ ID NO: 63) | |
| SP0494 (SEQ ID NO: 36) | | CRE0118 (SEQ ID NO: 54) | SKM_18 (SEQ ID NO: 55) | |
| SP0495 (SEQ ID NO: 37) | CRE0106 (SEQ ID NO: 47) | CRE0033 (SEQ ID NO: 41) | CRE0116 (SEQ ID NO: 63) | |
| SP0496 (SEQ ID NO: 38) | CRE0107 (SEQ ID NO: 48) | CRE0033 (SEQ ID NO: 41) | CRE0116 (SEQ ID NO: 63) | |

The CREs, CRMs, introns, UTRs, minimal/proximal promoters and promoters as disclosed herein can be active in various muscle tissues, particularly but not exclusively in skeletal muscle and/or cardiac muscle. CREs, CRMs, promoter elements or promoters which are active in at least one muscle tissue type or at least one muscle cell type may be referred to as ‘muscle-specific’. For ease, muscle-specific CREs, CRMs, promoter elements or promoters can be further subdivided in subtypes depending on whether the CREs, CRMs, promoter elements or promoters are predominantly active in skeletal or cardiac muscle.

In some embodiments the cis-regulatory elements and promoters of the present invention are skeletal muscle-specific. In some embodiments the cis-regulatory elements, CRMs, promoter elements and promoters of the present invention are active predominantly in skeletal muscle and less active or not active in cardiac muscle. These CREs, CRMs, promoter elements and promoters are called ‘skeletal muscle-specific’.

In some embodiments the cis-regulatory elements and promoters of the present invention are cardiac muscle-specific. In some embodiments the cis-regulatory elements, CRMs, promoter elements and promoters of the present invention are active predominantly in cardiac muscle and less active or not active in skeletal muscles. These CREs, CRMs, promoter elements and promoters are called ‘cardiac muscle-specific’.

In some embodiments, muscle-specific CREs, CRMs, promoter elements and promoters are active in both skeletal muscle and cardiac muscle. These CREs, CRMs, promoter elements and promoters may be preferred when promoter activity is required in both the skeletal muscle and the heart (in the cardiac muscles). In some embodiments, cardiac muscle-specific CREs, CRMs, promoter elements and promoters may be preferred. These CREs, CRMs, promoter elements and promoters may be preferred when promoter activity is required in the heart (in the cardiac muscles) with little or no activity in the skeletal muscles. Examples of synthetic cardiac muscle-specific promoters include SP0067, SP0075, SP0424, SP0425, SP0429, SP0430, SP0433, SP0436, SP0452, SP0344, SP0483, SP0496, SP0435, SP0449, SP0450, SP0451, SP0475, SP0476, SP0477, SP0478, SP0479, SP0480, SP0481, SP0482, SP0484, SP0485, SP0486, SP0487, SP0488, SP0489, SP0490, SP0491, SP0492, SP0493, SP0494 and SP0495. Examples of preferred synthetic cardiac muscle-specific promoters are SP0067, SP0433, SP0436, SP0452, SP0344 and SP0483.

The cardiac muscle-specific CREs, CRMs, promoter element and promoters of the present invention can be active in various cells of the heart. The predominant cell types in the heart are ventricular cardiomyocytes, atrial cardiomyocytes, cardiac fibroblasts, or endothelial cells (EC) in the heart, as well as peri-vascular cells and pacemaker cells. Additionally, the cardiac muscle-specific CREs, CRMs, promoter element and promoters of the present invention can be active in various regions of the heart, such as, for example, activity in any or all of the following heart regions: aortic arch arteries (AA); aorta; cardiomyocytes (CM); endothelial or endocardial cells (ECs); inferior caval vein (ICV); interventricular septum (IVS); left atrium (LA); left superior caval vein (LSCV); left ventricle (LV); outflow tract (OT); pulmonary arteries (PO); proepicardial organ (PEO); pulmonary vein (PV); right atrium (RA); right superior caval vein (RSCV); right ventricle (RV); superior caval vein (SCV); cardiac smooth muscle cells (SMs).

In some embodiments, muscle-specific CREs, CRMs, promoter elements and promoters which are active in both skeletal muscle and cardiac muscle are also encompassed for use in the AAVs for the methods of administration and treatment as disclosed herein. These CREs, CRMs, promoter elements and promoters may be preferred when promoter activity is required in both the skeletal muscle and the heart (in the cardiac muscles). Examples of muscle-specific promoters active in both skeletal and cardiac muscle include SP0010, SP0020, SP0033, SP0038, SP0040, SP0042, SP0051, SP0057, SP0058, SP0061, SP0062, SP0064, SP0065, SP0066, SP0068, SP0070, SP0071, SP0076, SP0132, SP0133, SP0134,SP0136, SP0146, SP0147, SP0148, SP0150, SP0153, SP0155, SP0156, SP0157, SP0158, SP0159, SP0160, SP0161, SP0162, SP0163, SP0164, SP0165, SP0166, SP0169, SP0170, SP0171, SP0173, SP0228, SP0229, SP0230, SP0231, SP0232, SP0257, SP0262, SP0264 SP0265, SP0266, SP0267, SP0268, SP0270, SP0271, SP0279, SP0286, SP0305, SP0306, SP0307, SP0309, SP0310, SP0311, SP0312, SP0313, SP0314, SP0315, SP0316, SP0320, SP0322, SP0323, SP0324, SP0325, SP0326, SP0327, SP0328, SP0329, SP0330, SP0331, SP0332, SP0333, SP0334, SP0335, SP0336, SP0337, SP0338, SP0339, SP0340, SP0341, SP0343, SP0345, SP0346, SP0347, SP0348, SP0349, SP0350, SP0351, SP0352, SP0353, SP0354, SP0355, SP0356, SP0358, SP0359, SP0361, SP0362, SP0363, SP0364, SP0365, SP0366, SP0367, SP0368, SP0369, SP0370, SP0371, SP0372, SP0373, SP0374, SP0375, SP0376, SP0377, SP0378, SP0379, SP0380, SP0381, SP0382, SKM_14, SKM_18, SKM_20, SP0357, SP0437-SP0445, SP0447 and SP0453-SP0471, 473-474. Examples of preferred synthetic muscle-specific promoters which are active in both skeletal and cardiac muscles are SP0057, SP0134, SP0173, SP0279, SP0286, SP0310, SP0316, SP0320 and SP0326.

### II. Functional Variants of the Cardiac Specific Promoters

In some embodiments, the promoter is a synthetic cardiac-specific promoter comprising a combination of the cis-regulatory elements (CREs), for example CRE0051 and CRE0042, or functional variants thereof. Typically, the CREs are operably linked to a promoter element. In some preferred embodiments, the cardiac-specific promoter comprises said CREs, or functional variants thereof, in the order recited, such as CRE0051, CRE0042, and then the promoter element (order is given in an upstream to downstream direction, as is conventional in the art). In some preferred embodiments, the cardiac-specific promoter comprises said CREs, or functional variants thereof, in a different order from that recited, such as CRE0042, CRE0051 and then the promoter element.

In some embodiments, the cardiac-specific promoter comprises said CREs, or functional variants thereof, in the order recited, such as CRE0033, and then any other CRE element, or the promoter element disclosed herein. For example, the promoter can comprise CRE0033 and at least one CRE, or at least 2 CREs, or at least 3 CREs, or at least 4 CREs or more than 4 CREs selected from any CRE disclosed in Tables 2B, 3, 5B or 6, as disclosed herein.

The promoter element can be any suitable proximal promoter or minimal promoter. In some embodiments, the promoter element is a minimal promoter. Where the promoter is a proximal promoter, it is generally preferred that the proximal promoter is cardiac-specific.

### SP0067 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0033 and then SKM_18.

CRE0033 has the nucleic acid sequence of SEQ ID NO: 41: Functional variants of SEQ ID NO: 41 thereof may have a sequence that is at least 60%, 65%, 70%, 75%, 80%, 85%, 90%, 95%, 96%, 97%, 98%, or 99% identical thereto.

Functional variants of CRE0033 are regulatory elements with sequences which vary from CRE0033, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0033 can be viewed as a CRE which, when substituted in place of CRE0033 in a promoter, substantially retains its activity. For example, a cardiac muscle-specific promoter which comprises a functional variant of CRE0033 substituted in place of CRE0033 preferably retains 80% of its activity, more preferably 90% of its activity, more preferably 95% of its activity, and yet more preferably 100% of its activity. For example, considering promoter SP0067 as an example, CRE0033 in SP0067 can be replaced with a functional variant of CRE0033, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0033 or functional variant thereof can be provided on either strand of a double stranded polynucleotide and can be provided in either orientation. As such, complementary and reverse complementary sequences of SEQ ID NO: 41 or a functional variant thereof fall within the scope of the invention. Single stranded nucleic acids comprising the sequence according to SEQ ID NO: 41 or a functional variant thereof also fall within the scope of the invention.

In some embodiments, the CRE033 or a functional variant thereof, has a length of 200 or fewer nucleotides, 150 or fewer nucleotides, 125 or fewer nucleotides, or 100 or fewer nucleotides.

SKM_18 has the nucleic acid sequence of SEQ ID NO: 55. Functional variants of SEQ ID NO: 51 may have a sequence that is at least 60%, 65%, 70%, 75%, 80%, 85%, 90%, 95%, 96%, 97%, 98%, or 99% identical thereto.

As discussed above, functional variants of SKM_18 substantially retain the ability of SKM_18 to act as a muscle-specific promoter element. For example, when a functional variant of SKM_18 is substituted into cardiac muscle-specific promoter SP0067, the modified promoter retains at least 80% of its activity, more preferably at least 90% of its activity, more preferably at least 95% of its activity, and yet more preferably 100% of the activity of SP0067. Suitably the functional variant of SKM_18 comprises a sequence which has at least 70%, 80%, 90%, 95% or 99% identity to SEQ ID NO: 55.

In some preferred embodiments, a promoter element comprising or consisting of SKM_18 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises SEQ ID NO: 3, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 3 is referred to as SP0067. The SP0067 promoter is particularly preferred in some embodiments. This promoter has been found to be very specific for cardiac muscle and is also very short, which is advantageous in some circumstances.

### SP0075 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_20 or functional variant thereof. SKM_20 is a muscle-specific proximal promoter.

In some embodiments the cardiac muscle-specific promoter comprises the following elements (or functional variants thereof): CRE0033 and then SKM_20.

The sequence of CRE0033 and variants thereof are set out above.

SKM_20 has the nucleic acid sequence of SEQ ID NO: 56. Functional variants of SEQ ID NO: 56 may have a sequence that is at least 60%, 65%, 70%, 75%, 80%, 85%, 90%, 95%, 96%, 97%, 98%, or 99% identical thereto.

As discussed above, functional variants of SKM_20 substantially retain the ability of SKM_20 to act as a muscle-specific promoter element. For example, when a functional variant of SKM_20 is substituted into cardiac muscle-specific promoter SP0075, the modified promoter retains at least 80% of its activity, more preferably at least 90% of its activity, more preferably at least 95% of its activity, and yet more preferably 100% of the activity of SP0075. Suitably the functional variant of SKM_20 comprises a sequence which has at least 70%, 80%, 90%, 95% or 99% identity to SEQ ID NO: 56.

In some preferred embodiments, a promoter element comprising or consisting of SKM_20 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises SEQ ID NO: 4, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 4 is referred to as SP0075. The SP0075 promoter is particularly preferred in some embodiments. This promoter has been found to be very specific for cardiac muscle and is also very short, which is advantageous in some circumstances.

### SP0424 and Variants Thereof

In some embodiments, the promoter is a synthetic cardiac muscle-specific promoter comprising CRE0004 operably linked to a promoter element. In some preferred embodiments, the synthetic cardiac muscle-specific promoter comprises CRE0004 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0004 and then CRE0082.

CRE0004 has the nucleic acid sequence of SEQ ID NO: 39. Functional variants of SEQ ID NO: 39 may have a sequence that is at least 60%, 65%, 70%, 75%, 80%, 85%, 90%, 95%, 96%, 97%, 98%, or 99% identical thereto.

Functional variants of CRE0004 are regulatory elements with sequences which vary from CRE0004, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0004 can be viewed as a CRE which, when substituted in place of CRE0004 in a promoter, substantially retains its activity. For example, a cardiac muscle-specific promoter which comprises a functional variant of CRE0004 substituted in place of CRE0004 preferably retains 80% of its activity, more preferably 90% of its activity, more preferably 95% of its activity, and yet more preferably 100% of its activity. For example, considering promoter SP0424 as an example, CRE0004 in SP00424 can be replaced with a functional variant of CRE0004, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0004 or functional variant thereof can be provided on either strand of a double stranded polynucleotide and can be provided in either orientation. As such, complementary and reverse complementary sequences of SEQ ID NO: 39 or a functional variant thereof fall within the scope of the invention. Single stranded nucleic acids comprising the sequence according to SEQ ID NO: 39 or a functional variant thereof also fall within the scope of the invention.

In some embodiments, the CRE004 or a functional variant thereof, has a length of 300 or fewer nucleotides, 200 or fewer nucleotides, 150 or fewer nucleotides, 125 or fewer nucleotides, or 100 or fewer nucleotides.

CRE0082 has the nucleic acid sequence of SEQ ID NO: 57. Functional variants of SEQ ID NO: 57 may have a sequence that is at least 60%, 65%, 70%, 75%, 80%, 85%, 90%, 95%, 96%, 97%, 98%, or 99% identical thereto.

As discussed above, functional variants of CRE0082 substantially retain the ability of CRE0082 to act as a cardiac muscle-specific promoter element. For example, when a functional variant of CRE0082 is substituted into cardiac muscle-specific promoter SP0424, the modified promoter retains at least 80% of its activity, more preferably at least 90% of its activity, more preferably at least 95% of its activity, and yet more preferably 100% of the activity of SP0424. Suitably the functional variant of CRE0082 comprises a sequence which has at least 70%, 80%, 90%, 95% or 99% identity to SEQ ID NO: 57.

In some preferred embodiments, a promoter element comprising or consisting of CRE0082 or a functional variant thereof has a length of 500 or fewer, 400 or fewer, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises SEQ ID NO: 5, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 5 is referred to as SP0424. The SP0424 promoter is particularly preferred in some embodiments. This promoter has been found to be specific for cardiac muscle, which is advantageous in some circumstances.

### SP0425 and Variants Thereof

In some embodiments, the promoter is a synthetic cardiac muscle-specific promoter comprising CRE0028 operably linked to a promoter element. In some preferred embodiments, the synthetic cardiac muscle-specific promoter comprises CRE0028 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0028 and then CRE0082.

CRE0028 has the nucleic acid sequence of SEQ ID NO: 40. Functional variants of SEQ ID NO: 40 may have a sequence that is at least 60%, 65%, 70%, 75%, 80%, 85%, 90%, 95%, 96%, 97%, 98%, or 99% identical thereto.

Functional variants of CRE0028 are regulatory elements with sequences which vary from CRE0028, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0028 can be viewed as a CRE which, when substituted in place of CRE0028 in a promoter, substantially retains its activity. For example, a cardiac muscle-specific promoter which comprises a functional variant of CRE0028 substituted in place of CRE0028 preferably retains 80% of its activity, more preferably 90% of its activity, more preferably 95% of its activity, and yet more preferably 100% of its activity. For example, considering promoter SP0425 as an example, CRE0028 in SP00425 can be replaced with a functional variant of CRE0028, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0028 or functional variant thereof can be provided on either strand of a double stranded polynucleotide and can be provided in either orientation. As such, complementary and reverse complementary sequences of SEQ ID NO: 40 or a functional variant thereof fall within the scope of the invention. Single stranded nucleic acids comprising the sequence according to SEQ ID NO: 40 or a functional variant thereof also fall within the scope of the invention.

In some embodiments, the CRE0028 or a functional variant thereof, has a length of 300 or fewer nucleotides, 200 or fewer nucleotides, 150 or fewer nucleotides, 125 or fewer nucleotides, or 100 or fewer nucleotides. The sequence of CRE0082 and variants thereof are set out above.

In some embodiments the cardiac muscle-specific promoter comprises the nucleic acid sequence of SEQ ID NO: 6, or a functional variant thereof. In some embodiments, functional variants of SEQ ID NO: 6 may have a sequence that is at least 60%, 65%, 70%, 75%, 80%, 85%, 90%, 95%, 96%, 97%, 98%, or 99% identical thereto. The promoter having a sequence according to SEQ ID NO: 6 is referred to as SP0425. The SP0425 promoter is particularly preferred in some embodiments. This promoter has been found to be specific for cardiac muscle, which is advantageous in some circumstances.

### SP0429 and Variants Thereof

In some embodiments, the promoter is a synthetic cardiac muscle-specific promoter comprising CRE0095 operably linked to a promoter element. In some preferred embodiments, the synthetic cardiac muscle-specific promoter comprises CRE0095 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0095 and then CRE0082.

CRE0095 has the nucleic acid sequence of SEQ ID NO: 44. Functional variants of SEQ ID NO: 44 may have a sequence that is at least 60%, 65%, 70%, 75%, 80%, 85%, 90%, 95%, 96%, 97%, 98%, or 99% identical thereto.

Functional variants of CRE0095 are regulatory elements with sequences which vary from CRE0095, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0095 can be viewed as a CRE which, when substituted in place of CRE0095 in a promoter, substantially retains its activity. For example, a cardiac muscle-specific promoter which comprises a functional variant of CRE0095 substituted in place of CRE0095 preferably retains 80% of its activity, more preferably 90% of its activity, more preferably 95% of its activity, and yet more preferably 100% of its activity. For example, considering promoter SP0429 as an example, CRE0095 in SP0429 can be replaced with a functional variant of CRE0095, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0095 or functional variant thereof can be provided on either strand of a double stranded polynucleotide and can be provided in either orientation. As such, complementary and reverse complementary sequences of SEQ ID NO: 44 or a functional variant thereof fall within the scope of the invention. Single stranded nucleic acids comprising the sequence according to SEQ ID NO: 44 or a functional variant thereof also fall within the scope of the invention.

In some embodiments, the CRE0095 or a functional variant thereof, has a length of 400 of fewer, 300 or fewer, 200 or fewer nucleotides, 150 or fewer nucleotides, 125 or fewer nucleotides, or 100 or fewer nucleotides.

The sequence of CRE0082 and variants thereof are set out above.

In some embodiments the cardiac muscle-specific promoter comprises the nucleic acid sequence of SEQ ID NO: 7, or a functional variant thereof. In some embodiments, functional variants of SEQ ID NO: 7 may have a sequence that is at least 60%, 65%, 70%, 75%, 80%, 85%, 90%, 95%, 96%, 97%, 98%, or 99% identical thereto. The promoter having a sequence according to SEQ ID NO: 7 is referred to as SP0429. The SP0429 promoter is particularly preferred in some embodiments. This promoter has been found to be specific for cardiac muscle, which is advantageous in some circumstances.

### SP0430 and Variants Thereof

In some embodiments, the promoter is a synthetic cardiac muscle-specific promoter comprising CRE0096 operably linked to a promoter element. In some preferred embodiments, the synthetic cardiac muscle-specific promoter comprises CRE0096 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0096 and then CRE0082.

CRE0096 has the nucleic acid sequence of SEQ ID NO: 45. Functional variants thereof may have a sequence that is at least 60%, 65%, 70%, 75%, 80%, 85%, 90%, 95%, 96%, 97%, 98%, or 99% identical thereto.

Functional variants of CRE0096 are regulatory elements with sequences which vary from CRE0096, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0096 can be viewed as a CRE which, when substituted in place of CRE0096 in a promoter, substantially retains its activity. For example, a cardiac muscle-specific promoter which comprises a functional variant of CRE0096 substituted in place of CRE0096 preferably retains 80% of its activity, more preferably 90% of its activity, more preferably 95% of its activity, and yet more preferably 100% of its activity. For example, considering promoter SP0430 as an example, CRE0096 in SP0430 can be replaced with a functional variant of CRE0096, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0096 or functional variant thereof can be provided on either strand of a double stranded polynucleotide and can be provided in either orientation. As such, complementary and reverse complementary sequences of SEQ ID NO: 45 or a functional variant thereof fall within the scope of the invention. Single stranded nucleic acids comprising the sequence according to SEQ ID NO: 45 or a functional variant thereof also fall within the scope of the invention.

In some embodiments, the CRE0096 or a functional variant thereof, has a length of 500 or fewer nucleotides, 400 or fewer nucleotides, 300 or fewer nucleotides, 200 or fewer nucleotides, 150 or fewer nucleotides, 125 or fewer nucleotides, or 100 or fewer nucleotides. The sequence of CRE0082 and variants thereof are set out above.

In some embodiments the cardiac muscle-specific promoter comprises the nucleic acid sequence of SEQ ID NO: 8, or a functional variant thereof. In some embodiments, functional variants of SEQ ID NO: 8 may have a sequence that is at least 60%, 65%, 70%, 75%, 80%, 85%, 90%, 95%, 96%, 97%, 98%, or 99% identical thereto. The promoter having a sequence according to SEQ ID NO: 8 is referred to as SP0430. The SP0430 promoter is particularly preferred in some embodiments. This promoter has been found to be specific for cardiac muscle, which is advantageous in some circumstances.

### SP0344 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0038 or functional variant thereof. CRE0038 is a cardiac muscle-specific proximal promoter.

In some embodiments the cardiac muscle-specific promoter comprises the following elements (or functional variants thereof): CRE0033 and then CRE0038.

The sequence of CRE0033 and variants thereof are set out above.

CRE0038 has the nucleic acid sequence of SEQ ID NO: 64. Functional variants of SEQ ID NO: 64 thereof may have a sequence that is at least 60%, 65%, 70%, 75%, 80%, 85%, 90%, 95%, 96%, 97%, 98%, or 99% identical thereto.

As discussed above, functional variants of CRE0038 substantially retain the ability of CRE0038 to act as a cardiac muscle-specific promoter element. For example, when a functional variant of CRE0038 is substituted into cardiac muscle-specific promoter SP0344, the modified promoter retains at least 80% of its activity, more preferably at least 90% of its activity, more preferably at least 95% of its activity, and yet more preferably 100% of the activity of SP0344. Suitably the functional variant of CRE0038 comprises a sequence which has at least 70%, 80%, 90%, 95% or 99% identity to SEQ ID NO: 64.

In some preferred embodiments, a promoter element comprising or consisting of CRE0038 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises nucleic acid sequence of SEQ ID NO: 9, or a functional variant thereof. In some embodiments, functional variants of SEQ ID NO: 9 may have a sequence that is at least 60%, 65%, 70%, 75%, 80%, 85%, 90%, 95%, 96%, 97%, 98%, or 99% identical thereto. The promoter having a sequence according to SEQ ID NO: 9 is referred to as SP0344. The SP0344 promoter is particularly preferred in some embodiments. This promoter has been found to be specific for cardiac muscle, which is advantageous in some circumstances.

### SP0433 and Variants Thereof

In some embodiments, the promoter is a synthetic cardiac muscle-specific promoter comprising a combination of the cis-regulatory elements CRE0033 and CRE0071.3, or functional variants thereof. Typically, the CREs are operably linked to a promoter element. In some preferred embodiments, the cardiac muscle-specific promoter comprises said CREs, or functional variants thereof, in the order CRE0033, CRE0071.3, and then the promoter element (order is given in an upstream to downstream direction, as is conventional in the art). In some embodiments, the cardiac muscle-specific promoter comprises said CREs, or functional variants thereof, in the order CRE0071.3, CRE0033, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is cardiac muscle-specific or cardiac muscle-specific.

In some preferred embodiments, the promoter element is CRE0070, or a functional variant thereof. CRE0070 is a muscle-specific proximal promoter.

Thus, in one embodiment the promoter comprises the following regulatory elements: CRE0033, CRE0071. 3 and CRE0070, or functional variants thereof. The sequence of CRE0033 and variants thereof are set out above. CRE0071.3 has nucleic acid sequence of SEQ ID NO: 43. Functional variants of SEQ ID NO: 43 may have a sequence that is at least 60%, 65%, 70%, 75%, 80%, 85%, 90%, 95%, 96%, 97%, 98%, or 99% identical thereto.

Functional variants of CRE0071.3 are regulatory elements with sequences which vary from CRE0071.3, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0071.3 can be viewed as a CRE which, when substituted in place of CRE0071.3 in a promoter, substantially retains its activity. For example, a cardiac muscle-specific promoter which comprises a functional variant of CRE0071.3 substituted in place of CRE0071.3 preferably retains 80% of its activity, more preferably 90% of its activity, more preferably 95% of its activity, and yet more preferably 100% of its activity. For example, considering promoter SP0433 as an example, CRE0071.3 in SP00433 can be replaced with a functional variant of CRE0071.3, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0071.3 or functional variant thereof can be provided on either strand of a double stranded polynucleotide and can be provided in either orientation. As such, complementary and reverse complementary sequences of SEQ ID NO: 43 or a functional variant thereof fall within the scope of the invention. Single stranded nucleic acids comprising the sequence according to SEQ ID NO: 43 or a functional variant thereof also fall within the scope of the invention.

In some embodiments, the CRE0071.3 or a functional variant thereof, has a length of 300 or fewer, 200 or fewer nucleotides, 150 or fewer nucleotides, 125 or fewer nucleotides, or 100 or fewer nucleotides.

CRE0070 has the nucleic acid sequence of SEQ ID NO: 42. Functional variants of SEQ ID NO: 42 may have a sequence that is at least 60%, 65%, 70%, 75%, 80%, 85%, 90%, 95%, 96%, 97%, 98%, or 99% identical thereto.

As discussed above, functional variants of CRE0070 substantially retain the ability of CRE0070 to act as a muscle-specific promoter element. For example, when a functional variant of CRE0070 is substituted into cardiac muscle-specific promoter SP0433, the modified promoter retains at least 80% of its activity, more preferably at least 90% of its activity, more preferably at least 95% of its activity, and yet more preferably 100% of the activity of SP0433. Suitably the functional variant of CRE0070 comprises a sequence which has at least 70%, 80%, 90%, 95% or 99% identity to SEQ ID NO: 42.

In some preferred embodiments, a promoter element comprising or consisting of CRE0070 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides, 85 or fewer nucleotides, 75 or fewer nucleotides, 50 or fewer nucleotides.

In some embodiments the cardiac muscle-specific promoter comprises the nucleic acid sequence of SEQ ID NO: 10, or a functional variant thereof. In some embodiments, functional variants of SEQ ID NO: 10 may have a sequence that is at least 60%, 65%, 70%, 75%, 80%, 85%, 90%, 95%, 96%, 97%, 98%, or 99% identical thereto. The promoter having a sequence according to SEQ ID NO: 10 is referred to as SP0433. The SP0433 promoter is particularly preferred in some embodiments. This promoter has been found to be specific for cardiac muscle, which is advantageous in some circumstances.

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0033 and then CRE0082. The sequence of CRE0033 and variants thereof are set out above. The sequence of CRE0082 and variants thereof are set out above.

### SP0435 and Variants Thereof

In some embodiments the cardiac muscle-specific promoter comprises the nucleic acid sequence of SEQ ID NO: 11, or a functional variant thereof. In some embodiments, functional variants of SEQ ID NO: 11 may have a sequence that is at least 60%, 65%, 70%, 75%, 80%, 85%, 90%, 95%, 96%, 97%, 98%, or 99% identical thereto. The promoter having a sequence according to SEQ ID NO: 11 is referred to as SP0435. The SP0435 promoter is particularly preferred in some embodiments. This promoter has been found to be specific for cardiac muscle, which is advantageous in some circumstances.

### SP0436 and Variants Thereof

In some embodiments, the promoter is a synthetic cardiac muscle-specific promoter comprising a combination of two cis-regulatory elements CRE0033, or functional variants thereof. Typically, the CREs are operably linked to a promoter element. In some preferred embodiments, the cardiac muscle-specific promoter comprises said CREs, or functional variants thereof, in the order first CRE0033, second CRE0033, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is SKM_18, or a functional variant thereof. SKM_18 is a muscle-specific proximal promoter.

Thus, in one embodiment the promoter comprises the following regulatory elements: a first CRE0033, a second CRE0033 and SKM_18, or functional variants thereof.

A synthetic promoter comprising a two identical CREs is predicted to have higher expression it its target tissue or cells than an equivalent promoter which comprises only one of the identical CREs. For example, promoter SP0436 which comprises a first CRE0033, a second CRE0033 and SKM_18 has higher expression in cardiac muscle cells than promoter SP0067 which comprises only CRE0033 and SKM_18.

The sequence of CRE0033 and variants thereof are set out above.

The sequence of SKM_18 and variants thereof are set out above.

In some embodiments the cardiac muscle-specific promoter comprises the nucleic acid sequence of SEQ ID NO: 12, or a functional variant thereof. In some embodiments, functional variants of SEQ ID NO: 12 may have a sequence that is at least 60%, 65%, 70%, 75%, 80%, 85%, 90%, 95%, 96%, 97%, 98%, or 99% identical thereto. The promoter having a sequence according to SEQ ID NO: 12 is referred to as SP0436. The SP0436 promoter is particularly preferred in some embodiments. This promoter has been found to be specific for cardiac muscle, which is advantageous in some circumstances.

### SP0449 and Variants Thereof

In some embodiments, the promoter is a synthetic cardiac muscle-specific promoter comprising a combination of the cis-regulatory elements CRE0004 and CRE0033, or functional variants thereof. Typically the CREs are operably linked to a promoter element. In some preferred embodiments, the cardiac muscle-specific promoter comprises said CREs, or functional variants thereof, in the order CRE0004, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0004, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is SKM_18, or a functional variant thereof. SKM_18 is a muscle-specific proximal promoter.

Thus, in one embodiment the promoter comprises the following regulatory elements: CRE0004, CRE0033 and SKM_18, or functional variants thereof.

The sequence of CRE0004 and variants thereof are set out above.

The sequence of CRE0033 and variants thereof are set out above.

The sequence of SKM_18 and variants thereof are set out above.

In some embodiments the cardiac muscle-specific promoter comprises the nucleic acid sequence of SEQ ID NO: 13, or a functional variant thereof. In some embodiments, functional variants of SEQ ID NO: 13 may have a sequence that is at least 60%, 65%, 70%, 75%, 80%, 85%, 90%, 95%, 96%, 97%, 98%, or 99% identical thereto. The promoter having a sequence according to SEQ ID NO: 13 is referred to as SP0449. The SP0449 promoter is particularly preferred in some embodiments. This promoter is predicted to be specific for cardiac muscle, which is advantageous in some circumstances.

### SP0450 and Variants Thereof

In some embodiments, the promoter is a synthetic cardiac muscle-specific promoter comprising a combination of the cis-regulatory elements CRE0095 and CRE0033, or functional variants thereof. Typically the CREs are operably linked to a promoter element. In some preferred embodiments, the cardiac muscle-specific promoter comprises said CREs, or functional variants thereof, in the order CRE0095, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0095, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is SKM_18, or a functional variant thereof. SKM_18 is a muscle-specific proximal promoter.

Thus, in one embodiment the promoter comprises the following regulatory elements: CRE0095, CRE0033 and SKM_18, or functional variants thereof.

The sequence of CRE0095 and variants thereof are set out above.

The sequence of CRE0033 and variants thereof are set out above.

The sequence of SKM_18 and variants thereof are set out above.

In some embodiments the cardiac muscle-specific promoter comprises the nucleic acid sequence of SEQ ID NO: 14, or a functional variant thereof. In some embodiments, functional variants of SEQ ID NO: 14 may have a sequence that is at least 60%, 65%, 70%, 75%, 80%, 85%, 90%, 95%, 96%, 97%, 98%, or 99% identical thereto. The promoter having a sequence according to SEQ ID NO: 14 is referred to as SP0450. The SP0450 promoter is particularly preferred in some embodiments. This promoter is predicted to be specific for cardiac muscle, which is advantageous in some circumstances.

### SP0451 and Variants Thereof

In some embodiments, the promoter is a synthetic cardiac muscle-specific promoter comprising a combination of the cis-regulatory elements CRE0096 and CRE0033, or functional variants thereof. Typically the CREs are operably linked to a promoter element. In some preferred embodiments, the cardiac muscle-specific promoter comprises said CREs, or functional variants thereof, in the order CRE0096, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0096, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is SKM_18, or a functional variant thereof. SKM_18 is a muscle-specific proximal promoter.

Thus, in one embodiment the promoter comprises the following regulatory elements: CRE0096, CRE0033 and SKM_18, or functional variants thereof.

The sequence of CRE0096 and variants thereof are set out above. The sequence of CRE0033 and variants thereof are set out above. The sequence of SKM_18 and variants thereof are set out above.

In some embodiments the cardiac muscle-specific promoter comprises the nucleic acid sequence of SEQ ID NO: 15, or a functional variant thereof. In some embodiments, functional variants of SEQ ID NO: 15 may have a sequence that is at least 60%, 65%, 70%, 75%, 80%, 85%, 90%, 95%, 96%, 97%, 98%, or 99% identical thereto. The promoter having a sequence according to SEQ ID NO: 15 is referred to as SP0451. The SP0451 promoter is particularly preferred in some embodiments. This promoter is predicted to be specific for cardiac muscle, which is advantageous in some circumstances.

### SP0452 and Variants Thereof

In some embodiments, the promoter is a synthetic cardiac muscle-specific promoter comprising a combination of the cardiac muscle-specific proximal promoter CRE0082 and cis-regulatory elements CRE0033, or functional variants thereof. Typically cardiac muscle-specific proximal promoter CRE0082 and cis-regulatory elements CRE0033 are operably linked to a further promoter element. In some preferred embodiments, the cardiac muscle-specific promoter comprises said proximal promoter and CRE, or functional variants thereof, in the order CRE0082, CRE0033, and then the further promoter element (order is given in an upstream to downstream direction, as is conventional in the art).

The further promoter element can be any suitable proximal promoter or minimal promoter. In some embodiments, the further promoter element is a minimal promoter. Where the promoter is a proximal promoter, it is generally preferred that the proximal promoter is muscle-specific or cardiac muscle-specific.

In some preferred embodiments, the further promoter element is SKM_18, or a functional variant thereof. SKM_18 is a muscle-specific proximal promoter.

Thus, in one embodiment the promoter comprises the following regulatory elements: CRE0082, CRE0033 and SKM_18, or functional variants thereof. This promoter comprises two proximal promoters used in tandem.

The sequence of CRE0082 and variants thereof are set out above. The sequence of CRE0033 and variants thereof are set out above. The sequence of SKM_18 and variants thereof are set out above.

In some embodiments the cardiac muscle-specific promoter comprises the nucleic acid sequence of SEQ ID NO: 16, or a functional variant thereof. In some embodiments, functional variants of SEQ ID NO: 16 may have a sequence that is at least 60%, 65%, 70%, 75%, 80%, 85%, 90%, 95%, 96%, 97%, 98%, or 99% identical thereto. The promoter having a sequence according to SEQ ID NO: 16 is referred to as SP0452. The SP0452 promoter is particularly preferred in some embodiments. This promoter is predicted to be specific for cardiac muscle, which is advantageous in some circumstances.

### SP0475 and Variants Thereof

In some embodiments, the promoter is a synthetic cardiac muscle-specific promoter comprising CRE0033 operably linked to a promoter element and a regulatory element such as a 5′UTR and/or an intron. In some preferred embodiments, the synthetic cardiac muscle-specific promoter comprises CRE0033 immediately upstream of the promoter element followed by the regulatory element such as a 5′UTR and/or an intron.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter.

The intron may be any suitable intron. The 5′UTR may be any suitable 5′UTR. A regulatory element may comprise an intron and a 5′UTR. In some preferred embodiments, the regulatory element is the CMV-IE 5′ UTR and intron

In some embodiments the cardiac muscle-specific promoter comprises the following elements (or functional variants thereof): CRE0033 followed by SKM_18 and then CMV-IE 5′UTR and intron.

The sequence of CRE0033 and variants thereof are set out above. The sequence of SKM_18 and variants thereof are set out above.

CMV-IE 5′UTR and intron has the nucleic acid sequence of SEQ ID NO: 65. Functional variants of SEQ ID NO: 65 may have a sequence that is at least 60%, 65%, 70%, 75%, 80%, 85%, 90%, 95%, 96%, 97%, 98%, or 99% identical thereto.

In some embodiments, a functional variant of CMV-IE 5′UTR and intron can be viewed as an intron which, when substituted in place of the CMV-IE 5′UTR and intron in a promoter, substantially retains its activity. For example, a cardiac muscle-specific promoter which comprises a functional variant of CMV-IE 5′ UTR and intron substituted in place of CMV-IE 5′UTR and intron preferably retains 80% of its activity, more preferably 90% of its activity, more preferably 95% of its activity, and yet more preferably 100% of its activity. For example, considering promoter SP0475 as an example, CMV-IE 5′ UTR and intron in SP0475 can be replaced with a functional variant of CMV-IE 5′UTR and intron, and the promoter substantially retains its activity. Retention of activity can be assessed by comparing expression of a suitable reporter under the control of the reference promoter with an otherwise identical promoter comprising the substituted intron under equivalent conditions.

A synthetic promoter comprising an intron such as the CMV-IE 5′ UTR and intron is predicted to have higher expression it its target tissue or cells than an equivalent promoter which does not comprise the intron. For example, promoter SP0475 which comprises CRE0033, SKM_18 and CMV-IE 5′UTR and intron is predicted to have higher expression in cardiac muscle tissue or cells than promoter SP0067 which only comprises CRE0033 and SKM_18.

In some embodiments the cardiac muscle-specific promoter comprises the nucleic acid sequence of SEQ ID NO: 17, or a functional variant thereof. In some embodiments, functional variants of SEQ ID NO: 17 may have a sequence that is at least 60%, 65%, 70%, 75%, 80%, 85%, 90%, 95%, 96%, 97%, 98%, or 99% identical thereto. The promoter having a sequence according to SEQ ID NO: 17 is referred to as SP0475. The SP0475 promoter is particularly preferred in some embodiments. This promoter is predicted to be specific for cardiac muscle, which is advantageous in some circumstances.

### SP0476 and Variants Thereof

In some embodiments, the promoter is a synthetic cardiac muscle-specific promoter comprising CRE0105 operably linked to a promoter element. In some preferred embodiments, the synthetic cardiac muscle-specific promoter comprises CRE0105 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0105 and then SKM_18.

CRE0105 has the nucleic acid sequence of SEQ ID NO: 46. Functional variants of SEQ ID NO: 46 may have a sequence that is at least 60%, 65%, 70%, 75%, 80%, 85%, 90%, 95%, 96%, 97%, 98%, or 99% identical thereto.

Functional variants of CRE0105 are regulatory elements with sequences which vary from CRE0105, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0105 can be viewed as a CRE which, when substituted in place of CRE0105 in a promoter, substantially retains its activity. For example, a cardiac muscle-specific promoter which comprises a functional variant of CRE0105 substituted in place of CRE0105 preferably retains 80% of its activity, more preferably 90% of its activity, more preferably 95% of its activity, and yet more preferably 100% of its activity. For example, considering promoter SP0476 as an example, CRE0105 in SP0476 can be replaced with a functional variant of CRE0105, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0105 or functional variant thereof can be provided on either strand of a double stranded polynucleotide and can be provided in either orientation. As such, complementary and reverse complementary sequences of SEQ ID NO: 46 or a functional variant thereof fall within the scope of the invention. Single stranded nucleic acids comprising the sequence according to SEQ ID NO: 46 or a functional variant thereof also fall within the scope of the invention.

In some embodiments, the CRE0105 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises the nucleic acid sequence of SEQ ID NO: 18, or a functional variant thereof. In some embodiments, functional variants of SEQ ID NO: 18 may have a sequence that is at least 60%, 65%, 70%, 75%, 80%, 85%, 90%, 95%, 96%, 97%, 98%, or 99% identical thereto. The promoter having a sequence according to SEQ ID NO: 18 is referred to as SP0476. The SP0476 promoter is particularly preferred in some embodiments. This promoter is predicted to be specific for cardiac muscle, which is advantageous in some circumstances.

### SP0477 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0106 and then SKM_18.

CRE0106 has the nucleic acid sequence of SEQ ID NO: 47. Functional variants of SEQ ID NO: 47 may have a sequence that is at least 60%, 65%, 70%, 75%, 80%, 85%, 90%, 95%, 96%, 97%, 98%, or 99% identical thereto.

Functional variants of CRE0106 are regulatory elements with sequences which vary from CRE0106, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0106 can be viewed as a CRE which, when substituted in place of CRE0106 in a promoter, substantially retains its activity. For example, a cardiac muscle-specific promoter which comprises a functional variant of CRE0106 substituted in place of CRE0106 preferably retains 80% of its activity, more preferably 90% of its activity, more preferably 95% of its activity, and yet more preferably 100% of its activity. For example, considering promoter SP0477 as an example, CRE0106 in SP0477 can be replaced with a functional variant of CRE0106, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0106 or functional variant thereof can be provided on either strand of a double stranded polynucleotide and can be provided in either orientation. As such, complementary and reverse complementary sequences of SEQ ID NO: 47 or a functional variant thereof fall within the scope of the invention. Single stranded nucleic acids comprising the sequence according to SEQ ID NO: 47 or a functional variant thereof also fall within the scope of the invention.

In some embodiments, the CRE0106 or a functional variant thereof, has a length of 300 or fewer nucleotides, 200 or fewer nucleotides, 150 or fewer nucleotides, 125 or fewer nucleotides, or 100 or fewer nucleotides. The sequence of SKM_18 and variants thereof are set out above.

In some embodiments the cardiac muscle-specific promoter comprises the nucleic acid sequence of SEQ ID NO: 19, or a functional variant thereof. In some embodiments, functional variants of SEQ ID NO: 19 may have a sequence that is at least 60%, 65%, 70%, 75%, 80%, 85%, 90%, 95%, 96%, 97%, 98%, or 99% identical thereto. The promoter having a sequence according to SEQ ID NO: 19 is referred to as SP0477. The SP0477 promoter is particularly preferred in some embodiments. This promoter is predicted to be specific for cardiac muscle, which is advantageous in some circumstances.

### SP0478 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0107 and then SKM_18.

CRE0107 has the nucleic acid sequence of SEQ ID NO: 48. Functional variants of SEQ ID NO: 48 thereof may have a sequence that is at least 60%, 65%, 70%, 75%, 80%, 85%, 90%, 95%, 96%, 97%, 98%, or 99% identical thereto.

Functional variants of CRE0107 are regulatory elements with sequences which vary from CRE0107, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0107 can be viewed as a CRE which, when substituted in place of CRE0107 in a promoter, substantially retains its activity. For example, a cardiac muscle-specific promoter which comprises a functional variant of CRE0107 substituted in place of CRE0107 preferably retains 80% of its activity, more preferably 90% of its activity, more preferably 95% of its activity, and yet more preferably 100% of its activity. For example, considering promoter SP0478 as an example, CRE0107 in SP0478 can be replaced with a functional variant of CRE0107, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0107 or functional variant thereof can be provided on either strand of a double stranded polynucleotide and can be provided in either orientation. As such, complementary and reverse complementary sequences of SEQ ID NO: 48 or a functional variant thereof fall within the scope of the invention. Single stranded nucleic acids comprising the sequence according to SEQ ID NO: 48 or a functional variant thereof also fall within the scope of the invention.

In some embodiments, the CRE0107 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides. The sequence of SKM_1 8 and variants thereof are set out above.

In some embodiments the cardiac muscle-specific promoter comprises the nucleic acid sequence of SEQ ID NO: 20, or a functional variant thereof. In some embodiments, functional variants of SEQ ID NO: 20 may have a sequence that is at least 60%, 65%, 70%, 75%, 80%, 85%, 90%, 95%, 96%, 97%, 98%, or 99% identical thereto. The promoter having a sequence according to SEQ ID NO: 20 is referred to as SP0478. The SP0478 promoter is particularly preferred in some embodiments. This promoter is predicted to be specific for cardiac muscle, which is advantageous in some circumstances.

### SP0479 and Variants Thereof

In some embodiments, the promoter is a synthetic cardiac muscle-specific promoter comprising CRE0108 operably linked to a promoter element. In some preferred embodiments, the synthetic cardiac muscle-specific promoter comprises CRE0108 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0108 and then SKM_18.

CRE0108 has the nucleic acid sequence of SEQ ID NO: 49. Functional variants of SEQ ID NO: 49 may have a sequence that is at least 60%, 65%, 70%, 75%, 80%, 85%, 90%, 95%, 96%, 97%, 98%, or 99% identical thereto.

Functional variants of CRE0108 are regulatory elements with sequences which vary from CRE0108, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0108 can be viewed as a CRE which, when substituted in place of CRE0108 in a promoter, substantially retains its activity. For example, a cardiac muscle-specific promoter which comprises a functional variant of CRE0108 substituted in place of CRE0108 preferably retains 80% of its activity, more preferably 90% of its activity, more preferably 95% of its activity, and yet more preferably 100% of its activity. For example, considering promoter SP0479 as an example, CRE0108 in SP0479 can be replaced with a functional variant of CRE0108, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0108 or functional variant thereof can be provided on either strand of a double stranded polynucleotide and can be provided in either orientation. As such, complementary and reverse complementary sequences of SEQ ID NO: 49 or a functional variant thereof fall within the scope of the invention. Single stranded nucleic acids comprising the sequence according to SEQ ID NO: 49 or a functional variant thereof also fall within the scope of the invention.

In some embodiments, the CRE0108 or a functional variant thereof, has a length of 250 or fewer nucleotides, 200 or fewer nucleotides, 150 or fewer nucleotides, 125 or fewer nucleotides, or 100 or fewer nucleotides. The sequence of SKM_18 and variants thereof are set out above.

In some embodiments the cardiac muscle-specific promoter comprises the nucleic acid sequence of SEQ ID NO: 21, or a functional variant thereof. In some embodiments, functional variants of SEQ ID NO: 21 may have a sequence that is at least 60%, 65%, 70%, 75%, 80%, 85%, 90%, 95%, 96%, 97%, 98%, or 99% identical thereto. The promoter having a sequence according to SEQ ID NO: 21 is referred to as SP0479. The SP0479 promoter is particularly preferred in some embodiments. This promoter is predicted to be specific for cardiac muscle, which is advantageous in some circumstances.

### SP0480 and Variants Thereof

In some embodiments, the promoter is a synthetic cardiac muscle-specific promoter comprising CRE0109 operably linked to a promoter element. In some preferred embodiments, the synthetic cardiac muscle-specific promoter comprises CRE0109 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0109 and then SKM_18.

CRE0109 has the nucleic acid sequence of SEQ ID NO: 50. Functional variants of SEQ ID NO: 50 may have a sequence that is at least 60%, 65%, 70%, 75%, 80%, 85%, 90%, 95%, 96%, 97%, 98%, or 99% identical thereto.

Functional variants of CRE0109 are regulatory elements with sequences which vary from CRE0109, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0109 can be viewed as a CRE which, when substituted in place of CRE0109 in a promoter, substantially retains its activity. For example, a cardiac muscle-specific promoter which comprises a functional variant of CRE0109 substituted in place of CRE0109 preferably retains 80% of its activity, more preferably 90% of its activity, more preferably 95% of its activity, and yet more preferably 100% of its activity. For example, considering promoter SP0480 as an example, CRE0109 in SP0480 can be replaced with a functional variant of CRE0109, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0109 or functional variant thereof can be provided on either strand of a double stranded polynucleotide and can be provided in either orientation. As such, complementary and reverse complementary sequences of SEQ ID NO: 50 or a functional variant thereof fall within the scope of the invention. Single stranded nucleic acids comprising the sequence according to SEQ ID NO: 50 or a functional variant thereof also fall within the scope of the invention.

In some embodiments, the CRE0109 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides. The sequence of SKM_1 8 and variants thereof are set out above.

In some embodiments the cardiac muscle-specific promoter comprises the nucleic acid sequence of SEQ ID NO: 22, or a functional variant thereof. In some embodiments, functional variants of SEQ ID NO: 22 may have a sequence that is at least 60%, 65%, 70%, 75%, 80%, 85%, 90%, 95%, 96%, 97%, 98%, or 99% identical thereto. The promoter having a sequence according to SEQ ID NO: 22 is referred to as SP0480. The SP0480 promoter is particularly preferred in some embodiments. This promoter is predicted to be specific for cardiac muscle, which is advantageous in some circumstances.

### SP0481 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0110 or functional variant thereof. CRE0110 is a cardiac muscle-specific proximal promoter.

In some embodiments the cardiac muscle-specific promoter comprises the following elements (or functional variants thereof): CRE0033 and then CRE0110. The sequence of CRE0033 and variants thereof are set out above.

CRE0110 has the nucleic acid sequence of SEQ ID NO: 59. Functional variants of SEQ ID NO: 59 may have a sequence that is at least 60%, 65%, 70%, 75%, 80%, 85%, 90%, 95%, 96%, 97%, 98%, or 99% identical thereto.

As discussed above, functional variants of CRE0110 substantially retain the ability of CRE0110 to act as a cardiac muscle-specific promoter element. For example, when a functional variant of CRE0110 is substituted into cardiac muscle-specific promoter SP0481, the modified promoter retains at least 80% of its activity, more preferably at least 90% of its activity, more preferably at least 95% of its activity, and yet more preferably 100% of the activity of SP0481. Suitably the functional variant of CRE0110 comprises a sequence which has at least 70%, 80%, 90%, 95% or 99% identity to SEQ ID NO: 59.

In some preferred embodiments, a promoter element comprising or consisting of CRE0110 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 23, or a functional variant thereof. In some embodiments, functional variants of SEQ ID NO: 23 may have a sequence that is at least 60%, 65%, 70%, 75%, 80%, 85%, 90%, 95%, 96%, 97%, 98%, or 99% identical thereto. The promoter having a sequence according to SEQ ID NO: 23 is referred to as SP0481. The SP0481 promoter is particularly preferred in some embodiments. This promoter is predicted to be specific for cardiac muscle, which is advantageous in some circumstances.

### SP0482 and Variants Thereof

In some embodiments, the promoter is a synthetic cardiac muscle-specific promoter comprising CRE0111 operably linked to a promoter element. In some preferred embodiments, the synthetic cardiac muscle-specific promoter comprises CRE0111 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0111 and then SKM_18.

CRE0111 has the nucleic acid sequence of SEQ ID NO: 51. Functional variants of SEQ ID NO: 51 may have a sequence that is at least 60%, 65%, 70%, 75%, 80%, 85%, 90%, 95%, 96%, 97%, 98%, or 99% identical thereto.

Functional variants of CRE0111 are regulatory elements with sequences which vary from CRE0111, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0111 can be viewed as a CRE which, when substituted in place of CRE0111 in a promoter, substantially retains its activity. For example, a cardiac muscle-specific promoter which comprises a functional variant of CRE0111 substituted in place of CRE0111 preferably retains 80% of its activity, more preferably 90% of its activity, more preferably 95% of its activity, and yet more preferably 100% of its activity. For example, considering promoter SP0482 as an example, CRE0111 in SP0482 can be replaced with a functional variant of CRE0111, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0111 or functional variant thereof can be provided on either strand of a double stranded polynucleotide and can be provided in either orientation. As such, complementary and reverse complementary sequences of SEQ ID NO: 51 or a functional variant thereof fall within the scope of the invention. Single stranded nucleic acids comprising the sequence according to SEQ ID NO: 51 or a functional variant thereof also fall within the scope of the invention.

In some embodiments, the CRE0111 or a functional variant thereof, has a length of 300 or fewer nucleotides, 200 or fewer nucleotides, 150 or fewer nucleotides, 125 or fewer nucleotides, or 100 or fewer nucleotides. The sequence of SKM_18 and variants thereof are set out above.

In some embodiments the cardiac muscle-specific promoter comprises the nucleic acid sequence of SEQ ID NO: 24, or a functional variant thereof. In some embodiments, functional variants of SEQ ID NO: 24 may have a sequence that is at least 60%, 65%, 70%, 75%, 80%, 85%, 90%, 95%, 96%, 97%, 98%, or 99% identical thereto. The promoter having a sequence according to SEQ ID NO: 24 is referred to as SP0482. The SP0482 promoter is particularly preferred in some embodiments. This promoter is predicted to be specific for cardiac muscle, which is advantageous in some circumstances.

### SP0483 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0112 or functional variant thereof. CRE0112 is a cardiac muscle-specific proximal promoter.

In some embodiments the cardiac muscle-specific promoter comprises the following elements (or functional variants thereof): CRE0033 and then CRE0112. The sequence of CRE0033 and variants thereof are set out above.

CRE0112 has the nucleic acid sequence of SEQ ID NO: 60. Functional variants of SEQ ID NO: 60 may have a sequence that is at least 60%, 65%, 70%, 75%, 80%, 85%, 90%, 95%, 96%, 97%, 98%, or 99% identical thereto.

As discussed above, functional variants of CRE0112 substantially retain the ability of CRE0112 to act as a cardiac muscle-specific promoter element. For example, when a functional variant of CRE0112 is substituted into cardiac muscle-specific promoter SP0483, the modified promoter retains at least 80% of its activity, more preferably at least 90% of its activity, more preferably at least 95% of its activity, and yet more preferably 100% of the activity of SP0483. Suitably the functional variant of CRE0112 comprises a sequence which has at least 70%, 80%, 90%, 95% or 99% identity to SEQ ID NO: 60.

In some preferred embodiments, a promoter element comprising or consisting of CRE0112 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 25, or a functional variant thereof. In some embodiments, functional variants of SEQ ID NO: 25 may have a sequence that is at least 60%, 65%, 70%, 75%, 80%, 85%, 90%, 95%, 96%, 97%, 98%, or 99% identical thereto. The promoter having a sequence according to SEQ ID NO: 25 is referred to as SP0483. The SP0483 promoter is particularly preferred in some embodiments. This promoter is predicted to be specific for cardiac muscle, which is advantageous in some circumstances.

### SP0484 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0113 or functional variant thereof. CRE0113 is a cardiac muscle-specific proximal promoter.

In some embodiments the cardiac muscle-specific promoter comprises the following elements (or functional variants thereof): CRE0033 and then CRE0113. The sequence of CRE0033 and variants thereof are set out above.

CRE0113 has the nucleic acid sequence of SEQ ID NO: 61. Functional variants of SEQ ID NO: 61 may have a sequence that is at least 60%, 65%, 70%, 75%, 80%, 85%, 90%, 95%, 96%, 97%, 98%, or 99% identical thereto.

As discussed above, functional variants of CRE0 113 substantially retain the ability of CRE0113 to act as a cardiac muscle-specific promoter element. For example, when a functional variant of CRE0113 is substituted into cardiac muscle-specific promoter SP0484, the modified promoter retains at least 80% of its activity, more preferably at least 90% of its activity, more preferably at least 95% of its activity, and yet more preferably 100% of the activity of SP0484. Suitably the functional variant of CRE0113 comprises a sequence which has at least 70%, 80%, 90%, 95% or 99% identity to SEQ ID NO: 61.

In some preferred embodiments, a promoter element comprising or consisting of CRE0113 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 26, or a functional variant thereof. In some embodiments, functional variants of SEQ ID NO: 26 may have a sequence that is at least 60%, 65%, 70%, 75%, 80%, 85%, 90%, 95%, 96%, 97%, 98%, or 99% identical thereto. The promoter having a sequence according to SEQ ID NO: 26 is referred to as SP0484. The SP0484 promoter is particularly preferred in some embodiments. This promoter is predicted to be specific for cardiac muscle, which is advantageous in some circumstances.

### SP0485 and Variants Thereof

In some embodiments, the promoter is a synthetic cardiac muscle-specific promoter comprising CRE0114 operably linked to a promoter element. In some preferred embodiments, the synthetic cardiac muscle-specific promoter comprises CRE0114 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific. In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0114 and then SKM_18.

CRE0114 has the nucleic acid sequence of SEQ ID NO: 52. Functional variants of SEQ ID NO: 52 may have a sequence that is at least 60%, 65%, 70%, 75%, 80%, 85%, 90%, 95%, 96%, 97%, 98%, or 99% identical thereto.

Functional variants of CRE0 114 are regulatory elements with sequences which vary from CRE0114, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0114 can be viewed as a CRE which, when substituted in place of CRE0114 in a promoter, substantially retains its activity. For example, a cardiac muscle-specific promoter which comprises a functional variant of CRE0114 substituted in place of CRE0114 preferably retains 80% of its activity, more preferably 90% of its activity, more preferably 95% of its activity, and yet more preferably 100% of its activity. For example, considering promoter SP0485 as an example, CRE0114 in SP0485 can be replaced with a functional variant of CRE0114, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0114 or functional variant thereof can be provided on either strand of a double stranded polynucleotide and can be provided in either orientation. As such, complementary and reverse complementary sequences of SEQ ID NO: 52 or a functional variant thereof fall within the scope of the invention. Single stranded nucleic acids comprising the sequence according to SEQ ID NO: 52 or a functional variant thereof also fall within the scope of the invention.

In some embodiments, the CRE0114 or a functional variant thereof, has a length of 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises the nucleic acid sequence of SEQ ID NO: 27, or a functional variant thereof. In some embodiments, functional variants of SEQ ID NO: 27 may have a sequence that is at least 60%, 65%, 70%, 75%, 80%, 85%, 90%, 95%, 96%, 97%, 98%, or 99% identical thereto. The promoter having a sequence according to SEQ ID NO: 27 is referred to as SP0485. The SP0485 promoter is particularly preferred in some embodiments. This promoter is predicted to be specific for cardiac muscle, which is advantageous in some circumstances.

### SP0486 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0115 or functional variant thereof. CRE0115 is a cardiac muscle-specific proximal promoter. In some embodiments the cardiac muscle-specific promoter comprises the following elements (or functional variants thereof): CRE0033 and then CRE0115.

The sequence of CRE0033 and variants thereof are set out above.

CRE0115 has the nucleic acid sequence of SEQ ID NO: 62. Functional variants of SEQ ID NO: 62 may have a sequence that is at least 60%, 65%, 70%, 75%, 80%, 85%, 90%, 95%, 96%, 97%, 98%, or 99% identical thereto.

As discussed above, functional variants of CRE0 115 substantially retain the ability of CRE0115 to act as a cardiac muscle-specific promoter element. For example, when a functional variant of CRE0115 is substituted into cardiac muscle-specific promoter SP0486, the modified promoter retains at least 80% of its activity, more preferably at least 90% of its activity, more preferably at least 95% of its activity, and yet more preferably 100% of the activity of SP0486. Suitably the functional variant of CRE0115 comprises a sequence which has at least 70%, 80%, 90%, 95% or 99% identity to SEQ ID NO: 62.

In some preferred embodiments, a promoter element comprising or consisting of CRE0115 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 28, or a functional variant thereof. In some embodiments, functional variants of SEQ ID NO: 28 may have a sequence that is at least 60%, 65%, 70%, 75%, 80%, 85%, 90%, 95%, 96%, 97%, 98%, or 99% identical thereto. The promoter having a sequence according to SEQ ID NO: 28 is referred to as SP0486. The SP0486 promoter is particularly preferred in some embodiments. This promoter is predicted to be specific for cardiac muscle, which is advantageous in some circumstances.

### SP0487 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific. In some preferred embodiments the promoter element is CRE0116 or functional variant thereof. CRE0116 is a cardiac muscle-specific proximal promoter. In some embodiments the cardiac muscle-specific promoter comprises the following elements (or functional variants thereof): CRE0033 and then CRE0116. The sequence of CRE0033 and variants thereof are set out above.

CRE0116 has the nucleic acid sequence of SEQ ID NO: 63. Functional variants of SEQ ID NO: 63 may have a sequence that is at least 60%, 65%, 70%, 75%, 80%, 85%, 90%, 95%, 96%, 97%, 98%, or 99% identical thereto.

As discussed above, functional variants of CRE0116 substantially retain the ability of CRE0116 to act as a cardiac muscle-specific promoter element. For example, when a functional variant of CRE0116 is substituted into cardiac muscle-specific promoter SP0487, the modified promoter retains at least 80% of its activity, more preferably at least 90% of its activity, more preferably at least 95% of its activity, and yet more preferably 100% of the activity of SP0487. Suitably the functional variant of CRE0116 comprises a sequence which has at least 70%, 80%, 90%, 95% or 99% identity to SEQ ID NO: 63.

In some preferred embodiments, a promoter element comprising or consisting of CRE0116 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 29, or a functional variant thereof. In some embodiments, functional variants of SEQ ID NO: 29 may have a sequence that is at least 60%, 65%, 70%, 75%, 80%, 85%, 90%, 95%, 96%, 97%, 98%, or 99% identical thereto. The promoter having a sequence according to SEQ ID NO: 29 is referred to as SP0487. The SP0487 promoter is particularly preferred in some embodiments. This promoter is predicted to be specific for cardiac muscle, which is advantageous in some circumstances.

### SP0488 and Variants Thereof

In some embodiments, the promoter is a synthetic cardiac muscle-specific promoter comprising CRE0117 operably linked to a promoter element. In some preferred embodiments, the synthetic cardiac muscle-specific promoter comprises CRE0117 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0117 and then SKM_18.

CRE0117 has the nucleic acid sequence of SEQ ID NO: 53. Functional variants of SEQ ID NO: 53 may have a sequence that is at least 60%, 65%, 70%, 75%, 80%, 85%, 90%, 95%, 96%, 97%, 98%, or 99% identical thereto.

Functional variants of CRE0117 are regulatory elements with sequences which vary from CRE0117, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0117 can be viewed as a CRE which, when substituted in place of CRE0117 in a promoter, substantially retains its activity. For example, a cardiac muscle-specific promoter which comprises a functional variant of CRE0117 substituted in place of CRE0117 preferably retains 80% of its activity, more preferably 90% of its activity, more preferably 95% of its activity, and yet more preferably 100% of its activity. For example, considering promoter SP0488 as an example, CRE0117 in SP0488 can be replaced with a functional variant of CRE0117, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0117 or functional variant thereof can be provided on either strand of a double stranded polynucleotide and can be provided in either orientation. As such, complementary and reverse complementary sequences of SEQ ID NO: 53 or a functional variant thereof fall within the scope of the invention. Single stranded nucleic acids comprising the sequence according to SEQ ID NO: 53 or a functional variant thereof also fall within the scope of the invention.

In some embodiments, the CRE0117 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises the nucleic acid sequence of SEQ ID NO: 30, or a functional variant thereof. In some embodiments, functional variants of SEQ ID NO: 30 may have a sequence that is at least 60%, 65%, 70%, 75%, 80%, 85%, 90%, 95%, 96%, 97%, 98%, or 99% identical thereto. The promoter having a sequence according to SEQ ID NO: 30 is referred to as SP0488. The SP0488 promoter is particularly preferred in some embodiments. This promoter is predicted to be specific for cardiac muscle, which is advantageous in some circumstances.

### SP0489 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0104 or functional variant thereof. CRE0104 is a cardiac muscle-specific proximal promoter. In some embodiments the cardiac muscle-specific promoter comprises the following elements (or functional variants thereof): CRE0033 and then CRE0104.

The sequence of CRE0033 and variants thereof are set out above.

CRE0104 has the nucleic acid sequence of SEQ ID NO: 58. Functional variants of SEQ ID NO: 58 may have a sequence that is at least 60%, 65%, 70%, 75%, 80%, 85%, 90%, 95%, 96%, 97%, 98%, or 99% identical thereto.

As discussed above, functional variants of CRE0104 substantially retain the ability of CRE0104 to act as a cardiac muscle-specific promoter element. For example, when a functional variant of CRE0104 is substituted into cardiac muscle-specific promoter SP0489, the modified promoter retains at least 80% of its activity, more preferably at least 90% of its activity, more preferably at least 95% of its activity, and yet more preferably 100% of the activity of SP0489. Suitably the functional variant of CRE0104 comprises a sequence which has at least 70%, 80%, 90%, 95% or 99% identity to SEQ ID NO: 58.

In some preferred embodiments, a promoter element comprising or consisting of CRE0104 or a functional variant thereof has a length of 400 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the nucleic acid sequence of SEQ ID NO: 31, or a functional variant thereof. In some embodiments, functional variants of SEQ ID NO: 31 may have a sequence that is at least 60%, 65%, 70%, 75%, 80%, 85%, 90%, 95%, 96%, 97%, 98%, or 99% identical thereto. The promoter having a sequence according to SEQ ID NO: 31 is referred to as SP0489. The SP0489 promoter is particularly preferred in some embodiments. This promoter is predicted to be specific for cardiac muscle, which is advantageous in some circumstances.

### SP0490 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific. In some preferred embodiments the promoter element is CRE01 10 or functional variant thereof. CRE0110 is a cardiac muscle-specific proximal promoter. In some embodiments the cardiac muscle-specific promoter comprises the following elements (or functional variants thereof): CRE0106 and then CRE0110. The sequence of CRE0106 and variants thereof are set out above. The sequence of CRE0110 and variants thereof are set out above.

In some embodiments the cardiac muscle-specific promoter comprises the nucleic acid sequence of SEQ ID NO: 32, or a functional variant thereof. In some embodiments, functional variants of SEQ ID NOL 32 may have a sequence that is at least 60%, 65%, 70%, 75%, 80%, 85%, 90%, 95%, 96%, 97%, 98%, or 99% identical thereto. The promoter having a sequence according to SEQ ID NO: 32 is referred to as SP0490. The SP0490 promoter is particularly preferred in some embodiments. This promoter is predicted to be specific for cardiac muscle, which is advantageous in some circumstances.

### SP0491 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0110 or functional variant thereof. CRE0110 is a cardiac muscle-specific proximal promoter. In some embodiments the cardiac muscle-specific promoter comprises the following elements (or functional variants thereof): CRE0107 and then CRE0110. The sequence of CRE0107 and variants thereof are set out above. The sequence of CRE0110 and variants thereof are set out above.

In some embodiments the cardiac muscle-specific promoter comprises the nucleic acid sequence of SEQ ID NO: 33, or a functional variant thereof. In some embodiments, functional variants of SEQ ID NO: 33 may have a sequence that is at least 60%, 65%, 70%, 75%, 80%, 85%, 90%, 95%, 96%, 97%, 98%, or 99% identical thereto. The promoter having a sequence according to SEQ ID NO: 33 is referred to as SP0491. The SP0491 promoter is particularly preferred in some embodiments. This promoter is predicted to be specific for cardiac muscle, which is advantageous in some circumstances.

### SP0492 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0116 or functional variant thereof. CRE0116 is a cardiac muscle-specific proximal promoter.

In some embodiments the cardiac muscle-specific promoter comprises the following elements (or functional variants thereof): CRE0106 and then CRE0116. The sequence of CRE0106 and variants thereof are set out above. The sequence of CRE0116 and variants thereof are set out above.

In some embodiments the cardiac muscle-specific promoter comprises the nucleic acid sequence of SEQ ID NO: 34, or a functional variant thereof. In some embodiments, functional variants of SEQ ID NO: 34 may have a sequence that is at least 60%, 65%, 70%, 75%, 80%, 85%, 90%, 95%, 96%, 97%, 98%, or 99% identical thereto. The promoter having a sequence according to SEQ ID NO: 34 is referred to as SP0492. The SP0492 promoter is particularly preferred in some embodiments. This promoter is predicted to be specific for cardiac muscle, which is advantageous in some circumstances.

### SP0493 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is CRE0116 or functional variant thereof. CRE0116 is a cardiac muscle-specific proximal promoter. In some embodiments the cardiac muscle-specific promoter comprises the following elements (or functional variants thereof): CRE0107 and then CRE0116. The sequence of CRE0107 and variants thereof are set out above. The sequence of CRE0116 and variants thereof are set out above.

In some embodiments the cardiac muscle-specific promoter comprises the nucleic acid sequence of SEQ ID NO: 35, or a functional variant thereof. In some embodiments, functional variants of SEQ ID NO: 35 may have a sequence that is at least 60%, 65%, 70%, 75%, 80%, 85%, 90%, 95%, 96%, 97%, 98%, or 99% identical thereto. The promoter having a sequence according to SEQ ID NO: 35 is referred to as SP0493. The SP0493 promoter is particularly preferred in some embodiments. This promoter has been found to be specific for cardiac muscle, which is advantageous in some circumstances.

### SP0494 and Variants Thereof

In some embodiments, the promoter is a synthetic cardiac muscle-specific promoter comprising CRE0118 operably linked to a promoter element. In some preferred embodiments, the synthetic cardiac muscle-specific promoter comprises CRE0118 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific or cardiac muscle-specific.

In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0118 and then SKM_18.

CRE0118 has the nucleic acid sequence of SEQ ID NO: 54. Functional variants of SEQ ID NO: 54 may have a sequence that is at least 60%, 65%, 70%, 75%, 80%, 85%, 90%, 95%, 96%, 97%, 98%, or 99% identical thereto.

Functional variants of CRE0118 are regulatory elements with sequences which vary from CRE0118, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0118 can be viewed as a CRE which, when substituted in place of CRE0118 in a promoter, substantially retains its activity. For example, a cardiac muscle-specific promoter which comprises a functional variant of CRE0118 substituted in place of CRE0118 preferably retains 80% of its activity, more preferably 90% of its activity, more preferably 95% of its activity, and yet more preferably 100% of its activity. For example, considering promoter SP0494 as an example, CRE0118 in SP0494 can be replaced with a functional variant of CRE0118, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0118 or functional variant thereof can be provided on either strand of a double stranded polynucleotide and can be provided in either orientation. As such, complementary and reverse complementary sequences of SEQ ID NO: 54 or a functional variant thereof fall within the scope of the invention. Single stranded nucleic acids comprising the sequence according to SEQ ID NO: 54 or a functional variant thereof also fall within the scope of the invention.

In some embodiments, the CRE0118 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises the nucleic acid sequence of SEQ ID NO: 36, or a functional variant thereof. In some embodiments, functional variants of SEQ ID NO: 36 may have a sequence that is at least 60%, 65%, 70%, 75%, 80%, 85%, 90%, 95%, 96%, 97%, 98%, or 99% identical thereto. The promoter having a sequence according to SEQ ID NO: 36 is referred to as SP0494. The SP0494 promoter is particularly preferred in some embodiments. This promoter has been found to be specific for cardiac muscle, which is advantageous in some circumstances.

### SP0495 and Variants Thereof

In some embodiments, the promoter is a synthetic cardiac muscle-specific promoter comprising a combination of the cis-regulatory elements CRE0106 and CRE0033, or functional variants thereof. Typically the CREs are operably linked to a promoter element. In some preferred embodiments, the cardiac muscle-specific promoter comprises said CREs, or functional variants thereof, in the order CRE0106, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0106, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is CRE0116, or a functional variant thereof. CRE0116 is a cardiac muscle-specific proximal promoter. Thus, in one embodiment the promoter comprises the following regulatory elements: CRE0106, CRE0033 and CRE0116, or functional variants thereof. The sequence of CRE0106 and variants thereof are set out above. The sequence of CRE0033 and variants thereof are set out above. The sequence of CRE0116 and variants thereof are set out above.

In some embodiments the cardiac muscle-specific promoter comprises the nucleic acid sequence of SEQ ID NO: 37, or a functional variant thereof. In some embodiments, functional variants of SEQ ID NO: 37 may have a sequence that is at least 60%, 65%, 70%, 75%, 80%, 85%, 90%, 95%, 96%, 97%, 98%, or 99% identical thereto. The promoter having a sequence according to SEQ ID NO: 37 is referred to as SP0495. The SP0495 promoter is particularly preferred in some embodiments. This promoter has been found to be specific for cardiac muscle, which is advantageous in some circumstances.

### SP0496 and Variants Thereof

In some embodiments, the promoter is a synthetic cardiac muscle-specific promoter comprising a combination of the cis-regulatory elements CRE0107 and CRE0033, or functional variants thereof. Typically the CREs are operably linked to a promoter element. In some preferred embodiments, the cardiac muscle-specific promoter comprises said CREs, or functional variants thereof, in the order CRE0107, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0107, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is CRE0116, or a functional variant thereof. CRE0116 is a cardiac muscle-specific proximal promoter.

Thus, in one embodiment the promoter comprises the following regulatory elements: CRE0107, CRE0033 and CRE0116, or functional variants thereof. The sequence of CRE0106 and variants thereof are set out above. The sequence of CRE0033 and variants thereof are set out above. The sequence of CRE0116 and variants thereof are set out above.

In some embodiments the cardiac muscle-specific promoter comprises the nucleic acid sequence of SEQ ID NO: 38, or a functional variant thereof. In some embodiments, functional variants of SEQ ID NO: 38 may have a sequence that is at least 60%, 65%, 70%, 75%, 80%, 85%, 90%, 95%, 96%, 97%, 98%, or 99% identical thereto. The promoter having a sequence according to SEQ ID NO: 38 is referred to as SP0496. The SP0496 promoter is particularly preferred in some embodiments. This promoter has been found to be specific for cardiac muscle, which is advantageous in some circumstances.

### Tandem Promoters

In some embodiments, a synthetic cardiac muscle-specific promoter comprises two or more promoter elements. Synthetic promoters comprising two or more promoter elements are referred to herein as ‘tandem promoters’. For example, SP0452 is a tandem promoter as it comprises promoter elements CRE0082 and SKM_18. In some embodiments, a tandem promoter may comprise a promoter element directly upstream of another promoter element. In some embodiments, a tandem promoter may comprise one or more CREs upstream of one or each of the promoter elements. In some embodiments, a tandem promoter may comprise one or more CREs between the promoter elements. In some embodiments, any one of the synthetic cardiac muscle-specific promoters disclosed herein may be operably linked to a further promoter element. For example, SP0452 is synthetic promoter SP0067 operably linked to a promoter element CRE0082. It will be appreciated that synthetic promoter SP0067 may be operably linked to any other promoter element disclosed herein. Similarly, any other synthetic promoter disclosed herein may be operably linked to any promoter element disclosed herein.

### Composite Promoters

In some embodiments, the muscle-specific, cardiac muscle-specific or the skeletal muscle-specific promoters as set out above are operably linked to one or more additional regulatory sequences. An additional regulatory sequence can, for example, enhance expression compared to a muscle-specific, a cardiac muscle-specific, or a skeletal muscle-specific promoter which is not operably linked the additional regulatory sequence. Generally, it is preferred that the additional regulatory sequence does not substantively reduce the specificity of a muscle-specific, a cardiac muscle-specific, or a skeletal muscle-specific promoter.

For example, a synthetic muscle-specific, cardiac muscle-specific or skeletal muscle-specific promoter according to the present invention can be operably linked to a sequence encoding a UTR (e.g. a 5′ and/or 3′ UTR), and/or an intron, or suchlike. In some embodiments, the cardiac muscle-specific promoter is operably linked to sequence encoding a UTR, e.g. a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′UTR can also be partially or entirely synthetic. In eukaryotes, 5′ UTRs have a median length of approximately 150 nucleotides, but in some cases they can be considerably longer. Regulatory sequences that can be found in 5′ UTRs include, but are not limited to: (i) Binding sites for proteins, that may affect the mRNA’s stability or translation; (ii) Riboswitches; (iii) Sequences that promote or inhibit translation initiation; and (iv) Introns within 5′ UTRs have been linked to regulation of gene expression and mRNA export.

When a regulatory sequence comprises both a 5′ UTR and an intron, it may be called 5′UTR and intron. In some embodiments, a cardiac muscle-specific promoter as set out above is operably linked to a sequence encoding a 5′ UTR and an intron derived from the CMV major immediate gene (CMV-IE gene). For example, the 5′ UTR and intron from the CMV-IE gene suitably comprises the CMV-IE gene exon 1 and the CMV-IE gene exon 1, or portions thereof. In some cases, the promoter element may be modified in view of the linkage to the 5 ‘UTR, for example sequences downstream of the transcription start site (TSS) in the promoter element can be removed (e.g. replaced with the 5′ UTR).

The CMV-IE 5′UTR and intron is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR and intron sequences discussed in Simari, et al. are also set out in WO2002/031137, incorporated by reference, and the regulatory sequences disclosed therein can also be used.

Other UTRs that can be used in combination with a promoter are known in the art, e.g. in Leppek, K., Das, R. & Barna, M. “Functional 5′ UTR mRNA structures in eukaryotic translation regulation and how to find them”. Nat Rev Mol Cell Biol 19, 158-174 (2018), incorporated by reference.

In some embodiments the sequence encoding the 5′ UTR and intron comprises the nucleic acid sequence of SEQ ID NO: 65, or a functional variant thereof. In some embodiments, functional variants of SEQ ID NO: 65 may have a sequence that is at least 60%, 65%, 70%, 75%, 80%, 85%, 90%, 95%, 96%, 97%, 98%, or 99% identical thereto. SEQ ID NO: 65 encodes a CMV-IE 5′ UTR and intron.

Table 8 discloses the sequence identifier numbers for other exemplary elements (e.g. introns/UTR, polyA sequences) for use in the promoter sequences or in the rAAV contructs as disclosed herein:

TABLE 8

| | |
|---|---|
| NAME | SEQUENCE ID NO: |
| HBB | SEQ ID NO: 283 |
| CMV-IE 5′UTR AND INTRON | SEQ ID NO: 65 |
| minimal poly A sequence | SEQ ID NO: 284 |
| minimal poly A sequence | SEQ ID NO: 285 |
| SV40 early poly A | SEQ ID NO: 286 |
| RBG poly A | SEQ ID NO: 287 |
| FULL synthetic poly A sequence | SEQ ID NO: 288 |
| poly A | SEQ ID NO: 445 |

In some embodiments the 5′ UTR suitably comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5′-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments, any one of the promoters described above, or variants thereof, is linked to a sequence encoding a 5′ UTR and or 5′UTR and an intron to provide a composite promoter. Herein, such composite promoter may be referred to simply as “composite promoters”, or in some cases simply “promoters” for brevity.

In some embodiments, the SP0067 promoter, or variants thereof, as discussed above is linked to a sequence encoding a 5′UTR and an intron to provide a composite promoter. In some embodiments, the composite promoter comprises SEQ ID NO: 17, or a functional variant thereof. This composite promoter construct comprises SP0067 operably linked to the 5′ UTR and intron from the CMV-IE gene. This composite promoter is referred to as SP0475 as described herein above.

### III.CREs and Functional Variants Thereof

Disclosed herein are various CREs that can be used in construction of cardiac-specific promoters. Suitably, the CREs are cardiac-specific. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain cardiac-specific regulatory activity. This is surprising because the removal of a regulatory sequence from the complex and “three dimensional” natural context in the genome often results in a significant loss of activity, so there is no reason to expect a given CRE to retain the levels of activity observed once removed from their natural environment. It is even more surprising when a CRE retain cardiac-specific activity in an AAV vector. This is particularly the case as an AAV vector comprises Inverted Terminal Repeat (ITR) and has a different DNA structure compared to the genome and both ITRs and the DNA structure are known to influence the activity of CREs.

It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

Table 3: Sequence Identifier number for the nucleic acid sequences of exemplary CREs (Cis-Regulatory Elements) for cardiac-specific promoters

TABLE 3

| | |
|---|---|
| CRM NAME | SEQUENCE ID NO: |
| CRE0004 | SEQ ID NO: 39 |
| CRE0028 | SEQ ID NO: 40 |
| CRE0033 | SEQ ID NO: 41 |
| CRE0070 | SEQ ID NO: 42 |
| CRE0071.3 | SEQ ID NO: 43 |
| CRE0095 | SEQ ID NO: 44 |
| CRE0096 | SEQ ID NO: 45 |
| CRE0105 | SEQ ID NO: 46 |
| CRE0106 | SEQ ID NO: 47 |
| CRE0107 | SEQ ID NO: 48 |
| CRE0108 | SEQ ID NO: 49 |
| CRE0109 | SEQ ID NO: 50 |
| CRE0111 | SEQ ID NO: 51 |
| CRE0114 | SEQ ID NO: 52 |
| CRE0117 | SEQ ID NO: 53 |
| CRE0118 | SEQ ID NO: 54 |

CREs of the present invention comprise certain cardiac-specific transcription factor binding sites (TFBS). It is generally desired that in functional variants of the CREs these Cardiac-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated positional weight matrices can be found in, for example, the Jaspar or Transfac databases http://jaspar.genereg.net/ and http://gene-regulation.com/ub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. It will be apparent that significant sequence modification in a CRE, even within TFBS in a CRE, can be made while retaining function.

CREs of the present invention can be used in combination with a wide range of suitable minimal promoters or Cardiac-specific proximal promoters.

Functional variants of a CRE include sequences which vary from the reference CRE element, but which substantially retain activity as Cardiac-specific CREs. It will be appreciated by the skilled person that it is possible to vary the sequence of a CRE while retaining its ability to recruit suitable Cardiac-specific transcription factors (TFs) and thereby enhance expression. A functional variant of a CRE can comprise substitutions, deletions and/or insertions compared to a reference CRE, provided they do not render the CRE substantially non-functional.

In some embodiments, a functional variant of a CRE can be viewed as a CRE which, when substituted in place of a reference CRE in a promoter, substantially retains its activity. For example, a cardiac-specific promoter which comprises a functional variant of a given CRE preferably retains at least 80% of its activity, more preferably at least 90% of its activity, more preferably at least 95% of its activity, and yet more preferably 100% of its activity (compared to the reference promoter comprising the unmodified CRE).

Suitably, functional variants of a CRE retain a significant level of sequence identity to a reference CRE. Suitably functional variants comprise a sequence that is at least 70% identical to the reference CRE, more preferably at least 80%, 90%, 95% or 99% identical to the reference CRE.

Retention of activity can be assessed by comparing expression of a suitable reporter under the control of the reference promoter with an otherwise identical promoter comprising the substituted CRE under equivalent conditions. Suitable assays for assessing cardiac-specific promoter activity are disclosed herein, e.g. in the examples.

In some embodiments, a CRE can be combined with one or more additional CREs to create a cis-regulatory module (CRM). Additional CREs can be provided upstream of the CREs according to the present invention, or downstream of the CRE according to the present invention. The additional CREs can be CREs disclosed herein, or they can be other CREs. Suitably, the additional CREs are cardiac-specific.

CREs according to the present invention or CRMs comprising CREs according to the present invention may comprise one or more additional regulatory elements. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRE or CRM substantially non-functional.

A promoter comprising CREs according to the present invention may comprise spacers between the CRM and the minimal or proximal promoter and/or between CREs. Additionally, or alternatively, a spacer may be present on the 5′ end of the CRM.

It will be apparent that a CRE according to the present invention or a CRM comprising a CRE according to this invention, or functional variants thereof, can be combined with any suitable promoter elements in order to provide a synthetic cardiac-specific promoter according to the present invention. Suitably, the promoter element is a Cardiac-specific proximal promoter.

In many instances, shorter promoter sequences are preferred, particularly for use in situations where a vector (e.g. a viral vector such as AAV) has limited capacity. Accordingly, in some embodiments the CREs according to the present invention or functional variants thereof have a length of 600 or fewer nucleotides, for example 600, 500, 450, 400, 350, 300, 250, 200, 150, 100, 75, 60, 50 or fewer nucleotides. Suitably, the synthetic cardiac specific CRM comprising at least one of the CREs according to SEQ ID NOs 19-24, 27, 28 or a functional variant thereof has length of 1000 or fewer nucleotides, for example 950, 900, 850, 800, 750, 700, 650, 600, 550, 500, 450, 400, 350, 300, 250, 200, 150, 100, 75, 60, 50 or fewer nucleotides.

### IV.CRMs and Functional Variants Thereof

Various synthetic Cardiac-specific CRMs are disclosed herein that can be used in the constructions of synthetic cardiac-specific promoters. CRMs of the present invention can be used in combination with a wide range of suitable minimal promoters or Cardiac-specific proximal promoters.

Functional variants of a CRM include sequences which vary from the reference CRM element, but which substantially retain activity as Cardiac-specific CRMs. It will be appreciated by the skilled person that it is possible to vary the sequence of a CRM while retaining its ability to recruit suitable Cardiac-specific transcription factors (TFs) and thereby enhance expression. A functional variant of a CRM can comprise substitutions, deletions and/or insertions compared to a reference CRM, provided they do not render the CRM substantially non-functional.

In some embodiments, a functional variant of a CRM can be viewed as a CRM which, when substituted in place of a reference CRM in a promoter, substantially retains its activity. For example, a cardiac-specific promoter which comprises a functional variant of a given CRM preferably retains at least 80% of its activity, more preferably at least 90% of its activity, more preferably at least 95% of its activity, and yet more preferably 100% of its activity (compared to the reference promoter comprising the unmodified CRM).

Suitably, functional variants of a CRM retain a significant level of sequence identity to a reference CRM. Suitably functional variants comprise a sequence that is at least 70% identical to the reference CRM, more preferably at least 80%, 90%, 95% or 99% identical to the reference CRM.

Retention of activity can be assessed by comparing expression of a suitable reporter under the control of the reference promoter with an otherwise identical promoter comprising the substituted CRM under equivalent conditions. Suitable assays for assessing cardiac-specific promoter activity are disclosed herein, e.g. in the examples.

Functional variants of a given CRM can, in some embodiments, comprise functional variants of one or more of the CREs present in the reference CRM. For example, functional variants of a given CRM can comprise functional variants of 1 or 2 of the CREs present in the reference CRM.

Functional variants of a given CRM can, in some embodiments, comprise the same combination CREs as a reference CRM, but the CREs can be present in a different order from the reference CRM. It is usually preferred that the CREs are present in the same order as the reference CRM (thus, the functional variant of a CRM suitably comprises the same permutation of the CREs as set out in a reference CRM).

Functional variants of a given CRM can, in some embodiments, comprise one or more additional CREs to those present in a reference CRM. Additional CREs can be provided upstream of the CREs present in the reference CRM, downstream of the CREs present in the reference CRM, and/or between the CREs present in the reference CRM. The additional CREs can be CREs disclosed herein, or they can be other CREs. Generally, it is preferred that a functional variant of a given CRM comprises the same CREs (or functional variants thereof) and does not comprise additional CREs.

Functional variants of a given CRM can comprise one or more additional regulatory elements compared to a reference CRM. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRM substantially non-functional.

Functional variants of a given CRM can comprise additional spacers between adjacent CREs or, if one or more spacer are present in the reference CRM, said one or more spacers can be longer or shorter than in the reference CRM. Spacers present in the reference CRM can be removed in the functional variant.

It will be apparent that the CRMs as disclosed herein, or functional variants thereof, can be combined with any suitable promoter elements in order to provide a synthetic cardiac-specific promoter according to the present invention. Suitably, the promoter element is a cardiac-specific proximal promoter.

In many instances, shorter promoter sequences are preferred, particularly for use in situations where a vector (e.g. a viral vector such as AAV) has limited capacity. Accordingly, in some embodiments the synthetic Cardiac-specific CRM has length of 500 or fewer nucleotides, for example 450, 400, 350, 300, 250, 200, 150, 100, 75, 60, 50 or fewer nucleotides.

### V.Synthetic Cardiac-Specific Promoters and Functional Variants Thereof

Various synthetic cardiac-specific promoters are disclosed herein. A functional variant of a reference synthetic cardiac-specific promoter is a promoter which comprises a sequence which varies from the reference synthetic cardiac-specific promoter, but which substantially retains cardiac-specific promoter activity. It will be appreciated by the skilled person that it is possible to vary the sequence of a synthetic cardiac-specific promoter while retaining its ability to recruit suitable Cardiac-specific transcription factors (TFs) and to recruit RNA polymerase II to provide cardiac-specific expression of an operably linked sequence (e.g. an open reading frame). A functional variant of a synthetic cardiac-specific promoter can comprise substitutions, deletions and/or insertions compared to a reference promoter, provided such substitutions, deletions and/or insertions do not render the synthetic cardiac-specific promoter substantially non-functional compared to the reference promoter.

Table 4: Exemplary minimal or proximal promoters used in some embodiments of the synthetic cardiac-specific promoters of Table 2A.

TABLE 4

| | |
|---|---|
| PROMOTER NAME | SEQUENCE ID NO: |
| SKM_18 | SEQ ID NO: 55 |
| SKM_20 | SEQ ID NO: 56 |
| CRE0082 | SEQ ID NO: 57 |
| CRE0104 | SEQ ID NO: 58 |
| CRE0110 | SEQ ID NO: 59 |
| CRE0112 | SEQ ID NO: 60 |
| CRE0113 | SEQ ID NO: 61 |
| CRE0115 | SEQ ID NO: 62 |
| CRE0116 | SEQ ID NO: 63 |
| CRE0038 | SEQ ID NO: 64 |

Accordingly, in some embodiments, a functional variant of a synthetic cardiac-specific promoter can be viewed as a variant which substantially retains the cardiac-specific promoter activity of the reference promoter. For example, a functional variant of a synthetic cardiac-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic cardiac-specific promoter often retain a significant level of sequence similarity to a reference synthetic cardiac-specific promoter. In some embodiments, functional variants comprise a sequence that is at least 70% identical to the reference synthetic cardiac-specific promoter, more preferably at least 80%, 90%, 95% or 99% identical to the reference synthetic cardiac-specific promoter.

Functional variants are defined herein below. Suitably, the synthetic cardiac-specific promoter may comprise a sequence which is at least 60%, 65%, 70%, 75%, 80%, 90%, 95%, 96%, 97%, 98% or 99% identical to any one of SEQ ID NOs: 3-64.

Activity in a functional variant can be assessed by comparing expression of a suitable reporter under the control of the reference synthetic cardiac-specific promoter with the putative functional variant under equivalent conditions. Suitable assays for assessing cardiac-specific promoter activity are disclosed herein, e.g. in the examples.

Functional variants of a given synthetic cardiac-specific promoter can comprise functional variants of a CRE present in the reference synthetic cardiac-specific promoter. Functional variants of a given synthetic cardiac-specific promoter can comprise functional variants of the CRM present in the reference synthetic cardiac-specific promoter. Functional variants of a given synthetic cardiac-specific promoter can comprise functional variants of the promoter element, or a different promoter element when compared to the reference synthetic cardiac-specific promoter.

Functional variants of a given synthetic cardiac-specific promoter can comprise one or more additional CREs to those present in a reference synthetic cardiac-specific promoter. Additional CREs can, for example, be provided upstream of the CREs present in the reference synthetic cardiac-specific promoter or downstream of the CREs present in the reference synthetic cardiac-specific promoter. The additional CREs can be CREs disclosed herein, or they can be other CREs.

Functional variants of a given synthetic cardiac-specific promoter can comprise additional spacers between adjacent elements (CREs, CRM or promoter element) or, if one or more spacers are present in the reference synthetic cardiac-specific promoter, said one or more spacers can be longer or shorter than in the reference synthetic cardiac-specific promoter. Alternatively, if one or more spacers are present in the reference synthetic cardiac-specific promoter, these spacers may be removed in the functional variant.

It will be apparent that synthetic cardiac-specific promoters of the present invention can comprise a CRE of the present invention or a CRM comprising a CRE of the present invention and additional regulatory sequences. For example, they may comprise one or more additional CREs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Preferred synthetic cardiac-specific promoters of the present invention exhibit cardiac-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by the CMV or myosin promoter in cardiac cells. In some embodiments, the expression level of the phosphatase inhibitor gene or other exemplary genes driven by synthetic cardiac specific promoters is equivalent to the expression level of those genes driven by CMV promoter in cardiac cells. In many cases higher levels of promoter activity is preferred, but this is not always the case; thus, in some cases more moderate levels of expression may be preferred. In some cases, it is desirable to have available a range of promoters of different activity levels to allow the level of expression to be tailored to requirements; the present disclose provides promoters with such a range of activities. Activity of a given synthetic cardiac-specific promoter of the present invention compared to a known promoter can be assessed by comparing Cardiac-specific expression of a reporter gene under control of the synthetic cardiac-specific promoter with expression of the same reporter under control of the known promoter, when the two promoters are provided in otherwise equivalent expression constructs and under equivalent conditions.

In addition to different activity levels, in some cases, it is desirable to have available a range of promoters with activity in different regions, such as different regions of the heart, e.g., ventricles versus atrium, or different heart cells, e.g., ventricular cardiomyocytes versus atrial cardiomyocytes or cardiac fibroblasts, or endothelial cells (EC) in the heart, as well as peri-vascular cells and pacemaker cells. Additionally, it may be desirable to have a range of promoters with different activity levels across different regions to allow the level of expression to be tailored to requirements. In some cases, expression in a specific region is desired. In some embodiments, expression in the cardiomyocytes is desired with little or no expression in the rest of the heart or rest of the body. Suitably, expression may be required in multiple regions within the heart. In some preferred embodiments, the cardiac-specific promoter according to the present invention shows activity in any or all of the following heart regions: aortic arch arteries (AA); aorta; cardiomyocytes (CM); endothelial or endocardial cells (ECs); inferior caval vein (ICV); interventricular septum (IVS); left atrium (LA); left superior caval vein (LSCV); left ventricle (LV); outflow tract (OT); pulmonary arteries (PO); proepicardial organ (PEO); pulmonary vein (PV); right atrium (RA); right superior caval vein (RSCV); right ventricle (RV); superior caval vein (SCV); cardiac smooth muscle cells (SMs). In some embodiments, the cardiac-specific promoter according to the present invention shows activity the heart areas mentioned above with little or no activity in other areas of the heart, other areas of the body.

In addition to different activity levels and different areas of activity, in some cases, it is desirable to have available a range of promoters with activity in different cells or combinations of cells, such as different cardiomyocytes populations. In some preferred embodiments, the cardiac-specific promoter according to the present invention shows activity in cardiomyocytes. In some preferred embodiments, the cardiac-specific promoter according to the present invention shows activity in ventricular cardiomyocytes or in conductive cardiomyocytes. In some preferred embodiments, the cardiac-specific promoter according to the present invention shows activity in cardiomyocytes and smooth muscle cells. In some preferred embodiments, the cardiac-specific promoter according to the present invention shows activity in ventricular cardiomyocytes, in conductive cardiomyocytes and smooth muscle cells in the heart. In some embodiments, the cardiac-specific promoter according to the present invention shows activity in cardiomyocytes with little or no expression in other heart cell types. In some embodiments, the cardiac-specific promoter according to the present invention shows activity in cardiomyocytes and smooth muscle cells in the heart with little or no expression in other heart cell types. In some embodiments, the cardiac-specific promoter according to the present invention shows activity in cardiomyocytes and in pacemaker cells with little or no expression in other heart cell types.

Alternatively, it might be preferred to have a widespread expression in all or almost all regions of the heart, suitably all areas of the contraction, e.g., in the cells that make up the ventricles and atrium.

Preferred synthetic cardiac-specific promoters of the present invention exhibit cardiac-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by MLC-2v cardiac specific promoter. Activity of a given synthetic cardiac-specific promoter of the present invention compared to the MLC-2v promoter can be assessed by comparing cardiac-specific expression of a reporter gene under control of the synthetic cardiac-specific promoter with expression of the same reporter under control of MLC-2v promoter in heart tissue or heart cells, e.g., cardiomyocytes, when the two promoters are provided in otherwise equivalent expression constructs and under equivalent conditions. In some embodiments a synthetic cardiac-specific promoter of the invention is able to increase expression of a gene (e.g. a therapeutic gene or gene of interest) in the neurones of a subject by at least 20%, at least 40%, at least 60%, at least 80%, at least 100%, at least 200%, at least 300%, at least 500%, at least 1000% or more relative to a known cardiac-specific promoter, suitably MLC-2v promoter. The cardiac-specific expression of a synthetic cardiac-specific promoter as disclosed herein can be compared with other known cardiac-specific promoters, e.g.,, but are not limited to, promoters from the following genes: α myosin heavy chain genes such as ventricular α myosin heavy chain gene, β myosin heavy chain genes such as ventricular β myosin heavy chain gene, ventricular myosin Myosin light chain 2v gene such as light chain 2 gene, myosin light chain 2a gene such as ventricular myosin light chain 2 gene, cardiac myocyte-restricted cardiac ankyrin repeat protein (CARP) gene, cardiac α-actin gene, cardiac m2 muscarinic / acetylcholine gene, There are ANP gene, BNP gene, cardiac troponin C gene, cardiac troponin I gene, cardiac troponin T gene, cardiac sarcoplasmic reticulum Ca-AT ase gene, skeletal α-actin gene, and artificial heart cellspecific promoter.

In addition, the synthetic cardiac-specific promoter as disclosed herein can be compared with other chamber specific promoters or enhancers can be used, for example, the quail slow myosin chain type 3 (MyHC3) or ANP promoter, or cGATA-6 enhancer for atrial specific expression. The Iroquois homeobox gene can be used for ventricular specific expression. Examples of ventricular myocyte specific promoters include the ventricular myosin light chain 2 promoter and the ventricular myosin heavy chain promoter. In some embodiments, the synthetic cardiac-specific promoter as disclosed herein can be compared to other promoters and / or enhancers include Csx / NKX2.5 gene, titin gene, α-actinin gene, myomesin gene, M protein gene, cardiac troponin T gene, RyR2 gene, Cx40 gene, Cx43 gene, and even Mef2, There are genes that bind dHAND, GATA, CarG, E-box, Csx / NKX2.5, or TGF-beta, or combinations thereof.

In many instances, shorter promoter sequences are preferred, particularly for use in situations where a vector (e.g. a viral vector such as AAV) has limited capacity. Accordingly, in some embodiments the synthetic cardiac-specific promoter has length of 1000 or fewer nucleotides, for example, 900, 800, 700,600, 500, 450, 400, 350, 300, 250, 200, 150, 100, or fewer nucleotides. Particularly preferred synthetic cardiac-specific promoters are those that are both short and which exhibit high levels of activity.

It is surprising when a cardiac-specific promoter retains cardiac-specific activity in an AAV vector as the AAV vector’s ITRs and different DNA structure compared to the genome are known to influence the activity of promoters, often the ITRs and different DNA structure negatively impact the activity of promoters.

It is generally preferred that a cardiac-specific promoter according to the present invention which comprises a variant CRE of any one of Table 2A, 5A or Tables 3 or 6 retains at least 25%, 50%, 75%, 80%, 85%, 90%, 95% or 100% of the activity of the reference CRE. Retention of activity can be assessed by comparing expression of a suitable reporter under the control of the reference promoter with an otherwise identical promoter comprising the substituted CRE under equivalent conditions. Suitably said activity is assessed using the examples as described herein, but other methods can be used. Suitably the or each CRE is a cardiac-specific cis-regulatory element.

Suitably the promoter element is a minimal or proximal promoter. Preferably, when present, the proximal promoter is a cardiac-specific proximal promoter.

In some embodiments, the synthetic cardiac-specific promoter comprises or consists of a sequence according to any one of SEQ ID NOs 3-64, or a functional variant thereof. In some embodiments the synthetic cardiac-specific promoter comprises or consists of a sequence which is at least 60%, 65%, 70%, 75%, 80%, 85%, 90%, 95%, 96%, 97%, 98% or 99% identical to any one of SEQ ID NOs 3-64.

The present invention thus provides various synthetic cardiac-specific promoters and functional variants thereof. It is generally preferred that a promoter according to the present invention which is a variant of any one of SEQ ID NO 3-38 or 55, 56, 80-200, 290-329 retains at least 25%, 50%, 75%, 80%, 85%, 90%, 95% or 100% of the activity of the reference promoter. Suitably said activity is assessed using the examples as described herein, but other methods can be used. Suitably the minimal or proximal promoter can be operably linked with a CRE or CRM. The CRE may be a CRE according to this invention or any other CRE. The CRM may be a CRM according to this invention or may comprise a CRE according to this invention. Suitably, the CRE or the CRM is cardiac-specific.

Suitably the proximal promoter according to the present invention may be operably linked with one or more proximal promoters. A synthetic cardiac-specific promoter according to the present invention may comprise or consist of two proximal promoters. Suitably, a synthetic cardiac-specific promoter according to the present invention may comprise or consist of two or more proximal promoters. Suitably, the proximal promoters are Cardiac-specific proximal promoters. Suitably, the at least two proximal promoters may be operably linked to a CRE or a CRM according to the present invention.

The CREs, minimal/proximal promoters or promoters of the present invention can be active in specific region of the heart, preferably in cardiomyocytes, or in specific heart cell type or in a combination of heart cell types or in a combination of both. Suitably therefore the CREs, minimal/proximal promoters, or promoters of the present invention are cardiac-specific.

The CREs, minimal/proximal promoters or promoters of the present invention can be active in one or more of the various parts of the heart. Suitably, the CREs, minimal/proximal promoter or promoters of the present invention may be active in the heart. Suitably, the CREs, minimal/proximal promoter or promoters of the present invention may be active in the cardiomyocytes but not in any other part of the heart. Suitably the CREs, minimal/proximal promoter or promoters of the present invention may be active in one or more of the various areas within the heart.

In some embodiments, it may be desirable that the CRE, CRM, minimal/proximal promoter or promoter of the present invention shows widespread activity in the heart. In some embodiments, the CRE, CRM, minimal/proximal promoter or promoter of the present invention is active in all parts of the heart (pan-heart). In some embodiments, the CRE, CRM, minimal/proximal promoter or promoter of the present invention is active in 1, 2, 3, 4, 5, 6, 7, 8 or 9 of the areas of the heart recited above.

In some embodiments, it may be desirable that the CRE, minimal/proximal promoter or promoter of the present invention shows predominant activity in one area of the heart. Suitably, it may be desirable that the CRE, minimal/proximal promoter or promoter of the present invention shows activity in one area of the heart but no, or only minimal, activity in the rest of the heart. In some embodiments, the CRE, minimal/proximal promoter or promoter of the present invention is active in only one area of the areas of the heart recited above.

The CREs, minimal/proximal promoters or promoters of the present invention can be active in various cells of the heart. The predominant cell types in the heart are ventricular cardiomyocytes, atrial cardiomyocytes, cardiac fibroblasts, or endothelial cells (EC) in the heart, as well as peri-vascular cells and pacemaker cells. Additionally, the CREs, minimal/proximal promoters or promoters of the present invention can be active in various regions of the heart, such as, for example, activity in any or all of the following heart regions: aortic arch arteries (AA); aorta; cardiomyocytes (CM); endothelial or endocardial cells (ECs); inferior caval vein (ICV); interventricular septum (IVS); left atrium (LA); left superior caval vein (LSCV); left ventricle (LV); outflow tract (OT); pulmonary arteries (PO); proepicardial organ (PEO); pulmonary vein (PV); right atrium (RA); right superior caval vein (RSCV); right ventricle (RV); superior caval vein (SCV); cardiac smooth muscle cells (SMs). In some embodiments, the cardiac-specific promoter according to the present invention shows activity the heart areas mentioned above with little or no activity in other areas of the heart, other areas of the body. Other cell types may be present, particularly in inflammatory condition. In some embodiments, the CRE, CRM, minimal/proximal promoter or promoter of the present invention is active in at least four, or at least three, or at least two, or at least one, heart cell types listed above, such as ventricular cardiomyocytes, atrial cardiomyocytes, cardiac fibroblasts, or endothelial cells (EC) in the heart, as well as peri-vascular cells and pacemaker cells.

In some embodiments, it may be desirable for the promoter to be active in a limited number of heart cell types, or in not more than one heart cell type.

In some embodiments, the CRE, CRM, minimal/proximal promoter or promoter of the present invention is active in specific subtypes of heart cell, such as for example, ventricular cardiomyocytes, atrial cardiomyocytes, cardiac fibroblasts, or endothelial cells (EC) in the heart, as well as peri-vascular cells and pacemaker cells.

Expression driven by a cardiac-specific promoter of the present invention in a desired heart tissue or heart cell may be for a period of at least 1 hour, 2 hours, 3 hours, 4 hours, 5 hours, 6 hours, 7 hours, 8 hours, 9 hours, 10 hours, 1 1 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, 100 years. Expression driven by a promoter of the present invention in a desired tissue or cell may be for a period of more than 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 20 years, 30 years, 40 years, 50 years, 60 years, 70 years, 80 years, 90 years, 100 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, 5-10 years, 10-15 years, 15-20 years, 20-30 years, 30-40 years, 40-50 years, 50-60 years, 60-70 years, 80-90 years or 90-100 years.

In a further aspect, there is provided a pharmaceutical composition comprising a rAAV vector comprising a synthetic cardiac-specific promoter, operatively linked to a transgene for the treatment of heart disease (e.g., but not limited to an inhibitor of PP1 and/or angiogenesis protein or peptide) according to the present invention. For example, rAAV vector particles may be prepared as pharmaceutical compositions for use in the methods of administration herein. It will be understood that such compositions necessarily comprise one or more active ingredients and, most often, a pharmaceutically acceptable excipient.

### VI.Muscle-Specific Promoters Active in Cardiac and Skeletal Muscle

In some embodiments, the rAAV vector comprises a nucleic acid encoding the therapeutic agent, e.g., inhibitor of PP1 or other agent, operatively linked to a muscle-specific promoter, wherein the muscle specific promoter is active in both skeletal muscle and cardiac muscle. Exemplary muscle-specific promoters are disclosed in Tables 5A, 5B, 6 and 7 herein. In some embodiments, the cardiac specific promoter is a synthetic cardiac specific promoter.

### Muscle-Specific Promoters That Are Active in Cardiac and Skeletal Muscle.

In some embodiments, the promoter is a synthetic muscle-specific promoter active in both skeletal and cardiac muscle. Examples of muscle-specific promoters active in both skeletal and cardiac muscle include SP0010, SP0020, SP0033, SP0038, SP0040, SP0042, SP0051, SP0057, SP0058, SP0061, SP0062, SP0064, SP0065, SP0066, SP0068, SP0070, SP0071, SP0076, SP0132, SP0133, SP0134, SP0136, SP0146, SP0147, SP0148, SP0150, SP0153, SP0155, SP0156, SP0157, SP0158, SP0159, SP0160, SP0161, SP0162, SP0163, SP0164, SP0165, SP0166, SP0169, SP0170, SP0171, SP0173, SP0228, SP0229, SP0230, SP0231, SP0232, SP0257, SP0262, SP0264, SP0265, SP0266, SP0267, SP0268, SP0270, SP0271, SP0279, SP0286, SP0305, SP0306, SP0307, SP0309, SP0310, SP0311, SP0312, SP0313, SP0314, SP0315, SP0316, SP0320, SP0322, SP0323, SP0324, SP0325, SP0326, SP0327, SP0328, SP0329, SP0330, SP0331, SP0332, SP0333, SP0334, SP0335, SP0336, SP0337, SP0338, SP0339, SP0340, SP0341, SP0343, SP0345, SP0346, SP0347, SP0348, SP0349, SP0350, SP0351, SP0352, SP0353, SP0354, SP0355, SP0356, SP0358, SP0359, SP0361, SP0362, SP0363, SP0364, SP0365, SP0366, SP0367, SP0368, SP0369, SP0370, SP0371, SP0372, SP0373, SP0374, SP0375, SP0376, SP0377, SP0378, SP0379, SP0380, SP0381, SP0382, SKM_14, SKM_18, SKM_20, SP0357, SP0437-SP0445, SP0447 and SP0453-SP0471,SP0473-SP0474. Examples of preferred synthetic muscle-specific promoters which are active in both skeletal and cardiac muscles are SP0057, SP0134, SP0173, SP0279, SP0286, SP0310, SP0316, SP0320 and SP0326.

In some embodiments, the synthetic muscle-specific promoter that is active in skeletal muscle and cardiac muscle comprises or consists of a sequence according to any one of SEQ ID Nos: 55, 56, 80-200, 290-329, or a functional variant thereof. In some embodiments the synthetic muscle-specific promoter that is active in skeletal muscle and cardiac muscle comprises or consists of a sequence which is at least 60%, 65%, 70%, 75%, 80%, 85%, 90%, 95%, 96%, 97%, 98% or 99% identical to any one of SEQ ID NOs 55, 56, 80-200, 290-329.

Table 5A shows the Sequence Identifier number of the nucleic acid sequences of exemplary muscle-specific promoters active in cardiac and skeletal muscle for use in the methods and composition as disclosed herein.

TABLE 5A

| NAME | SEQUENCE ID NO: | LENG TH | NAME | SEQUENCE ID NO: | LENGTH |
|---|---|---|---|---|---|
| SP0010 | SEQ ID NO: 80 | 298 | SP0333 | SEQ ID NO: 162 | 543 |
| SP0020 | SEQ ID NO: 81 | 354 | SP0334 | SEQ ID NO: 163 | 362 |
| SP0033 | SEQ ID NO: 82 | 270 | SP0335 | SEQ ID NO: 164 | 715 |
| SP0038 | SEQ ID NO: 83 | 286 | SP0336 | SEQ ID NO: 165 | 521 |
| SP0040 | SEQ ID NO: 84 | 315 | SP0337 | SEQ ID NO: 166 | 618 |
| SP0042 | SEQ ID NO: 85 | 421 | SP0338 | SEQ ID NO: 167 | 729 |
| SP0051 | SEQ ID NO: 86 | 524 | SP0339 | SEQ ID NO: 168 | 610 |
| SP0057 | SEQ ID NO: 87 | 601 | SP0340 | SEQ ID NO: 169 | 654 |
| SP0058 | SEQ ID NO: 88 | 531 | SP0341 | SEQ ID NO: 170 | 924 |
| SP0061 | SEQ ID NO: 89 | 528 | SP0343 | SEQ ID NO: 171 | 652 |
| SP0062 | SEQ ID NO: 90 | 454 | SP0345 | SEQ ID NO: 172 | 693 |
| SP0064 | SEQ ID NO: 91 | 484 | SP0346 | SEQ ID NO: 173 | 576 |
| SP0065 | SEQ ID NO: 92 | 465 | SP0347 | SEQ ID NO: 174 | 606 |
| SP0066 | SEQ ID NO: 93 | 484 | SP0348 | SEQ ID NO: 175 | 575 |
| SP0068 | SEQ ID NO: 94 | 448 | SP0349 | SEQ ID NO: 176 | 907 |
| SP0070 | SEQ ID NO: 95 | 444 | SP0350 | SEQ ID NO: 177 | 727 |
| SP0071 | SEQ ID NO: 96 | 404 | SP0351 | SEQ ID NO: 178 | 365 |
| SP0076 | SEQ ID NO: 97 | 438 | SP0352 | SEQ ID NO: 179 | 365 |
| SP0132 | SEQ ID NO: 98 | 538 | SP0353 | SEQ ID NO: 180 | 568 |
| SP0133 | SEQ ID NO: 99 | 528 | SP0354 | SEQ ID NO: 181 | 376 |
| SP0134 | SEQ ID NO: 100 | 655 | SP0355 | SEQ ID NO: 182 | 296 |
| SP0136 | SEQ ID NO: 101 | 588 | SP0356 | SEQ ID NO: 183 | 654 |
| SP0146 | SEQ ID NO: 102 | 660 | SP0358 | SEQ ID NO: 184 | 659 |
| SP0147 | SEQ ID NO: 103 | 806 | SP0359 | SEQ ID NO: 185 | 332 |
| SP0148 | SEQ ID NO: 104 | 938 | SP0361 | SEQ ID NO: 186 | 483 |
| SP0150 | SEQ ID NO: 105 | 814 | SP0362 | SEQ ID NO: 187 | 535 |
| SP0153 | SEQ ID NO: 106 | 418 | SP0363 | SEQ ID NO: 188 | 598 |
| SP0155 | SEQ ID NO: 107 | 508 | SP0364 | SEQ ID NO: 189 | 683 |
| SP0156 | SEQ ID NO: 108 | 718 | SP0365 | SEQ ID NO: 190 | 453 |
| SP0157 | SEQ ID NO: 109 | 202 | SP0366 | SEQ ID NO: 191 | 591 |
| SP0158 | SEQ ID NO: 110 | 705 | SP0367 | SEQ ID NO: 192 | 429 |
| SP0159 | SEQ ID NO: 111 | 615 | SP0368 | SEQ ID NO: 193 | 550 |
| SP0160 | SEQ ID NO: 112 | 586 | SP0369 | SEQ ID NO: 194 | 388 |
| SP0161 | SEQ ID NO: 113 | 740 | SP0370 | SEQ ID NO: 195 | 514 |
| SP0162 | SEQ ID NO: 114 | 650 | SP0371 | SEQ ID NO: 196 | 354 |
| SP0163 | SEQ ID NO: 115 | 621 | SP0372 | SEQ ID NO: 197 | 354 |
| SP0164 | SEQ ID NO: 116 | 764 | SP0373 | SEQ ID NO: 198 | 362 |
| SP0165 | SEQ ID NO: 117 | 480 | SP0374 | SEQ ID NO: 199 | 362 |
| SP0166 | SEQ ID NO: 118 | 894 | SP0375 | SEQ ID NO: 200 | 376 |
| SP0169 | SEQ ID NO: 119 | 248 | SP0376 | SEQ ID NO: 290 | 434 |
| SP0170 | SEQ ID NO: 120 | 482 | SP0377 | SEQ ID NO: 291 | 436 |
| SP0171 | SEQ ID NO: 121 | 534 | SP0378 | SEQ ID NO: 292 | 522 |
| SP0173 | SEQ ID NO: 122 | 728 | SP0379 | SEQ ID NO: 293 | 524 |
| SP0228 | SEQ ID NO: 123 | 885 | SP0380 | SEQ ID NO: 294 | 522 |
| SP0229 | SEQ ID NO: 124 | 1003 | SP0381 | SEQ ID NO: 295 | 524 |
| SP0230 | SEQ ID NO: 125 | 953 | SP0382 | SEQ ID NO: 296 | 524 |
| SP0231 | SEQ ID NO: 126 | 773 | SKM_14 | SEQ ID NO: 297 | 240 |
| SP0232 | SEQ ID NO: 127 | 683 | SKM_18 | SEQ ID NO: 55 | 242 |
| SP0257 | SEQ ID NO: 128 | 710 | SKM_20 | SEQ ID NO: 56 | 232 |
| SP0262 | SEQ ID NO: 129 | 943 | SP0357 | SEQ ID NO: 298 | 335 |
| SP0264 | SEQ ID NO: 130 | 724 | SP0437 | SEQ ID NO: 299 | 340 |
| SP0265 | SEQ ID NO: 131 | 822 | SP0438 | SEQ ID NO: 300 | 365 |
| SP0266 | SEQ ID NO: 132 | 1016 | SP0439 | SEQ ID NO: 301 | 365 |
| SP0267 | SEQ ID NO: 133 | 560 | SP0440 | SEQ ID NO: 302 | 585 |
| SP0268 | SEQ ID NO: 134 | 728 | SP0441 | SEQ ID NO: 303 | 546 |
| SP0270 | SEQ ID NO: 135 | 562 | SP0442 | SEQ ID NO: 304 | 585 |
| SP0271 | SEQ ID NO: 136 | 451 | SP0443 | SEQ ID NO: 305 | 328 |
| SP0279 | SEQ ID NO: 137 | 883 | SP0444 | SEQ ID NO: 306 | 328 |
| SP0286 | SEQ ID NO: 138 | 616 | SP0445 | SEQ ID NO: 307 | 436 |
| SP0305 | SEQ ID NO: 139 | 562 | SP0447 | SEQ ID NO: 308 | 291 |
| SP0306 | SEQ ID NO: 140 | 500 | SP0453 | SEQ ID NO: 309 | 761 |
| SP0307 | SEQ ID NO: 141 | 554 | SP0454 | SEQ ID NO: 310 | 720 |
| SP0309 | SEQ ID NO: 142 | 636 | SP0455 | SEQ ID NO: 311 | 551 |
| SP0310 | SEQ ID NO: 143 | 441 | SP0456 | SEQ ID NO: 312 | 688 |
| SP0311 | SEQ ID NO: 144 | 318 | SP0457 | SEQ ID NO: 313 | 621 |
| SP0312 | SEQ ID NO: 145 | 501 | SP0458 | SEQ ID NO: 314 | 759 |
| SP0313 | SEQ ID NO: 146 | 395 | SP0459 | SEQ ID NO: 315 | 837 |
| SP0314 | SEQ ID NO: 147 | 334 | SP0460 | SEQ ID NO: 316 | 298 |
| SP0315 | SEQ ID NO: 148 | 204 | SP0461 | SEQ ID NO: 317 | 365 |
| SP0316 | SEQ ID NO: 149 | 376 | SP0462 | SEQ ID NO: 318 | 356 |
| SP0320 | SEQ ID NO: 150 | 944 | SP0463 | SEQ ID NO: 319 | 772 |
| SP0322 | SEQ ID NO: 151 | 661 | SP0464 | SEQ ID NO: 320 | 837 |
| SP0323 | SEQ ID NO: 152 | 613 | SP0465 | SEQ ID NO: 321 | 772 |
| SP0324 | SEQ ID NO: 153 | 407 | SP0466 | SEQ ID NO: 322 | 764 |
| SP0325 | SEQ ID NO: 154 | 409 | SP0467 | SEQ ID NO: 323 | 671 |
| SP0326 | SEQ ID NO: 155 | 483 | SP0468 | SEQ ID NO: 324 | 671 |
| SP0327 | SEQ ID NO: 156 | 538 | SP0469 | SEQ ID NO: 325 | 506 |
| SP0328 | SEQ ID NO: 157 | 822 | SP0470 | SEQ ID NO: 326 | 365 |
| SP0329 | SEQ ID NO: 158 | 324 | SP0471 | SEQ ID NO: 327 | 624 |
| SP0330 | SEQ ID NO: 159 | 365 | SP0473 | SEQ ID NO: 328 | 718 |
| SP0331 | SEQ ID NO: 160 | 365 | SP0474 | SEQ ID NO: 329 | 465 |
| SP0332 | SEQ ID NO: 161 | 565 | | | |

Table 5B: CRE and minimal/proximal promoters of the embodiments of muscle-specific promoters active in cardiac and skeletal muscle shown in Table 1C

| | | | | | | |
|---|---|---|---|---|---|---|
| Promoter (SEQ ID NO) | CRE (SEQ ID NO) | CRE (SEQ ID NO) | CRE (SEQ ID NO) | CRE (SEQ ID NO) | Promoter element (SEQ ID NO) | 5′UTR and/or intron (SEQ ID NO) |
| SP0010 (80) | | | | | CRE0010 (264) | |
| SP0020 (81) | | | | CRE0020 (203) | CRE0053.2 SRL_mp (271) | |
| SP0033 (82) | | | | CRE0035 (208) | CRE0053.2 SRL_mp (271) | |
| SP0038 (83) | | | | CRE0031 (207) | CRE0053.2 SRL_mp (271) | |
| SP0040 (84) | | | | CRE0036 (209) | CRE0053.2 SRL_mp (271) | |
| SP0042 (85) | | | | CRE0036 (209) | CRE0037 (267) | |
| SP0051 (86) | | | | CRE0020 (203) | SKM_14 (277) | |
| SP0057 (87) | | | CRE0029 (206) | CRE0071 (216) | CRE0070 (42) | |
| SP0058 (88) | | | | CRE0016 (201) | CRE0005 (262) | |
| SP0061 (89) | | | | CRE0016 (201) | SKM_18 (55) | |
| SP0062 (90) | | | | CRE0018 (202) | SKM_18 (55) | |
| SP0064 (91) | | | | CRE0027 (205) | SKM_18 (55) | |
| SP0065 (92) | | | | CRE0028 (40) | SKM_18 (55) | |
| SP0066 (93) | | | | CRE0029 (206) | SKM_18 (55) | |
| SP0068 (94) | | | | CRE0035 (208) | SKM_18 (55) | |
| SP0070 (95) | | | | CRE0018 (202) | SKM_20 (56) | |
| SP0071 (96) | | | | CRE0025 (204) | SKM_20 (56) | |
| SP0076 (97) | | | | CRE0035 (208) | SKM_20 (56) | |
| SP0132 (98) | | | | CRE0020 (203) | SKM_18 (55) | |
| SP0133 (99) | | | | CRE0020 (203) | SKM_20 (56) | |
| SP0134 (100) | | | CRE0020 (203) | CRE0071 (216) | CRE0070 (42) | |
| SP0136 (101) | | | | CRE0020 (203) | CRE0010 (264) | |
| SP0146 (102) | | | | CRE0050 (211) | CRE0049 (270) | HBB intron (283) |
| SP0147 (103) | | | | CRE0020 (203) | CRE0049 (270) | HBB intron (283) |
| SP0148 (104) | | | | CRE0020 (203) | RSV (279) | |
| SP0150 (105) | | | | CRE0025 (204) | RSV (279) | |
| SP0153 (106) | | | | CRE0035 (208) | CRE0046 (268) | |
| SP0155 (107) | | CRE0035 (208) | 48 bp (224) | 48 bp (224) | CRE0046 (268) | |
| SP0156 (108) | | | CRE0035 (208) | CRE0020 (203) | SKM_14 (277) | |
| SP0157 (109) | | | | CRE0050 (211) | CRE0053.2 SRL_mp (271) | |
| SP0158 (110) | | | CRE0020 (203) | CRE0036 (209) | CRE0037 (267) | |
| SP0159 (111) | | | CRE0035 (208) | CRE0036 (209) | CRE0037 (267) | |
| SP0160 (112) | | | CRE0035 (208) | CRE0031 (207) | CRE0037 (267) | |
| SP0161 (113) | | | CRE0020 (203) | CRE0036 (209) | CRE0009 (263) | |
| SP0162 (114) | | | CRE0035 (208) | CRE0036 (209) | CRE0009 (263) | |
| SP0163 (115) | | | CRE0035 (208) | CRE0031 (207) | CRE0009 (263) | |
| SP0164 (116) | | | CRE0047 (210) | CRE0020 (203) | CRE0048 (269) | |
| SP0165 (117) | | | | CRE0047 (210) | CRE0048 (269) | |
| SP0166 (118) | | | | CRE0051 (212) | RSV (279) | |
| SP0169 (119) | | | | | SKM_18 (55) | |
| SP0170 (120) | | | | CRE0051 (212) | SKM_18 (55) | |
| SP0171 (121) | | | | CRE0010 (264) | SKM_18 (55) | |
| SP0173 (122) | | | CRE0010 (264) | CRE0035 (208) | SKM_18 (55) | |
| SP0228 (123) | | CRE0020 (203) | CRE0029 (206) | CRE0071 (216) | CRE0070 (42) | |
| SP0229 (124) | | CRE0020 (203) | CRE0029 (206) | CRE0071 (216) | SKM_18 (55) | |
| SP0230 (125) | | CRE0020 (203) | CRE0020 (203) | CRE0071 (216) | CRE0070 (42) | |
| SP0231 (126) | | | CRE0020 (203) | CRE0071 (216) | SKM_18 (55) | |
| SP0232 (127) | | | CRE0035 (208) | CRE0071 (216) | SKM_18 (55) | |
| SP0257 (128) | | | CRE0010 (264) | CRE0035 (208) | CRE0046 (268) | |
| SP0262 (129) | | | CRE0010 (264) | CRE0035 (208) | CRE0054 (272) | |
| SP0264 (130) | | | | CRE0035 (208) | CRE0010 (264) | |
| SP0265 (131) | | | | CRE0010 (264) | CRE0010_ALD OA (265) | |
| SP0266 (132) | | | CRE0010 (264) | CRE0035 (208) | CRE0010_ALD OA (265) | |
| SP0267 (133) | | | CRE0033 (41) | CRE0071 (216) | CRE0070 (42) | |
| SP0268 (134) | | | CRE0035 (208) | CRE0010 (264) | SKM_18 (55) | |
| SP0270 (135) | | | CRE0035 (208) | CRE0055 (273) | DES_mp_v1 (280) | |
| SP0271 (136) | | | | CRE0035 (208) | CRE0056 (274) | |
| SP0279 (137) | | | CRE0020 (203) | CRE0071 (216) | CRE0070.2 (275) | CMV-IE (65) |
| SP0286 (138) | | | | CRE0071 (216) | CRE0070.2 (275) | CMV-IE (65) |
| SP0305 (139) | | | CRE0010 (264) | CRE0035 (208) | CRE0053.2 SRL_mp (271) | |
| SP0306 (140) | | | CRE0029 (206) | CRE0035 (208) | CRE0053.2 SRL_mp (271) | |
| SP0307 (141) | | | CRE0020 (203) | CRE0035 (208) | CRE0053.2 SRL_mp (271) | |
| SP0309 (142) | | | CRE0035 (208) | CRE0035 (208) | SKM_18 (55) | |
| SP0310 (143) | | | | CRE0035 (208) | SKM_18 (55) | |
| SP0311 (144) | | | CRE0035 (208) | 48 bp (224) | CRE0053.2 SRL_mp (271) | |
| SP0312 (145) | | | CRE0047 (210) | CRE0035 (208) | CRE0053.2 SRL_mp (271) | |
| SP0313 (146) | | | CRE0035 (208) | CRE0059 (213) | CRE0053.2 SRL_mp (271) | |
| SP0314 (147) | | CRE0035 (208) | CRE0060 (214) | CRE0060 (214) | CRE0053.2 SRL_mp (271) | |
| SP0315 (148) | | | | CRE0050 (211) | CRE0053.2 SRL_mp (271) | |
| SP0316 (149) | | | | CRE0050 (211) | SKM_18 (55) | |
| SP0320 (150) | | | CRE0010 (264) | CRE0035 (208) | SKM_18 (55) | CMV-IE (65) |
| SP0322 (151) | | | CRE0069 (215) | CRE0051 (212) | SKM_18 (55) | |
| SP0323 (152) | | | CRE0069.2 (242) | CRE0051 (212) | SKM_18 (55) | |
| SP0324 (153) | | | | CRE0069 (215) | SKM_14 (277) | |
| SP0325 (154) | | | | CRE0069 (215) | SKM_18 (55) | |
| SP0326 (155) | | | | CRE0071 (216) | SKM_18 (55) | |
| SP0327 (156) | | | CRE0069 (215) | CRE0071 (216) | CRE0070 (42) | |
| SP0328 (157) | | CRE0020 (203) | CRE0069 (215) | CRE0071 (216) | CRE0070 (42) | |
| SP0329 (158) | | | | CRE0071.13 (243) | CRE0070 (42) | |
| SP0330 (159) | | | | CRE0071.3 (43) | CRE0070 (42) | |
| SP0331 (160) | | | | CRE0071.4 (236) | CRE0070 (42) | |
| SP0332 (161) | | | CRE0035 (208) | CRE0071 (216) | CRE0070 (42) | |
| SP0333 (162) | | | | CRE0035 (208) | CRE0072 (276) | |
| SP0334 (163) | | | | CRE0035 (208) | DES_mp_v1 (280) | |
| SP0335 (164) | | | CRE0035 (208) | CRE0055 (273) | CRE0034 (266) | |
| SP0336 (165) | | | | CRE0055 (273) | CRE0034 (266) | |
| SP0337 (166) | | | CRE0035 (208) | CRE0055 (273) | CRE0046 (268) | |
| SP0338 (167) | | CRE0069 (215) | CRE0035 (208) | CRE0055 (273) | DES_mp_v1 (280) | |
| SP0339 (168) | | CRE0035 (208) | 48 bp (224) | CRE0055 (273) | DES_mp_v1 (280) | |
| SP0340 (169) | | | CRE0035 (208) | CRE0046 (268) | SKM_18 (55) | |
| SP0341 (170) | | | CRE0035 (208) | CRE0055 (273) | CRE0010_ALD OA (265) | |
| SP0343 (171) | | | | CRE0035 (208) | SKM_18.2 (278) | CMV-IE (65) |
| SP0345 (172) | | | CRE0020 (203) | CRE0071 (216) | DES_mp_v1 (280) | |
| SP0346 (173) | | | CRE0069 (215) | CRE0071 (216) | DES_mp_v1 (280) | |
| SP0347 (174) | | | CRE0029 (206) | CRE0050 (211) | SKM_18 (55) | |
| SP0348 (175) | | | CRE0029.2 (241) | CRE0050 (211) | SKM_18 (55) | |
| SP0349 (176) | | CRE0029 (206) | CRE0035 (208) | CRE0071 (216) | SKM_18 (55) | |
| SP0350 (177) | | CRE0020 (203) | 72 bp (246) | CRE0071 (216) | CRE0070 (42) | |
| SP0351 (178) | | | | CRE0071.14 (244) | CRE0070 (42) | |
| SP0352 (179) | | | | CRE0071.15 (245) | CRE0070 (42) | |
| SP0353 (180) | | | | CRE0073 (218) | CRE0046 (268) | |
| SP0354 (181) | | | | CRE0074 (219) | CRE0046 (268) | |
| SP0355 (182) | | | | CRE0075 (220) | CRE0046 (268) | |
| SP0356 (183) | | | | CRE0076 (221) | CRE0046 (268) | |
| SP0358 (184) | | | | CRE0078 (222) | CRE0046 (268) | |
| SP0359 (185) | | | | CRE0079 (223) | CRE0046 (268) | |
| SP0361 (186) | | | | CRE0071.14 (244) | SKM_18 (55) | |
| SP0362(187) | | | CRE0069 (215) | CRE0071.5 (217) | DES_mp_v1 (280) | |
| SP0363 (188) | | | CRE0029 (206) | CRE0071.5 (217) | DES_mp_v1 (280) | |
| SP0364 (189) | | | CRE0029 (206) | CRE0071 (216) | CRE0046 (268) | |
| SP0365 (190) | | | | CRE0071 (216) | CRE0046 (268) | |
| SP0366 (191) | | | CRE0079 (223) | CRE0071 (216) | SKM_18 (55) | |
| SP0367 (192) | | | | CRE0079 (223) | CRE0034 (266) | |
| SP0368 (193) | | | CRE0079 (223) | CRE0035 (208) | SKM_18 (55) | |
| SP0369 (194) | | | | CRE0071.6 (237) | CRE0070 (42) | |
| SP0370 (195) | | | | CRE0071.7 (225) | CRE0070 (42) | |
| SP0371 (196) | | | | CRE0071.8 (238) | CRE0070 (42) | |
| SP0372 (197) | | | | CRE0071.9 (239) | CRE0070 (42) | |
| SP0373 (198) | | | | CRE0071.10 (226) | CRE0070 (42) | |
| SP0374 (199) | | | | CRE0071.11 (227) | CRE0070 (42) | |
| SP0375 (200) | | | | CRE0071.12 (228) | CRE0070 (42) | |
| SP0376 (290) | | CRE0035 (208) | HTMB ev_4 (282) | DES_MT_enha ncer_48bp_v2 (229) | DES_mp_v1 (280) | |
| SP0377 (291) | | CRE0035 (208) | HTMB ev_4 (282) | DES_MT_enha ncer_48bp_v3 (230) | DES_mp_v1 (280) | |
| SP0378 (292) | | | CRE0020 (203) | DES_MT_enha ncer_72bp_v2 (231) | DES_mp_v1 (280) | |
| SP0379 (293) | | | CRE0020 (203) | DES_MT_enha ncer_72bp_v3 (232) | DES_mp_v1 (280) | |
| SP0380 (294) | | | CRE0020 (203) | DES_MT_enha ncer_72bp_v4 (233) | DES_mp_v1 (280) | |
| SP0381 (295) | | | CRE0020 (203) | DES_MT_enha ncer_72bp_v5 (234) | DES_mp_v1 (280) | |
| SP0382 (296) | | | CRE0020 (203) | DES_MT_enha ncer_72bp_v6 (235) | DES_mp_v1 (280) | |
| SKM_14 (297) | | | | | SKM_14 (277) | |
| SKM_18 (55) | | | | | SKM_18 (55) | |
| SKM_20 (56) | | | | | SKM_20 (56) | |
| SP0357 (298) | | | | CRE0077 (240) | CRE0046 (268) | |
| SP0437 (299) | | | | CRE0071.16 (249) | CRE0070 (42) | |
| SP0438 (300) | | | | CRE0071.17 (250) | CRE0070 (42) | |
| SP0439 (301) | | | | CRE0071.18 (251) | CRE0070 (42) | |
| SP0440 (302) | | | CRE0020 (203) | CRE0071.13 (243) | CRE0070 (42) | |
| SP0441 (303) | | | CRE0020 (203) | CRE0071.19 (252) | CRE0070 (42) | |
| SP0442 (304) | | | CRE0020 (203) | CRE0071.5 (217) | CRE0070 (42) | |
| SP0443 (305) | | | | CRE0071.20 (253) | CRE0070 (42) | |
| SP0444 (306) | | | | CRE0071.21 (254) | CRE0070 (42) | |
| SP0445 (307) | | | | CRE0071.5 (217) | SKM_18 (55) | |
| SP0447 (308) | | | | CRE0071.22 (255) | CRE0070 (42) | |
| SP0453 (309) | | | CRE0020 (203) | CRE0071 (216) | SKM_18 (55) | |
| SP0454 (310) | | | CRE0020 (203) | CRE0071.5 (217) | SKM_18 (55) | |
| SP0455 (311) | | CRE0093 (247) | CRE0094 (248) | CRE0071 (216) | CRE0070 (42) | |
| SP0456 (312) | | CRE0093 (247) | CRE0094 (248) | CRE0071 (216) | SKM_18 (55) | |
| SP0457 (313) | CREO 093 (247) | CNTRL_001 (259) | CRE0094 (248) | CRE0071 (216) | CRE0070 (42) | |
| SP0458 (314) | | | CRE0020 (203) | CRE0071 (216) | SKM_14 (277) | |
| SP0459 (315) | | | CRE0020 (203) | CRE0071 (216) | CRE0049 (270) | |
| SP0460 (316) | | | | CRE0071.23 (256) | CRE0070 (42) | |
| SP0461 (317) | | | CRE0071.23 (256) | CNTRL_001 (67 bp) (257) | CRE0070 (42) | |
| SP0462 (318) | | | CRE0060 (214) | CRE0071.13 (243) | CRE0070 (42) | |
| SP0463 (319) | | | CRE0020 (203) | CRE0071 (216) | CRE0099 (281) | |
| SP0464 (320) | | | CRE0071 (216) | CRE0020 (203) | CRE0049 (270) | |
| SP0465 (321) | | | CRE0071 (216) | CRE0020 (203) | CRE0099 (281) | |
| SP0466 (322) | | CRE0071 (216) | CRE0093 (247) | CRE0094 (248) | CRE0049 (270) | |
| SP0467 (323) | | | CRE0035 (208) | CRE0071 (216) | SKM_18 (55) | |
| SP0468 (324) | | | CRE0071 (216) | CRE0035 (208) | SKM_18 (55) | |
| SP0469 (325) | | CRE0093.2 (260) | CRE0094.2 (261) | CRE0071 (216) | CRE0070 (42) | |
| SP0470 (326) | | | | CRE0071.24 (258) | CRE0070 (42) | |
| SP0471 (327) | | | CRE0071 (216) | CRE0020 (203) | CRE0070 (42) | |
| SP0473 (328) | | | CRE0020 (203) | CRE0071.5 (217) | SKM_14 (277) | |
| SP0474 (329) | | CRE0093.2 (260) | CRE0094.2 (261) | CRE0071.5 (217) | CRE0070 (42) | |

Table 13A shows the sequence identifier numbers for shortened nucleic acid sequences of exemplary muscle-specific promoters active in cardiac and skeletal muscle for use in the methods and composition as disclosed herein.

| NAME and SEQ ID NO: | LENGTH |
|---|---|
| SP0502 (SEQ ID NO: 336) | 251 |
| SP0515 (SEQ ID NO: 337) | 235 |
| SP0521 (SEQ ID NO: 338) | 263 |
| SP4169 (SEQ ID NO: 339) | 287 |
| SP0522 (SEQ ID NO: 340) | 237 |
| SP0523 (SEQ ID NO: 341) | 221 |
| SP0524 (SEQ ID NO: 342) | 249 |

Table 13B: CRE and minimal/proximal promoters of the embodiments of shortened muscle-specific promoters active in cardiac and skeletal muscle shown in Table 13A.

| | | | | | | |
|---|---|---|---|---|---|---|
| Promoter (SEQ ID NO) | CRE (SEQ ID NO) | CRE (SEQ ID NO) | CRE (SEQ ID NO) | CRE (SEQ ID NO) | Promoter element (SEQ ID NO) | 5′UTR and/or intron (SEQ ID NO) |
| SP0502 (SEQ ID NO: 336) | | | | CRE0050 (SEQ ID NO: 211) | CRE0053 (SEQ ID NO: 344) | |
| SP0515 (SEQ ID NO: 337) | | | | CRE0050 (SEQ ID NO: 211) | BG mp (SEQ ID NO: 345) | |
| SP0521 (SEQ ID NO: 338) | | | | CRE0050 (SEQ ID NO: 211) | SCPI (SEQ ID NO: 346) | |
| SP4169 (SEQ ID NO: 339) | | | | CRE0050 (SEQ ID NO: 211) | CRE0070 (SEQ ID NO: 42) | |
| SP0522 (SEQ ID NO: 340) | | | | CRE0145 (SEQ ID NO: 343) | CRE0053 (SEQ ID NO: 344) | |
| SP0523 (SEQ ID NO: 341) | | | | CRE0145 (SEQ ID NO: 343) | BG mp (SEQ ID NO: 345) | |
| SP0524 (SEQ ID NO: 342) | | | | CRE0145 (SEQ ID NO: 343) | SCPI (SEQ ID NO: 346) | |

### VII. Functional Variants of Muscle-Specific Promoters That Are Active in Cardiac and Skeletal Muscle:

### SP0057 and Variants Thereof

In some embodiments, the promoter is a synthetic muscle-specific promoter comprising a combination of the cis-regulatory elements CRE0029 and CRE0071, or functional variants thereof. Typically, the CREs are operably linked to a promoter element. In some preferred embodiments, the muscle-specific promoter comprises said CREs, or functional variants thereof, in the order CRE0029, CRE0071, and then the promoter element (order is given in an upstream to downstream direction, as is conventional in the art). In some preferred embodiments, the muscle-specific promoter comprises said CREs, or functional variants thereof, in the order CRE0071, CRE0029 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0029, CRE0071 and CRE0070, or functional variants thereof.

CRE0029 has a sequence according to SEQ ID NO: 206. Functional variants thereof may have a sequence that is at least 60%, 65%, 70%, 75%, 80%, 85%, 90%, 95%, 96%, 97%, 98%, or 99% identical thereto.

Functional variants of CRE0029 are regulatory elements with sequences which vary from CRE0029, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0029 can be viewed as a CRE which, when substituted in place of CRE0029 in a promoter, substantially retains its activity. For example, a muscle-specific promoter which comprises a functional variant of CRE0029 substituted in place of CRE0029 preferably retains 80% of its activity, more preferably 90% of its activity, more preferably 95% of its activity, and yet more preferably 100% of its activity. For example, considering promoter SP0057 as an example, CRE0029 in SP0057 can be replaced with a functional variant of CRE0029, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0029 or functional variant thereof can be provided on either strand of a double stranded polynucleotide and can be provided in either orientation. As such, complementary and reverse complementary sequences of SEQ ID NO: 206 or a functional variant thereof fall within the scope of the invention. Single stranded nucleic acids comprising the sequence according to SEQ ID NO: 206 or a functional variant thereof also fall within the scope of the invention.

CRE0071 has a sequence according to SEQ ID NO: 216. Functional variants thereof may have a sequence that is at least 60%, 65%, 70%, 75%, 80%, 85%, 90%, 95%, 96%, 97%, 98%, or 99% identical thereto.

Functional variants of CRE0071 are regulatory elements with sequences which vary from CRE0071, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0071 can be viewed as a CRE which, when substituted in place of CRE0071 in a promoter, substantially retains its activity. For example, a muscle-specific promoter which comprises a functional variant of CRE0029 substituted in place of CRE0071 preferably retains 80% of its activity, more preferably 90% of its activity, more preferably 95% of its activity, and yet more preferably 100% of its activity. For example, considering promoter SP0057 as an example, CRE0071 in SP0057 can be replaced with a functional variant of CRE0071, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0071 or functional variant thereof can be provided on either strand of a double stranded polynucleotide and can be provided in either orientation. As such, complementary and reverse complementary sequences of SEQ ID NO: 216 or a functional variant thereof fall within the scope of the invention. Single stranded nucleic acids comprising the sequence according to SEQ ID NO: 216 or a functional variant thereof also fall within the scope of the invention.

The sequence of CRE0070 and variants thereof are set out elsewhere herein.

In some embodiments the muscle-specific promoter comprises a sequence according to SEQ ID NO: X, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: X is referred to as SP0057. The SP0057 promoter is particularly preferred in some embodiments. This promoter has been found to be very specific for muscle, which is advantageous in some circumstances.

### SP0134 and Variants Thereof

In some embodiments, the promoter is a synthetic muscle-specific promoter comprising a combination of the cis-regulatory elements CRE0020 and CRE0071, or functional variants thereof. Typically the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some embodiments, the muscle-specific promoter comprises said CREs, or functional variants thereof, in the order CRE0071, CRE0020 and then the promoter element

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071 and CRE0070, or functional variants thereof.

CRE0020 has a sequence according to SEQ ID NO: 203. Functional variants thereof may have a sequence that is at least 60%, 65%, 70%, 75%, 80%, 85%, 90%, 95%, 96%, 97%, 98%, or 99% identical thereto.

Functional variants of CRE0020 are regulatory elements with sequences which vary from CRE0020, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0020 can be viewed as a CRE which, when substituted in place of CRE0020 in a promoter, substantially retains its activity. For example, a skeletal muscle-specific promoter which comprises a functional variant of CRE0020 substituted in place of CRE0020 preferably retains 80% of its activity, more preferably 90% of its activity, more preferably 95% of its activity, and yet more preferably 100% of its activity. For example, considering promoter SP0227 as an example, CRE0020 in SP0227 can be replaced with a functional variant of CRE0020, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0020 or functional variant thereof can be provided on either strand of a double stranded polynucleotide and can be provided in either orientation. As such, complementary and reverse complementary sequences of SEQ ID NO: 203 or a functional variant thereof fall within the scope of the invention. Single stranded nucleic acids comprising the sequence according to SEQ ID NO: 203 or a functional variant thereof also fall within the scope of the invention.

In some embodiments, the CRE0020 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides.

The sequence of CRE0071 and variants thereof are set out above.

The sequence of CRE0070 and variants thereof are set out elsewhere herein.

In some embodiments the muscle-specific promoter comprises a sequence according to SEQ ID NO: 100, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 100 is referred to as SP0134. The SP0134 promoter is particularly preferred in some embodiments. This promoter has been found to be very specific for muscle, which is advantageous in some circumstances.

### SP0173 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element.

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035 and SKM _18, or functional variants thereof.

CRE0010 has a sequence according to SEQ ID NO: 264. Functional variants thereof may have a sequence that is at least 60%, 65%, 70%, 75%, 80%, 85%, 90%, 95%, 96%, 97%, 98%, or 99% identical thereto.

As discussed above, functional variants of CRE0010 substantially retain the ability of CRE0010 to act as a muscle-specific promoter element. For example, when a functional variant of CRE0010 is substituted into muscle-specific promoter SP0320, the modified promoter retains at least 80% of its activity, more preferably at least 90% of its activity, more preferably at least 95% of its activity, and yet more preferably 100% of the activity of SP0320. Suitably the functional variant of CRE0010 comprises a sequence which is at least 70%, 80%, 90%, 95% or 99% identity to SEQ ID NO: 264.

In some preferred embodiments, a promoter element comprising or consisting of CRE0010 or a functional variant thereof has a length of 400 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

CRE0035 has a sequence according to SEQ ID NO: 208. Functional variants thereof may have a sequence that is at least 60%, 65%, 70%, 75%, 80%, 85%, 90%, 95%, 96%, 97%, 98%, or 99% identical thereto.

Functional variants of CRE0035 are regulatory elements with sequences which vary from CRE0035, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0035 can be viewed as a CRE which, when substituted in place of CRE0035 in a promoter, substantially retains its activity. For example, a muscle-specific promoter which comprises a functional variant of CRE0035 substituted in place of CRE0035 preferably retains 80% of its activity, more preferably 90% of its activity, more preferably 95% of its activity, and yet more preferably 100% of its activity. For example, considering promoter SP0173 as an example, CRE0035 in SP0173 can be replaced with a functional variant of CRE0035, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0035 or functional variant thereof can be provided on either strand of a double stranded polynucleotide and can be provided in either orientation. As such, complementary and reverse complementary sequences of SEQ ID NO: 208 or a functional variant thereof fall within the scope of the invention. Single stranded nucleic acids comprising the sequence according to SEQ ID NO: 208 or a functional variant thereof also fall within the scope of the invention.

The sequence of SKM_18 (SEQ ID NO: 55) and variants thereof are set out elsewhere herein.

In some embodiments the muscle-specific promoter comprises a sequence according to SEQ ID NO: 122, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 122 is referred to as SP0173. The SP0173 promoter is particularly preferred in some embodiments. This promoter has been found to be very specific for muscle, which is advantageous in some circumstances.

### SP0279 and Variants Thereof

In some embodiments, the promoter is a synthetic muscle-specific promoter comprising a combination of the cis-regulatory elements CRE0020 and CRE0071, or functional variants thereof. Typically the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some preferred embodiments, the muscle-specific promoter comprises said CREs, or functional variants thereof, in the order CRE0071, CRE0020 and then the promoter element. In some preferred embodiments, the muscle-specific promoter comprises said CREs, or functional variants thereof, in the order CRE0020, CRE0071, the promoter element and the CMV-IE 5′UTR and Intron (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071, CRE0070 and CMV-IE 5′UTR and intron, or functional variants thereof.

The sequence of CRE0020 and variants thereof are set out above.

The sequence of CRE0071 and variants thereof are set out above.

The sequence of CRE0070 and variants thereof are set out elsewhere herein.

The sequence of CMV-IE 5′UTR and intron and variants thereof are set out elsewhere herein.

In some embodiments the muscle-specific promoter comprises a sequence according to SEQ ID NO: 137, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 137 is referred to as SP0279. The SP0279 promoter is particularly preferred in some embodiments. This promoter has been found to be very specific for muscle, which is advantageous in some circumstances.

### SP0286 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element. In some preferred embodiments, the synthetic muscle-specific promoter comprises CRE0071 immediately upstream of the promoter element and CMV-IE 5′UTR and intron.

The promoter element can be any suitable proximal or minimal promoter. In some embodiments, the promoter element is a minimal promoter. Where the promoter is a proximal promoter, it is generally preferred that the proximal promoter is muscle- specific.

In some preferred embodiments the promoter element is CRE0070 or functional variant thereof. CRE0070 is a muscle-specific proximal promoter.

In some embodiments the synthetic muscle-specific promoter comprises the following elements (or functional variants thereof): CRE0071, CRE0070 and then CMV-IE 5′UTR and intron.

The sequence of CRE0071 and variants thereof are set out above.

The sequence of CRE0070 and variants thereof are set out elsewhere herein.

The sequence of CMV-IE 5′UTR and intron and variants thereof are set out elsewhere herein.

In some embodiments the muscle-specific promoter comprises a sequence according to SEQ ID NO: 138, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 138 is referred to as SP0286. The SP0286 promoter is particularly preferred in some embodiments. This promoter has been found to be very specific for muscle, which is advantageous in some circumstances.

### SP0310 and Variants Thereof

In some embodiments, the promoter is a synthetic muscle-specific promoter comprising CRE0035 operably linked to a promoter element. In some preferred embodiments, the synthetic muscle-specific promoter comprises CRE0035 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0035 and then SKM_18.

The sequence of CRE0035 and variants thereof are set out above.

The sequence of SKM_18 and variants thereof are set out elsewhere herein.

In some embodiments the muscle-specific promoter comprises a sequence according to SEQ ID NO: 143, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 143 is referred to as SP0310. The SP0310 promoter is particularly preferred in some embodiments. This promoter has been found to be very specific for muscle, which is advantageous in some circumstances.

### SP0316 and Variants Thereof

In some embodiments, the promoter is a synthetic muscle-specific promoter comprising CRE0050 operably linked to a promoter element. In some preferred embodiments, the synthetic muscle-specific promoter comprises CRE0050 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0050 and then SKM_18.

CRE0050 has a sequence according to SEQ ID NO: 211. Functional variants thereof may have a sequence that is at least 60%, 65%, 70%, 75%, 80%, 85%, 90%, 95%, 96%, 97%, 98%, or 99% identical thereto.

Functional variants of CRE0050 are regulatory elements with sequences which vary from CRE0050, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0050 can be viewed as a CRE which, when substituted in place of CRE0050 in a promoter, substantially retains its activity. For example, a muscle-specific promoter which comprises a functional variant of CRE0035 substituted in place of CRE0050 preferably retains 80% of its activity, more preferably 90% of its activity, more preferably 95% of its activity, and yet more preferably 100% of its activity. For example, considering promoter SP0316 as an example, CRE0050 in SP0316 can be replaced with a functional variant of CRE0050, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0050 or functional variant thereof can be provided on either strand of a double stranded polynucleotide and can be provided in either orientation. As such, complementary and reverse complementary sequences of SEQ ID NO: 211 or a functional variant thereof fall within the scope of the invention. Single stranded nucleic acids comprising the sequence according to SEQ ID NO: 211 or a functional variant thereof also fall within the scope of the invention.

The sequence of SKM_18 and variants thereof are set out elsewhere herein.

In some embodiments the muscle-specific promoter comprises a sequence according to SEQ ID NO: 149, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 149 is referred to as SP0316. The SP0316 promoter is particularly preferred in some embodiments. This promoter has been found to be very specific for muscle, which is advantageous in some circumstances.

### SP0320 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element. In some preferred embodiments, the synthetic muscle-specific promoter comprises said proximal promoter and CRE, or functional variants thereof, in the order CRE0010, CRE0035, the further promoter element followed by the CMV-IE 5′UTR and Intron,

The further promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035, SKM_18 and CMV-IE 5′UTR and intron, or functional variants thereof.

The sequence of CRE0010 and variants thereof are set out above.

The sequence of CRE0035 and variants thereof are set out above.

The sequence of SKM_18 and variants thereof are set out elsewhere herein.

The sequence of the CMV-IE 5′UTR and intron and variants thereof are set out elsewhere herein.

In some embodiments the muscle-specific promoter comprises a sequence according to SEQ ID NO: 150, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 150 is referred to as SP0320. The SP0320 promoter is particularly preferred in some embodiments. This promoter has been found to be very specific for muscle, which is advantageous in some circumstances.

### SP0326 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle- specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0071 and then SKM_18.

The sequence of CRE0071 and variants thereof are set out above.

The sequence of SKM_18 and variants thereof are set out elsewhere herein.

In some embodiments the muscle-specific promoter comprises a sequence according to SEQ ID NO: 155, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 155 is referred to as SP0326. The SP0326 promoter is particularly preferred in some embodiments. This promoter has been found to be very specific for muscle, which is advantageous in some circumstances.

Table 6: The Sequence identifier numbers for the nucleic acid sequences of exemplary CREs (Cis-Regulatory Elements) for muscle-specific promoters that are active in both cardiac and skeletal muscle. which are disclosed in Table 5A.

TABLE 6

| | |
|---|---|
| NAME (SEQ ID NO:) | NAME (SEQ ID NO:) |
| CRE0016 (SEQ ID NO: 201) | DES_MT_ENHANCER_72 BP_V2 (SEQ ID NO: 231) |
| CRE0018 (SEQ ID NO: 202) | DES_MT_ENHANCER_72 BP_V3 (SEQ ID NO: 232) |
| CRE0020 (SEQ ID NO: 203) | DES_MT_ENHANCER_72 BP_V4 (SEQ ID NO: 233) |
| CRE0025 (SEQ ID NO: 204) | DES_MT_ENHANCER_72 BP_V5 (SEQ ID NO: 234) |
| CRE0027 (SEQ ID NO: 205) | DES_MT_ENHANCER_72 BP_V6 (SEQ ID NO: 235) |
| CRE0028 (SEQ ID NO: 40) | CRE0071.3 (SEQ ID NO: 43) |
| CRE0029 (SEQ ID NO: 206) | CRE0071.4 (SEQ ID NO: 236) |
| CRE0031 (SEQ ID NO: 207) | CRE0071.6 (SEQ ID NO: 237) |
| CRE0033 (SEQ ID NO: 41) | CRE0071.8 (SEQ ID NO: 238) |
| CRE0035 (SEQ ID NO: 208) | CRE0071.9 (SEQ ID NO: 239) |
| CRE0036 (SEQ ID NO: 209) | CRE0077 (SEQ ID NO: 240) |
| CRE0047 (SEQ ID NO: 210) | CRE0029.2 (SEQ ID NO: 241) |
| CRE0050 (SEQ ID NO: 211) | CRE0069.2 (SEQ ID NO: 242) |
| CRE0051 (SEQ ID NO: 212) | CRE0071.13 (SEQ ID NO: 243) |
| CRE0059 (SEQ ID NO: 213) | CRE0071.14 (SEQ ID NO: 244) |
| CRE0060 (SEQ ID NO: 214) | CRE0071.15 (SEQ ID NO: 245) |
| CRE0069 (SEQ ID NO: 215) | 72 BP (SEQ ID NO: 246) |
| CRE0071 (SEQ ID NO: 216) | CRE0093 (SEQ ID NO: 247) |
| CRE0071.5 (SEQ ID NO: 217) | CRE0094 (SEQ ID NO: 248) |
| CRE0073 (SEQ ID NO: 218) | CRE0071.16 (SEQ ID NO: 249) |
| CRE0074 (SEQ ID NO: 219) | CRE0071.17 (SEQ ID NO: 250) |
| CRE0075 (SEQ ID NO: 220) | CRE0071.18 (SEQ ID NO: 251) |
| CRE0076 (SEQ ID NO: 221) | CRE0071.19 (SEQ ID NO: 252) |
| CRE0078 (SEQ ID NO: 222) | CRE0071.20 (SEQ ID NO: 253) |
| CRE0079 (SEQ ID NO: 223) | CRE0071.21 (SEQ ID NO: 254) |
| 48 BP (SEQ ID NO: 224) | CRE0071.22 (SEQ ID NO: 255) |
| CRE0071.7 (SEQ ID NO: 225) | CRE0071.23 (SEQ ID NO: 256) |
| CRE0071.10 (SEQ ID NO: 226) | CNTRL_001 (67 BP) (SEQ ID NO: 257) |
| CRE0071.11 (SEQ ID NO: 227) | CRE0071.24 (SEQ ID NO: 258) |
| CRE0071.12 (SEQ ID NO: 228) | CNTRL_001 (SEQ ID NO: 259) |
| DES_MT_ENHANCER_48BP_V2 (SEQ ID NO: 229) | CRE0093.2 (SEQ ID NO: 260) |
| DES_MT_ENHANCER_48BP_V3 (SEQ ID NO: 230) | CRE0094.2 (SEQ ID NO: 261) |

Table 7: Exemplary minimal or proximal promoters used in some embodiments of the synthetic muscle-specific promoters that are active in cardiac and skeletal muscle of Table 5A.

TABLE 7

| | |
|---|---|
| Name | Name |
| CRE0005 (SEQ ID NO: 262) | CRE0056 (SEQ ID NO: 274) |
| CRE0009 (SEQ ID NO: 263) | CRE0070 (SEQ ID NO: 42) |
| CRE0010_ITGB1BP2 (SEQ ID NO: 264) | CRE0070.2 (SEQ ID NO: 275) |
| CRE0010_ALDOA (SEQ ID NO: 265) | CRE0072 (SEQ ID NO: 276) |
| CRE0034 (SEQ ID NO: 266) | SKM_14 (SEQ ID NO: 277) |
| CRE0037 (SEQ ID NO: 267) | SKM_18.2 (SEQ ID NO: 278) |
| CRE0046 (SEQ ID NO: 268) | SKM_18 (SEQ ID NO: 55) |
| CRE0048 (SEQ ID NO: 269) | SKM_20 (SEQ ID NO: 56) |
| CRE0049 (SEQ ID NO: 270) | RSV promoter (SEQ ID NO: 279) |
| CRE0053.2 SRL_mp (SEQ ID NO: 271) | DES_mp_v1 (SEQ ID NO: 280) |
| CRE0054 (SEQ ID NO: 272) | CRE0099 (SEQ ID NO: 281) |
| CRE0055 (SEQ ID NO: 273) | HTMB ev_4 (SEQ ID NO: 282) |

Table 14: Sequence identifier numbers of nucleic acid sequences of exemplary cis-regulatory modules (CRM) for shortened muscle-specific promoters that are active in both cardiac and skeletal muscle. which are disclosed in Table 13A.

TABLE 14

| CRM | SEQ ID NO: | length |
|---|---|---|
| CRM SP0502, SP0515, SP0521, and SP4169 | SEQ ID NO: 347 | 176 |
| CRM SP0522, SP0523, and SP0524 | SEQ ID NO: 351 | 162 |

Table 15: Sequence identifier numbers of nucleic acid sequences of exemplary CREs (Cis-Regulatory Elements) for shortened muscle-specific promoters that are active in both cardiac and skeletal muscle. which are disclosed in Table 13A.

TABLE 15

| CRM | SEQ ID NO: | length |
|---|---|---|
| CRE0145 | SEQ ID NO: 343 | 114 |

Table 16: Sequence identifier numbers of nucleic acid sequences of exemplary Promoter elements from synthetic promoters for shortened muscle-specific promoters that are active in both cardiac and skeletal muscle. which are disclosed in Table 13A.

TABLE 16

| CRM | SEQ ID NO: | length |
|---|---|---|
| BG mp | SEQ ID NO: 345 | 53 |
| SCP1 | SEQ ID NO: 346 | 81 |
| CRE0053 | SEQ ID NO: 344 | 69 |

### VIII. Synthetic Cardiac-Specific Expression Cassettes

The present invention also provides a synthetic cardiac-specific expression cassette comprising a synthetic cardiac-specific promoter of the present invention operably linked to a sequence encoding an expression product, suitably a gene (e.g. a transgene), e.g., an inhibitor of PP1 as disclosed herein, and/or an angiogenesis protein or peptide as disclosed herein.

In some preferred embodiments of the present invention, the gene encodes a therapeutic expression product, preferably a therapeutic polypeptide suitable for use in treating a disease or condition associated with aberrant gene expression, optionally in the heart tissue.

In some embodiments, therapeutic expression products include those useful in the treatment of a cardiovascular condition or heart disease and disorders, as disclosed herein. In some embodiments, the heart disorder is heart failure or CHF.

### C. Secretory Signal Peptides and Intron Sequences

In some embodiments, the nucleic acid encoding an inhibitor of PP1 can comprise a secretory peptide 5′ of the nucleic acid encoding the inhibitor of the PP1 (e.g., I-1, I-1c or a variant thereof). Suitable signal peptides are disclosed in WO2020/102645, which is incorporated herein in its entirety by reference. For example, a polynucleotide containing a suitable secretory sequence can be fused 5′ to the first codon of the selected inhibitor of PP1, or angiogenic protein gene. In some embodiments, a suitable secretory signal sequences include signal sequences of the FGF-4, FGF-5, FGF-6 genes or a signal sequence of a different secreted protein such as IL-1-beta. In some embodiments, a suitable secretory sequence is a secretory signal sequence derived from a protein that is normally secreted from cardiac myocytes. In some embodiments, the nucleic acid encoding the inhibitor of PP1 or angiogenic protein or peptide comprises a targeting peptide, e.g., a suitable cardiac targeting peptide is disclosed in WO2018/170310 or US20170166926A1, which is incorporated herein in its entirety by reference. Other suitable targeting peptides include the peptides targeting coronary artery endothelial cells identified by Muller et al., Nature Biotechnology 21:1040-1046 (2003), and disclosed as SEQ ID NO: 2-126 as disclosed in WO2019/216932A1, which is incorporated herein in its entirety by reference.

In some embodiments, the rAAV genotype comprises an intron sequence located 3′ of the promoter sequence and 5′ of a secretory signal peptide, if present or 5′ of the nucleic acid sequence encoding the PP1 inhibitor. Intron sequences serve to increase one or more of: mRNA stability, mRNA transport out of nucleus and/or expression and/or regulation of the expressed polypeptide.

In some embodiments, the intron sequence is a MVM intron sequence, for example, but not limited to and intron sequence of SEQ ID NO: 13 as disclosed in WO2020/102645, or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 14 as disclosed in WO2020/102645, or nucleic acid sequence having at least 80%, 85%, 90%, 95%, 96%, 97%, 98% or 99% nucleotide sequence identity thereto.

In some embodiments of the methods and compositions disclosed herein, a recombinant AAV vector comprises a heterologous nucleic acid sequence that further comprises an intron sequence located 5′ of the sequence encoding the secretory signal peptide, and 3′ of the promoter. In some embodiments, the intron sequence comprises a MVM sequence or a HBB2 sequence, wherein the MVN sequence comprises the nucleic acid sequence of SEQ ID NO: 13 as disclosed in WO2020/102645, or a nucleic acid sequence at least about 75%, or 80%, or 85%, or 90%, or 95%, or 98%, or 99% sequence identity to SEQ ID NO: 13 as disclosed in WO2020/102645, and the HBB2 sequence comprises the nucleic acid sequence of SEQ ID NO: 14 as disclosed in WO2020/102645, or a nucleic acid sequence at least about 75%, or 80%, or 85%, or 90%, or 95%, or 98%, or 99% sequence identity to SEQ ID NO: 14 as disclosed in WO2020/102645.

In some embodiments, the rAAV genotype comprises an intron sequence selected in the group consisting of a human beta globin b2 (or HBB2) intron, a FIX intron, a chicken beta-globin intron, and a SV40 intron. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### D. Poly A

In some embodiments, an rAAV vector genome includes at least one poly-A tail that is located 3′ and downstream from the heterologous nucleic acid gene encoding the PP1 inhibitor (e.g., I-1 or I-1c). In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences. In some embodiments, the rAAV genome comprises 3′ of the nucleic acid encoding the PP1 inhibitor (e.g., I-1 or I-1c), the following elements; a first polyA sequence, a spacer nucleic acid sequence (of between 100-400 bp, or about 250 bp), a second poly A sequence, a spacer nucleic acid sequence, and the 3′ ITR. In some embodiments, the first and second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa - that is, in alternative embodiments, the first poly A sequence is a synthetic poly A sequence and the second poly A sequence is a hGH polyA sequence. An exemplary poly A sequence is, for example, hGH poly A sequence (SEQ ID NO: 66), or a poly A nucleic acid sequence having at least 80%, 85%, 90%, 95%, 96%, 97%, 98% or 99% nucleotide sequence identity to SEQ ID NO: 66. In some embodiments, the hGHpoly sequence encompassed for use is described in Anderson et al. J. Biol. Chem 264(14); 8222-8229, 1989 (See, e.g. p. 8223, 2nd column, first paragraph) which is incorporated herein in its entirety by reference.

In some embodiments, a poly-A tail can be engineered to stabilize the RNA transcript that is transcribed from an rAAV vector genome, including a transcript for a heterologous gene, which in one embodiment the PP1 inhibitor (e.g., I-1 or I-1c), and in alternative embodiments, the poly-A tail can be engineered to include elements that are destabilizing.

In some embodiments of the methods and compositions disclosed herein, a recombinant AAV vector comprises at least one polyA sequence located 3′ of the nucleic acid encoding the PP1 inhibitor (e.g., I-1 or I-1c) peptide and 5′ of the 3′ ITR sequence.

In an embodiment, a poly-A tail can be engineered to become a destabilizing element by altering the length of the poly-A tail. In an embodiment, the poly-A tail can be lengthened or shortened. In some embodiments, the 3′ untranslated region comprises a 3′ UTR.

In some embodiments, a destabilizing element is a microRNA (miRNA) that has the ability to silence (repress translation and promote degradation) the RNA transcripts the miRNA bind to that encode a heterologous gene. In an embodiment, addition or deletion of seed regions within the poly-A tail can increase or decrease expression of a protein, such as the PP1 inhibitor (e.g., I-1 or I-1c) as disclosed herein.

In some embodiments, seed regions can also be engineered into the 3′ untranslated regions located between the heterologous gene and the poly-A tail. In a further embodiment, the destabilizing agent can be an siRNA. The coding region of the siRNA can be included in an rAAV vector genome and is generally located downstream, 3′ of the poly-A tail.

In all aspects of the methods and compositions as disclosed herein, the rAAV genome may also comprise a Stuffer DNA nucleic sequence. An exemplary stuffer DNA sequence is any non-coding DNA sequence. An exemplary stuffer DNA sequences are shown Table 9, or a nucleic acid sequence having at least 80%, 85%, 90%, 95%, 96%, 97%, 98% or 99% nucleotide sequence identity to a sequence shown in Table 9. In some embodiments, the stuffer sequence is located 3 of the poly A tail, for example, and is located 5′ of the ‘3 ITR sequence. In some embodiments, the stuffer DNA sequence comprises a synthetic polyadenylation signal in the reverse orientation.

In some embodiments, a stuffer nucleic acid sequence can be located between the poly A sequence and the 3′ ITR (i.e., a stuffer nucleic acid sequence is located 3′ of the polyA sequence and 5′ of the 3′ ITR). Such a stuffer nucleic acid sequence can be about 30 bp, 50 pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence comprise Exemplary stuffer (or spacer) nucleic acid sequence comprise the Sequences shown in Table 9, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to the sequences shown in Table 9.

Table 9 Exemplary spacer or stuffer sequences:

| | |
|---|---|
| Exemplary Stuffer sequences | SEQ ID NO: |
| Stuffer sequence 1 | SEQ ID NO: 67 |
| Stuffer sequence 2 | SEQ ID NO: 68 |
| Stuffer sequence 3 | SEQ ID NO: 69 |

Suitably the rAAV vector as disclosed herein comprising a synthetic cardiac-specific promoter can further comprises sequences providing or coding for one or more of, and preferably all of, a ribosomal binding site, a start codon, a stop codon, and a transcription termination sequence. Suitably the rAAV vector comprises an expression cassette comprises a nucleic acid encoding a posttranscriptional regulatory element. Suitably the expression cassette comprises a nucleic acid encoding a polyA element.

### Poly A, Double Stranded RNA Termination Element and Reverse Poly A

As described herein, the rAAV comprising nucleic acid sequence encoding phosphatase inhibitor gene e.g, I I c gene as disclosed herein, the nucleic acid sequence further comprises a polyadenylation signal or, polyA signal after the coding sequence of I1c gene or, codon optimized I1c gene as disclosed herein. In some embodiments, the poly A signal is homologous. In some embodiments, the poly A signal is heterologous. In some embodiments, the poly A signal may comprise a double stranded RNA termination element and/or, a reverse poly A. In some embodiments, the poly A signal is double stranded RNA termination element and/or, a reverse poly A. In some embodiments, the reverse poly A or, double stranded RNA terminator is located after the homologous or, heterologous poly A signal sequence.

A ‘double stranded RNA termination element’ is an element that inhibits transcription of double stranded RNA, e.g. from the 3′ ITR. In some embodiments, the double stranded RNA termination element is located downstream of the I1c gene and upstream of 3′ITR, in 3′ to 5′ orientation. In 3′ to 5′ orientation, the termination element does not allow the transcription from 3′ITR and hence double stranded RNA is not transcribed from 3′ITR. Any termination element can be used including e.g. inverted natural polyA sequences from any species or synthetic polyA signals, or, fragments thereof; or other nucleic acid structure terminators known in the art. Exemplary polyA signal and/or, transcription terminators include, but are not limited to polyA signals of BGH, SV40, HGH, Betaglobin, RNA polymerase II transcriptional pause signal from alpha 2 globin gene, transcription termination signal for pol III, fragments thereof and any combination thereof.

A ‘reverse poly A′ is a polyA signal sequence placed in a 3′-5’ orientation downstream of the I1c transgene and upstream of 3′ITR. Any natural or synthetic poly A in 3′-5′ orientation can be used as reverse poly A. In some embodiments, the reverse poly A is the poly A (pA) as described in International publication no. WO2019143950 and U.S. Application Publication No. US20200340013, which are incorporated herein by reference in entirety. In several embodiments, the ‘reverse poly A’ and ‘the double stranded RNA termination element’ are used interchangeably. In 3′ to 5′ orientation, the reverse poly A or, the termination element does not allow the transcription from 3′ITR and hence double stranded RNA is not transcribed from 3′ITR. The reverse poly A or, the double stranded RNA termination element can be heterologous e.g, from a different gene than the gene of interest, or, homologous, e.g., the same gene as the gene of interest. In the instant application, the gene of interest is I1c.

In various embodiments, the poly A signal comprise the double stranded RNA transcription element or reverse poly A. In some embodiments, the 5′ end of double stranded RNA termination element or, reverse poly A sequence and the 3′ end of poly A signal is right next to each other or, 1 neucleotide apart, or, 2 neucleotides apart, or, 3 neucleotides apart, or, 4 neucleotides apart, or,5 neucleotides apart, or, 6 neucleotides apart, or, 7 neucleotides apart, or, 8 neucleotides apart, or, 9 neucleotides apart, or, 10 neucleotides apart, or more than 10 neucleotides apart. In some embodiments, the poly A signal does not comprise double stranded RNA transcription element or reverse poly A. n some embodiments the poly A signal comprises AATAAA or, AAUAAA. In some embodiments, the poly A signal comprises 1, or, 2, or, 3, or, 4, or 5, or, 6 or, 7, or, 8, or, 9 or, 10 or, more repeats of AATAAA or, AAUAAA. In some embodiments, the poly A signal comprises transcription termination signal for Pol III as described in “Delineation of the Exact Transcription Termination Signal for Type 3 Polymerase III. Mol Ther Nucleic Acids. 2018 Mar 2;10:36-44, which is incorporated herein by reference in its entirety. In some embodiments, one or, more of transcription termination signal for Pol III is in 3′ to 5′ orientation. In some embodiments, the poly A signal comprises TTTT. In some embodiments, poly A signal comprises AAAAAAA.

Exemplary Double stranded RNA Termination element sequence is (RNA polymerase II transcription pause signal) is SEQ ID NO: 332 or a sequence with at least 80% sequence identity to SEQ ID NO: 332.

Exemplary poly A sequences for use in the rAAV and linear DNA as disclosed herein include, but are not limited to (i) a hGH poly(A) signal and terminator sequence with a nucleic acid sequence of SEQ ID NO: 331 or a nucleic acid sequence having at least 80% sequence identity to SEQ ID NO: 331; (ii) a SV40 poly A sequence having a nucleic acid comprising SEQ ID NO: 334 or a nucleic acid sequence having at least 80% sequence identity to SEQ ID NO: 331; (iii) a full synthetic poly A sequence comprising a nucleic acid of SEQ ID NO: 335 or a nucleic acid sequence having at least 80% sequence identity to SEQ ID NO: 335; (iv) a minimal synthetic poly A sequence comprising a nucleic acid of SEQ ID NO: 284 or a nucleic acid sequence having at least 80% sequence identity to SEQ ID NO: 284, (v) a minimal synthetic poly A sequence comprising a nucleic acid of SEQ ID NO: 285 or a nucleic acid sequence having at least 80% sequence identity to SEQ ID NO: 285; (vi) a shortest naturally occurring SV40 early poly A signal (SV40 early poly A) comprising a nucleic acid of SEQ ID NO: 286 or a nucleic acid sequence having at least 80% sequence identity to SEQ ID NO: 286; (vii) a RBG poly A comprising a nucleic acid of SEQ ID NO: 287 or a nucleic acid sequence having at least 80% sequence identity to SEQ ID NO: 287. The poly A sequences as described in “Definition of an efficient synthetic poly(A) site” Genes Dev. 1989 Jul;3(7):1019-25. doi: 10.1101/gad.3.7.1019., which is incorporated by reference in its entirety. All the above described poly A sequences and or, terminator sequences can be used as inverted sequence e.g., in 3′ to 5′ orientation.

### E. AA V ITRS

The rAAV genome as disclosed here comprises AAV ITRs that have desirable characteristics and can be designed to modulate the activities of, and cellular responses to vectors that incorporate the ITRs. In some embodiments, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447433, which is incorporated herein by reference.

In some embodiments, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs.

In some embodiments, the synthetic ITR comprises, consists essentially of, or consists of one of the nucleotide sequences listed below. In other embodiments, the synthetic ITR comprises, consist essentially of, or consist of a nucleotide sequence that is at least 80% identical, e.g., at least 85%, 90%, 95%, 96%, 97%, 98%, or 99% identical to one of the nucleotide sequences listed in Table 10.

Table 10: Exemplary synthetic ITR sequences:

TABLE 10

| | |
|---|---|
| ITR Name | SEQ ID NO: |
| MH-257 | SEQ ID NO: 70 |
| MH-258 | SEQ ID NO: 71 |
| MH Delta 258 | SEQ ID NO: 72 |
| MH Telomere-1 ITR | SEQ ID NO: 73 |
| MH Telomere-2 ITR | SEQ ID NO: 74 |
| MH PolII 258 ITR | SEQ ID NO: 75 |
| MH 258 Delta D conservative | SEQ ID NO: 76 |
| 5′ AAV2-ITR | SEQ ID NO: 77 |
| 3′ AAV2 ITR | SEQ ID NO: 78 |

### IV. Vectors and Virions

The present invention further provides a vector comprising a synthetic cardiac-specific promoter, or expression cassette according to the present invention.

In some embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic cardiac-specific promoter of the invention that encodes a therapeutic product, suitably a therapeutic protein, e.g., an inhibitor of PP1, as disclosed herein. In some embodiments, the inhibitor of PP1, e.g., I-1 or I-1c, or a variant thereof is a secretable protein, or fused to a signal peptide for secretion.

In some embodiments the vector is an AAV vector. In some embodiments the AAV has a serotype suitable or specifically optimised for cardiac transduction. In some embodiments, the AAV is selected from the group consisting of: AAV1, AAV2, AAV4, AAV5, AAV8, AAV9, AAVrh 10, AAV2i8, AAVDJ8 and AAV2g9, or derivatives thereof.

AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce cardiac cells, e.g., cardiomyocytes and cardiac tissue particularly effectively, and thus AAV9 and derivatives thereof are of particular interest for targeting cardiac cells and tissue. AAV2g9 is known to effectively transduce cardiac cells and tissue particularly effectively, and thus AAV2g9 and derivatives thereof are of particular interest for targeting cardiac cells and cardiac tissue. AAVrh10 is known to effectively transduce cardiac cells and tissue particularly effectively, and thus AAVrh 10 and derivatives thereof are of particular interest for targeting cells and cardiac tissue.

In one embodiment, the rAAV vector (also referred to as a rAAV virion) as disclosed herein comprises a capsid protein, and a rAAV genome in the capsid protein.

In one embodiment, the AAV vector as disclosed herein comprises a capsid protein from any of those disclosed in WO2019/241324, which is specifically incorporated herein in its entirety by reference. In some embodiments, the rAAV vector comprises a cardiac specific capsid, e.g., a cardiac specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

Exemplary chimeric or variant capsid proteins that can be used as the AAV capsid in the rAAV vector described herein can be selected from Table 2 from U.S. Provisional Application 62,937,556, filed on Nov. 19, 2019, which is specifically incorporated herein in its reference, or can be used with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and US 7,892,809, which are incorporated herein in their entirety by reference.

In some embodiments, the rAAV vector is a haploid rAAV vector, as disclosed in WO2018/170310A1 or polyploid rAAV vector, e.g., as disclosed in WO2019/216932A1 and in U.S. Application 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

In a particular embodiment, the rAAV is a AAVXL32 or AAVXL32.1 AAV vector as disclosed in WO2019/241324, which is incorporated herein in its entirety by reference.

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. Pat. Application No. 11/486,254 and the other biological sequence files are to be read in a similar manner): 11486254, 11932017, 12172121, 12302206, 12308959, 12679144, 13036343, 13121532, 13172915, 13583920, 13668120, 13673351, 13679684, 14006954, 14149953, 14192101, 14194538, 14225821, 14468108, 14516544, 14603469, 14680836, 14695644, 14878703, 14956934, 15191357, 15284164, 15368570, 15371188, 15493744, 15503120, 15660906, and 15675677.

In one embodiment, the rAAV vector (also referred to as a rAAV virion) as disclosed herein comprises a capsid protein, and a rAAV genome in the capsid protein. A rAAV capsid of the rAAV virion used to treat cardiovascular diseases or heart diseases or heart failure is any of those listed in Table 11, or any combination thereof.

Table 11: AAV Serotypes and exemplary Published corresponding capsid sequence.

TABLE 11

| | |
|---|---|
| Serotype and where capsid sequence is published | Serotype and where capsid sequence is published |
| AAV3.3b (See SEQ ID NO:72 in US20030138772) | AAV3-3 (See SEQ ID NO: 200 US20150315612) |
| AAV3-3 (See SEQ ID NO:217 US20150315612) | AAV3a ((See SEQ ID NO: 5 in US6156303) |
| AAV3a (See SEQ ID NO: 9 in US6156303) | AAV3b (See SEQ ID NO: 6 in US6156303) |
| AAV3b (See SEQ ID NO: 10 in US6156303) | AAV3b (See SEQ ID NO: 1 in US6156303) |
| AAV4 (See SEQ ID NO: 17 US20140348794) | AAV4 ((See SEQ ID NO:5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO:63 in US20030138772) and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO:4 in US20030138772;US20150315612 SEQ |
| ID NO: 182 | AAV8 (See SEQ ID NO: 95 in US20030138772), US20140359799 SEQ |
| AAV8 (See SEQ ID NO: 31 in US20150159173) | AAV8 (See, e.g., SEQ ID NO: 8 in US20160017295, or SEQ ID NO:7 in US7198951, or SEQ ID NO: 223 in US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in US7198951) |
| AAV9 (See SEQ ID NO: 9 in US20160017295) | AAV9 (See SEQ ID NO: 100 in US20030138772), US7198951 SEQ ID NO: 2 |
| | AAV9 (See SEQ ID NO: 3 in US7198951) |
| AAV9 (AAVhu.14) (See SEQ ID NO: 3 in US20150315612) | AAV9 (AAVhu.14) (See SEQ ID NO: 123 in US20150315612) |
| AAVA3.1 (See SEQ ID NO: 120 in US20030138772) | AAVA3.3 (See SEQ ID NO: 57 in US20030138772) |
| AAVA3.3 (See SEQ ID NO: 66 in US20030138772) | AAVA3.4 (See SEQ ID NO: 54 in US20030138772) |
| AAVA3.4 (See SEQ ID NO: 68 in US20030138772) | AAVA3.5 (See SEQ ID NO: 55 in US20030138772) |
| AAVA3.5 (See SEQ ID NO: 69 in US20030138772) | AAVA3.7 (See SEQ ID NO: 56 in US20030138772) |
| AAVA3.7 (See SEQ ID NO: 67 in US20030138772) | AAV29. (See SEQ ID NO: 11 in (AAVbb. 1) 161 US20030138772) |
| AAVC2 (See SEQ ID NO: 61 in US20030138772) | AAVCh.5 (See SEQ ID NO:46 in US20150159173); US20150315612 SEQ |
| ID NO: 234 | AAVcy.2 (AAV 13.3) (See SEQ ID NO: 15 in US20030138772) |
| AAV24.1 (See SEQ ID NO: 101 in US20030138772) | AAVcy.3 (AAV24.1) (See SEQ ID NO: 16 in US20030138772) |
| AAV27.3 (See SEQ ID NO: 104 in US20030138772) | AAVcy.4 (AAV27.3) (See SEQ ID NO: 17 in US20030138772) |
| AAVcy.5 (See SEQ ID NO: 227 in US20150315612) | AAV7.2 (See SEQ ID NO: 103 in US20030138772) |
| AAVcy.5 (AAV7.2) (See SEQ ID NO: 18 in US20030138772) | AAV 16.3 (See SEQ ID NO: 105 in US20030138772) |
| AAVcy.6 (AAV 16.3) (See SEQ ID NO: 10 in US20030138772) | AAVcy.5 (See SEQ ID NO: 8 in US20150159173) |
| AAVcy.5 (See SEQ ID NO: 24 in US20150159173) | AAVCy.5Rl (See SEQ ID NO: in US20150159173 |
| AAVCy.5R2 (See SEQ ID NO: in US20150159173) | AAVCy.5R3 (See SEQ ID NO: in US20150159173 |
| AAVCy.5R4 (See SEQ ID NO: in US20150159173) | AAVDJ (See SEQ ID NO: 3 in US20140359799) and SEQ ID NO: 2 in US7588772) |
| | AAVDJ (See SEQ ID NO: 2 in US20140359799; and SEQ ID NO: 1 in US7588772) |
| | AAVDJ-8 (See SEQ ID NO: in US7588772; Grimm et al 2008 |
| AAVDJ-8 (See SEQ ID NO: in US7588772; Grimm et al 2008 | AAVF5 (See SEQ ID NO: 110 in US20030138772) |
| AAVH2 (See SEQ ID NO: 26 in US20030138772) | AAVH6 (See SEQ ID NO: 25 in US20030138772) |
| AAVhE1.1 (See SEQ ID NO: 44 in US9233131) | AAVhEr1.14 (See SEQ ID NO: 46 in US9233131) |
| AAVhEr1.16(See SEQ ID NO: 48 in US9233131) | AAVhEr1.18 (See SEQ ID NO: 49 in US9233131) |
| AAVhEr1.23 (AAVhEr2.29) (See SEQ ID NO: 53 in US9233131) | AAVhEr1.35 (See SEQ ID NO: 50 in US9233131) |
| AAVhEr1.36 (See SEQ ID NO: 52 in US9233131) | AAVhEr1.5 (See SEQ ID NO: 45 in US9233131) |
| AAVhEr1.7 (See SEQ ID NO: 51 in US9233131) | AAVhEr1.8 (See SEQ ID NO: 47 in US9233131) |
| AAVhEr2.16 (See SEQ ID NO: 55 in US9233131) | AAVhEr2.30 (See SEQ ID NO: 56 in US9233131) |
| AAVhEr2.31 (See SEQ ID NO: 58 in US9233131) | AAVhEr2.36 (See SEQ ID NO: 57 in US9233131) |
| AAVhEr2.4 (See SEQ ID NO: 54 in US9233131) | AAVhEr3.1 (See SEQ ID NO: 59 in US9233131) |
| AAVhu.1 (See SEQ ID NO: 46 in US20150315612) | AAVhu.1 (See SEQ ID NO: 144 in US20150315612) |
| AAVhu.1O(AAV16.8) (See SEQ ID NO: 56 in US20150315612) | AAVhu.1O(AAV 16.8) (See SEQ ID NO: 156 in US20150315612) |
| AAVhu.11(AAV 16.12) (See SEQ ID NO: 57 in US20150315612) | AAVhu.11 (AAV 16.12) (See SEQ ID NO: 153 in US20150315612) |
| AAVhu.12 (See SEQ ID NO: 59 in US20150315612) | AAVhu.12 (See SEQ ID NO: 154 in US20150315612) |
| AAVhu.13 (See SEQ ID NO: 16 in US2015015917 and ID NO: 71 in US20150315612) | |
| AAVhu.13 (See SEQ ID NO: 32 in US20150159173 and ID NO: 129 US20150315612) | |
| AAVhu.136.1 (See SEQ ID NO: 165 in US20150315612) | AAVhu.140.1 (See SEQ ID NO: 166 in US20150315612) |
| AAVhu.140.2 (See SEQ ID NO: 167 in US20150315612) | AAVhu.145.6 (See SEQ ID NO: 178 in US20150315612) |
| AAVhu.15 (See SEQ ID NO: 147 in US20150315612) | AAVhu.15 (AAV33.4) (See SEQ ID NO: 50 in US20150315612) |
| AAVhu.156.1 (See SEQ ID NO: 179 in US20150315612) | AAVhu.16 (See SEQ ID NO: 148 in US20150315612) |
| AAVhu.16 (AAV33.8) (See SEQ ID NO: 51 in US20150315612) | AAVhu.17 (See SEQ ID NO: 83 in US20150315612) |
| AAVhu.17 (AAV33.12) (See SEQ ID NO: 4 in US20150315612) | AAVhu.172.1 (See SEQ ID NO: 171 in US20150315612) |
| AAVhu.172.2 (See SEQ ID NO: 172 in US20150315612) | AAVhu.173.4 (See SEQ ID NO: 173 in US20150315612) |
| AAVhu.173.8 (See SEQ ID NO: 175 in US20150315612) | AAVhu.18 (See SEQ ID NO: 52 in US20150315612) |
| AAVhu.18 (See SEQ ID NO: 149 in US20150315612) | AAVhu.19 (See SEQ ID NO: 62 in US20150315612) |
| AAVhu.19 (See SEQ ID NO: 133 in US20150315612) | AAVhu.2 (See SEQ ID NO: 48 in US20150315612) |
| AAVhu.2 (See SEQ ID NO: 143 in US20150315612) | AAVhu.20 (See SEQ ID NO: 63 in US20150315612) |
| AAVhu.20 (See SEQ ID NO: 134 in US20150315612) | AAVhu.21 (See SEQ ID NO: 65 in US20150315612) |
| AAVhu.21 (See SEQ ID NO: 135 in US20150315612) | AAVhu.22 (See SEQ ID NO: 67 in US20150315612) |
| AAVhu.22 239 (See SEQ ID NO: 138 in US20150315612) | AAVhu.23 (See SEQ ID NO: 60 in US20150315612) |
| AAVhu.23.2 (See SEQ ID NO: 137 in US20150315612) | AAVhu.24 (See SEQ ID NO: 66 in US20150315612) |
| AAVhu.24 (See SEQ ID NO: 136 in US20150315612) | AAVhu.25 (See SEQ ID NO: 49 in US20150315612) |
| AAVhu.25 (See SEQ ID NO: 146 in US20150315612) | AAVhu.26 (See SEQ ID NO: 17 in US20150159173 and SEQ ID NO: 61 in US20150315612) |
| | AAVhu.26 (See SEQ ID NO: 33 in US20150159173), US20150315612 SEQ |
| | AAVhu.27 (See SEQ ID NO: 64 in US20150315612) |
| AAVhu.27 (See SEQ ID NO: 140 in US20150315612) | AAVhu.28 (See SEQ ID NO: 68 in US20150315612) |
| AAVhu.28 (See SEQ ID NO: 130 in US20150315612) | AAVhu.29 (See SEQ ID NO: 69 in US20150315612) |
| AAVhu.29 (See SEQ ID NO: 42 in US20150159173 and SEQ ID NO: 132 in US20150315612) | |
| AAVhu.29 (See SEQ ID NO: 225 in US20150315612) | AAVhu.29R (See SEQ ID NO: in US20150159173 |
| AAVhu.3 (See SEQ ID NO: 44 in US20150315612) | AAVhu.3 (See SEQ ID NO: 145 in US20150315612) |
| AAVhu.30 (See SEQ ID NO: 70 in US20150315612) | AAVhu.30 (See SEQ ID NO: 131 in US20150315612) |
| AAVhu.31 (See SEQ ID NO: 1 in US20150315612) | AAVhu.31 (See SEQ ID NO: 121 in US20150315612) |
| AAVhu.32 (See SEQ ID NO: 2 in US20150315612) | AAVhu.32 (See SEQ ID NO: 122 in US20150315612) |
| AAVhu.33 (See SEQ ID NO: 75 in US20150315612) | AAVhu.33 (See SEQ ID NO: 124 in US20150315612) |
| AAVhu.34 (See SEQ ID NO: 72 in US20150315612) | AAVhu.34 (See SEQ ID NO: 125 in US20150315612) |
| AAVhu.35 (See SEQ ID NO: 73 in US20150315612) | AAVhu.35 (See SEQ ID NO: 164 in US20150315612) |
| AAVhu.36 (See SEQ ID NO: 74 in US20150315612) | AAVhu.36 (See SEQ ID NO: 126 in US20150315612) |
| AAVhu.37 (See SEQ ID NO: 34 in US20150159173 and SEQ ID NO: 88 in US20150315612) | |
| AAVhu.37 (AAV106.1) (See SEQ ID NO: 10 in US20150315612 and SEQ ID NO: 18 in US20150159173) | |
| AAVhu.38 (See SEQ ID NO: 161 in US20150315612) | AAVhu.39 (See SEQ ID NO: 102 in US20150315612) |
| AAVhu.39 (AAVLG-9) (See SEQ ID NO: 24 in US20150315612) | AAVhu.4 (See SEQ ID NO: 47 in US20150315612) |
| AAVhu.4 (See SEQ ID NO: 141 in US20150315612) | AAVhu.40 (See SEQ ID NO: 87 in US20150315612) |
| AAVhu.40 (AAV114.3) (See SEQ ID NO: 11 in US20150315612) | AAVhu.41 (See SEQ ID NO: 91 in US20150315612) |
| AAVhu.41 (AAV127.2) (See SEQ ID NO: 6 in US20150315612) | AAVhu.42 (See SEQ ID NO: 85 in US20150315612) |
| AAVhu.42 (AAV127.5) (See SEQ ID NO:8 in US20150315612) | AAVhu.43 (See SEQ ID NO: 160 in US20150315612) |
| AAVhu.43 (See SEQ ID NO: 236 in US20150315612) | AAVhu.43 (AAV128.1) (See SEQ ID NO: 80 in US20150315612) |
| AAVhu.44 (See SEQ ID NO: 45 in US20150159173 and SEQ ID NO: 158 in US20150315612) | |
| AAVhu.44 (AAV128.3) (See SEQ ID NO: 81 in US20150315612) | AAVhu.44Rl (See SEQ ID NO: in US20150159173 |
| AAVhu.44R2 (See SEQ ID NO: in US20150159173 | AAVhu.44R3 (See SEQ ID NO: in US20150159173 |
| AAVhu.45 (See SEQ ID NO: 76 in US20150315612) | AAVhu.45 (See SEQ ID NO: 127 in US20150315612) |
| AAVhu.46 (See SEQ ID NO: 82 in US20150315612) | AAVhu.46 (See SEQ ID NO: 159 in US20150315612) |
| AAVhu.46 (See SEQ ID NO: 224 in US20150315612) | AAVhu.47 (See SEQ ID NO: 77 in US20150315612) |
| AAVhu.47 (See SEQ ID NO: 128 in US20150315612) | AAVhu.48 (See SEQ ID NO: 38 in US20150159173) |
| AAVhu.48 (See SEQ ID NO: 157 in US20150315612) | AAVhu.48 (AAV130.4) (See SEQ ID NO: 78 in US20150315612) |
| AAVhu.48Rl (See SEQ ID NO: in US20150159173 | AAVhu.48R2 (See SEQ ID NO: in US20150159173 |
| AAVhu.48R3 (See SEQ ID NO: in US20150159173 | AAVhu.49 (See SEQ ID NO: 209 in US20150315612) |
| AAVhu.49 (See SEQ ID NO: 189 in US20150315612) | AAVhu.5 (See SEQ ID NO: 45 in US20150315612) |
| AAVhu.5 (See SEQ ID NO: 142 in US20150315612) | AAVhu.51 (See SEQ ID NO: 208 in US20150315612) |
| AAVhu.51 (See SEQ ID NO: 190 in US20150315612) | AAVhu.52 (See SEQ ID NO: 210 in US20150315612) |
| AAVhu.52 (See SEQ ID NO: 191 in US20150315612) | AAVhu.53 (See SEQ ID NO: 19 in US20150159173) |
| AAVhu.53 (See SEQ ID NO: 35 in US20150159173) | AAVhu.53 (AAV145.1) (See SEQ ID NO: 176 in US20150315612) |
| AAVhu.54 (See SEQ ID NO: 188 in US20150315612) | AAVhu.54 (AAV145.5) (See SEQ ID NO: 177 in US20150315612) |
| AAVhu.55 (See SEQ ID NO: 187 in US20150315612) | AAVhu.56 (See SEQ ID NO: 205 in US20150315612) |
| AAVhu.56 (AAV 145.6) (See SEQ ID NO: 168 in US20150315612) | AAVhu.56 (AAV145.6) (See SEQ ID NO: 192 in US20150315612) |
| AAVhu.57 (See SEQ ID NO: 206 in US20150315612) | AAVhu.57 (See SEQ ID NO: 169 in US20150315612) |
| AAVhu.57 (See SEQ ID NO: 193 in US20150315612) | AAVhu.58 (See SEQ ID NO: 207 in US20150315612) |
| AAVhu.58 (See SEQ ID NO: 194 in US20150315612) | AAVhu.6 (AAV3.1) (See SEQ ID NO: 5 in US20150315612) |
| AAVhu.6 (AAV3.1) (See SEQ ID NO: 84 in US20150315612) | AAVhu.60 (See SEQ ID NO: 184 in US20150315612) |
| AAVhu.60 (AAV161.10) (See SEQ ID NO: 170 in US20150315612) | AAVhu.61 (See SEQ ID NO: 185 in US20150315612) |
| AAVhu.61 (AAV161.6) (See SEQ ID NO: 174 in US20150315612) | AAVhu.63 (See SEQ ID NO: 204 in US20150315612) |
| AAVhu.63 (See SEQ ID NO: 195 in US20150315612) | AAVhu.64 (See SEQ ID NO: 212 in US20150315612) |
| AAVhu.64 (See SEQ ID NO: 196 in US20150315612) | AAVhu.66 (See SEQ ID NO: 197 in US20150315612) |
| AAVhu.67 (See SEQ ID NO: 215 in US20150315612) | AAVhu.67 (See SEQ ID NO: 198 in US20150315612) |
| AAVhu.7 (See SEQ ID NO: 226 in US20150315612) | AAVhu.7 (See SEQ ID NO: 150 in US20150315612) |
| AAVhu.7 (AAV7.3) (See SEQ ID NO: 55 in US20150315612) | AAVhu.71 (See SEQ ID NO: 79 in US20150315612) |
| AAVhu.8 (See SEQ ID NO: 53 in US20150315612) | AAVhu.8 (See SEQ ID NO: 12 in US20150315612) |
| AAVhu.8 (See SEQ ID NO: 151 in US20150315612) | AAVhu.9 (AAV3.1) (See SEQ ID NO: 58 in US20150315612) |
| AAVhu.9 (AAV3.1) (See SEQ ID NO: 155 in US20150315612) | AAV-LK01 (See SEQ ID NO: 2 in US20150376607) |
| AAV-LK01 (See SEQ ID NO: 29 in US20150376607) | AAV-LK02 (See SEQ ID NO: 3 in US20150376607) |
| AAV-LK02 (See SEQ ID NO: 30 in US20150376607) | AAV-LK03 (See SEQ ID NO: 4 in US20150376607) |
| AAV-LK03 (See SEQ ID NO: 12 in WO2015121501 and SEQ ID NO: 31 in US20150376607) | |
| AAV-LK04 (See SEQ ID NO: 5 in US20150376607) | AAV-LK04 (See SEQ ID NO: 32 in US20150376607) |
| AAV-LK05 (See SEQ ID NO: 6 in US20150376607) | AAV-LK05 (See SEQ ID NO: 33 in US20150376607) |
| AAV-LK06 (See SEQ ID NO: 7 in US20150376607) | AAV-LK06 (See SEQ ID NO: 34 in US20150376607) |
| AAV-LK07 (See SEQ ID NO: 8 in US20150376607) | AAV-LK07 (See SEQ ID NO: 35 in US20150376607) |
| AAV-LK08 (See SEQ ID NO: 9 in US20150376607) | AAV-LK08 (See SEQ ID NO: 36 in US20150376607) |
| AAV-LK09 (See SEQ ID NO: 10 in US20150376607) | AAV-LK09 (See SEQ ID NO: 37 in US20150376607) |
| AAV-LK10 (See SEQ ID NO: 11 in US20150376607) | AAV-LK10 (See SEQ ID NO: 38 in US20150376607) |
| AAV-LK11 (See SEQ ID NO: 12 in US20150376607) | AAV-LK11 (See SEQ ID NO: 39 in US20150376607) |
| AAV-LK12 (See SEQ ID NO: 13 in US20150376607) | AAV-LK12 (See SEQ ID NO: 40 in US20150376607) |
| AAV-LK13 (See SEQ ID NO: 14 in US20150376607) | AAV-LK13 (See SEQ ID NO: 41 in US20150376607) |
| AAV-LK14 (See SEQ ID NO: 15 in US20150376607) | AAV-LK14 (See SEQ ID NO: 42 in US20150376607) |
| AAV-LK15 (See SEQ ID NO: 16 in US20150376607) | AAV-LK15 (See SEQ ID NO: 43 in US20150376607) |
| AAV-LK16 (See SEQ ID NO: 17 in US20150376607) | AAV-LK16 (See SEQ ID NO: 44 in US20150376607) |
| AAV-LK17 (See SEQ ID NO: 18 in US20150376607) | AAV-LK17 (See SEQ ID NO: 45 in US20150376607) |
| AAV-LK18 (See SEQ ID NO: 19 in US20150376607) | AAV-LK18 (See SEQ ID NO: 46 in US20150376607) |
| AAV-LK19 (See SEQ ID NO: 20 in US20150376607) | AAV-LK19 (See SEQ ID NO: 47 in US20150376607) |
| AAV-PAEC (See SEQ ID NO: 1 in US20150376607) | AAV-PAEC (See SEQ ID NO: 48 in US20150376607) |
| AAV-PAEC11 (See SEQ ID NO: 26 in US20150376607) | AAV-PAEC11 (See SEQ ID NO: 54 in US20150376607) |
| AAV-PAEC 12 (See SEQ ID NO: 27 in US20150376607) | AAV-PAEC 12 (See SEQ ID NO: 51 in US20150376607) |
| AAV-PAEC 13 (See SEQ ID NO: 28 in US20150376607) | AAV-PAEC 13 (See SEQ ID NO: 49 in US20150376607) |
| AAV-PAEC2 (See SEQ ID NO: 21 in US20150376607) | AAV-PAEC2 (See SEQ ID NO: 56 in US20150376607) |
| AAV-PAEC4 (See SEQ ID NO: 22 in US20150376607) | AAV-PAEC4 (See SEQ ID NO: 55 in US20150376607) |
| AAV-PAEC6 (See SEQ ID NO: 23 in US20150376607) | AAV-PAEC6 (See SEQ ID NO: 52 in US20150376607) |
| AAV-PAEC7 (See SEQ ID NO: 24 in US20150376607) | AAV-PAEC7 (See SEQ ID NO: 53 in US20150376607) |
| AAV-PAEC8 (See SEQ ID NO: 25 in US20150376607) | AAV-PAEC8 (See SEQ ID NO: 50 in US20150376607) |
| AAVpi.l (See SEQ ID NO: 28 in US20150315612) | AAVpi.l (See SEQ ID NO: 93 in US20150315612; AAVpi.2 408, see SEQ ID NO: 30 in US20150315612) |
| AAVpi.2 (See SEQ ID NO: 95 in US20150315612) | AAVpi.3 (See SEQ ID NO: 29 in US20150315612) |
| AAVpi.3 (See SEQ ID NO: 94 in US20150315612) | AAVrh.10 (See SEQ ID NO: 9 in US20150159173) |
| AAVrh.10 (See SEQ ID NO: 25 in US20150159173) | AAV44.2 (See SEQ ID NO: 59 in US20030138772) |
| AAVrh.10 (AAV44.2) (See SEQ ID NO: 81 in US20030138772) | AAV42.1B (See SEQ ID NO: 90 in US20030138772) |
| AAVrh.l2 (AAV42.1b) (See SEQ ID NO: 30 in US20030138772) | AAVrh.13 (See SEQ ID NO: 10 in US20150159173) |
| AAVrh.13 (See SEQ ID NO: 26 in US20150159173) | AAVrh.13 (See SEQ ID NO: 228 in US20150315612) |
| AAVrh.13R (See SEQ ID NO: in US20150159173 | AAV42.3A (See SEQ ID NO: 87 in US20030138772) |
| AAVrh.14 (AAV42.3a) (See SEQ ID NO: 32 in US20030138772) | AAV42.5A (See SEQ ID NO: 89 in US20030138772) |
| AAVrh.17 (AAV42.5a) (See SEQ ID NO: 34 in US20030138772) | AAV42.5B (See SEQ ID NO: 91 in US20030138772) |
| AAVrh.18 (AAV42.5b) (See SEQ ID NO: 29 in US20030138772) | AAV42.6B (See SEQ ID NO: 112 in US20030138772) |
| AAVrh.19 (AAV42.6b) (See SEQ ID NO: 38 in US20030138772) | AAVrh.2 (See SEQ ID NO: 39 in US20150159173) |
| AAVrh.2 (See SEQ ID NO: 231 in US20150315612) | AAVrh.20 (See SEQ ID NO: 1 in US20150159173) |
| AAV42.10 (See SEQ ID NO: 106 in US20030138772) | AAVrh.21 (AAV42.10) (See SEQ ID NO: 35 in US20030138772) |
| AAV42.11 (See SEQ ID NO: 108 in US20030138772) | AAVrh.22 (AAV42.11) (See SEQ ID NO: 37 in US20030138772) |
| AAV42.12 (See SEQ ID NO: 113 in US20030138772) | AAVrh.23 (AAV42.12) (See SEQ ID NO: 58 in US20030138772) |
| AAV42.13 (See SEQ ID NO: 86 in US20030138772) | AAVrh.24 (AAV42.13) (See SEQ ID NO: 31 in US20030138772) |
| AAV42.15 (See SEQ ID NO: 84 in US20030138772) | AAVrh.25 (AAV42.15) (See SEQ ID NO: 28 in US20030138772) |
| AAVrh.2R (See SEQ ID NO: in US20150159173 | AAVrh.31 (AAV223.1) (See SEQ ID NO: 48 in US20030138772) |
| AAVC1 (See SEQ ID NO: 60 in US20030138772) | AAVrh.32 (AAVC1) (See SEQ ID NO: 19 in 446 US20030138772) |
| AAVrh.32/33 (See SEQ ID NO: 2 in US20150159173) | AAVrh.51 (AAV2-5) (See SEQ ID NO: 104 in US20150315612) |
| AAVrh.52 (AAV3-9) (See SEQ ID NO: 18 in US20150315612) | AAVrh.52 (AAV3-9) (See SEQ ID NO: 96 in US20150315612) |
| AAVrh.53 (See SEQ ID NO: in US20150315612) | AAVrh.53 (AAV3-11) (See SEQ ID NO: 17 in US20150315612) |
| AAVrh.53 (AAV3-11) (See SEQ ID NO: 186 in US20150315612) | AAVrh.54 (See SEQ ID NO: 40 in US20150315612) |
| AAVrh.54 (See SEQ ID NO: 49 in US20150159173 and SEQ ID NO: 116 in US20150315612) | |
| AAVrh.55 (See SEQ ID NO: 37 in US20150315612) | AAVrh.55 (AAV4-19) (See SEQ ID NO: 117 in US20150315612) |
| AAVrh.56 (See SEQ ID NO: 54 in US20150315612) | AAVrh.56 (See SEQ ID NO: 152 in US20150315612) |
| AAVrh.57 (See SEQ ID NO: in 497 US20150315612 SEQ ID NO: 26 | AAVrh.57 (See SEQ ID NO: 105 in US20150315612) |
| AAVrh.58 (See SEQ ID NO: 27 in US20150315612) | AAVrh.58 (See SEQ ID NO: 48 in US20150159173 and SEQ ID NO: 106 in US20150315612) |
| | AAVrh.58 (See SEQ ID NO: 232 in US20150315612) |
| AAVrh.59 (See SEQ ID NO: 42 in US20150315612) | AAVrh.59 (See SEQ ID NO: 110 in US20150315612) |
| AAVrh.60 (See SEQ ID NO: 31 in US20150315612) | AAVrh.60 (See SEQ ID NO: 120 in US20150315612) |
| AAVrh.61 (See SEQ ID NO: 107 in US20150315612) | AAVrh.61 (AAV2-3) (See SEQ ID NO: 21 in US20150315612) |
| AAVrh.62 (AAV2-15) (See SEQ ID NO: 33 in US20150315612) | AAVrh.62 (AAV2-15) (See SEQ ID NO: 114 in US20150315612) |
| AAVrh.64 (See SEQ ID NO: 15 in US20150315612) | AAVrh.64 (See SEQ ID NO: 43 in US20150159173 and SEQ ID NO: 99 in US20150315612) |
| | AAVrh.64 (See SEQ ID NO: 233 in US20150315612) |
| AAVRh.64Rl (See SEQ ID NO: in US20150159173 | AAVRh.64R2 (See SEQ ID NO: in US20150159173 |
| AAVrh.65 (See SEQ ID NO: 35 in US20150315612) | AAVrh.65 (See SEQ ID NO: 112 in US20150315612) |
| AAVrh.67 (See SEQ ID NO: 36 in US20150315612) | AAVrh.67 (See SEQ ID NO: 230 in US20150315612) |
| AAVrh.67 (See SEQ ID NO: 47 in US20150159173 and SEQ ID NO: 47 in US20150315612) | |
| AAVrh.68 (See SEQ ID NO: 16 in US20150315612) | AAVrh.68 (See SEQ ID NO: 100 in US20150315612) |
| AAVrh.69 (See SEQ ID NO: 39 in US20150315612) | AAVrh.69 (See SEQ ID NO: 119 in US20150315612) |
| AAVrh.70 (See SEQ ID NO: 20 in US20150315612) | AAVrh.70 (See SEQ ID NO: 98 in US20150315612) |
| AAVrh.71 (See SEQ ID NO: 162 in US20150315612) | AAVrh.72 (See SEQ ID NO: 9 in US20150315612) |
| AAVrh.73 (See SEQ ID NO: 5 in US20150159173) | AAVrh.74 (See SEQ ID NO: 6 in US20150159173) |
| AAVrh.8 (See SEQ ID NO: 41 in US20150159173) | AAVrh.8 (See SEQ ID NO: 235 in US20150315612) |
| AAVrh.8R (See SEQ ID NO: 9 in US20150159173, WO2015168666) | AAVrh.8R A586R mutant (See SEQ ID NO: 10 in WO2015168666) |
| AAVrh.8R R533A mutant (See SEQ ID NO: 11 in WO2015168666) | BAAV (bovine AAV) (See SEQ ID NO: 8 in US9193769) |
| BAAV (bovine AAV) (See SEQ ID NO: 10 in US9193769) | BAAV (bovine AAV) (See SEQ ID NO: 4 in US9193769) |
| BAAV (bovine AAV) (See SEQ ID NO: 2 in US9193769) | BAAV (bovine AAV) (See SEQ ID NO: 6 in US9193769) |
| BAAV (bovine AAV) (See SEQ ID NO: 1 in US9193769) | BAAV (bovine AAV) (See SEQ ID NO: 5 in US9193769) |
| BAAV (bovine AAV) (See SEQ ID NO: 3 in US9193769) | BAAV (bovine AAV) (See SEQ ID NO: 11 in US9193769) |
| BAAV (bovine AAV) (See SEQ ID NO: 5 in US7427396) | BAAV (bovine AAV) (See SEQ ID NO: 6 in US7427396) |
| BAAV (bovine AAV) (See SEQ ID NO: 7 in US9193769) | BAAV (bovine AAV) (See SEQ ID NO: 9 in US9193769) |
| BNP61 AAV (See SEQ ID NO: 1 in US20150238550) | BNP61 AAV (See SEQ ID NO: 2 in US20150238550) |
| BNP62 AAV (See SEQ ID NO: 3 in US20150238550) | BNP63 AAV (See SEQ ID NO: 4 in US20150238550) |
| caprine AAV (See SEQ ID NO: 3 in US7427396) | caprine AAV (See SEQ ID NO: 4 in US7427396) |
| true type AAV (ttAAV) (See SEQ ID NO: 2 in WO2015121501) | AAAV (Avian AAV) (See SEQ ID NO: 12 in US9238800) |
| AAAV (Avian AAV) (See SEQ ID NO: 2 in US9238800) | AAAV (Avian AAV) (See SEQ ID NO: 6 in US9238800) |
| AAAV (Avian AAV) (See SEQ ID NO: 4 in US9238800) | AAAV (Avian AAV) (See SEQ ID NO: 8 in US9238800) |
| AAAV (Avian AAV) (See SEQ ID NO: 14 in US9238800) | AAAV (Avian AAV) (See SEQ ID NO: 10 in US9238800) |
| AAAV (Avian AAV) (See SEQ ID NO: 15 in US9238800) | AAAV (Avian AAV) (See SEQ ID NO: 5 in US9238800) |
| AAAV (Avian AAV) (See SEQ ID NO: 9 in US9238800) | AAAV (Avian AAV) (See SEQ ID NO: 3 in US9238800) |
| AAAV (Avian AAV) (See SEQ ID NO: 7 in US9238800) | AAAV (Avian AAV) (See SEQ ID NO: 11 in US9238800) |
| AAAV (Avian AAV) (See SEQ ID NO: in US9238800) | AAAV (Avian AAV) (See SEQ ID NO: 1 in US9238800) |
| AAV Shuffle 100-1 (See SEQ ID NO: 23 in US20160017295) | AAV Shuffle 100-1 (See SEQ ID NO: 11 in US20160017295) |
| AAV Shuffle 100-2 (See SEQ ID NO: 37 in US20160017295) | AAV Shuffle 100-2 (See SEQ ID NO: 29 in US20160017295) |
| AAV Shuffle 100-3 (See SEQ ID NO: 24 in US20160017295) | AAV Shuffle 100-3 (See SEQ ID NO: 12 in US20160017295) |
| AAV Shuffle 100-7 (See SEQ ID NO: 25 in US20160017295) | AAV Shuffle 100-7 (See SEQ ID NO: 13 in US20160017295) |
| AAV Shuffle 10-2 (See SEQ ID NO: 34 in US20160017295) | AAV Shuffle 10-2 (See SEQ ID NO: 26 in US20160017295) |
| AAV Shuffle 10-6 (See SEQ ID NO: 35 in US20160017295) | AAV Shuffle 10-6 (See SEQ ID NO: 27 in US20160017295) |
| AAV Shuffle 10-8 (See SEQ ID NO: 36 in US20160017295) | AAV Shuffle 10-8 (See SEQ ID NO: 28 in US20160017295) |
| AAV SM 100-10 (See SEQ ID NO: 41 in US20160017295) | AAV SM 100-10 (See SEQ ID NO: 33 in US20160017295) |
| AAV SM 100-3 (See SEQ ID NO: 40 in US20160017295) | AAV SM 100-3 (See SEQ ID NO: 32 in US20160017295) |
| AAV SM 10-1 (See SEQ ID NO: 38 in US20160017295) | AAV SM 10-1 (See SEQ ID NO: 30 in US20160017295) |
| AAV SM 10-2 (See SEQ ID NO: 10 in US20160017295) | AAV SM 10-2 (See SEQ ID NO: 22 in US20160017295) |
| AAV SM 10-8 (See SEQ ID NO: 39 in US20160017295) | AAV SM 10-8 (See SEQ ID NO: 31 in US20160017295) |
| AAV CBr-7.1 (See SEQ ID NO: 4 in WO2016065001) | AAV CBr-7.1 (See SEQ ID NO: 54 in WO2016065001) |
| AAV CBr-7.10 (See SEQ ID NO: 11 in WO2016065001) | AAV CBr-7.10 (See SEQ ID NO: 61 in WO2016065001) |
| AAV CBr-7.2 (See SEQ ID NO: 5 in WO2016065001) | AAV CBr-7.2 (See SEQ ID NO: 55 in WO2016065001) |
| AAV CBr-7.3 (See SEQ ID NO: 6 in WO2016065001) | AAV CBr-7.3 (See SEQ ID NO: 56 in WO2016065001) |
| AAV CBr-7.4 (See SEQ ID NO: 7 in WO2016065001) | AAV CBr-7.4 (See SEQ ID NO: 57 in WO2016065001) |
| AAV CBr-7.5 (See SEQ ID NO: 8 in WO2016065001) | AAV CHt-6.6 (See SEQ ID NO: 35 in WO2016065001) |
| AAV CHt-6.6 (See SEQ ID NO: 85 in WO2016065001) | AAV CHt-6.7 (See SEQ ID NO: 36 in WO2016065001) |
| AAV CHt-6.7 (See SEQ ID NO: 86 in WO2016065001) | AAV CHt-6.8 (See SEQ ID NO: 37 in WO2016065001) |
| AAV CHt-6.8 (See SEQ ID NO: 87 in WO2016065001) | AAV CHt-P1 (See SEQ ID NO: 29 in WO2016065001) |
| AAV CHt-Pl (See SEQ ID NO: 79 in WO2016065001) | AAV CHt-P2 (See SEQ ID NO: 1 in WO2016065001) |
| AAV CHt-P2 (See SEQ ID NO: 51 in WO2016065001) | AAV CHt-P5 (See SEQ ID NO: 2 in WO2016065001) |
| AAV CHt-P5 (See SEQ ID NO: 52 in WO2016065001) | AAV CHt-P6 (See SEQ ID NO: 30 in WO2016065001) |
| AAV CHt-P6 (See SEQ ID NO: 80 in WO2016065001) | AAV CHt-P8 (See SEQ ID NO: 31 in WO2016065001) |
| AAV CHt-P8 (See SEQ ID NO: 81 in WO2016065001) | AAV CHt-P9 (See SEQ ID NO: 3 in WO2016065001) |
| AAV CHt-P9 (See SEQ ID NO: 53 in WO2016065001) | AAV CKd-1 (See SEQ ID NO: 57 in US8734809) |
| AAV CKd-1 (See SEQ ID NO: 131 in US8734809) | AAV CKd-10 (See SEQ ID NO: 58 in US8734809) |
| AAV CKd-10 (See SEQ ID NO: 132 in US8734809) | AAV CKd-2 (See SEQ ID NO: 59 in US8734809) |
| AAV CKd-2 (See SEQ ID NO: 133 in US8734809) | AAV CKd-3 (See SEQ ID NO: 60 in US8734809) |
| AAV CKd-3 (See SEQ ID NO: 134 in US8734809) | AAV CKd-4 (See SEQ ID NO: 61 in US8734809) |
| AAV CKd-4 (See SEQ ID NO: 135 in US8734809) | AAV CKd-6 (See SEQ ID NO: 62 in US8734809) |
| AAV CKd-6 (See SEQ ID NO: 136 in US8734809) | AAV CKd-7 (See SEQ ID NO: 63 in US8734809) |
| AAV CKd-7 (See SEQ ID NO: 137 in US8734809) | AAV CKd-8 (See SEQ ID NO: 64 in US8734809) |
| AAV CKd-8 (See SEQ ID NO: 138 in US8734809) | AAV CKd-B 1 (See SEQ ID NO: 73 in US8734809) |
| AAV CKd-B 1 (See SEQ ID NO: 147 in US8734809) | AAV CKd-B2 (See SEQ ID NO: 74 in US8734809) |
| AAV CKd-B2 (See SEQ ID NO: 148 in US8734809) | AAV CKd-B3 (See SEQ ID NO: 75 in US8734809) |
| AAV CKd-B3 (See SEQ ID NO: in US8734809 | AAV CKd-B3 (See SEQ ID NO: 149 in US8734809) |
| AAV CLv-1 (See SEQ ID NO: 65 in US8734809) | AAV CLv-1 (See SEQ ID NO: 139 in US8734809) |
| AAV CLvl-1 (See SEQ ID NO: 171 in US8734809) | AAV Civ 1-10 (See SEQ ID NO: 178 in US8734809) |
| AAV CLvl-2 (See SEQ ID NO: 172 in US8734809) | AAV CLv-12 (See SEQ ID NO: 66 in US8734809) |
| AAV CLv-12 (See SEQ ID NO: 140 in US8734809) | AAV CLvl-3 (See SEQ ID NO: 173 in US8734809) |
| AAV CLv-13 (See SEQ ID NO: 67 in US8734809) | AAV CLv-13 (See SEQ ID NO: 141 in US8734809) |
| AAV CLvl-4 (See SEQ ID NO: 174 in US8734809) | AAV Civ 1-7 (See SEQ ID NO: 175 in US8734809) |
| AAV Civ 1-8 (See SEQ ID NO: 176 in US8734809) | AAV Civ 1-9 (See SEQ ID NO: 177 in US8734809) |
| AAV CLv-2 (See SEQ ID NO: 68 in US8734809) | AAV CLv-2 (See SEQ ID NO: 142 in US8734809) |
| AAV CLv-3 (See SEQ ID NO: 69 in US8734809) | AAV CLv-3 (See SEQ ID NO: 143 in US8734809) |
| AAV CLv-4 (See SEQ ID NO: 70 in US8734809) | AAV CLv-4 (See SEQ ID NO: 144 in US8734809) |
| AAV CLv-6 (See SEQ ID NO: 71 in US8734809) | AAV CLv-6 (See SEQ ID NO: 145 in US8734809) |
| AAV CLv-8 (See SEQ ID NO: 72 in US8734809) | AAV CLv-8 (See SEQ ID NO: 146 in US8734809) |
| AAV CLv-Dl (See SEQ ID NO: 22 in US8734809) | AAV CLv-Dl (See SEQ ID NO: 96 in US8734809) |
| AAV CLv-D2 (See SEQ ID NO: 23 in US8734809) | AAV CLv-D2 (See SEQ ID NO: 97 in US8734809) |
| AAV CLv-D3 (See SEQ ID NO: 24 in US8734809) | AAV CLv-D3 (See SEQ ID NO: 98 in US8734809) |
| AAV CLv-D4 (See SEQ ID NO: 25 in US8734809) | AAV CLv-D4 (See SEQ ID NO: 99 in US8734809) |
| AAV CLv-D5 (See SEQ ID NO: 26 in US8734809) | AAV CLv-D5 (See SEQ ID NO: 100 in US8734809) |
| AAV CLv-D6 (See SEQ ID NO: 27 in US8734809) | AAV CLv-D6 (See SEQ ID NO: 101 in US8734809) |
| AAV CLv-D7 (See SEQ ID NO: 28 in US8734809) | AAV CLv-D7 (See SEQ ID NO: 102 in US8734809) |
| AAV CLv-D8 (See SEQ ID NO: 29 in US8734809) | AAV CLv-D8 (See SEQ ID NO: 103 in US8734809); AAV CLv-Kl 762, see SEQ ID NO: 18 in WO2016065001) |
| AAV CLv-Kl (See SEQ ID NO: 68 in WO2016065001) | AAV CLv-K3 (See SEQ ID NO: 19 in WO2016065001) |
| AAV CLv-K3 (See SEQ ID NO: 69 in WO2016065001) | AAV CLv-K6 (See SEQ ID NO: 20 in WO2016065001) |
| AAV CLv-K6 (See SEQ ID NO: 70 in WO2016065001) | AAV CLv-L4 (See SEQ ID NO: 15 in WO2016065001) |
| AAV CLv-L4 (See SEQ ID NO: 65 in WO2016065001) | AAV CLv-L5 (See SEQ ID NO: 16 in WO2016065001) |
| AAV CLv-L5 (See SEQ ID NO: 66 in WO2016065001) | AAV CLv-L6 (See SEQ ID NO: 17 in WO2016065001) |
| AAV CLv-L6 (See SEQ ID NO: 67 in WO2016065001) | AAV CLv-Ml (See SEQ ID NO: 21 in WO2016065001) |
| AAV CLv-Ml (See SEQ ID NO: 71 in WO2016065001) | AAV CLv-Mll (See SEQ ID NO: 22 in WO2016065001) |
| AAV CLv-Ml 1 (See SEQ ID NO: 72 in WO2016065001) | AAV CLv-M2 (See SEQ ID NO: 23 in WO2016065001) |
| AAV CLv-M2 (See SEQ ID NO: 73 in WO2016065001) | AAV CLv-M5 (See SEQ ID NO: 24 in WO2016065001) |
| AAV CLv-M5 (See SEQ ID NO: 74 in WO2016065001) | AAV CLv-M6 (See SEQ ID NO: 25 in WO2016065001) |
| AAV CLv-M6 (See SEQ ID NO: 75 in WO2016065001) | AAV CLv-M7 (See SEQ ID NO: 26 in WO2016065001) |
| AAV CLv-M7 (See SEQ ID NO: 76 in WO2016065001) | AAV CLv-M8 (See SEQ ID NO: 27 in WO2016065001) |
| AAV CLv-M8 (See SEQ ID NO: 77 in WO2016065001) | AAV CLv-M9 (See SEQ ID NO: 28 in WO2016065001) |
| AAV CLv-M9 (See SEQ ID NO: 78 in WO2016065001) | AAV CLv-Rl (See SEQ ID NO: 30 in US8734809) |
| AAV CLv-RJ (See SEQ ID NO: 104 in US8734809) | AAV CLv-R2 (See SEQ ID NO: 31 in US8734809) |
| AAV CLv-R2 (See SEQ ID NO: 105 in US8734809) | AAV CLv-R3 (See SEQ ID NO: 32 in US8734809) |
| AAV CLv-R3 (See SEQ ID NO: 106 in US8734809) | AAV CLv-R4 (See SEQ ID NO: 33 in US8734809) |
| AAV CLv-R4 (See SEQ ID NO: 107 in US8734809) | AAV CLv-R5 (See SEQ ID NO: 34 in US8734809) |
| AAV CLv-R5 (See SEQ ID NO: 108 in US8734809) | AAV CLv-R6 (See SEQ ID NO: 35 in US8734809) |
| AAV CLv-R6 (See SEQ ID NO: 109 in US8734809); AAV CLv-R7 802 (see SEQ ID NO: 36 in US8734809) | AAV CLv-R7 (See SEQ ID NO: 110 in US8734809) |
| AAV CLv-R8 (See SEQ ID NO: 37 in US8734809) | AAV CLv-R8 (See SEQ ID NO: 111 in US8734809) |
| AAV CLv-R9 (See SEQ ID NO: 38 in US8734809) | AAV CLv-R9 (See SEQ ID NO: 112 in US8734809) |
| AAV CSp-1 (See SEQ ID NO: 45 in US8734809) | AAV CSp-1 (See SEQ ID NO: 119 in US8734809) |
| AAV CSp-10 (See SEQ ID NO: 46 in US8734809) | AAV CSp-10 (See SEQ ID NO: 120 in US8734809) |
| AAV CSp-11 (See SEQ ID NO: 47 in US8734809) | AAV CSp-11 (See SEQ ID NO: 121 in US8734809) |
| AAV CSp-2 (See SEQ ID NO: 48 in US8734809) | AAV CSp-2 (See SEQ ID NO: 122 in US8734809) |
| AAV CSp-3 (See SEQ ID NO: 49 in US8734809) | AAV CSp-3 (See SEQ ID NO: 123 in US8734809) |
| AAV CSp-4 (See SEQ ID NO: 50 in US8734809) | AAV CSp-4 (See SEQ ID NO: 124 in US8734809) |
| AAV CSp-6 (See SEQ ID NO: 51 in US8734809) | AAV CSp-6 (See SEQ ID NO: 125 in US8734809) |
| AAV CSp-7 (See SEQ ID NO: 52 in US8734809) | AAV CSp-7 (See SEQ ID NO: 126 in US8734809) |
| AAV CSp-8 (See SEQ ID NO: 53 in US8734809) | AAV CSp-8 (See SEQ ID NO: 127 in US8734809) |
| AAV CSp-8.10 (See SEQ ID NO: 38 in WO2016065001) | AAV CSp-8.10 (See SEQ ID NO: 88 in WO2016065001) |
| AAV CSp-8.2 (See SEQ ID NO: 39 in WO2016065001) | AAV CSp-8.2 (See SEQ ID NO: 89 in WO2016065001) |
| AAV CSp-8.4 (See SEQ ID NO: 40 in WO2016065001) | AAV CSp-8.4 (See SEQ ID NO: 90 in WO2016065001) |
| AAV CSp-8.5 (See SEQ ID NO: 41 in WO2016065001) | AAV CSp-8.5 (See SEQ ID NO: 91 in WO2016065001) |
| AAV CSp-8.6 (See SEQ ID NO: 42 in WO2016065001) | AAV CSp-8.6 (See SEQ ID NO: 92 in WO2016065001) |
| AAV CSp-8.7 (See SEQ ID NO: 43 in WO2016065001) | AAV CSp-8.7 (See SEQ ID NO: 93 in WO2016065001) |
| AAV CSp-8.8 (See SEQ ID NO: 44 in WO2016065001) | AAV CSp-8.8 (See SEQ ID NO: 94 in WO2016065001) |
| AAV CSp-8.9 (See SEQ ID NO: 45 in WO2016065001) | AAV CSp-8.9 (See SEQ ID NO: 95 in WO2016065001) |
| AAV CSp-9 842 (See SEQ ID NO: 54 in US8734809) | AAV CSp-9 (See SEQ ID NO: 128 in US8734809) |
| AAV.hu.48R3 (See SEQ ID NO: 183 in US8734809) | AAV.VR-355 (See SEQ ID NO: 181 in US8734809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in WO2016049230) | AAVF1/HSC1 (See SEQ ID NO: 2 in WO2016049230) |
| AAVF11/HSC11 (See SEQ ID NO: 26 in WO2016049230) | AAVF11/HSC11 (See SEQ ID NO: 4 in WO2016049230) |
| AAVF12/HSC12 (See SEQ ID NO: 30 in WO2016049230) | AAVF12/HSC12 (See SEQ ID NO: 12 in WO2016049230) |
| AAVF13/HSC13 (See SEQ ID NO: 31 in WO2016049230) | AAVF13/HSC13 (See SEQ ID NO: 14 in WO2016049230) |
| AAVF14/HSC14 (See SEQ ID NO: 32 in WO2016049230) | AAVF14/HSC14 (See SEQ ID NO: 15 in WO2016049230) |
| AAVF15/HSC15 (See SEQ ID NO: 33 in WO2016049230) | AAVF15/HSC15 (See SEQ ID NO: 16 in WO2016049230) |
| AAVF16/HSC16 (See SEQ ID NO: 34 in WO2016049230) | AAVF16/HSC16 (See SEQ ID NO: 17 in WO2016049230) |
| AAVF17/HSC17 (See SEQ ID NO: 35 in WO2016049230) | AAVF17/HSC17 (See SEQ ID NO: 13 in WO2016049230) |
| AAVF2/HSC2 (See SEQ ID NO: 21 in WO2016049230) | AAVF2/HSC2 (See SEQ ID NO: 3 in WO2016049230) |
| AAVF3/HSC3 (See SEQ ID NO: 22 in WO2016049230) | AAVF3/HSC3 (See SEQ ID NO: 5 in WO2016049230) |
| AAVF4/HSC4 (See SEQ ID NO: 23 in WO2016049230) | AAVF4/HSC4 (See SEQ ID NO: 6 in WO2016049230) |
| AAVF5/HSC5 (See SEQ ID NO: 25 in WO2016049230) | AAVF5/HSC5 (See SEQ ID NO: 11 in WO2016049230) |
| AAVF6/HSC6 (See SEQ ID NO: 24 in WO2016049230) | AAVF6/HSC6 (See SEQ ID NO: 7 in WO2016049230) |
| AAVF7/HSC7 (See SEQ ID NO: 27 in WO2016049230) | AAVF7/HSC7 (See SEQ ID NO: 8 in WO2016049230) |
| AAVF8/HSC8 (See SEQ ID NO: 28 in WO2016049230) | AAVF8/HSC8 (See SEQ ID NO:9 in WO2016049230) |
| AAVF9/HSC9 (See SEQ ID NO: 10 in WO2016049230) | AAVF9/HSC9 882 (see SEQ ID NO: 29 in WO2016049230) |
| 28m - 2vp3 (see SEQ ID NO: 139-142 in US 10,550,405, and SEQ ID NOs in WO2018/170310 or WO2019/216932) | AAV83 (see SEQ ID NO: 139-142 in US 10,550,405, and SEQ ID NOs in WO2018/170310 or WO2019/216932) |
| AAV82 (see SEQ ID NO: 139-142 in US 10,550,405, and SEQ ID NOs in WO2018/170310 or WO2019/216932) | AAV93(see SEQ ID NO: 139-142 in US 10,550,405, and SEQ ID NOs in WO2018/170310 or WO2019/216932) |
| AAV92 (see SEQ ID NO: 139-142 in US 10,550,405, and SEQ ID NOs in WO2018/170310 or WO2019/216932) | AAVrh10-3 (see SEQ ID NO: 139-142 in US 10,550,405, and SEQ ID NOs in WO2018/170310 or WO2019/216932) |
| AAV82G9 (see SEQ ID NO: 139-142 in US 10,550,405, and SEQ ID NOs in WO2018/170310 or WO2019/216932) | |

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. Pat. Application No. 11/486,254 and the other biological sequence files are to be read in a similar manner): 11486254.raw, 11932017.raw, 12172121.raw, 12302206.raw, 12308959.raw, 12679144.raw, 13036343.raw, 13121532.raw, 13172915.raw, 13583920.raw, 13668120.raw, 13673351.raw, 13679684.raw, 14006954.raw, 14149953.raw, 14192101.raw, 14194538.raw, 14225821.raw, 14468108.raw, 14516544.raw, 14603469.raw, 14680836.raw, 14695644.raw, 14878703.raw, 14956934.raw, 15191357.raw, 15284164.raw, 15368570.raw, 15371188.raw, 15493744.raw, 15503120.raw, 15660906.raw, and 15675677.raw.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference.

In some embodiments, an rAAV vector genome is single stranded or a monomeric duplex as described in U.S. Pat. No. 8,784,799, which is incorporated herein.

In some embodiments, the rAAV vector comprises nucleic acid that is devoid of bacterial sequence, and/or, lacks alternative open reading frames, and/or, lacks CpGs from the coding sequence, and/or, has double stranded RNA blocker or, double stranded RNA termination element. In some embodiments, the recombinant AAV of the invention is generated from closed ended linear duplexed DNA template. In some embodiments, the recombinant AAV of the invention is generated from plasmid DNA template.

In some embodiments, the recombinant AAV is produced from plasmid DNA. In some embodiments, the recombinant AAV is produced form close ended linear duplexed DNA. Closed linear DNA molecules typically comprise covalently closed ends also described as hairpin loops, where base-pairing between complementary DNA strands is not present. The hairpin loops join the ends of complementary DNA strands. Structures of this type typically form at the telomeric ends of chromosomes in order to protect against loss or damage of chromosomal DNA by sequestering the terminal nucleotides in a closed structure. In examples of closed linear DNA molecules described herein, hairpin loops flank complementary base-paired DNA strands, forming a closed linear (cl) DNA shaped structure. Closed linear DNA molecules include barbell shaped DNA.

Close ended linear duplex nucleic acid can be generated by a variety of known methods, including in vitro cell-free synthesis and in vivo methods. One method of generating the covalently closed ended linear duplex nucleic acids is by incorporation of protelomerase binding sites in a precursor molecule such that the protelomerase binding sites flank the nucleic acid of interest. Exposure of the nucleic acid molecule of interest to protelomerase thereby cleave and ligate the nucleic acid at the site. Examples of making close ended linear duplexed DNA are well known in the art e.g., as described in Nucleic Acids Res. 2015 Oct 15; 43(18): e120; Antisense & nucleic acid drug development 11:149-153 (2001); U.S. Pats. US 9109250, US 9499847, US 10501782, US 10286399; and/or, U.S. Publication No.s US 20190185924, US20190203282; the content of all of which are incorporated herein by reference in entirety.

Alternate methods of generating covalently closed end linear DNA that lack prokaryotic sequence or, bacterial sequences, are known in the art e.g., by formation of mini-circle DNA from plasmids (e.g. as described in U.S. Pat. 8,828,726, and U.S. Pat. 7,897,380, the contents of each of which are incorporated by reference in their entirety). For example, one method of cell-free synthesis combines the use of two enzymes - Phi29 DNA polymerase and a protelomerase, and generates high fidelity, covalently closed, linear DNA constructs. The constructs contain no antibiotic resistance markers, and therefore eliminate the packaging of these sequences. The process can amplify AAV genome DNA in a 2-week process at commercial scale and maintain the ITR sequences required for virus production.

In certain embodiments, the in vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g. a heterologous gene for expression); wherein the two DD-ITRs flank the nucleic acid in the context of covalently closed non-viral DNA and wherein the closed linear vector comprises a ½ protelomerase binding site on each end as for example as described in International publication no. WO 2019246544, which is incorporated herein by reference in its entirety.

In alternative embodiments, generation of rAAV vectors disclosed herein, e.g.., for the methods and compositions disclosed as disclosed herein can be performed using closed ended linear duplex DNA, including but not limited to Doggybone technology (dbDNA™), as disclosed in U.S. Application 2018/0037943 and Karbowniczek et al., Bioinsights, 2017, both of which are incorporated herein in its entirety by reference. In brief, a plasmid for AAV production using a closed ended linear duplex DNA technology can comprise the ITRs, promoter and gene of interest is flanked by a 56bp palindromic protelomerase recognition sequence. In some aspects of the embodiment, the ITR is 145 bp or less. In certain aspects of the embodiment, the ITR is 130 bp. The plasmid is denatured, and in the presence of a Phi29 DNA polymerase, and appropriate primers, Phi29 initiates rolling circle amplification (RCA), creating a double stranded cancatameric repeats of the original construct. When protelomerase is added, binding of the palindromic protelomerase recognition sequences occurs and cleavage-joining reaction occurs to result in a monomeric double stranded (ds) linear covalently closed DNA construct. Addition of common restriction enzymes remove the undesired DNA plasmid backbone sequence and digestion with exonuclease activity, resulting in barbell shaped DNA which can be size fractionated to isolate the barbell shaped DNA sequence encoding the ITRs, promoter and gene of interest. An exemplary plasmid for generation of rAAV vectors using closed ended linear duplex DNA including barbell shaped DNA, comprises in the following 5′ to 3′ direction: 5′-protelomerase RS, 5′ITR, LSP promoter, hGAA, 3′UTR, hGH poly(A), 3′ ITR, 3′-protelomerase RS (sense strand), where the sense strand is linked to the complementary antisense strand for a stranded (ds) linear covalently closed DNA construct. The use of closed ended linear duplex DNA, e.g., barbell shaped DNA as a starting material for the manufacturing of an AAV vector for use in the methods and composition as disclosed herein eliminates the bacterial backbone used to propagate the plasmid containing AAV vector with an inability for the product to trigger Toll-like receptor 9 (TLR9) responses. In some embodiments, methods of making the rAAV vectors disclosed herein are disclosed in US 9109250, US 9499847, US 10501782, US 10286399; and/or, U.S. Publication No’s US 2019/0185924, US2019/0203282, each of which are incorporated herein in their entirty by reference.

In an embodiment, an rAAV vector useful in the treatment of cardiovascular diseases or heart diseases or heart failure as disclosed herein is an AAV3b capsid. AAV3b capsids encompassed for use are described in 2017/106236, and 9,012,224 and 7,892,809, which are incorporated herein in its entirety by reference.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference. In some embodiments, an rAAV vector genome is single stranded or a monomeric duplex as described in U.S. Pat. No. 8,784,799, which is incorporated herein.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in PCT/US18/22725, PCT/US2018/044632, or US 10,550,405 which are incorporated by reference.

In an embodiment, an rAAV vector useful in the treatment of a cardiac disease or disorder, e.g., heart failure or CHF as disclosed herein is an AAV3b capsid. AAV3b capsids encompassed for use are described in 2017/106236, and 9,012,224 and 7,892,809, and International application PCT/US2019/061653 (WO2020/102645), which are incorporated herein in its entirety by reference. In some embodiments the AAV3b capsid is selected from any of: a AAV3b265D capsid, AAV3b265D549A capsid, AAV3bSASTG capsid, AAV3b265D549A capsid, AAV3b549A capsid, AAV3bQ263Y capsid, a AAV3bSASTG capsid comprising a AAV3b Q263A/T265 capsid as disclosed in WO2020/102645 which is incorporated herein in its entirety by reference. In some embodiments, the AAV3b capsid is selected from any of: SEQ ID NO: 44, 46, 50, 52, or 54 as disclosed in WO2020/102645 which is incorporated herein in its entirety by reference.

In order to facilitate their introduction into a cell, an rAAV vector genome useful in the invention are recombinant nucleic acid constructs that include (1) a heterologous sequence to be expressed (in one embodiment, a polynucleotide encoding an inhibitor of PP1, e.g., I-1 or I-1c, or a variant thereof as disclosed herein) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes an inhibitor of PP1, e.g., I-1 or I-1c, or a variant thereof as disclosed herein, for expressing the inhibitor of PP1 in a patient suffering from a cardiac disease or disorder, e.g., heart failure or CHF as disclosed herein. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

In one embodiment, an rAAV vector as disclosed herein useful in the treatment of a cardiac disease or disorder, e.g., heart failure or CHF as disclosed herein comprises a rAAV genome as disclosed herein, encapsulated by an AAV3b capsid or AAV2i8 capsid. In some embodiments, an rAAV vector as disclosed herein useful in the treatment of a cardiac disease or disorder, e.g., heart failure or CHF as disclosed herein comprises a rAAV genome as disclosed herein, encapsulated by any AAV3b capsid selected from: AAV3b capsid (SEQ ID NO: 44 as disclosed in WO2020/102645); AAV3b265D capsid (SEQ ID NO: 46 as disclosed in WO2020/102645), AAV3b ST (S663V+T492V as disclosed in WO2020/102645) capsid (SEQ ID NO: 48 as disclosed in WO2020/102645), AAV3b265D549A capsid (SEQ ID NO: 50 as disclosed in WO2020/102645); AAV3b549A capsid (SEQ ID NO: 52 as disclosed in WO2020/102645); AAV3bQ263Y capsid (SEQ ID NO: 54 as disclosed in WO2020/102645), or a AAV3bSASTG (i.e., Q263A/T265 as disclosed in WO2020/102645) capsid.

AAV vectors have been extensively discussed in the art. AAV vectors are of particular interest as AAV vectors do not typically integrate into the genome and do not elicit immune response. AAV serotypes 1, 2, 4, 5, 8, 9, rh10, DJ8 and 2g9 (AAV1, AAV2, AAV4, AAV5, AAV8, AAV9, AAVrh10, AAVDJ8 and AAV2g9) have been noted to achieve efficient transduction in the heart. Therefore, AAV1, AAV2, AAV4, AAV5, AAV8, AAV9, AAVrh10, AAVVDJ8, AAV2g9, AAV2i8 and derivatives thereof are particularly preferred AAV serotypes. In some embodiments, AAV9 is a preferred AAV vector. In other embodiments, AAV2g9 is a particularly preferred AAV vector (WO2014/144229). In yet other embodiments, a particularly preferred AAV vector is AAVDJ8. In some embodiments, AAVrh10 is particularly preferred AAV vector. In yet another embodiment, AAV2i8 is particularly preferred vector. AAV2i8 is disclosed in Patent 8,889,641, which is incorporated herein in its entirety by reference. In some embodiments, the AAV is a hybrid-AAV2ITR/AAV, as disclosed in U.S. Pat. 7,172,893, which is incorporated herein in its entirety by reference. Suitably an AAV vector comprises a viral genome which comprises a nucleic acid sequence of the present invention positioned between two inverted terminal repeats (ITRs). WO2019/028306, for example discloses various wild type and modified AAV vectors that can be used in the heart. In one embodiment, AAV vectors of the present invention are recombinant AAV viral vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ or an organism. Suitably AAV vectors for use herein comprise a virus that has been reduced to the minimum components necessary for transduction of a nucleic acid payload or cargo of interest. In this manner, AAV vectors are engineered as vehicles for specific delivery while lacking the deleterious replication and/or integration features found in wild-type viruses. In one embodiment, the AAV particle of the present invention is an scAAV. In some embodiments, the AAV particle of the present invention is an ssAAV. Methods for producing and/or modifying AAV particles are disclosed extensively in the art (see e.g. WO2000/28004; WO2001/23001; WO2004/112727; WO 2005/005610 and WO 2005/072364, which are incorporated herein by reference). In one embodiment the AAV vector comprises a capsid that allows for blood brain barrier penetration following intravascular (e.g. intravenous or intraarterial) administration (see e.g. WO2014/144229, which discusses, for example, capsids engineered for efficient crossing of the blood brain barrier, e.g. capsids or peptide inserts including VOY 101, VOY201, AAVPHP.N, AAVPHP.A, AAVPHP.B, PHP.B2, PHP.B3, G2A3, G2B4, G2B5, PHP.S, and variants thereof).

Methods of making AAV vectors are well known in the art and are described in e.g., U.S. Pat. Nos. US6204059, US5756283, US6258595, US6261551, US6270996, US6281010, US6365394, US6475769, US6482634, US6485966, US6943019, US6953690, US7022519, US7238526, US7291498 and US7491508, US5064764, US6194191, US6566118, US8137948; or International Publication Nos. WO1996039530, WO1998010088, WO 1999014354, WO1999/015685, WO1999/047691, WO2000/055342, WO2000/075353 and WO2001/023597; Methods In Molecular Biology, ed. Richard, Humana Press, NJ (1995); O’Reilly et al, Baculovirus Expression Vectors, A Laboratory Manual, Oxford Univ. Press (1994); Samulski et al.,J Fir.63:3822-8 (1989); Kajigaya et al, Proc. Nat′l. Acad. Sci. USA 88: 4646-50 (1991); Ruffing et al., J. Vir. 66:6922-30 (1992); Kimbauer et al, Vir., 219:37-44 (1996); Zhao et al, Vir.272: 382-93 (2000); the contents of each of which are herein incorporated by reference. Viral replication cells commonly used for production of recombinant AAV viral particles include but are not limited to HEK293 cells, COS cells, HeLa cells, KB cells, and other mammalian cell lines.

It is to be understood that a viral expression system will further be modified to include any necessary elements required to complement a given viral vector during its production using methods described herein. For example, in certain embodiment, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

A viral expression system for manufacturing an AAV vector (e.g., an AAV expression system) could further comprise Replication (Rep) genes and/or Capsid (Cap) genes in trans, for example, under the control of an inducible promoter. Expression of Rep and Cap can be under the control of one inducible promoter, such that expression of these genes are turned “on” together, or under control of two separate inducible promoters that are turned “on” by distinct inducers. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. US5478745A; US5622856A; US5658776A; US6440742B1; US6632670B1; US6156303A; US8007780B2; US6521225B1; US7629322B2; US6943019B2; US5872005A; and U.S. Pat. Application Nos. US 2017/0130245; US20050266567A1; US20050287122A1; the contents of each are incorporated herein by reference in their entireties.

In one embodiment, the cells for producing an AAV vector are cultured in suspension. In some embodiments, the cells are cultured in animal component-free conditions. The animal component-free medium can be any animal component-free medium (e.g., serum-free medium) compatible with a given cell line, for example, HEK293 cells. Any cell line known in the art to be capable of propagating an AAV vector can be used for AAV production using methods described herein. Exemplary cell lines that can be used to generate an AAV vector include, without limitation, HEK293, CHO, Cos-7, and NSO.

In one embodiment, a cell line for producing an AAV vector stably expresses any of the components required for AAV vector production, e.g., Rep, Cap, VP1, etc. In one embodiment, a cell line for producing an AAV vector transiently expresses any of the components required for AAV vector production, e.g., Rep, Cap, VP1, etc.

In the event that a cell line for producing an AAV vectors does not stably or transiently express rep or cap, these sequences are to be provided to the AAV expression system. AAV rep and cap sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/ cap sequences will not be flanked by the TRs, to prevent rescue and/or packaging maintain of these sequences.

A viral expression system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase the host chromosome. LTRs and methods for manufacturing lentiviral vectors are further described, e.g., in U.S. Pat. Nos. US7083981B2; US6207455B1; US6555107B2; US8349606B2; US7262049B2; and U.S. Pat. Application No. US20070025970A1; US20170067079A1; US20110028694A1; the contents of each are incorporated herein by reference in their entireties.

A viral expression system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. Nos. US7510875B2; US7820440B2; US7749493B2; US7820440B2; US10041049B2; International Patent Application Numbers WO2000070071A1; and U.S. Pat. Application Nos. WO2000070071A1; US20030022356A1; US20080050770A1 the contents of each are incorporated herein by reference in their entireties.

In one embodiment, the viral expression system can be a host cell, such as a virus, a mammalian cell or an insect cell. Exemplary insect cells include but are not limited to Sf9, Sf21, Hi-5, and S2 insect cell lines. For example, a viral expression system for manufacturing an AAV vector could further comprise a baculovirus expression system, for example, if the viral expression system is an insect cell. The baculovirus expression system is designed for efficient large-scale viral production and expression of recombinant proteins from baculovirus-infected insect cells. Baculovirus expression systems are further described in, e.g., U.S. Pat. Nos. US6919085B2; US6225060B1; US5194376A; the contents of each are incorporated herein by reference in their entireties.

In some embodiments, the viral expression system is a cell-free system. Cell-free systems for viral vector production are further described in, for example, Cerqueira A., et al. Journal of Virology, 2016; Sheng J., et al. The Royal Society of Chemistry, 2017; and Svitkin Y.V., and Sonenberg N. Journal of Virology, 2003; the contents of which are incorporated herein by reference in their entireties.

Viral vectors produced in a cell can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

Provided herein is a viral vector expressing a nucleic acid having a sequence selected from SEQ ID NOs 385-412. In one embodiment, the nucleic acid sequence has a sequence that is at least 75%, 80%, 85%, 86%, 87%, 88%, 89%, 90%, 91%, 92%, 93%, 94%, 95%, 96%, 97%, 98%, 99% identical to the sequence of SEQ ID NOs 385-412. In one embodiment, the viral vector is an AAV.

One aspect herein is an rAAV expressing a nucleic acid having a sequence selected from SEQ ID NOs 385-412. One aspect herein is an rAAV comprising a nucleic acid having a sequence selected from SEQ ID NOs 413-440.

Further provided herein is a composition comprising a viral vector, e.g., an rAAV, expressing a nucleic acid having a sequence selected from SEQ ID NOs 385-412. Further provided herein is a nucleic acid sequence encoding a I1c transgene, wherein the nucleic acid sequence is selected from SEQ ID NOs 385-412. In one embodiment, the nucleic acid sequence has a sequence that is at least 75%, 80%, 85%, 86%, 87%, 88%, 89%, 90%, 91%, 92%, 93%, 94%, 95%, 96%, 97%, 98%, 99% or more identical to a sequence selected from SEQ ID NOs 385-412.

In one embodiment, the nucleic acid sequence is a codon-optimized sequence. In one embodiment, the nucleic acid sequence as set forth in SEQ ID No.s 385-412, or encoding other optimized I-1c transgene is operatively linked with a promoter selected from a CMV promoter, a cardiac-specific promoter selected from Table 2A or a variant thereof, or a muscle-specific promoter active in cardiac and skeletal muscle selected from Table 5A or a variant thereof, or a shortened muscle-specific promoter active in cardiac and skeletal muscle selected from Table 13A, or a variant thereof.

Also provided are closed linear DNA constructs comprising a nucleic acid sequence of any of 357-384. The closed linear DNA can be used in methods for making rAAV that lack bacterial DNA sequences. Thus, also provided herein are pharmaceutical compositions for treatment of heart failure that comprise rAAV encoding constitutively active I-1c, wherein the rAAV compositions lack bacterial nucleic acid sequences.

### V. Cardiac Cells and Muscle Cells

As a non-limiting example, the suitable cardiac cells include, but are not limited to, embryonic stem cells; cardiac fibroblasts; skeletal myoblasts; cardiomyocytes, including ventricular cardiomyocytes, and the like. In other embodiments, the cardiac cell is a non-cardiomyocyte somatic cell. In some embodiments, the cardiac cell is an iPS- or other stem-cell derived cardiac cell, that is transfected with the rAAV vector disclosed herein ex vivo and transplanted into a subject. In some embodiments, the iPSC or other stem-cell derived cardiac cell is a human cell. In some embodiments, the cardiac cell is derived from an adult stem cell. Methods to differentiate iPSC into mature cardiac cells, including cardiomyocytes are known in the art, and include, without limitation, Uosaki et al. (PLOS One, 2011, 6(8): e23657) describes methods for in vitro differentiation of human iPS cells to cardiomyocytes.

In some embodiments, cardiomyocytes as disclosed herein can be derived from Isl1+ multipotent progenitor cells such as those disclosed in U.S. Provisional Application 60/856,490 and 60/860,354 and in International Application PCT/US07/23155, which is incorporated herein in its entirety by reference.

Cardiomyocytes for transfection with the rAAV vectors as disclosed herein can be identified and isolated by using agents reactive to markers typical of the cardiomyocytes lineage, including but without limitation, the positive expression of Mef2c, Nxk2.5, Tbx20, Isl1, GATA4, GATA6; Tropinin T (TnT), Troponin C (TnI), BMP7, BMP4, BMP2, miR-208, miR-143, miR-133a, miR-133b, miR-1, miR-143, miR-689 and smooth muscle actin (smActin), or homologues or variants thereof. Alternatively, cardiomyocytes are positive for the expression of Mef2c and Nxk2.5. To be more precise, cardiomyocytes can be selected or identified based on the positive expression of Mef2c and Nxk2.5 and the lack of or low level expression of at least one of the following markers: Tbx5; Snai2; miR-200a; miR-200b; miR-199a; miR-199b; miR-126-3p; miR-322 and CD31 or homologues or variants thereof.

In some embodiments, the cell marker, SIRPA (signal-regulatory protein alpha), can be used to identify a population of cardiomyocytes differentiated from stem cells and permits, for example, the isolation of a population that is 98% cardiac troponin T positive (Dubois et al., Nat. Biotech, (2011) 29;1011-1018). In the methods described by Dubois et al., negative selection of stem cells for PECAM, THY 1, PDGFRB and ITGA1 can be used to remove the non-myocyte population.

In some embodiments of the invention, the cell is ex vivo, e.g. in cell culture. In other embodiments of the invention the cell may be part of a tissue or multicellular organism.

In a preferred embodiment, the cell is a muscle cell (myocyte), which may be ex vivo or in vivo. In a preferred embodiment, the cell is a cardiac muscle cell, which may be ex vivo or in vivo. In an alternative preferred embodiment, the cell is a skeletal muscle cell, which may be ex vivo or in vivo. The muscle cell may be a primary muscle cell or a cell of a muscle-derived cell line, e.g. an immortalised cell line. The cell may be present within a muscle tissue environment (e.g. within a muscle of an animal) or may be isolated from muscle tissue, e.g. it may be in cell culture. Suitably the cell is a human cell.

The skeletal muscle cells may be from fast twitch or slow twitch muscles.

The cardiac muscle cells may be selected from ventricular cardiomyocytes, atrial cardiomyocytes, cardiac fibroblasts, or endothelial cells (EC) in the heart, as well as peri-vascular cells and pacemaker cells. In one embodiment, the muscle cell/s are cardiac muscle cell/s. In one embodiment, the muscle cell/s are skeletal muscle cell/s.

### VI. Pharmaceutical Compositions

The rAAV vectors as disclosed herein for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. Pharmaceutical compositions comprising the rAAV vectors as disclosed herein for use in the methods of administration as disclosed herein and uses thereof are known in the art.

Accordingly, a further aspect of the invention provides a pharmaceutical composition comprising a rAAV vector as disclosed herein for use in the methods of administration as disclosed herein. Relative amounts of the active ingredient (e.g. a rAAV vectors aa disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 5 and 50 percent, between 1-30 percent, between 5- 80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The rAAV vectors as disclosed herein for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, posttreatment, or during remission of the disorder.

When administered in combination, the rAAV vectors as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a rAAV vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the rAAV vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a rAAV vector as disclosed herein can deliver a rAVV vector disclosed herein alone, or in combination with an additional agent, for example, an immune modulator as disclosed herein. In some embodiments, the additional agent is a muscle enhancing protein or peptide, e.g., to improve blood flow and enhance muscle function in the treated muscle. Furthermore, if desired, in some embodiments, the additional agent a vasoactive agent which can be employed in conjunction with these methods and compositions, as described herein, in order to further enhance gene delivery at the target site. Exemplary vasoactive agent include but are not limited to histamine, a histamine agonist, a nitric oxide donor, or a VEGF protein, and can be used to increase the efficiency of gene transfer at a gene vector dose. In some embodiments, a vasoactive agent is useful the to limit the amount of vector required to be administered in order to achieve a given therapeutic effect.

Compounds which may be used in combination with the AAV particles described herein include, but are not limited to, agents currently used for treatment of congestive heart failure include angiotensin converting enzyme (ACE) inhibitors, beta-blockers, compounds that induce inotropic effects (e.g., increase of force of contraction of the heart) and compounds that increase urine flow, or diuretics. In some embodiments, the rAAV vector is administered according to the methods as disclosed herein, in combination with another active agent, such as a food-intake-reducing, or plasma glucose-lowering or plasma lipid-lowering agent, such as amylin, an amylin agonist, a CCK, or a leptin, or a cardiac treatment agent such as angiotensin converting enzyme (ACE) inhibitors. In one embodiment, a rAAV vector as disclosed herein, administered according to the methods as disclosed herein is administered in combination with captopril (CAPOTEN®). In other embodiments, a rAAV vector as disclosed herein, administered according to the methods as disclosed herein is administered in combination with one or more additional ACE inhibitors, such as benazepril (LOTENSIN®), enalapril (VASOTEC®), lisinopril (PRMIVIL® ZESTRIL®), fosinopril (MONOPRIL®), ramipril (ALTACE®), perindopril (ACEON®), quinapril (ACCUPRIL®), moexipril (UNIVASC®), and trandolapril (MAVIK®).

In some embodiments, a rAAV vector as disclosed herein, administered according to the methods as disclosed herein may be used for treatment of congestive heart failure in combination with one or more beta blockers, such as sotalol (BETAPACE®), timolol (BLOCADREN®), esmolol (BREVIBLOC®), carteolol (CARTROL®), carvedilol (COREG®), nadolol (CORGARD®), propranolol (INDERAL®), propranolol (MDERAL-LA®), betaxolol (KERLONE®), penbutolol (LEVATOL®), metoprolol (LOPRESSOR®), labetalol (NORMODYNE®), acebutolol (SECTRAL®), atenolol (TENORMIN®), metoprolol (TOPROL-XL®), labetalol (TRAND ATE®), pindolol (VISKEN®), and bisoprolol ( ZEBET A®).

In some embodiments, a rAAV vector as disclosed herein, administered according to the methods as disclosed herein may be used for treatment of congestive heart failure in combination with one or more angiotensin π receptor blockers (ARB), such as candesartan cilexetil (ATACAND®), irbesartan (AVAPRO®), losartan (COZAAR®), valsartan (DIO V AN®), telmisartan (MICARDIS®), and eprosartan mesylate (TEVETEN®). In some embodiments, a rAAV vector as disclosed herein, administered according to the methods as disclosed herein may be used for treatment of congestive heart failure in combination with one or more aldosterone antagonists, such as spironolactone (ALDACTAZIDE®) and eplerenone (INSPRA®). In some embodiments, a rAAV vector as disclosed herein, administered according to the methods as disclosed herein may be used in combination with an IL-18 inhibitor, e.g., or IL-18BP or a variant thereof, as disclosed in U.S. Pat. 7,799,541, which us incorporated herein in its entirety by reference.

In some embodiments, a rAAV vector as disclosed herein, administered according to the methods as disclosed herein may be used for treatment of congestive heart failure in combination with one or more vasopeptidase inhibitors. Vasopeptidase inhibitors include NEP/ACE inhibitors that possess natural endopeptidase (NEP) and ACE inhibitory activity. Examples of NEP/ACE inhibitors include, but are not limited to, tricyclic benzazepinone thiols, omapatrilat, gemopatrilat, mixanpril, racecadotril, fasidotril, sampatrilat, MDL 100.240 Z13752A, BMS1 89921, BMS1 82657, and CGS 30008. Examples of NEP/ACE inhibitors suitable for use herein include those disclosed in U.S. Pat. Nos. 5,362,727, 5,366,973, 5,225,401, 4,722,810, 5,223,516, 4,749,688, and 5,552,397, which are incorporated herein in their entirety by reference. Administration of such additional agents or compounds can be at the same time (i.e., during administration) of the rAAV vector administered according to the methods as disclosed herein, or the subject can be undergoing treatment with the additional agent before, or during, or after administration with the rAAV vector.

In some embodiments, a rAAV vector as disclosed herein, administered according to the methods as disclosed herein may be used for treatment of congestive heart failure in combination with any therapy for congestive heart failure that is known in the art. For example, in one embodiment, administration of a rAAV vector according to the methods as disclosed herein can be used for treatment of congestive heart failure in combination with therapeutic devices such as cardiac resynchronization

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In a further aspect, a rAAV vector as disclosed herein is prepared for use as medicament for use in the methods of administration as disclosed herein.

In a further aspect, a rAAV vector as disclosed herein is prepared for use as medicament for use in the methods of administration as disclosed herein for use in therapy, i.e. the prevention or treatment of a medical condition or disease, e.g., a cardiovascular disease or heart disease or disorder as disclosed herein. Exemplary medical conditions or diseases relevant to the present aspect are discussed below.

In a further aspect, there is provided a cell comprising a rAAV vector as disclosed herein comprising a synthetic cardiac-specific promoter selected from Tables 1-3, or a variant thereof. In some embodiments the cell is a mammalian cell, optionally a human cell. Suitably, the cell is a cardiac cell. Suitably the cell may be a cardiomyocyte, e.g., ventricular cardiomyocyte. Suitably the cell may be a human cardiomyocyte, e.g., human ventricular cardiomyocyte.

In some embodiments, the pharmaceutical composition comprises recombinant AAV vector in a buffer (e.g., excipient) of about pH 7.0 to about pH 8.0. In some embodiments, the pH of the buffer is from about 7.0 to about 7.5. In preferred embodiment, the pH of the buffer is less than 7.5. In several embodiments, the buffer is phosphate buffer saline (PBS) or a phosphate buffer (e.g., 10 mM Phosphate pH 7.4, 350 mM NaCl, 2.7 mM KCl, 5% Sorbitol, 0.001% (w/v) poloxamer 188). In certain embodiments, the buffer or, excipient comprises ions selected from the group consisting of sodium, potassium, phosphate, chloride, calcium, magnesium, sulfate, citrate and any combination thereof. The pharmaceutical composition further comprises polyol, sugar or, similar. In some embodiment, the pharmaceutical composition comprises glycerol or, propylene glycol, or, polyethylene glycol, or, sorbitol, or, mannitol. In several embodiments, the sorbitol concentration ranges from about 1% (w/v) to about 10% (w/v). In some embodiments, the sorbitol concentration ranges from about 2%(w/v) to about 8%(w/v). In preferred embodiments, the sorbitol concentration ranges from about 3%(w/v) to about 6% (w/v). In certain embodiments, the sorbitol concentration is 1% (w/v), 2% (w/v), 3% (w/v), 4% (w/v), 5% (w/v), 6% (w/v), 7% (w/v), 8% (w/v), 9% (w/v), or, 10% (w/v). The pharmaceutical composition further comprises a non-ionic surfactant. In some embodiments, the non-ionic surfactant is selected from the group consisting of polyoxyethylene-polyoxypropylene block copolymers, alkylglucosides, alkyl phenol ethoxylates, polysorbates, polyoxyethylene alkyl phenyl ethers, and any combinations thereof. In some embodiments, the non-ionic surfactant is poloxamer 188 or, Ecosurf SA-15. In ceratin embodiments, poloxamer 188 or, Ecosurf SA-15 concentration is 0.0005% (w/v), 0.0008% (w/v), 0.0009% (w/v), 0.001% (w/v), 0.002% (w/v), 0.0025% (w/v), 0.003% (w/v), 0.0035% (w/v), 0.004% (w/v), 0.0045% (w/v), 0.005% (w/v), 0.006% (w/v), 0.007% (w/v), 0.008% (w/v), 0.009% (w/v), or, 0.01% (w/v).

The pharmaceutical composition comprises at least 1×10⁹ vg/ml recombinant AAV vector as disclosed in the present invention. In some embodiments the pharmaceutical composition comprises about 1×10⁹ vg/ml to about 1×10¹³ vg/ml recombinant AAV vector. In some embodiments, the pharmaceutical composition comprises about 1×10¹¹ vg/ml to about 1×10¹³ vg/ml recombinant AAV vector. In several embodiments, the pharmaceutical composition comprises about 1×10¹¹ vg/ml to about 1×10¹³ vg/ml recombinant AAV2i8 vector comprising nucleic acid encoding phosphatase inhibitor polypeptide wherein the nucleic acid is operatively linked with a promoter selected from a CMV promoter, a cardiac-specific promoter selected from Table 2A or a variant thereof, or a muscle-specific promoter active in cardiac and skeletal muscle selected from Table 5A or a variant thereof, or a shortened muscle-specific promoter active in cardiac and skeletal muscle selected from Table 13A, or a variant thereof.

### VII. Other Methods and Uses

In one embodiment, the technology also provides a rAAV vector as disclosed herein for use in the methods of administration as disclosed herein for use in the treatment of a cardiovascular disease or heart disease or heart failure, preferably CHF. Relevant conditions, diseases and therapeutic expression products are discussed above. In one embodiment, the technology also provides a rAAV vector as disclosed herein for use in the treatment of a cardiovascular disease or heart disease or heart failure, preferably CHF.

In one embodiment, the technology also provides a rAAV vector as disclosed herein for use in the methods of administration as disclosed herein for use as a medicament. In one embodiment, the technology also provides a rAAV vector as disclosed herein for use as medicament.

In one embodiment, the technology also provides a rAAV vector as disclosed herein for use in the methods of administration as disclosed herein for use the manufacture of a pharmaceutical composition for treatment of any condition or disease mentioned herein. In one embodiment, the technology also provides a rAAV vector as disclosed herein for use in the treatment of any condition or disease mentioned herein.

In one embodiment, the technology also provides a cell comprising rAAV vector as disclosed herein for use in the methods of administration as disclosed herein. Suitably the cell is a eukaryotic cell. The eukaryotic cell can suitably be an animal (metazoan) cell (e.g. a mammalian cell). Suitably, the cell is a human cell. In some embodiments of the invention, the cell is ex vivo, e.g. in cell culture. In other embodiments of the invention the cell may be part of a heart tissue or a heart tissue. In one embodiment, the technology also provides a cell comprising rAAV vector as disclosed herein for use as a medicament.

In a preferred embodiment, the cell is a heart cell, which may be ex vivo or in vivo. The heart cell may be a cardiomyocyte, e.g., ventricular cardiomyocyte, atrial cardiomyocyte, smooth muscle cell, pacemaker cell, or other heart cell. Alternatively, the heart cell may be a heart-derived cell line, e.g. immortalised cell line. In one embodiment, the cell is a muscle cell, e.g., a cardiomyocyte or smooth muscle cell in the heart. The cell may be present within a heart tissue environment (e.g. within the heart of an animal) or may be isolated from heart tissue, e.g. it may be in cell culture. Suitably the primary cell or the cell line is a human cell.

In a further aspect the present invention provides a method for producing a rAAV vector as disclosed herein in a cell, preferably a cardiac cell. The method suitably comprises maintaining said cardiac cell under suitable conditions for expression of the gene. In culture this may comprise incubating the cardiac cell, or tissue comprising the cardiac cell, under suitable culture conditions. The expression may of course be in vivo, e.g. in one or more cells in the heart of a subject.

Suitably the method comprises the step of introducing the rAAV vector as disclosed herein into the cardiac cell. A wide range of methods of transfecting cardiac cells are well-known in the art. A preferred method of transfecting cardiac cells is transducing the cells with a rAAV vector as disclosed herein.

It will be evident to the skilled person that rAAV vector as disclosed herein can be used in the methods of administration as disclosed herein for gene therapy.

The present invention also provides a method of administrating a rAAV vector as disclosed herein for expressing a therapeutic transgene in a cardiac cell, the method comprising introducing into the cardiac cell a rAAV vector as disclosed herein according to the methods of administration as disclosed herein. The cardiac cell can be in vivo or ex vivo.

All aspects of the compositions and methods of the technology disclosed herein can be defined in any one or more of the following numbered paragraphs:

A. A method of treating a patient having a heart failure, comprising: administering into heart cells of the patient, at least one total dose of a rAAV vector comprising a nucleic acid sequence encoding a phosphatase inhibitor protein that inhibits phosphatase activity,wherein, at least one dose of the rAAV is selected from a total dose-range of about 10¹³ vg to about 10¹⁵ vg, and wherein, six months post-administration NT-proBNP level in serum of the patient is below 900 pg/ml.

B. The method of paragraph A, wherein the rAAV vector further comprises CMV promoter or a synthetic promoter, operatively linked to the phosphatase inhibitor protein.

C. The method of any of paragraphs A-B, wherein the total dose is administered over a period of time of about 20 minutes to about 30 minutes.

D. The method of any of paragraphs A-C, wherein the administration of the total dose is performed in sub-doses, wherein each sub-dose is administered over a period of time of 1-5 minutes.

E. The method of any of paragraphs A-D, wherein the administration of the total dose is performed in five sub-doses, each sub-dose is administered over a period of time of 1-5 minutes.

F. The method of paragraph 5, wherein the five sub-doses are administered over a period of about 20 minutes to about 30 minutes.

G. The method of any of paragraphs A-E, wherein the rAAV is selected from the group consisting of AAV2, AAV6, AAV8, AAV9, AAV2i8, rh10, AAV2.5 and AAV2G9.

H. The method of any of paragraph 1-7, wherein at least one total dose of the rAAV is 10¹³ vg, 3×10¹³ vg, 10¹⁴ vg, 3×10¹⁴ vg, or, 10¹⁵ vg.

I. A method of treating a patient having a heart failure, comprising: administering into heart cells of the patient, at least one total dose of a rAAV vector comprising (i) a nucleic acid sequence encoding a phosphatase inhibitor protein that inhibits phosphatase activity, (ii) a synthetic promoter, operatively linked to the phosphatase inhibitor (I-1) protein.

J. The method of paragraph I, wherein the total dose is administered over a period of time of about 20 minutes to about 30 minutes.

K. The method of paragraph I-J, wherein the administration of the total dose is performed in sub-doses, wherein each sub-dose is administered over a period of time of 1-5 minutes.

L. The method of paragraph I to K, wherein the administration of the total dose is performed in five sub-doses, each sub-dose is administered over a period of time of 1-5 minutes

M. The method of paragraph I to L, wherein the five sub-doses are administered over a period of about 20 minutes to about 30 minutes.

N. The method of any of paragraphs A-M, wherein the rAAV is selected from the group consisting of AAV2, AAV6, AAV8, AAV9, AAV2i8, rh10, AAV2.5 and AAV2G9.

O.. The method of any of paragraph A-N, wherein at least one total dose of the rAAV is selected from a dose-range of about 10¹³ vg to about 10¹⁵ vg.

P. The method of any of paragraphs A-O, wherein at least one dose of the rAAV is 10¹³ vg, 3×10¹³ vg, 10¹⁴ vg, 3×10¹⁴ vg, or, 10¹⁵ vg.

Q. The method of any of paragraphs A-P, wherein six-months post administration of the rAAV dose, NT-proBNP level in serum of the patient is below 900 pg/ml.

R. The method of any of paragraphs A-Q, wherein the method further comprises administering an immune modulator.

S. The method of any of the preceding paragraphs A to R, wherein the rAAV vector comprises a nucleic acid sequence encoding a polypeptide comprising amino acids 1-65 of SEQ ID NO: 2, wherein threonine at position 35 of SEQ ID NO: 1 is replaced with aspartic acid (T35D).

T. The method of any of the preceding paragraphs A to S, wherein the rAAV is selected from Table 11.

U. The method of any of the preceding paragraphs A to T, the administration further comprises nitroprusside or nitroglycerin.

V. The method of any of paragraphs A-U, wherein the synthetic promoter causes expression of the phosphatase inhibitor protein preferentially in smooth muscle cells.

W. The method of any of paragraphs A-V, wherein the synthetic promoter causes expression of the phosphatase inhibitor protein preferentially in cardiac cells.

X. The method of any of paragraphs A-W, wherein the expression is equivalent to the expression caused by CMV promoter.

Y. The method of any of paragraphs A-X, wherein the administration is into the lumen of the coronary artery of the heart of the patient or systemic administration.

Z. The method of any of paragraphs A-Y, wherein the synthetic promoter is a cardiac-specific promoter selected from Table 2A or a variant thereof, or a muscle-specific promoter active in cardiac and skeletal muscle selected from Table 5A, or, Table 13A or a variant thereof.

AA. A method of treating a patient having a cardiovascular condition or a heart disease, comprising: administering into heart cells of the patient, at least one total dose of a rAAV vector comprising a therapeutic nucleic acid operatively linked to a cardiac-specific promoter selected from Table 2A or a variant thereof, or a muscle-specific promoter active in cardiac and skeletal muscle selected from Table 5A, or, Table 13A, or a variant thereof, wherein the therapeutic nucleic acid is RNA or DNA, wherein the therapeutic nucleic acid expresses a therapeutic protein selected from Table 18A or 18B.

BB. The methods of paragraph AA, wherein the cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

CC. The method of any paragraphs of AA-BB, wherein the total dose is administered over a period of time of about 20 minutes to about 30 minutes.

DD. The method of any of paragraphs AA-CC, wherein the administration of the total dose is performed in sub-doses, wherein each sub-dose is administered over a period of time of 1-5 minutes.

EE. A method of treating a patient having a heart failure, comprising: administering into heart cells of the patient, at least one total dose of a rAAV vector comprising a nucleic acid sequence encoding a phosphatase inhibitor protein that inhibits phosphatase activity, wherein, at least one total dose of the rAAV is selected from a dose-range of about 10¹³ vg to about 10¹⁵ vg, wherein, the total dose is administered over a period of time of about 20 minutes to about 30 minutes, wherein, the administration of the total dose is performed in sub-doses, wherein each sub-dose is administered over a period of time of 1-5 minutes.

FF. The method of any of paragraph AA-EE, wherein the rAAV vector further comprises CMV promoter or a synthetic promoter, operatively linked to the phosphatase inhibitor protein.

GG. The method of any of paragraphs AA-FF, wherein the rAAV is selected from the group consisting of AAV2, AAV6, AAV8, AAV9, AAV2i8, rh10, AAV2.5 and AAV2G9.

HH. The method of any of paragraphs AA-GG, wherein the administration of the total dose is performed in five sub-doses, each sub-dose is administered over a period of time of 1-5 minutes.

II. The method of any of paragraph AAHH, the five sub-doses are administered over a period of about 20 minutes to about 30 minutes.

JJ. The method of any of paragraphs AA-II, wherein at least one total dose of the rAAV is 10¹³ vg, 3×10¹³ vg, 10¹⁴ vg, 3×10¹⁴ vg, or, 10¹⁵ vg.

KK. The method of any of the preceding paragraphs, wherein at least one subdose of the rAAV is 10¹³ vg, 3×10¹³ vg, 10¹⁴ vg, 3×10¹⁴ vg, or, 10¹⁵ vg.

LL. A method of treating a patient having congestive heart failure, comprising: administering to a patient, at least one dose of a rAAV vector, wherein the rAAV vector is AAV2i8 and comprises a nucleic acid encoding phosphatase inhibitor 1 (I-1) operatively linked to a promoter selected from: a CMV promoter, a cardiac-specific promoter selected from Table 2A or a variant thereof, or a muscle-specific promoter active in cardiac and skeletal muscle selected from Table 5A, or, Table 13A,or a variant thereof.

MM. The method of paragraph LL, wherein the I-1 comprises amino acids 1-65 of SEQ ID NO: 1 or a functional fragment thereof, wherein the threonine at position 35 of SEQ ID NO: 1 is replaced with an aspartate acid (T35D).

NN. The method of paragraphs LL to MM, wherein the nucleic acid encoding phosphatase inhibitor encodes a constitutively active fragment of I-1 (I-1c) comprising a fragment of SEQ ID NO: 1, wherein the fragment is selected from: amino acids 1-54 of SEQ ID NO: 1, 1-61 of SEQ ID NO: 1, 1-65 of SEQ ID NO: 1, 1-66 of SEQ ID NO: 1, 1-67 of SEQ ID NO: 1 or 1-77 of SEQ ID NO: 1, or a functional variant thereof, wherein the threonine at position 35 of SEQ ID NO: 1 is replaced with an aspartate acid (T35D).

OO. The method of any of paragraphs LL-NN, wherein the heart failure comprises ischemia, arrhythmia, myocardial infarction, abnormal heart contractibility, or abnormal Ca2+ metabolism.

PP. The method of any of paragraphs LL-OO, wherein the administration is into the lumen of the coronary artery of the heart of the patient.

QQ. The method of any of paragraphs LL-PP, wherein the at least one dose is a total dose-range of about 10¹³ vg to about 10¹⁵ vg., administered in 2 to 5 sub-doses.

RR. An adeno-associated virus (AAV) vector comprising a nucleic acid sequence encoding a polypeptide comprising at least amino acids 1-54 of SEQ ID NO: 1, wherein threonine at amino acid 35 of SEQ ID NO: 1 is replaced with an aspartic acid, and wherein said nucleic acid sequence is operably linked to a promoter selected from any of: a CMV promoter, a cardiac-specific promoter selected from Table 2A or a variant thereof, or a muscle-specific promoter active in cardiac and skeletal muscle selected from Table 5A, or, Table 13A, or a variant thereof.

SS. The AAV vector of paragraph RR, wherein the polypeptide is selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D).

TT. The AAV vector of paragraph RR, wherein the AAV is selected from the group consisting of adeno-associated virus-1 (AAV1), AAV2, AAV3, AAV4, AAV5, AAV6, AAV7, AAV8, AAV9 and AAV2i8.

UU. A pharmaceutical composition comprising: (i) adeno-associated virus (AAV) vector comprising a nucleic acid sequence encoding a polypeptide comprising at least amino acids 1-54 of SEQ ID NO: 1, wherein threonine at amino acid 35 of SEQ ID NO: 1 is replaced with an aspartic acid (T35D), and wherein said nucleic acid sequence is operably linked to a cardiac-specific promoter selected from Table 2A or a variant thereof, or a muscle-specific promoter active in cardiac and skeletal muscle selected from Table 5A, or, Table 13A, or a variant thereof; and (ii) a pharmaceutically acceptable carrier.

VV. The pharmaceutical composition of paragraph UU, wherein the AAV vector comprises a nucleic acid sequence encoding a polypeptide selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D).

WW. The AAV vector of paragraph UU or VV, wherein the AAV is selected from the group consisting of adeno-associated virus-1 (AAV1), AAV2, AAV3, AAV4, AAV5, AAV6, AAV7, AAV8, AAV9 and AAV2i8.

XX. The AAV vector of paragraph 49, wherein the AAV is AAV9 or AAV2i8.

All aspects of the compositions and methods of the technology disclosed herein can be defined in any one or more of the following numbered paragraphs:

1. A method of treating a patient having a heart failure, comprising:

administering into heart cells of the patient having a classification of congestive heart failure (CHF), at least one total dose of a rAAV vector comprising a nucleic acid sequence encoding a phosphatase inhibitor (I-1) protein that inhibits phosphatase activity, wherein, at least one dose of the rAAV is selected from a total dose-range of about 10¹³ vg to about 10¹⁵ vg, and wherein, at least twelve months post-administration, there is an improvement in the classification of congestive heart failure.

2. The method of paragraph 1, wherein the classification is based upon a classification system used by the American Heart Association (AHA), the American College of Cardiology (ACC), Minnesota LIVING WITH HEART FAILURE® Questionnaire (MLHFQ), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA).

3. The method of paragraph 1 or 2, wherein there is an improvement in the classification of at least one level 12 months after administration of the rAAV.

4. The method of paragraph 1 or 2, wherein there is an improvement in the classification of at least one level within six months after administration of the rAAV.

5. The method of paragraph 1, wherein twelve months post-administration, there is an improvement of at least 2 levels in the Classification.

6. The method of any of paragraphs 1-5, wherein the classification system is NYHA and the level of classification is selected from the group consisting of: Class I, Class II, Class III, and Class IV.

7. The method of any of paragraphs 1-5, wherein the classification system is the American College of Cardiology/American Heart Association (ACC/AHA) complementary staging system and the level of classification is selected from the group consisting of: Stages A, Stage B, Stage C, Stage D.

8. The method of any of paragraphs 1-5, wherein the classification system is KCCQ and the level of classification is a KQQC overall summary score range selected from the group consisting of: KCCQ fair to excellent scores of 50 to 100, very poor to fair scores of 0 to 49, good to excellent scores of 75 to 100, and very poor to good scores of 0 to 74. ***

9. A method of treating a patient having cardiomyopathy, comprising:

administering into heart cells of the patient at least one total dose of a rAAV vector comprising a nucleic acid sequence encoding a phosphatase inhibitor (I-1) protein that inhibits phosphatase activity, wherein, at least one dose of the rAAV is selected from a total dose-range of about 10¹³ vg to about 10¹⁵ vg, and wherein, at least twelve months post-administration there is an improvement in the at least one parameter from a baseline level in the patient, where the at least one parameter is selected from the group consisting essentially of: ejection fraction (EF), end systolic volume (ESV), cardiac contractility, selected from ejection fraction (EF) and fractional shortening (FS), cardiac volumes selected from any of: end diastolic volume (DV) and end systolic volume (ESV), functional criteria, selected from any of: a 6-minute walk test (6MWT), exercise and VO2max; BNP level, Pro-BNP level, biomarker level, wherein the biomarker level is selected from the group of: troponin, serum creatinine, cystatin-C, or hepatic transaminases, Patient-reported outcomes (PROs), such as reduced symptoms, health-related quality of life (HRQOL), or patient perceived health status, and decrease in any of: mortality risk due to heart failure, reduced hospitalization due to heart failure symptoms, or therapeutic intervention for treatment of heart failure.

10. The method of paragraph 9, wherein there is an improvement of at least 2 parameters at least 12 months after administration.

11. The method of paragraph 10, wherein there is an improvement of at least 3 parameters at least 12 months after administration.

12. The method of paragraph 11, wherein there is an improvement of at least 4 parameters at least 12 months after administration.

13. The method of paragraph 12, wherein there is an improvement of at least 5 parameters at least 12 months after administration.

14. The method of paragraph 9, wherein the improvement is selected from any of: at least a 5% or more increase in ejection fraction from baseline, at least a 10% decrease, or at least a 20 ml decrease in end systolic volume from baseline, at least a 50-meter increase in 6-minute walk test from baseline,at least a 40% decrease in BNP levels (pg/ml) in the blood from baseline, at least a 35% decrease in pro-BNP levels (pg/ml) in the blood from baseline, at least a 10% reduction in a biomarker selected from: troponin, serum creatinine, cystatin-C, or hepatic transaminases from a baseline level of the same biomarker, at least a 1.5 ml/kg/min increase in myocardial oxygen consumption (MVO2) from baseline,or a discharge from hospital due to improved HF symptoms, or a reduced intervention selected from a decrease in the use of any of: inotropes, vasodilators, diuretcis due to improved HF symptoms in the subject.

15. The method of paragraph 1 or 14, wherein the rAAV vector further comprises CMV promoter or a synthetic promoter, operatively linked to the phosphatase inhibitor protein.

16. The method of any of paragraphs 1-15, wherein the total dose is administered as any of the following administration methods: over a period of time of about 20 minutes to about 30 minutes, administered in a series of sub-doses, wherein each sub-dose is administered over a period of time of about 1 minute to about 5 minutes, administered in a series of five sub-doses, each sub-dose is administered over a period of time of about minute to about 5 minutes, and wherein the five sub-doses are administered over a period of about 20 minutes to about 30 minutes.

17. The method of any of paragraphs 1-16, wherein there rAAV vector comprises a capsid that detargets the liver.

18. The method of any of paragraphs 1-17, wherein the rAAV is selected from the group consisting of AAV 1, AAV2, AAV6, AAV8, AAV9, AAV2i8, rh 10, AAV2.5 and AAV2G9.

19. The method of any of paragraphs 1-18, wherein the rAAV vector is AAV2i8.

20. The method of any of paragraphs 1-19, wherein at least one total dose of the rAAV is 10¹³ vg, 3×10¹³ vg, 10¹⁴ vg, 3×10¹⁴ vg, or, 10¹⁵ vg.

21. The method of any of paragraphs 1-20, wherein the phosphatase inhibitor (I-1) protein is a constitutively active protein (I-1c).

22. The method of paragraph 21, wherein the s I-1c is selected from any of: (a) a polypeptide comprises at least amino acid residues 1-54 of SEQ ID NO: 1, wherein SEQ ID NO: 1 is truncated at the C-terminus at amino acid 70, 67, 66, 65, or 61, or 54, and where the there is an aspartic acid at position 35 (T35D), (b) a polypeptide comprising amino acids 1-54 of SEQ ID NO:1 or a functional fragment thereof, wherein the functional fragment has at least 85% sequence identity to amino acid residues 1-54 of SEQ ID NO: 1, or truncated at the C-terminus at amino acid 70, 67, 66, 65, or 61, or 54, and where the there is an aspartic acid at position 35 (T35D).

23. The method of any of paragraphs 1-21, wherein the rAAV genome comprises nucleic acid sequence selected from the group consisting of: SEQ ID NO: 413-441.

24. The method of paragraph 21, wherein the nucleic acid sequence encoding the I-1 polypeptide is selected from: (a0 a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

25. The method of any of paragraphs 1-24, wherein the nucleic acid sequence encoding the I-1 protein is a codon optimized nucleic acid sequence.

26. The method of any of paragraphs 1-25, wherein the nucleic acid sequence encoding the I-1 protein is selected from any of SEQ ID NO: 385-412, or a nucleic acid sequence having at least 80% sequence identity to SEQ ID NO: 385-412.

27. The method of any of paragraphs 1-26, wherein the subject with cardiomyopathy has non-ischemic heart failure and/or non-ischemic cardiomyopathy.

28. The method of any of paragraphs 1-27, wherein the subject with cardiomyopathy has a congenital cardiomyopathy or a genetic disorder with a cardiac manifestation.

29. The method of paragraph 29, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

30. The method of any of paragraphs 1-29, wherein the subject with cardiomyopathy has an ischemic cardiomyopathy.

31. The method of any of paragraphs 1-30, wherein the subject with cardiomyopathy has heart failure.

32. The method of paragraph 31, wherein the subject with heart failure has a classification that is equivalent to class III or above in the New York Heart Association (NYHA) classification system.

33. The method of any of paragraphs 30-32, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

34. The method of any of paragraphs 1-33, wherein the subject with cardiomyopathy has reduced ejection fraction (rEF or HFrEF), or, preserved ejection fraction (HFpEF).

35. The method of any of paragraphs 1-34, wherein at least twelve months post-administration of the rAAV there is an improvement of at least one class in a classification of heart failure from a baseline level, wherein the classification of heart failure is assessed by at least one of the following: (a) a classification system used by the American Heart Association (AHA), the American College of Cardiology (ACC), the 2016 European Society of Cardiology guidelines (ESCG), the Japanese heart failure Society (JHFS) guidelines, The Japanese Circulation Society (JCS) Guidelines, or, the New York Heart Association (NYHA); or equivalent thereof, or (b) a health-related quality of life (HRQL) questionnaire selected from the group consisting from any of: Minnesota LIVING WITH HEART FAILURE® Questionnaire (MLHFQ), or Kansas City Cardiomyopathy questionnaire (KCCQ), Chronic Heart Failure Questionnaire (CHFQ), Quality of Life Questionnaire for Severe Heart Failure (QLQ-SHF), Left Ventricular Dysfunction (LVD-36) questionnaire, and the Left Ventricular Disease Questionnaire (LVDQ).

36. The method of paragraph 35, wherein there is an improvement in the classification of at least one level within six months after administration of the rAAV.

37. The method of paragraph 35, wherein there is an improvement in the classification of at least two levels within twelve months after administration of the rAAV.

38. The method of paragraph 35, wherein there is an improvement of at least a 10 point decrease in quality of life MLWHFQ or KCCQ from the baseline level.

39. The method of any of paragraphs 1-38, wherein the subject is administered a vasodilator concurrent with and/or, before, and/or, after the administration of the at least one total dose of a rAAV vector.

40. The method of any of paragraphs 1-39, wherein the subject is administered an immune modulator concurrent with, or before, or after the administration of the at least one total dose of a rAAV vector.

41. A pharmaceutical composition comprising an AAV vector that comprises a codon optimized I-Ic nucleic sequence selected from any of SEQ ID NO: 385-412, or nucleic acid sequence having at least 80% sequence identity to SEQ ID NOS: 385-412.

42. The pharmaceutical composition of paragraph 41, wherein the codon optimized nucleic acid sequence is operably linked to a CMV promoter or a synthetic promoter.

43. The pharmaceutical composition of paragraph 41, comprising a nucleic acid sequence selected from the group consisting of: SEQ ID NO: 41-42, or a nucleic acid sequence having at least 80% sequence identity to SEQ ID NOS: 385-412.

44. The pharmaceutical composition of any of paragraphs 41-43, for the use in a method according to any of paragraphs 1-40.

45. An adeno-associated virus (AAV) vector comprising a nucleic acid sequence encoding a Phosphatase inhibitor (I-1) polypeptide operably linked to a promoter selected from any of a cardiac-specific promoter selected from Table 2A or a variant thereof, a muscle-specific promoter active in cardiac and skeletal muscle, or a variant thereof, or any promoter when a cardiac tissue specific enhancer is present.

46. The AAV vector of paragraph 45, wherein muscle-specific promoter active in cardiac and skeletal muscle is selected from Table 5A or Table 13A or a variant thereof.

47. The AAV vector of any of paragraphs 45-46, wherein the AAV is selected from the group consisting of adeno-associated virus-1 (AAV1), AAV2, AAV3, AAV4, AAV5, AAV6, AAV7, AAV8, AAV9 and AAV2i8.

48. The AAV vector of any of paragraphs 45-47, wherein the AAV comprises a capsid that detargets the liver.

49. The AAV vector of any of paragraph 45-48, wherein the AAV is AAV2i8.

50. The AAV vector of any of paragraphs 45-49, wherein the phosphatase inhibitor (I-1) polypeptide is a constitutively active protein (I-1c).

51. The AAV vector of any of paragraphs 45-50, wherein the I-1c is selected from any of: (a) a polypeptide comprises at least amino acid residues 1-65 of SEQ ID NO: 1 or a functional equivalent thereof, (b) a polypeptide comprising at least amino acids 1-54 of SEQ ID NO: 1, wherein the polypeptide is truncated at a C-terminus at amino acid selected from residue 70, 67, 66, 65, or 61, or 54, and where the there is an aspartic acid at position 35 (T35D), (c) a polypeptide comprising amino acids 1-65 of SEQ ID NO:1 or a functional equivalent thereof having at least 85% sequence identity to amino acid residues 1-65 of SEQ ID NO: 1, or, (d) a polypeptide selected from any of: SEQ ID NOS: 507 or 527-532 or a functional equivalent thereof having at least 85% sequence identity to amino acid residues of any of SEQ ID NOS: 507 or 527-532.

52. The AAV vector of any of paragraphs 45-51, wherein the nucleic acid sequence encoding a I-1 polypeptide is selected from: (a) a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

53. The AAV vector of any of paragraphs 45-52, wherein the polypeptide is selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D), or a conservative amino acid of aspartate.

54. The AAV vector of any of paragraphs 45-50, wherein the nucleic acid sequence encoding the I-1 polypeptide is a codon optimized nucleic acid sequence.

55. The AAV vector of any of paragraphs 45-54, wherein the codon optimized nucleic acid sequence has reduced CpG content or reduced CpG islands as compared to the wild-type reference sequence of a SEQ ID NO: 1, or a fragment thereof.

56. The AAV vector of any of paragraphs 45-54, wherein the nucleic acid sequence encoding the I-1 polypeptide is a codon optimized nucleic acid sequence selected from any of: SEQ ID NO: 385-412 or a nucleic acid sequence at least 80% sequence identity to SEQ ID NO: 385-412

57. The AAV vector of any of paragraph 45-56, further comprising at least one ITR located 5′ of the nucleic acid sequence encoding a Phosphatase inhibitor (I-1) polypeptide operably linked to the cardiac-specific promoter or muscle-specific promoter.

58. The AAV vector of any of paragraph 45-57, further comprising at least two ITRs flanking the nucleic acid sequence encoding a Phosphatase inhibitor (I-1) polypeptide operably linked to the cardiac-specific promoter or muscle-specific promoter.

59. The AAV vector of any of paragraph 45-58, wherein the ITR sequences are selected from any one or more of: SEQ ID NO: 70-78, or a nucleic acid having at least 85% sequence identity to SEQ ID NO: 70-78.

60. The AAV vector of any of paragraphs 45-59, further comprising a reverse poly A sequence or double stranded RNA termination element, wherein the reverse polyA sequence or double stranded termination element are located 3′ of the nucleic acid sequence encoding a Phosphatase inhibitor (I-1) polypeptide.

61. The AAV vector of paragraph 60, wherein the reverse poly A sequence, or double stranded RNA termination element is located between 3′ of the nucleic acid sequence encoding a Phosphatase inhibitor (I-1) polypeptide and 5′ of the right ITR.

62. The AAV vector of any of paragraphs 45-61, wherein the nucleic acid sequence can further comprise a nucleic acid sequence encoding at least one immune modulator.

63. The AAV vector of any of paragraphs 45-61, present in a composition or solution, further comprising an immune modulator.

64. The AAV vector of any of paragraphs 45-63, further comprising a polyA sequence selected from any of SV40 polyA (SEQ ID NO: 334), HGH poly A (SEQ ID NO: 66), SEQ ID NO: 284-287, SEQ ID NO 331-335, wherein the polyA sequence is located 3′ of the nucleic acid sequence encoding a Phosphatase inhibitor (I-1) polypeptide.

65. A pharmaceutical composition comprising: (i) adeno-associated virus (AAV) vector comprising : (i) a nucleic acid sequence encoding a Phosphatase inhibitor (I-1) polypeptide operably linked to any one of: (a) a cardiac-specific promoter selected from Table 2A or a variant thereof, (b) a muscle-specific promoter active in cardiac and skeletal muscle, or (c) any promoter when a cardiac tissue specific enhancer is present, or a variant thereof; and (ii) a pharmaceutically acceptable carrier.

66. The pharmaceutical composition of paragraph 65, wherein muscle-specific promoter active in cardiac and skeletal muscle is selected from Table 5A or Table 13A or a variant having at least 85% sequence identity thereof.

67. The pharmaceutical composition of paragraphs 65-66, wherein the AAV is selected from the group consisting of adeno-associated virus-1 (AAV1), AAV2, AAV3, AAV4, AAV5, AAV6, AAV7, AAV8, AAV9 and AAV2i8.

68. The pharmaceutical composition of paragraphs 65-67, wherein the AAV comprises a capsid that detargets the liver.

69. The pharmaceutical composition of any of paragraphs 65-68, wherein the AAV is AAV2i8.

70. The pharmaceutical composition of any of paragraphs 65-69, wherein the AAV comprises a nucleic acid selected from the group consisting of SEQ ID NO: 413-440, or a nucleic acid sequence at least 80% sequence identity to a sequence selected from SEQ ID NO: 413-440, wherein the nucleic acids seet forth in SEQ ID NO: 413-440 comprise a CMV promoter of SEQ ID NO: 330, wherein the CMV promoter of SEQ ID NO: 330 is replaced by any of: (a) a cardiac-specific promoter selected from Table 2A or a variant having at least 85% sequence identity thereof, (b) a muscle-specific promoter active in cardiac and skeletal muscle (e.g., a muscle-specific promoter active in cardiac and skeletal muscle is selected from Table 5A or Table 13A or a variant having at least 85% sequence identity thereof), or (c) any promoter when a cardiac tissue specific enhancer is present, or a variant thereof 71. The pharmaceutical composition of any of paragraphs 64-69, further comprises a vasodilator.

72. The pharmaceutical composition of any of paragraphs 64-69, further comprises an immune modulator.

73. The pharmaceutical composition of paragraph 65, wherein the phosphatase inhibitor (I-1) polypeptide is a constitutively active protein (I-1c).

74. The pharmaceutical composition of paragraph 73, wherein the I-1c is selected from any of: (a) a polypeptide comprises at least amino acid residues 1-65 of SEQ ID NO: 1 or a functional equivalent thereof; (b) a polypeptide comprising at least amino acids 1-54 of SEQ ID NO: 1, wherein the polypeptide is truncated at the C-terminus at amino acid 70, 67, 66, 65, or 61, or 54, and where the there is an aspartic acid at position 35 (T35D), (c) a polypeptide comprising amino acids 1-65 of SEQ ID NO:1 or a functional equivalent thereof having at least 85% sequence identity to amino acid residues 1-65 of SEQ ID NO: 1, or (d) a polypeptide selected from any of: SEQ ID NOS: 507 or 527-532 or a functional equivalent thereof having at least 85% sequence identity to amino acid residues of any of SEQ ID NOS: 507 or 527-532.

75. The pharmaceutical composition of any of paragraphs 65-74, wherein the nucleic acid sequence encoding a I-1 polypeptide is selected from: (a) a nucleic acid sequence encoding a polypeptide comprising amino acids 1-65 of SEQ ID NO: 1, wherein threonine (T) at position 35 of SEQ ID NO: 1 is replaced with an amino acid that is not T, (b) a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q), (c) a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

76. The pharmaceutical composition of any of paragraphs 65-745, wherein the nucleic acid sequence encoding the I-1 protein is a codon optimized nucleic acid sequence.

77. The pharmaceutical composition of paragraph 76, wherein the codon optimized nucleic acid sequence has reduced CpG content as compared to a reference wild type sequence.

78. The pharmaceutical composition of any of paragraphs 65-77, wherein the codon optimized nucleic acid sequence encoding the I-1 polypeptide is selected from any of SEQ ID NO: 385-412, or a nucleic acid sequence having at least 80% sequence identity to a sequence selected from any of SEQ ID NOS: 385-412.

79. Use of an AAV vector according to any one of paragraphs 45-64, for the manufacturer of a pharmaceutical composition for the treatment of a subject having cardiomyopathy.

80. The use of the AAV vector of paragraph 79, wherein the subject with cardiomyopathy has non-ischemic heart failure and/or non-ischemic cardiomyopathy.

81. The use of the AAV vector of any of paragraphs 79-80, wherein the subject with cardiomyopathy has a congenital cardiomyopathy or a genetic disorder with a cardiac manifestation.

82. The use of the AAV vector of paragraph 81, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM 1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

83. The use of the AAV vector of paragraph 79, wherein the subject with cardiomyopathy has an ischemic cardiomyopathy.

84. The use of the AAV vector of paragraph 79, wherein the subject with cardiomyopathy has heart failure.

85. The use of the AAV vector of paragraph 84, wherein the subject with heart failure has a classification of heart failure based upon a classification system used by the American Heart Association (AHA), the American College of Cardiology (ACC) or the New York Heart Association (NYHA).

86. The use of the AAV vector of paragraph 85, wherein the subject with heart failure has a classification of a class III or above class III in the New York Heart Association (NYHA) classification system.

87. Use of an AAV vector according to any one of paragraphs 45-64, for the manufacturer of a pharmaceutical composition for the treatment of a subject having a condition or disease associated with heart failure.

88. The use of paragraph 87, wherein the subject has a classification of congestive heart failure (CHF).

89. The use of paragraph 87, wherein the classification is based upon a classification system used by the American Heart Association (AHA), the American College of Cardiology (ACC) or the New York Heart Association (NYHA).

90. The use of paragraph 87, wherein the subject has non-ischemic heart failure or non-ischemic cardiomyopathy.

91. The use of paragraph 87, wherein the subject has ischemic heart failure or ischemic cardiomyopathy.

92. The use of any of paragraphs 79 or 87, wherein the subject has reduced ejection fraction (rEF or HFrEF).

93. A cell comprising the AAV vector of any of paragraphs 45-64.

94. The cell of paragraph 93, wherein the cell is a cardiac cell or muscle cells.

95. The cell of any of paragraphs 93-94, wherein the cell is in cell culture or a cell present in a subject.

96. An AAV vector according to paragraphs 45-64, a pharmaceutical formulation of any of paragraphs 65-78, or a cell according any of paragraphs 93-95 for use in the treatment of a subject having cardiomyopathy.

97. The AAV vector of paragraph 96, wherein the subject with cardiomyopathy has non-ischemic heart failure and/or non-ischemic cardiomyopathy.

98. The AAV vector of paragraph 96, wherein the subject with cardiomyopathy has a congenital cardiomyopathy or a genetic disorder with a cardiac manifestation.

99. The AAV vector of paragraph 98, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

100. The AAV vector of paragraph 96, wherein the subject with cardiomyopathy has an ischemic cardiomyopathy.

101. The AAV vector of paragraph 96, wherein the subject with cardiomyopathy has heart failure.

102. The AAV vector of paragraph 101, wherein the subject with heart failure has a classification of heart failure based upon a classification system used by the American Heart Association (AHA), the American College of Cardiology (ACC) or the New York Heart Association (NYHA).

103. The AAV vector of paragraph 102, wherein the subject with heart failure has a classification of a class III or above class III in the New York Heart Association (NYHA) classification system.

104. An AAV vector according to paragraphs 45-64, a pharmaceutical formulation of any of paragraphs 65-78, or a cell according any of paragraphs 93-95 for use in the treatment of a patient having heart failure.

105. The AAV vector of paragraph 104, wherein the subject has a classification of congestive heart failure (CHF).

106. The AAV vector of paragraph 105, wherein the classification is based upon a classification system used by the American Heart Association (AHA), the American College of Cardiology (ACC) or the New York Heart Association (NYHA).

107. The AAV vector of paragraph 104, wherein the subject has non-ischemic heart failure or non-ischemic cardiomyopathy.

108. The AAV vector of paragraph 104, wherein the subject has ischemic heart failure or ischemic cardiomyopathy.

109. The AAV vector of paragraph 96 or 104, wherein the subject has reduced ejection fraction (rEF or HFrEF).

110. The AAV vector of paragraph 104, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

111. The AAV vector of paragraph 104, wherein the subject has one or more of: (a) non-ischemic heart failure; (b) non-ischemic cardiomyopathy, (c) a classification of congestive heart failure (CHF) is based upon a classification system used by the American Heart Association (AH), the American College of Cardiology (ACC) or the New York Heart Association (NYHA) or an equivalent classification system thereof, or (d) a reduced ejection fraction (rEFor HFrEF).

112. A method of expressing a phosphatase inhibitor (I-1) polypeptide in a subject with cardiomyopathy, the method comprising introducing into the subject with cardiomyopathy, at least one dose of the AAV vector according to any of paragraphs 45-63, wherein the subject with cardiomyopathy has a classification of heart failure, wherein the at least one dose of the rAAV is selected from a total dose-range of about 10¹³ vg to about 10¹⁵ vg, and wherein at least twelve months post-administration there is an improvement in the classification of heart failure.

113. The method of paragraph 112, wherein the classification of heart failure is based upon a classification system used by the American Heart Association (AH), the American College of Cardiology (ACC) or the New York Heart Association (NYHA) or an equivalent classification system thereof.

114. The method of paragraph 112, wherein there is an improvement of classification of at least one level 12 months after administration of the rAAV.

115. The method of paragraph 112, wherein there is an improvement of classification of at least one level within six months after administration of the rAAV.

116. The method of paragraph 112, wherein twelve months post-administration there is an improvement of at least 2 levels in the classifications in any one or more of: the American Heart Association (AH), the American College of Cardiology (ACC), or the New York Heart Association (NYHA), or an equivalent classification system thereof.

117. The method of any of paragraphs 112-116, further comprising administering an immune modulator concurrent with, or before, or after the administration of the at least one total dose of a rAAV vector.

118. The method of any of paragraphs 112-116, further comprising administering a vasodilator concurrent with, and/or before, and/or after the administration of the at least one total dose of a rAAV vector.

119. The method of any of paragraphs 112-118, wherein the subject has non-ischemic heart failure or non-ischemic cardiomyopathy.

120. The method of any of paragraphs 112-119, wherein the subject with non-ischemic heart failure or non-ischemic cardiomyopathy is has a congenital cardiomyopathy or a genetic disorder with a cardiac manifestation.

121. The method of paragraph 120, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

122. The method of any of paragraphs 111-117, wherein the subject with heart failure is an ischemic cardiomyopathy.

123. The method of any of paragraphs 111-120, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

124. The method of any of paragraphs 111-122, wherein the subject has reduced ejaculation fraction (rEFor HFrEF).

125. The method of any of paragraphs 111-123, wherein the heart failure comprises ischemia, arrhythmia, myocardial infarction, abnormal heart contractibility, or abnormal Ca2+ metabolism.

126. The method of any of paragraphs 111-124, wherein the administration is into the lumen of the coronary artery of the heart of the patient.

127. The method of any of paragraphs 111-125, wherein the at least one dose is a total dose-range of about 10¹³ vg to about 10¹⁵ vg., administered in one dose or 2 to 5 sub-doses.

128. The method of any of paragraphs 111-126, wherein the total dose is administered as any of the following administration methods: (a) over a period of time of about 20 minutes to about 30 minutes, (b) administered in a series of sub-doses, wherein each sub-dose is administered over a period of time of about 1 minute to about 5 minutes, (c) administered in a series of five sub-doses, each sub-dose is administered over a period of time of about1 minute to about 5 minutes, and wherein the five sub-doses are administered over a period of about 20 minutes to about 30 minutes.

### Definitions and General Points

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols.154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

The term “muscle” is well understood by the skilled person. Preferably, the muscle is a skeletal muscle (including the diaphragm) or a heart muscle. The promoters of the present invention can be active in skeletal muscle and/or cardiac muscle. Preferably, the muscle is a muscle of a vertebrate, more preferably of a mammal, even more preferably of a human subject. Preferably, the muscle is a striated muscle.

In some cases, the promoter is heart specific. As used herein, heart-specific means that the promoter has a preference for cardiac tissue. Such a promoter can also be expressed in other tissues, including other muscles, as long as there is an overall preference for heart cells or heart muscle cells.

The term “muscle cell” or “myocyte” relates in the present to cells which are found in muscles (muscle tissue) or which are derived from muscle tissue. Muscle cells can be primary cells or a cell line (such as C2C12 or H2K cells (skeletal muscle cell line) or H9C2 cells (cardiac cell line)). The muscle cells can in in vivo (e.g. in muscle tissue) or in vitro (e.g. in cell culture). Myocytes as found in muscle tissue are typically long, tubular cells that develop from myoblasts to form muscles in a process known as myogenesis. The term muscle cells or myocytes as used herein includes myocytes from skeletal muscle and from cardiac muscle (cardiomyocytes). The promoters disclosed herein can be active in skeletal muscle cells and/or cardiac muscle cells.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are muscle-specific, cardiac muscle-specific, or skeletal muscle-specific enhancer elements (often referred to as muscle-specific, cardiac muscle-specific, or skeletal muscle-specific CREs, or muscle-specific, cardiac muscle-specific, or skeletal muscle-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 500 nucleotides or less in length, for example they may be 400, 300, 200, 175, 150, 90, 80, 70, 60 or 50 nucleotides or less in length. The CREs of the present invention are typically provided in combination with an operably linked promoter element, which can be a minimal promoter or proximal promoter; the CREs of the present invention enhance muscle-specific, cardiac muscle-specific, or skeletal muscle-specific activity of the promoter element. In any of the combinations of CREs, or functional variants thereof, disclosed herein, some or all of the recited CREs and promoter elements may suitably be positioned adjacent to one other in the promoter (i.e. without any intervening CREs or other regulatory elements). The CREs may be contiguous or non-contiguous (i.e. they can be positioned immediately adjacent to one another or they can be separated by a spacer or other sequence). The CRE’s may be in any order. In some preferred embodiments, the CREs, or functional variants thereof, are provided in the recited order and are adjacent to one another. For example, the synthetic muscle-specific regulatory nucleic acid may comprise CRE0107 immediately upstream of CRE0033, and so forth. In some embodiments it is preferred that some or all of the CREs are contiguous.

The term “cis-regulatory module” or “CRM” means a functional regulatory nucleic acid module, which usually comprises two or more CREs; in the present invention the CREs are typically cardiac-specific enhancers, e.g., cardiac muscle-specific or skeletal muscle-specific enhancers, and thus the CRM is a synthetic cardiac-specific regulatory nucleic acid. A CRM may comprise a plurality of cardiac-specific CREs. Suitably, at least one of the CREs comprised in the CRM is a CRE according to SEQ ID NO: 19-24, 27, 28 or a functional variant thereof. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered. In the case of a tandem promoter, CRM may be used to describe the combination of promoter element and one or more CREs which are operably linked to a further promoter element. For example, in tandem promoter SP0268, the combination of CRE CRE0035 and promoter element CRE0010 may be considered a CRM.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art. In some cases, the term “promoter” or “composite promoter” is used herein to refer to a combination of a promoter and additional regulatory elements, e.g. regulatory sequences located immediately downstream of the transcription start site (TSS), for example a 5′ UTR and or a 5′UTR and an intron. Such sequences downstream of the TSS can contribute to regulation of expression at the transcriptional and/or translational stages. In some cases, the term “promoter” or “composite promoter” is used herein to refer to a ‘tandem promoter’ as defined elsewhere herein.

The term “synthetic promoter” as used herein relates to a promoter that does not occur in nature. In the present context it typically comprises a CRE and/or CRM of the present invention operably linked to a minimal (or core) promoter or Cardiac-specific proximal promoter (promoter element). The CREs and/or CRMs of the present invention serve to enhance Cardiac-specific transcription of a gene operably linked to the synthetic promoter. Parts of the synthetic promoter may be naturally occurring (e.g. the minimal promoter or one or more CREs in the promoter), but the synthetic promoter as an entity is not naturally occurring. Alternatively, the synthetic promoter may be a shorter, truncated version of a promoter which occurs in nature.

As used herein, “minimal promoter” (also known as the “core promoter”) refers to a typically short DNA segment which is inactive or largely inactive by itself, but can mediate transcription when combined with other transcription regulatory elements. Minimal promoter sequences can be derived from various different sources, including prokaryotic and eukaryotic genes. Examples of minimal promoters include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus at least some additional regulatory sequence, typically the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a shorter, truncated version of naturally occurring Cardiac-specific proximal promoter. The proximal promoters of the present invention may be combined with one or more CREs or CRMs of the present invention. However, the proximal promoter can also be synthetic.

As used herein, “promoter element” refers to either a minimal promoter or proximal promoter as defined above. In the context of the present invention a promoter element may be combined with one or more CREs in order to provide a synthetic cardiac-specific promoter of the present invention.

A “functional variant” of a CRE, CRM, promoter element, promoter or other regulatory nucleic acid in the context of the present invention is a variant of a reference sequence that retains the ability to function in the same way as the reference sequence, e.g. as a Cardiac-specific CRE, Cardiac-specific CRM or cardiac-specific promoter. Alternative terms for such functional variants include “biological equivalents” or “equivalents”.

It will be appreciated that the ability of a given CRE, CRM, promoter or other regulatory sequence to function as a Cardiac-specific enhancer is determined significantly by the ability of the sequence to bind the same Cardiac-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE, CRM or promoter is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“Muscle-specific” or “Muscle-specific expression” refers to the ability of a cis-regulatory element, cis-regulatory module or promoter to enhance or drive expression of a gene in the muscle (or in muscle-derived cells) in a preferential or predominant manner as compared to other tissues (e.g. spleen, liver,lung, and brain). Expression of the gene can be in the form of mRNA or protein. In preferred embodiments, muscle-specific expression is such that there is negligible expression in other (i.e. non-muscle) tissues or cells, i.e. expression is highly muscle-specific.

“Cardiac muscle-specific” or “Cardiac muscle-specific expression” refers to the ability of a cis-regulatory element, cis-regulatory module or promoter to enhance or drive expression of a gene in the cardiac muscle in a preferential or predominant manner as compared to other tissues (e.g. spleen, liver, lung, and brain) and compared to the skeletal muscle tissue. Cardiac-specificity can be identified wherein the expression of a gene (e.g. a therapeutic or reporter gene) occurs preferentially or predominantly in muscle cells, including smooth muscle cells in the heart as well as cardiomyocytes. Preferential or predominant expression can be defined, for example, where the level of expression is significantly greater in cardiac-derived cells than in other types of cells (i.e. non-cardiac-derived cells). For example, expression in cardiac-derived cells is suitably at least 5-fold higher than in non-cardiac cells, preferably at least 10-fold higher than in non-cardiac cells, and it may be 50-fold higher or more in some cases. For convenience, muscle-specific expression can suitably be demonstrated via a comparison of expression levels in a hepatic muscle cell line (e.g. muscle-derived cell line such as C2C12 or H2K cells (skeletal muscle) or H9C2 cells (cardiac), compared with expression levels in a liver-derived cell line (e.g. Huh7 or HepG2), kidney-derived cell line (e.g. HEK-293), a cervical tissue-derived cell line (e.g. HeLa) and/or a lung-derived cell line (e.g. A549). Cardiac muscle-specific expression can suitably be demonstrated via a comparison of expression levels in a cardiac muscle cell line (e.g. cardiac muscle derived cell line such as H9C2) or primary cardiomyocyte compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or skeletal muscle-derived cells (e.g. C2C12 or H2K). Skeletal muscle-specific expression can suitably be demonstrated via a comparison of expression levels in a skeletal muscle-derived cells (e.g. C2C12 or H2K) or primary skeletal muscle cells compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or cardiac muscle cell line (e.g. H9C2).

The synthetic muscle-specific, cardiac muscle-specific or skeletal muscle-specific promoters of the present invention preferably exhibit reduced expression in non-muscle-derived cells, suitably in Huh7, HEK-293, HeLa, and/or A549 cells when compared to a non-tissue specific promoter such as CMV-IE. The synthetic muscle-specific, cardiac muscle-specific or skeletal muscle-specific promoters of the present invention preferably have an activity of 50% or less than the CMV-IE promoter in non-muscle-derived cells (suitably in Huh7, HEK-293, HeLa, and/or A549 cells), suitably 25% or less, 20% or less, 15% or less, 10% or less, 5% or less or 1% or less. Generally, it is preferred that expression in non-muscle-derived cells is minimized, but in some cases this may not be necessary. Even if a synthetic promoter of the present invention has higher expression in, e.g., one or two non-muscle cells, as long as it generally has higher expression overall in a range of muscle cells versus non-muscle cell, it can still a muscle-specific promoter. In some embodiments, a muscle-specific promoter expresses a gene at least 25%, or at least 35%, or at least 45%, or at least 55%, or at least 65%, or at least 75%, or at least 80%, or at least 85%, or at least 90%, or at least 95%, or any integer between 25%-95% higher in muscle cells as compared to non-muscle cells.

The synthetic muscle-specific promoters of the present invention are preferably suitable for promoting expression in the muscle of a subject, e.g. driving muscle-specific expression of a transgene, preferably a therapeutic transgene. The synthetic cardiac muscle-specific promoters of the present invention are preferably suitable for promoting expression in the heart of a subject, e.g. driving cardiac muscle-specific expression of a transgene, preferably a therapeutic transgene. The synthetic skeletal muscle-specific promoters of the present invention are preferably suitable for promoting expression in the skeletal muscles of a subject, e.g. driving skeletal muscle-specific expression of a transgene, preferably a therapeutic transgene. Preferred synthetic muscle-specific promoters of the present invention are suitable for promoting muscle-specific transgene expression and have an activity in muscle cells which is at least 15%, 20%, 30%, 40%, 50%, 60%, 70%, 80%, 90%, 100%, 125%, 150%, 175%, 200%, 250%, 300%, 350% or 400% of the activity of the CBA promoter. In some embodiments, the synthetic muscle-specific promoters of the invention are suitable for promoting muscle-specific transgene expression at a level at least 100% of the activity of the CBA promoter, preferably 150%, 200%, 300% or 500% of the activity of the CBA or the spc5-12 promoter. In some embodiments, the synthetic cardiac muscle-specific promoters of the invention are suitable for promoting cardiac muscle-specific transgene expression at a level at least 100% of the activity of the Tnnt2 or My12 promoter, preferably 150%, 200%, 300% or 500% of the activity of the Tnnt2 or My12 promoter. In some embodiments, the synthetic skeletal muscle-specific promoters of the invention are suitable for promoting skeletal muscle-specific transgene expression at a level at least 100% of the activity of the Tnnt2 or My12 promoter, preferably 150%, 200%, 300% or 500% of the activity of the spc5-12 promoter. Such muscle-specific expression is suitably determined in muscle-derived cells, e.g. as C2C12 or H2K cells (skeletal muscle) or H9C2 cells (cardiac) or primary muscle cells (suitably primary human myocytes).

Synthetic muscle-specific, cardiac muscle-specific or skeletal muscle-specific promoters of the present invention may also be able to promote muscle-specific, cardiac muscle-specific or skeletal muscle-specific expression of a gene at a level at least 50%, 100%, 150% or 200% compared to CMV-IE in muscle-derived cells (e.g. c2c12 or H2K cells (skeletal muscle) or h9C2 cells (cardiac)).

By “enhancer element” is meant a nucleic acid sequence that, when placed in close proximity to a promoter, confers an increase in transcriptional activity relative to that obtained from the promoter in the absence of an enhancer domain. Thus, an “enhancer” includes a polynucleotide sequence that facilitates transcription of an operably linked gene or coding sequence. A number of enhancers from a variety of different sources are well known in the art. Some polynucleotides with promoter sequences (such as the commonly used CMV promoter) also have enhancer sequences.

The ability of a CRE, CRM or promoter to function as a cardiac-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive cardiac-specific expression of a gene (typically a reporter gene) is measured. Alternatively, a variant of a CRE or CRM can be substituted into a synthetic cardiac-specific promoter in place of a reference CRE or CRM, and the effects on cardiac-specific expression driven by said modified promoter can be determined and compared to the unmodified form. Similarly, the ability of a promoter to drive cardiac-specific expression can be readily assessed by the skilled person (e.g. as described in the examples below). Expression levels of a gene driven by a variant of a reference promoter can be compared to the expression levels driven by the reference promoter. In some embodiments, where cardiac-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess cardiac-specific expression enhancement can be easily constructed, and the examples set out below gives suitable methodologies.

The ability of a CRE, CRM or promoter to function as a muscle-specific or cardiac muscle-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive muscle-specific or cardiac muscle-specific expression of a gene (typically a reporter gene) is measured. Alternatively, a variant of a CRE can be substituted into a synthetic cardiac muscle-specific promoter in place of a reference CRE, and the effects on cardiac muscle-specific expression driven by said modified promoter can be determined and compared to the unmodified form. Similarly, the ability of a CRM or promoter to drive muscle-specific or cardiac muscle-specific expression can be readily assessed by the skilled person (e.g. as described in the examples below). Expression levels of a gene driven by a variant of a reference promoter can be compared to the expression levels driven by the reference sequence. In some embodiments, where cardiac muscle-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess muscle or cardiac muscle-specific expression enhancement can easily constructed.

Muscle-specificity or cardiac muscle-specificity can be identified wherein the expression of a gene (e.g. a therapeutic or reporter gene) occurs preferentially or predominantly in muscle-derived cells or cardiac muscle derived cells. Preferential or predominant expression can be defined, for example, where the level of expression is significantly greater in muscle-derived or cardiac muscle-derived cells than in other types of cells (i.e. non-muscle-derived or non-cardiac muscle-derived cells). For example, expression in muscle-derived or cardiac muscle-derived cells is suitably at least 5-fold higher than in non-muscle cells, preferably at least 10-fold higher than in non-muscle cells, and it may be 50-fold higher or more in some cases. For convenience, cardiac muscle-specific expression can suitably be demonstrated via a comparison of expression levels in a cardiac muscle cell line (e.g. cardiac muscle derived cell line such as H9C2) or primary cardiomyocyte compared with expression levels in in a liver-derived cell line (e.g. Huh7 or HepG2), a kidney-derived cell line (e.g. HEK-293), a cervical tissue-derived cell line (e.g. HeLa) and/or a lung-derived cell line (e.g. A549). Cardiac muscle-specific expression can suitably be demonstrated via a comparison of expression levels in a cardiac muscle cell line (e.g. cardiac muscle derived cell line such as H9C2) or primary cardiomyocyte compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or skeletal muscle-derived cells (e.g. C2C12 or H2K). Skeletal muscle-specific expression can suitably be demonstrated via a comparison of expression levels in a skeletal muscle-derived cells (e.g. C2C12 or H2K) or primary skeletal muscle cells compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or cardiac muscle cell line (e.g. H9C2).

The synthetic cardiac muscle-specific promoters of the present invention preferably exhibit reduced expression in non-muscle-derived cells, suitably in Huh7, HEK-293, HeLa, and/or A549 cells when compared to a non-tissue specific promoter such as CMV-IE. The synthetic cardiac muscle-specific promoters of the present invention preferably have an activity of 50% or less than the CMV-IE promoter in non-muscle-derived cells (suitably in HEK-293, HeLa, and/or A549 cells), suitably 25% or less, 20% or less, 15% or less, 10% or less, 5% or less or 1% or less. Generally, it is preferred that expression in non-muscle-derived cells is minimized, but in some cases this may not be necessary. In some embodiments, the synthetic cardiac muscle-specific promoters of the present invention are suitable for promoting gene expression at a level of at 50% or less than an LP 1 or CMV-IE promoter in non-liver-derived cells (e.g. HEK-293, HeLa, and/or A549 cells). Even if a synthetic promoter of the present invention has higher expression in, e.g., one or two non-cardiac muscle cells, as long as it generally has higher expression overall in a range of cardiac muscle cells versus non-cardiac muscle cell, it can still be considered a cardiac muscle-specific promoter.

The synthetic cardiac muscle-specific promoters of the present invention are preferably suitable for promoting expression in the heart of a subject, e.g. driving cardiac muscle-specific expression of a transgene, preferably a therapeutic transgene. The synthetic cardiac muscle-specific promoters of the present invention are preferably suitable for promoting expression in the heart of a subject, e.g. driving cardiac muscle-specific expression of a transgene, preferably a therapeutic transgene. The synthetic skeletal muscle-specific promoters of the present invention are preferably suitable for promoting expression in the skeletal muscles of a subject, e.g. driving skeletal muscle-specific expression of a transgene, preferably a therapeutic transgene. Preferred synthetic cardiac muscle-specific promoters of the present invention are suitable for promoting cardiac muscle-specific transgene expression and have an activity in cardiomyocytes which is at least 15%, 20%, 30%, 40%, 50%, 60%, 70%, 80%, 90%, 100%, 125%, 150%, 175%, 200%, 250%, 300%, 350% or 400% of the activity of the CBA promoter or spc5-12 promoter. In some embodiments, the synthetic cardiac muscle-specific promoters of the invention are suitable for promoting cardiac muscle-specific transgene expression at a level at least 50% of the activity of the CBA Tnnt2, My12, or spc5-12 promoter, preferably 100%, 150%, 200%, 300% or 500% of the activity of the CBA, Tnnt2, My12, or the spc5-12 promoter. Such cardiac muscle-specific expression is suitably determined in cardiac muscle-derived cells, e.g. in H9C2 cells or primary cardiomyocytes (suitably primary human cardiomyocytes).

Synthetic cardiac muscle-specific promoters of the present invention may also be able to promote cardiac muscle-specific expression of a gene at a level at least 50%, 100%, 150% or 200% as compared to CMV-IE in cardiac muscle-derived cells (e.g. H9C2 cardiac cells), or C12C12 or H2K cells (skeletal muscle cells).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “isolated” is meant, when referring to a nucleic acid is a nucleic acid molecule or a nucleic acid sequence devoid, in whole or part, of sequences normally associated with it in nature; or a sequence, as it exists in nature, but having heterologous sequences in association therewith; or a molecule disassociated from the chromosome.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: -3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “transcription factor binding site” (TFBS) is well known in the art. It will be apparent to the skilled person that TFBS sequences can be modified, provided that they are bound by the intended transcription factor (TF). Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:

A[CT]N{A}YR

A means that an A is always found in that position; [CT] stands for either C or T in that position; N stands for any base in that position; and {A} means any base except A is found in that position. Y represents any pyrimidine, and R indicates any purine.

The term “synthetic” in the present application means a nucleic acid molecule that does not occur in nature. Synthetic nucleic acids of the present invention are produced artificially, typically by recombinant technologies or de novo synthesis. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base.

The term “administration” as used herein refers to introduction of a foreign substance into the human or animal body. Administration can be, for example, intravenous, intraarterial or intracranial.

“Transfection” in the present application refers broadly to any process of deliberately introducing nucleic acids into cells, and covers introduction of viral and non-viral vectors, and includes or is equivalent to transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes useful nucleic acid such as an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non- structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric’ capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in PCT/US18/22725, which is incorporated herein.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence). In some embodiments, the AAV is a hybrid-AA V2ITR/AA V, as disclosed in U.S. Pat. 7,172,893, which is incorporated herein in its entirety by reference.

The term “polyploid AAV” refers to a AAV vector which is composed of capsids from two or more AAV serotypes, e.g., and can take advantages from individual serotypes for higher transduction but not in certain embodiments eliminate the tropism from the parents.

As used herein, a “phosphatase inhibitor-1 protein” or “I-1 protein” is a protein, described, for example, by GenBank Accession No. NM_006741, that regulates cardiac contractility by inhibiting the activity of Protein Phosphatase-1. In the context of the phosphatase inhibitor-1 protein or I-1 protein, the term “wild-type” refers to the nucleotide sequence of SEQ ID NO: 2 encoding Phosphotase Inhibitor Protein-1 (I-1), subunit 1A, and the polypeptide sequence of SEQ ID NO: 1, and any other nucleotide sequence that encodes an I-1 protein (having the same functional properties and binding affinities as the aforementioned polypeptide sequences), such as allelic variants.

Wild-type I-1 includes so-called “functional derivatives” of the protein. By “functional derivative” is meant a “chemical derivative,” “fragment,” “polymorph” or “variant” of the polypeptide or nucleic acid of the invention. A functional derivative retains at least a portion of the function of the protein, which permits its utility in accordance with the invention. It is well known in the art that, due to the degeneracy of the genetic code, numerous different nucleic acid sequence can code for the same amino acid sequence. It is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end, provided that its addition, deletion or substitution does not alter the amino acid sequence described herein, which is encoded by the nucleotide sequence. For example, the nucleic acid molecule of the present invention may have restriction endonuclease recognition sites added to its 5′-end and/or 3′-end.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

A “chemical derivative” of I-1 contains additional chemical moieties not normally a part of the protein. Covalent modifications of the protein or peptides may be introduced into the molecule by reacting targeted amino acid residues of the peptide with an organic derivatizing agent that is capable of reacting with selected side chains or terminal residues.

An “angiogenic protein or peptide” as used herein refers to any protein or peptide capable of promoting angiogenesis or angiogenic activity, i.e. blood vessel development.

The term “angiogenesis” alone or in combination with other agents induces angiogenesis, including but not limited to fibroblast growth factor (FGF), vascular endothelial growth factor (VEGF), Hepatocyte growth factor, angiogenin, transforming growth factor (TGF), tissue necrosis factor (TNF such as TNF-α), platelet-induced growth factor (PDGF), granulocyte colony stimulating factor (GCSF), placenta GF, IL -8, means an agent including angiopoietin such as proliferin, angiopoietin-1 and angiopoietin-2, thrombospondin, ephrin-A 1, E-selectin, leptin, heparin affinity regulatory peptide.

By “growth factor” is meant an agent that at least promotes cell growth or induces expression changes.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum. In preferred aspects of the present invention for treating a cardiovascular condition, heart disease, vectors comprising angiogenic transgenes are introduced directly into vascular conduits supplying blood to the myocardium. Such vascular conduits include the coronary arteries as well as vessels such as saphenous veins or internal mammary artery grafts.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

The term “fragment” is used to indicate a polypeptide derived from the amino acid sequence of I-1 having a length less than the full-length polypeptide from which it has been derived. Such a fragment may, for example, be produced by proteolytic cleavage of the full-length protein. Such a fragment may also be obtained recombinantly by appropriately modifying the DNA sequence encoding the proteins to delete one or more amino acids at one or more sites of the C-terminus, N-terminus, and/or within the native sequence. Such fragments retain the functional portion of the native I-1.

Another functional derivative intended to be within the scope of the present invention is a “variant” polypeptide, which either lacks one or more amino acids or contains additional or substituted amino acids relative to the native polypeptide. Such variants having added, substituted and/or additional amino acids retain the functional portion of the native I-1. A functional derivative of a protein with deleted, inserted and/or substituted amino acid residues may be prepared using standard techniques well-known to those of ordinary skill in the art (for example, using site-directed mutagenesis (Adelman et al., 1983, DNA 2:183). Alternatively, proteins with amino acid deletions, insertions and/or substitutions may be conveniently prepared by direct chemical synthesis, using methods well-known in the art.

As used herein, the term “mutant” refers to an I-1 polypeptide translated from a gene containing a genetic mutation that results in an amino acid sequence that is altered in comparison to the wild-type sequence and results in an altered function of the I-1 polypeptide.

As used herein, the term “phosphatase activity” refers to the activity of phosphatase on the commonly used model protein substrate, MyBP. Herein, Myelin Basic Protein (MyBP) is employed (labeled with 32P) as a substrate (binding partner) in measuring change in protein phosphatase activity.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, a promoter, a CRE (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position- independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer. In some embodiments, spacers may have a length of greater than 75 nucleotides, e.g., 75-100, 100-200, 200-250, 250-300, or greater than 300 nucleotides.

The term “pharmaceutically acceptable” as used herein is consistent with the art and means compatible with the other ingredients of the pharmaceutical composition and not deleterious to the recipient thereof.

The phrase a “therapeutically effective amount” and like phrases mean a dose or plasma concentration in a subject that provides the desired specific pharmacological effect, e.g. to express a therapeutic gene in cardiac tissue or the heart, and/or secretion into the plasma. It is emphasized that a therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated. A therapeutically effective amount may not always be effective in treating the conditions described herein, even though such dosage is deemed to be a therapeutically effective amount by those of skill in the art.

By the terms “treat,” “treating” or “treatment of (and grammatical variations thereof) it is meant that the severity of the subject’s condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

As used herein, the term “heart disorder” refers to a structural or functional abnormality of the heart that impairs its normal functioning. For example, the heart disorder can be heart failure, ischemia, myocardial infarction, congestive heart failure (CHF), arrhythmia, cardiomyopathy, defect in cardiac contractility, transplant rejection and the like. The term includes disorders characterized by abnormalities of contraction, abnormalities in Ca2+ metabolism, and disorders characterized by arrhythmia.

The term “Heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure. The term relates to a disease amenable to treatment and/or prevention by administration of an active compound to the heart, in particular to cardiac cells or cardiomyocytes. In some embodiments, the heart disorder is heart failure, or congestive heart failure (CHF).

The term “cardiomyopathies” refers to a group of diseases giving rise to congestive heart failure. Cardiomyopathies are a heterogeneous group of diseases of the myocardium associated with mechanical and/or electrical dysfunction that usually exhibit inappropriate ventricular hypertrophy or dilatation and are due to a variety of causes, including genetic causes.

As used herein, the term “cardiomyopathy” refers to a deterioration of function of the myocardium (i.e., heart muscle). Cardiomyopathy can be extrinsic (e.g., wherein the primary pathology resides outside of the myocardium itself, for example, caused by ischemia) or intrinsic (e.g., wherein the weakness in the heart muscle is not due to an identifiable external cause).

The term “congestive heart failure” or “CHF” is also interchangeably with “heart failure” or “chronic heart failure” is a manifestation of pathological conditions affecting the heart, and refers to the inability of the heart to pump blood at an adequate rate to meet the metabolic demands of the body.

The term “heart failure” is clinically defined as a condition in which the heart does not provide adequate blood flow to the body to meet metabolic demands. Symptoms include breathlessness, fatigue, weakness, leg swelling, and exercise intolerance. On physical examination, patients with heart failure tend to have elevations in heart and respiratory rates, rales (an indication of fluid in the lungs), edema, jugular venous distension, and, in many cases, enlarged hearts. Patients with severe heart failure suffer a high mortality; typically 50% of the patients die within two years of developing the condition. In some cases, heart failure is associated with severe coronary artery disease (“CAD”), typically resulting in myocardial infarction and either progressive chronic heart failure or an acute low output state, as described herein and in the art. In other cases, heart failure is associated with dilated cardiomyopathy without associated severe coronary artery disease. Stated differently, the term “heart failure” refers to any of a number of disorders in which the heart has a defect in its ability to pump adequately to meet the body’s needs. In many cases, heart failure is the result of one or more abnormalities at the cellular level in the various steps of excitation-contraction coupling of the cardiac cells. One such abnormality is a defect in SR function. Heart failure is most frequently due to a defect in myocardial contraction, which can occur for many reasons, the most common of which include: ischemic damage to the myocardium, excessive mechanical resistance to the outflow of blood from the heart, overloading of the cardiac chambers due to defective valve function, infection or inflammation of the myocardium, or congenitally poor myocardial contractile function. (Braunwald, E. 2001 Harrison’s Principles of Internal Medicine, 15th ed., pp1318-29).

The term “peripheral vascular disease” refers to acute or chronic dysfunction of the peripheral (i.e., non-cardiac) vasculature and/or the tissues supplied thereby. As with heart disease, peripheral vascular disease typically results from an inadequate blood flow to the tissues supplied by the vasculature, which lack of blood may result, for example, in ischemia or, in severe cases, in tissue cell death. Aspects of peripheral vascular disease include, without limitation, peripheral arterial occlusive disease (PAOD) and peripheral muscle ischemia. Frequently, symptoms of peripheral vascular disease are manifested in the extremities of the patient, especially the legs.

The term “cardiovascular conditions” include, but are not limited to, coronary artery disease / ischemia, coronary artery disease (CAD), ischemia, angina (chest pain), thrombosis, coronary thrombosis, myocardial infarction (MI), none Symptom ischemia, stenosis / restenosis, transient cerebral ischemic attack (TIA), atherosclerosis, peripheral vascular disease such as bradyarrhythmia, bradycardia, sick sinus rhythm (sinus dysfunction syndrome), Sinus bradycardia, sinoatrial block, asystole, sinus arrest, syncope, first degree atrial-ventricular (AV) block, second degree atrial-ventricular (AV) block, third degree atrial-ventricular (AV) block, abnormal Bradyarrhythmias that are dysfunctional, such as tachyarrhythmia, tachycardia, fibrillation, flutter, atrial fibrillation, atrial flutter, familial atrial fibrillation, paroxysmal atrial fibrillation, permanent atrial fibrillation, Persistent atrial fibrillation, upper ventricular tachyarrhythmia, sinus tachycardia, reentry (reentrant arrhythmia), AV connection Reentry, focal arrhythmia, translocation, ventricular fibrillation (VF), ventricular tachycardia (VT), Wolf Parkinson-White syndrome (WPW), sudden cardiac death, tachyarrhythmia such as heart failure, cardiomyopathy, congestion Heart failure, hypertrophic cardiomyopathy, remodeling, non-ischemic cardiomyopathy, diastolic cardiomyopathy, restrictive cardiomyopathy, diastolic heart failure, systolic heart failure, heart failure that is chronic heart failure, e.g. atrial ventricular (AV) blockade, bundle Branch block (BBB), left bundle branch block (LBBB), right bundle branch block (RBBB), long-term QT syndrome (LQTS), premature ventricular contraction (PVC), electrical remodeling, intraventricular conduction deficit, hemiblock Certain heart blocks / electrical disorders, such as hypertension, hypotension, left ventricular dysfunction, low ejection fraction, low cardiac output, low cardiac output hemodynamic deficiency, sudden cardiac death, cardiac arrest Suddenly heart (SCD), ventricular fibrillation, pump failure, bacterial endocarditis, viral myocarditis, pericarditis, rheumatic heart disease, there is syncope. Specifically, cardiovascular conditions include, but are not limited to, arrhythmias that are hyperplasias not associated with, for example, atrial fibrillation, ventricular fibrillation or bradycardia, ischemia, heart failure, neoplastic disease,, Ventricular remodeling, diastolic dysfunction, abnormal body temperature, such as altered vein, left ventricle, or left atrial pressure, abnormal or changing pressure, abnormal or changing heartbeat or heart sound, abnormal or changing electrogram, e.g.,changing blood pH, glucose, pO 2, pCO 2, minute ventilation, creatine, CRP, MEF2A, creatine kinase or creatine kinase MB level abnormal or altered cardiac metabolism, abnormal or altered pulmonary impedance or thoracic impedance, abnormal or altered Stroke output, abnormal or altered neurohormonal levels, abnormal or altered electrical activity, abnormal or altered Sensitive nerve activity, abnormal or altered renal output, abnormal or altered filtration rate, may be associated with such abnormal or altered angiotensin II levels, or abnormal or altered breathing sounds.

The term “Myocardial ischemia” or “MI” is a condition in which the heart muscle does not receive adequate levels of oxygen and nutrients, which is typically due to inadequate blood supply to the myocardium (e.g., as a result of coronary artery disease).

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

As used herein, the term “heart cell” refers to a cell which can be: (a) part of a heart present in a subject, (b) part of a heart which is maintained in vitro, (c) part of a heart tissue, or (d) a cell which is isolated from the heart of a subject. For example, the cell can be a muscle cell, such as a cardiac myocyte (cardiomyocyte) or smooth muscle cell. Heart cells of the invention can also include endothelial cells within the heart, for example, cells of a capillary, artery, or other vessel. A heart cell includes pacemaker cells and the like.

As used herein, the term “heart” refers to a heart present in a subject or to a heart which is maintained, ex vivo outside a subject.

As used herein, the term “heart tissue” refers to tissue which is derived from the heart of a subject.

As used herein, the term “contractility” (as in myocardial contractility) refers to the performance of cardiac muscle. It is often defined as: the intrinsic ability of a cardiac muscle fiber to contract at a given fiber length.

As used herein, the term “restricting blood flow” refers to substantially blocking the flow of blood through a vessel, e.g., flow of blood into the distal aorta and its branches. For example, at least 50% of the blood flowing out of the heart is restricted, preferably 75% and more preferably 80, 90, or 100% of the blood is restricted from flowing out of the heart. The blood flow can be restricted by obstructing the aorta and the pulmonary artery, e.g., with clamps.

The term “AAV vector” as used herein is well known in the art, and generally refers to an AAV vector nucleic acid sequence including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle. The ITRs may be derived from the same serotype as the capsid, selected from any of the serotypes listed in Table 11, or may be from a different serotype than the capsid. The two ITRs do not have to be the same. In addition, synthetic ITRs can be used. The AAV vector typically has more than one ITR. In a non-limiting example, the AAV vector has a viral genome comprising two ITRs. In one embodiment, the ITRs are of the same serotype as one another. In some embodiments, the ITRs are of different serotypes. Non-limiting examples include zero, one or both of the ITRs having the same serotype as the capsid. Independently, each ITR may be about 100 to about 150 nucleotides in length. An ITR may be about 100-105 nucleotides in length, 106-110 nucleotides in length, 111 -115 nucleotides in length, 116-120 nucleotides in length, 121 -125 nucleotides in length, 126-130 nucleotides in length, 131 -135 nucleotides in length, 136-140 nucleotides in length, 141-145 nucleotides in length or 146-150 nucleotides in length. In one embodiment, the ITRs are 140-142 nucleotides in length. Non-limiting examples of ITR length are 102, 105, 130, 140, 141, 142, 145 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome capable of modulating the productive utilization of mRNA. An artificial microRNA can be any type of RNA sequence, other than endogenous microRNA, capable of modulating the activity of an mRNA. A microRNA sequence can be an RNA molecule composed of any one or more of these sequences. MicroRNA (or “miRNA”) sequences have been described in publications such as Lim, et al, 2003, Genes & Development, 17, 991-1008, Lim et al, 2003, Science, 299, 1540, Lee and Ambrose, 2001, Science, 294, 862, Lau et al, 2001, Science 294, 858-861, Lagos -Quintana et al, 2002, Current Biology, 12, 735-739, Lagos- Quintana et al., 2001, Science, 294, 853-857, and Lagos-Quintana et al., 2003, RNA, 9, 175- 179. Examples of microRNAs include any RNA fragment of a larger RNA or is a miRNA, siRNA, stRNA, sncRNA, tncRNA, snoRNA, smRNA, shRNA, snRNA, or other small non-coding RNA. See, e.g., U.S. Pat. Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA cleaved from a microRNA precursor (a “pre-miRNA”), or synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

In some embodiments, the terms “treat” or “treatment” or “treating” refers to therapeutic treatment, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health. In some embodiments, the term to treat also encompasses preventative measures and/or prophylactic treatment, which includes administering a pharmaceutical composition as disclosed herein to prevent the onset of a disease or disorder.

The term “effective amount” as used herein refers to the amount of therapeutic agent of pharmaceutical composition, e.g., an amount of the synthetic modified RNA to express sufficient amount of the protein to reduce at least one or more symptom(s) of the disease or disorder, and relates to a sufficient amount of pharmacological composition to provide the desired effect. The phrase “therapeutically effective amount” as used herein, e.g., of a synthetic modified RNA as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to, for example, effect a therapeutically or prophylactically significant reduction in a symptom or clinical marker associated with a cardiac dysfunction or disorder when administered to a typical subject who has a cardiovascular condition, disease or disorder.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

With reference to the treatment of, for example, a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick JP, Am J Physiol Heart circ Physiol 285;H1797;2003 ) and animal models acute myocardial infarction. (Yang Z, Am J Physiol Heart Circ. Physiol 282:H949:2002; Guo Y, J Mol Cell Cardiol 33;825-830, 2001), or models of heart failure (disclosed in Mann, Douglas L., and G. Michael Felker. “Mechanisms and models in heart failure: a translational approach.” Circulation research 128.10 (2021): 1435-1450), e.g., α-actin transgenic mice (mActin-Tg mice) which is a model of cardiomyopathy.

When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a treatment of a cardiovascular disease or disorder is used to refer to the reduction of a symptom and/or a biochemical marker of a cardiovascular disease or disorder, for example a reduction in at least one biochemical marker of a cardiovascular disease by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

As used herein, the terms “having therapeutic effect” and “successful treatment” carry essentially the same meaning. In particular, a patient suffering from a cardiovascular disease, or heart disease is successfully “treated” for the condition if the patient shows observable and/or measurable reduction in or absence of one or more of the symptoms of heart disease after receiving a rAAV vector as disclosed herein according to the methods of the present invention. Reduction of these signs or symptoms may also be felt by the patient. Thus, indicators of successful treatment of heart disease conditions include the patient showing or feeling a reduction in any one of the symptoms of angina pectoris, fatigue, weakness, breathlessness, leg swelling, rales, heart or respiratory rates, edema or jugular venous distension. The patient may also show greater exercise tolerance, have a smaller heart with improved ventricular and cardiac function, and in general, require fewer hospital visits related to the heart condition. The improvement in cardiovascular function may be adequate to meet the metabolic needs of the patient and the patient may not exhibit symptoms under mild exertion or at rest. Many of these signs and symptoms are readily observable by eye and/or measurable by routine procedures familiar to a physician. Indicators of improved cardiovascular function include increased blood flow and/or contractile function in the treated tissues. As described below, blood flow in a patient can be measured by thallium imaging (as described by Braunwald in Heart Disease, 4th ed., pp. 276-311 (Saunders, Philadelphia, 1992)) or by echocardiography (described in Examples 1 and 5 and in Sahn, D J., et al., Circulation. 58:1072-1083, 1978). Blood flow before and after treatment with the rAAV vector as disclosed herein according to the administration methods disclosed herein can be compared using these methods. Improved heart function is associated with decreased signs and symptoms, as noted above. In addition to echocardiography, one can measure ejection fraction (LV) by nuclear (non-invasive) techniques as is known in the art. Blood flow and contractile function can likewise be measured in peripheral tissues treated according to the present invention.

The “administration” of an agent to a subject includes any route of introducing or delivering to a subject the agent to perform its intended function. Administration can be carried out by any suitable route, including orally, intranasally, intraocularly, ophthalmically, parenterally (intravascularly, intramuscularly, intraperitoneally, or subcutaneously), or topically. Administration includes self-administration and the administration by another. Intravenous or intraarterial administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

The term “specifically active in an area or in a tissue” refers to a promoter which is predominantly active in that area or tissue, i.e. more active in that area or tissue than in other areas or tissues.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the inhibitor of PP1.

Furthermore, the term “about,” as used herein when referring to a measurable value such as an amount of the length of a polynucleotide or polypeptide sequence, dose, time, temperature, and the like, is meant to encompass the exact specified amount and variations of ± 20%, ± 10%, ± 5%, ± 1%, ± 0.5%, or event 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

### EXAMPLES

### Materials and Methods

DNA preparations were transfected into H9C2 (a rat BDIX heart myoblast cell line, available from ATCC), C2C12 (an immortalized mouse myoblast cell line, available from ATCC), or H2K 2B4 (an immortal satellite cell-derived cell-line, see PLoS One. 2011; 6(9): e24826) to assess transcriptional activity.

### H9C2 Cell Culture and Transfection

H9C2 are a rat BDIX heart myoblast cell line (rat cardiomyocyte cells). They have skeletal muscle properties, e.g. myotubes formed at confluency respond to acetylcholine.

H9C2 Cell Maintenance: H9C2 cells were cultured in DMEM (High Glucose, D6546, Sigma) with 1% FBS (Heat inactivated -Gibco 10270-106, lot number 42G2076K), 1% Glutamax (35050-038, Gibco), 1% Penicillin-streptomycin solution (15140-122, Gibco), in T-75 flasks. Cells were passaged at a sub confluent stage (70-80%) to avoid risk of the cells becoming confluent and fusing to form myotubes.

For passaging during cell maintenance, culture media was removed, cells were washed twice with 5 ml DPBS without CaC12, without MgC12 (14190-094, Gibco). The cells were detached from the flask by incubating with 1 ml Trypsin EDTA (25200-056, Gibco) for approximately 5 minutes. Then, 4 ml of culture medium was added to the flask and the mixture was gently pipetted up and down to help detach the cells from the flask surface. Cells were pelleted at 100 g for 3 minutes. Supernatant was disposed and cells were resuspended in 3 ml of culture medium. Cells were counted on the Countess automated cell counter, seeded at 1:3 to 1:10 i.e. seeding 1-3x10,000 cells/cm2 and incubated at 37° C. 5% C02.

H9C2 Cell Transfection and differentiation: H9C2 cells were collected from two T-75 flasks of approximately 70-80% confluency, by washing with DPBS, detaching from the flask using 1 ml Trypsin EDTA, washing off the flask’s surface with 4 ml of culture medium and pelleting at 100 g for 3 minutes, as described above. Cells were resuspended in 45 ml culture medium and seed at a density of 40,000 cells/well in a 48 well flat bottom plate (300 µl/well) (353230, Corning). Cells in 48 -well plates were incubated at 37° C. 5% CO2.

Twenty-four hours later, the culture medium on the cells was replaced with 300 µl antibiotic-free culture medium (i.e DMEM (High Glucose, D6546, Sigma) with 1% FBS (Heat inactivated -Gibco 10270-106, lot number 42G2076K), 1% Glutamax (35050-038, Gibco)). 300 ng of DNA per well was transfected with viafect (E4981, Promega) in a total complex volume of 30 µl per well. Plates were gently mixed following transfection and incubated at 37° C. 5% CO2.

Twenty-four hours later, culture medium was removed from transfected cells and replaced with 300 µl differentiation media consisting of DMEM (High Glucose, D6546, Sigma), 1% Glutamax (35050-038, Gibco), 1% FBS (Heat inactivated -Gibco 10270-106, lot number 42G2076K), 1% Penicillin/streptomycin solution (15140-122, Gibco) and 0.1% Retinoid Acid (Sigma-R2625). Plates were incubated at 37° C. 5% CO2 for 7 days to induce differentiation. After differentiation, cell morphology was observed to confirm differentiation into myotubes.

Cells were then washed with 500 µl DPBS, and lysed with 100 µl Luciferase Cell Culture Lysis 5X Reagent (E1531, Promega) diluted to IX using Milli-Q water. Cell lysis reagent was pipetted up and down ten times and plates were then vortexed on a medium power for 30 minutes to promote cell lysis. Plates were sealed and stored at -80° C. prior to completing a luciferase assay. All data collected from luciferase assays following transfections in H9C2 cells is based on three technical replicates and three biological replicates.

### H2K 2B4 (H2K) Cell Culture and Transfection

H2K Cell Maintenance: H2K cells are mouse skeletal muscle cells, and were cultured in DMEM (High Glucose, D6546, Sigma) with 20% FBS (Heat inactivated -Gibco 10500-064, lot number 08Q2771K), 1% Glutamax (35050-038, Gibco), 1% Penicillin-streptomycin solution (15140-122, Gibco), 0.5% Chicken embryo extract (MD-004E-UK, LSP, lot number A20418), 0.2% Mouse IFN-γ (315-05, Peprotech, lot number 061798C2918) in T-75 flasks. Cells were passaged every 3-4 days when the cells had reached a confluency of 4 — 6.7 x 104 cells/cm2. To passage, culture media was removed, cells were washed twice with 5 ml DPBS without CaC12, without MgC12 (14190-094, Gibco) and cells were detached from the flask using 1 ml Trypsin EDTA (25200-056, Gibco). Cells were incubated with Trypsin EDTA for approximately 2 minutes, before adding 4 ml of culture medium to the flask and gently pipetting up and down to wash the cells from the flask surface. Cells were pelleted at 100 g for 3 minutes. Supernatant was disposed and cells were resuspended in 6 ml of culture medium. Cells were counted on the Countess automated cell counter, seeded at 4 densities of 4000, 2700, 2000 or 1300 cells/cm2 and incubated at 33° C. 10% CO2.

H2K Cell Transfection and Differentiation: H2K cells were collected from three T-75 flasks of approximately 20-40% confluency, by washing with DPBS, detaching from the flask using 1 ml Trypsin EDTA for approximately 2 minutes, washing off flask surface with 4 ml of culture medium and pelleting at 100 g for 3 minutes. Cells were resuspended in 45 ml culture medium at a density of 8000 cells/100 µl culture medium. 100 µl of cell suspension was then dispensed into each well of a 96-well Matrigel-coated (Corning, ref. 354234, lot. 8085009) plate using a BioFill Solo Reagent Dispenser (Brooks Automation Ltd, Catalog #34-1000). Cells in 96-well plates were incubated at 33° C. 10% CO2.

Twenty-four hours later, the culture medium on the cells was replaced with 100 µl antibiotic-free culture medium (i.e. DMEM (High Glucose, D6546, Sigma) with 20% FBS (Heat inactivated -Gibco 10500-064, lot number 08Q2771K), 1% Glutamax (35050-038, Gibco), 0.5% Chicken embryo extract (MD-OO4E-UK, LSP, lot number A20418), 0.2% Mouse IFN-γ (315-05, Peprotech, lot number 061798C2918). 150 ng of DNA per well was transfected with 0.3 µl Lipofectamine 3000 in a total complex volume of 10 µl per well. Plates were gently mixed following transfection and incubated at 33° C. 10% CO2. Twenty-four hours later, culture medium was removed from transfected cells and replaced with 200 µl differentiation media consisting of DMEM (High Glucose, D6546, Sigma), 0.1% Glutamax (35050-038, Gibco), 0.2% Horse serum (GIBCO, ref. 16050-122, lot.1671317), 0.02% Chicken embryo extract (MD-004E-UK, LSP, lot number A20418), 0.1% Penicillin/streptomycin solution (15140-122, Gibco). Plates were incubated at 37° C. 5% CO2 for 72 hours to induce differentiation. After differentiation, cell morphology was observed to confirm differentiation into myotubes. Cells were then washed with 250 µl DPBS, followed by lysis with 50 µl Luciferase Cell Culture Lysis 5X Reagent (E1531, Promega) diluted to 1X using Milli-Q water. Cell lysis reagent was pipetted up and down ten times and plates were then vortexed on a medium power for 10 minutes to promote cell lysis. Plates were sealed and stored at -80° C. prior to completing a luciferase assay.

### Luciferase Assay Preparation

96-well plates containing lysed cells were thawed at room temperature, vortexed on a medium power for 10 minutes and centrifuged for 1 minute at 2250 g. 10µl of lysate was transferred from each well into a white Microplate FluoroNunc 96 well flat bottom (Fisher Scientific, 10346331). Luciferase read-outs were generated using LAR (Promega, catalog# E4550) injections on a BMG Labtech FLUOstar Omega plate reader as described below. All data collected from luciferase assays following transfections in H2K cells is based on four technical replicates and three biological replicates.

### C2C12 Cell Culture and Transfection

C2C12 Cell maintenance: C2C12 cells were cultured in DMEM (High Glucose, D6546, Sigma) with 10% FBS (Heat inactivated -Gibco 10500-064), 1% Glutamax (35050-038, Gibco), 1% Penicillin-streptomycin solution (15140-122, Gibco) in T-75 flasks. Cells were fed every 2-3 days with fresh medium and passaged when they reached 70% confluency. To passage, culture media was removed, cells were washed twice with 5 ml DPBS without CaC12, without MgC12 (14190-094, Gibco) and cells were detached from the flask (T-75) using 1 ml Trypsin EDTA (25200-056, Gibco). Cells were incubated at 37° C. (in CO2 incubator) for 3 to 5 mins, until the cells detached as determined under the microscope. 4 ml of complete culture medium was added to the flask to inactivate Trypsin and cell suspension was transferred to a 15 ml tube. Cells were pelleted at 250 g for 3 minutes. Supernatant was disposed of and cells were resuspended in 6 ml of culture medium. Cells were counted on the Countess automated cell counter, seeded at a 1:10 dilution and incubated at 37° C. 5% CO2.

C2C12 Cell Transfection and Differentiation: C2C12 cells were collected from T-75 flasks once they reached 80% confluency by washing with DPBS, detaching from the flask using 1 ml Trypsin EDTA for approximately 3-5 minutes, washing off the flask surface with 4 ml of culture medium and pelleting at 250 g for 3 minutes. Cells were resuspended at specific densities depending on the passage number (see Table 12 for details).

TABLE 12

| | |
|---|---|
| Passages of C2C12 | SEEDING CELL DENSITY PRIOR TO TRANSFECTION (48 WELL-PLATE) |
| p.4, p.5, p.6 | 45,000 cells/300 µl media |
| p.7, p.8, p.9 | 40,000 cells/300 µl media |
| p.10, p.11, p.12 | 38,000 cells/300 µl media |

300 µl of appropriate cell suspension (based on passage number) was then dispensed into each well of a 48-well plate. Cells in 48-well plates were incubated at 37° C. 5% CO2.

Twenty-four hours later, the culture medium on the cells was replaced with 300 µl of prewarmed transfection medium containing DMEM (High Glucose, D6546, Sigma) and 1% Glutamax. 300 ng of DNA was transfected with 0.9 µl Viafect (E4981, Promega) in a total complex volume of 30 µl per well. Plates were gently mixed following transfection and incubated at 37° C. 5% CO2.

Twenty-four hours later, culture medium was removed from transfected cells and replaced with differentiation media consisting of DMEM (high glucose, no sodium pyruvate, 11960-044, Gibco), 1% Glutamax, 2% Horse Serum (Heat Inactivated, 16050-122, Gibco). Plates were incubated at 37° C. 5% CO2 for a further 5.5 days to induce differentiation. After differentiation, cell morphology was observed to confirm differentiation into myotubes. Cells were then washed with 300 µl DPBS, followed by lysis with 100 µl Luciferase Cell Culture Lysis 5X Reagent (E1531, Promega) diluted to 1X using Milli-Q water. Plates were sealed and stored at -80° C. prior to completing a luciferase assay.

C2C12 Luciferase Assay preparation: 48-well plates containing lysed cells were thawed at room temperature, vortexed on a medium power for 10 minutes and centrifuged for 1 minute, 2250xg. 10 µl of lysate was transferred from each well into a white Microplate FluoroNunc 96 well flat bottom (Fisher Scientific, 10346331). Luciferase read-outs were generated using LAR (Promega, catalog# E4550) injections on a BMG Labtech FLUOstar Omega plate reader as described below. All data collected from luciferase assays following transfections in C2C12 cells is based on three technical replicates and three biological replicates.

### Measurement of Luciferase Activity

Luciferase activity was measured using LARII (Dual Luciferase Reporter 1000 assay system, Promega, E1980). 24 h after transfection, the media was removed from the cells. The cells were washed once in 300 µl of DPBS. Cells were lysed using 100 µl of passive lysis buffer and incubated with rocking for 15 minutes. The cell debris was pelleted by centrifugation of the plate at max speed in a benchtop centrifuge for 1 min. 10 µl sample was transferred into white 96-well plate and luminescence measured by injection of 50 µl of LARII substrate on a BMG Labtech FLUOstar Omega plate reader, according to manufacturing instructions.

### Exemplary Formulation Pharmaceutical Compositions

In various aspects of the present invention, the pharmaceutical composition comprises recombinant AAV vector comprising rAAV-I1c (e.g. AAV2i8sc.CMV.I1c), in 10 mM Phosphate pH 7.4, 200 mM NaCl, 5 mM KCl, 1% (w/v) mannitol, 0.0005% (w/v) IGEPAL® CA 720 (polyoxyethylene (12) isooctylphenyl ether) to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In one aspect of the present invention, the pharmaceutical composition comprises recombinant AAV vector comprising rAAV-I1c (e.g. AAV2i8sc.CMV.I1c), in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In several aspects of the present invention, the pharmaceutical composition comprises recombinant AAV vector comprising rAAV-I1c (e.g. AAV2i8sc.CMV.I1c), in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In various aspects of the present invention, the pharmaceutical composition comprises recombinant AAV vector comprising rAAV-I1c (e.g. AAV2i8sc.CMV.I1c), in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

Several aspects of the present invention provided herein, the pharmaceutical composition comprises recombinant AAV vector comprising rAAV-I1c (e.g. AAV2i8sc.CMV.I1c), in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In one of the aspects of the present invention, the pharmaceutical composition comprises recombinant AAV vector comprising rAAV-I1c (e.g. AAV2i8sc.CMV.I1c), in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

### Example 1

In vivo assessment of muscle specific promoters: An expression cassette comprising SP0067 or the control promoters CBA, CK8 driving the luciferase gene was created and a rAAV2/9 vector comprising these expression cassettes were purified through high performance liquid chromatography (HPLC). rAAVs were diluted in 0.9% saline and delivered via tail vein into 8 8-week old male Balb/c (wild type) mice at 200 µl/mouse at a dose of 1e+12 vg/mouse. Mice were sacrificed 6 weeks after and the diaphragm (skeletal muscle), heart (cardiac muscle), intestine (skeletal muscle), kidney (specificity control tissue), liver (specificity control tissue), lung (specificity control tissue), quadriceps (skeletal muscle), spleen (specificity control tissue), and tibialis anterior (skeletal muscle) were collected, and divided into 3 parts. Samples were snap-frozen in liquid nitrogen, immediately after dissection, and stored at -80° C. The readouts for Diaphragm, Heart, Intestine, Liver, Quadriceps, and Tibialis anterior (TA) were created by protein quantification (using BCA Pierce protein assay kit; Promega 23225) and Luciferase quantification (using ONE-Glo Luciferase Assay System; Promega E6120). RLU values, were calculated as pg/mg extracted protein.

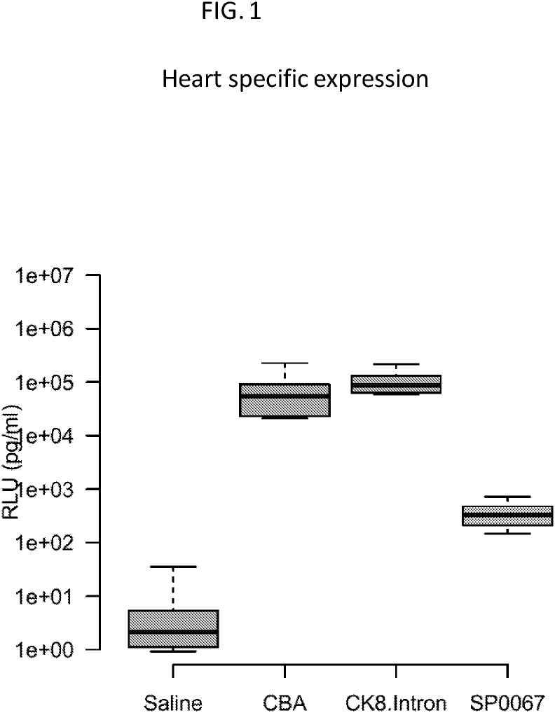

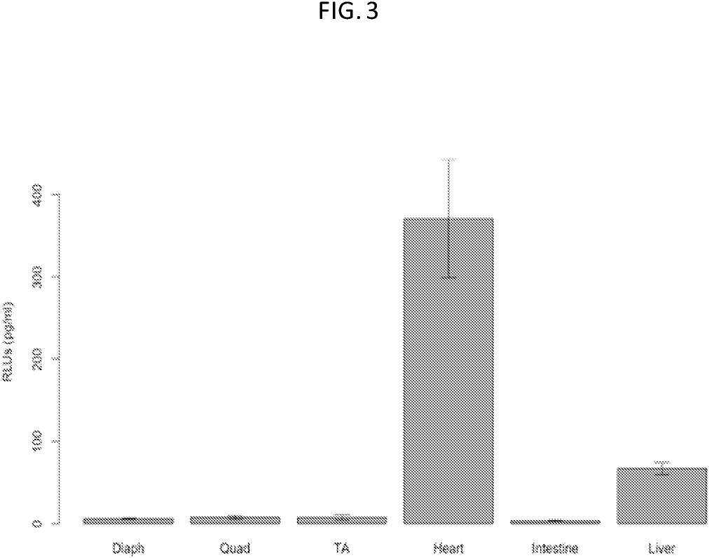

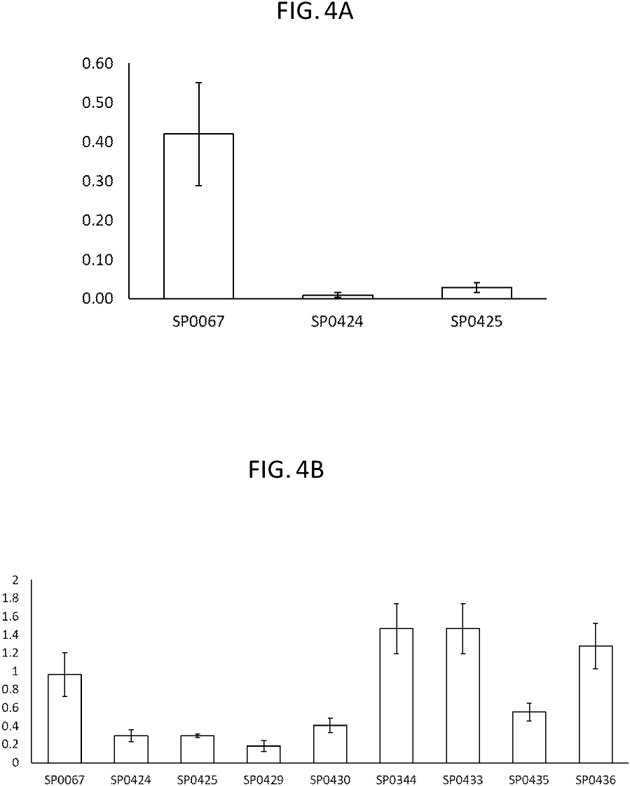

FIGS. 1-3 show that cardiac-specific promoter SP0067 active in vivo in the heart, but is not very active in vivo in skeletal muscle (diaphragm, quadriceps, tibialis anterior, intestine). While in this embodiment, SP0067 is less active in the heart muscle than the CBA and CK8.intron control promoters, unlike these generic control promoters, SP0067 is highly specific for cardiac muscle in vivo as compared to skeletal muscle. SP0067 also has some activity in vivo in liver. FIGS. 4A-4B show in vitro expression of luciferase from rAAV2/9 operatively linked to SP0067 and other synthetic cardiac-specific promoters in cardiac myotubes and skeletal muscle myotubes in vitro.

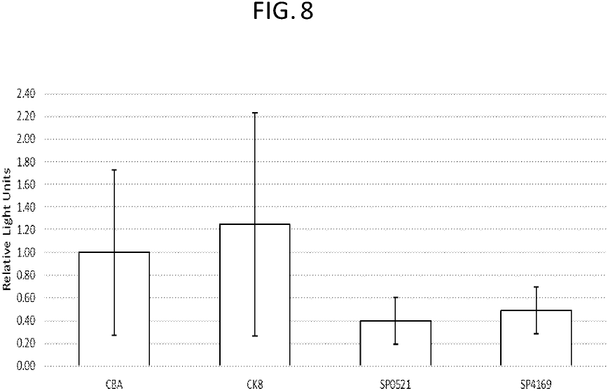

H9C2 cells were used to assess the in vitro activity of shortened nucleic acid sequences of exemplary muscle-specific promoters active in cardiac and skeletal muscle. FIG. 8 shows the in vitro activity of synthetic short muscle specific promoters SP0521 and SP4169 in the muscle cell line H9C2 which is differentiated into heart myotubes, as compared to CBA and CK8 control promoters. FIG. 8 shows that synthetic promoters SP0521 and SP4169 show good activity in the muscle cell line H9C2. Additional muscle-specific promoters active in cardiac and skeletal muscle SP0502, SP0515, SP0522, SP0523 and SP524 were also tested experimentally in the H9C2 cell line but showed lower activity (data not shown).

### Example 2

### In Vivo Testing

Expression cassettes comprising each of SP0173, SP0270, SP0268, SP0320, SP0134, SP0279, SP0057, SP0229, SP0310, SP0067, and SP0267, or the control promoters CBA and CK8 driving the luciferase gene were created and AAV2/9 comprising these expression cassettes were purified through high performance liquid chromatography (HPLC). AAVs were diluted in 0.9% saline and delivered via tail vein into 8 8-week old male Balb/c (wild type) mice at 200 µl/mouse at a dose of le+ 12 vg/mouse. Mice were sacrificed 6 weeks after, and the diaphragm (skeletal muscle), heart (cardiac muscle), intestine (skeletal muscle), kidney (specificity control tissue), liver (specificity control tissue), lung (specificity control tissue), quadriceps (skeletal muscle), spleen (specificity control tissue), and tibialis anterior (skeletal muscle) were collected, and divided into 3 parts. Samples were snap-frozen in liquid nitrogen, immediately after dissection, and stored at -80° C. The readouts for Diaphragm, Heart, Intestine, Liver, Quadriceps, and Tibialis anterior were created by protein quantification (using BCA Pierce protein assay kit; Promega 23225) and Luciferase quantification (using ONE-Glo Luciferase Assay System; Promega E6120). RLU values were calculated as pg/ml.

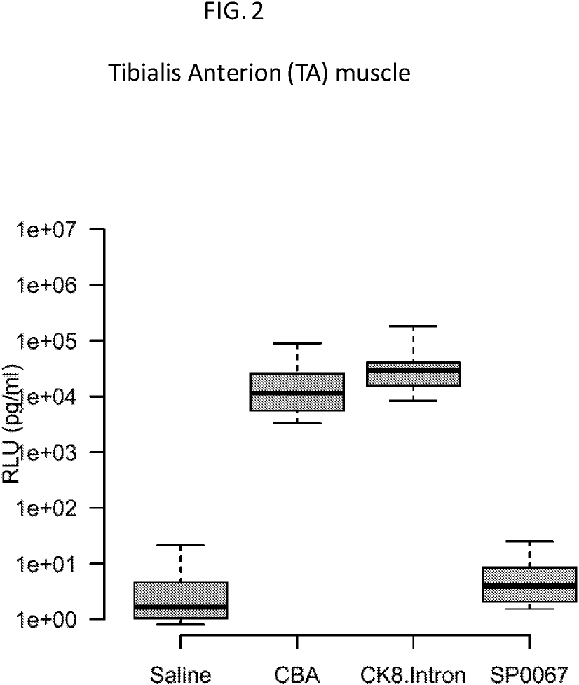

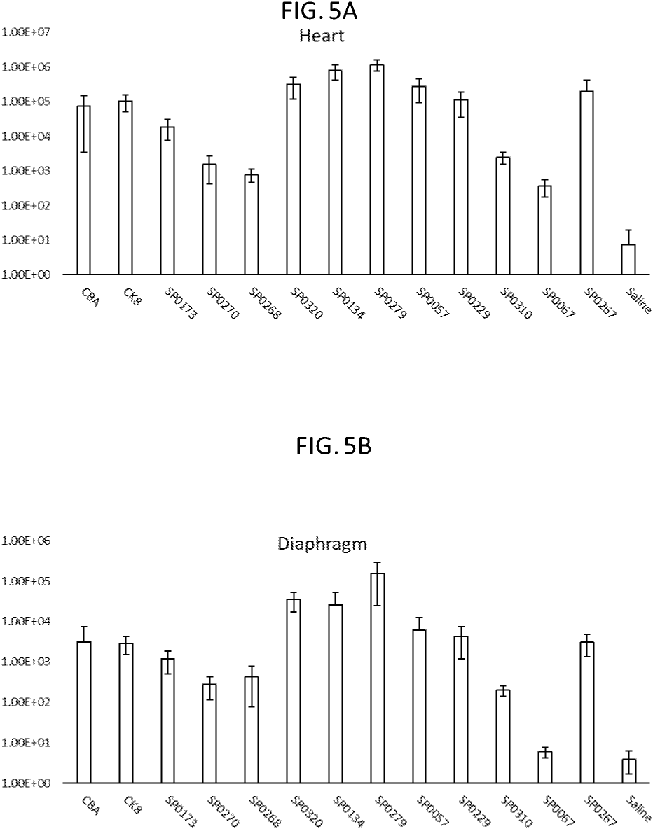

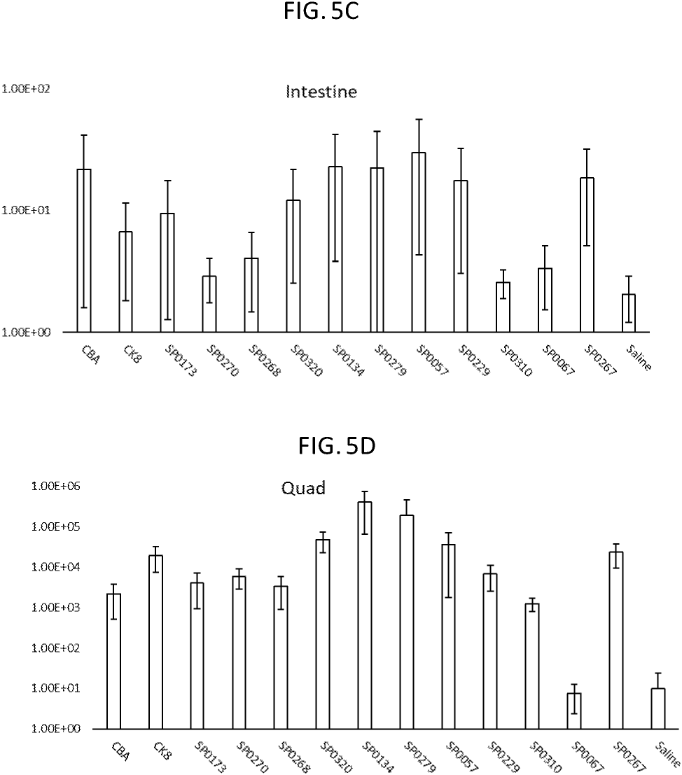

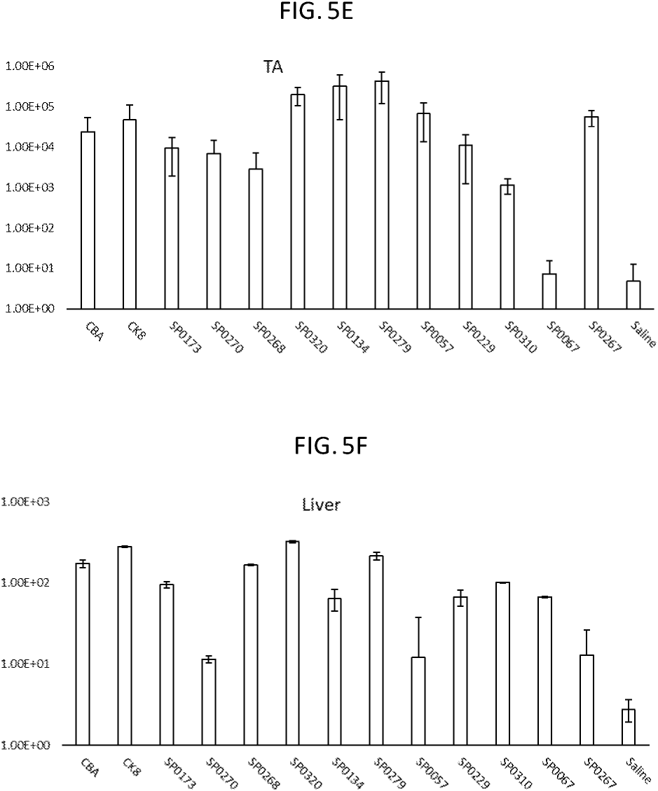

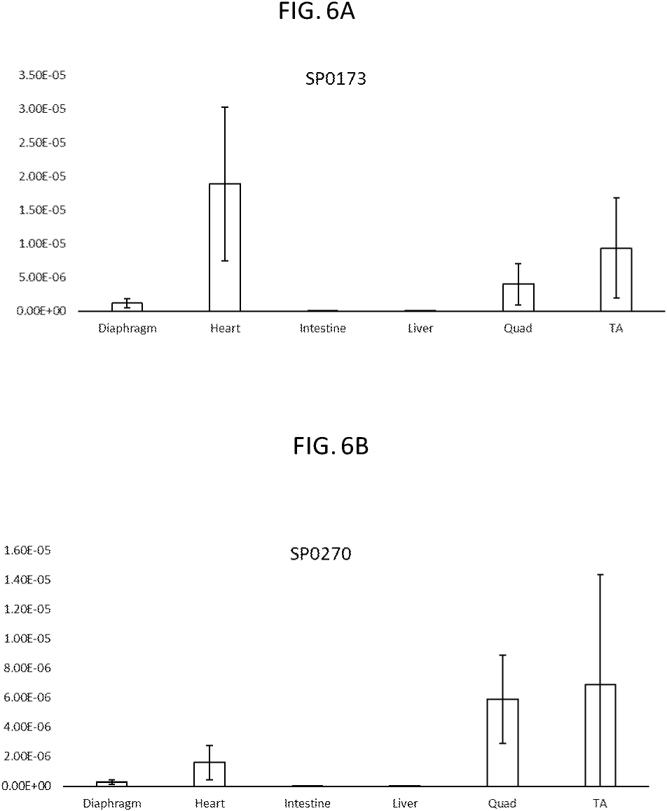

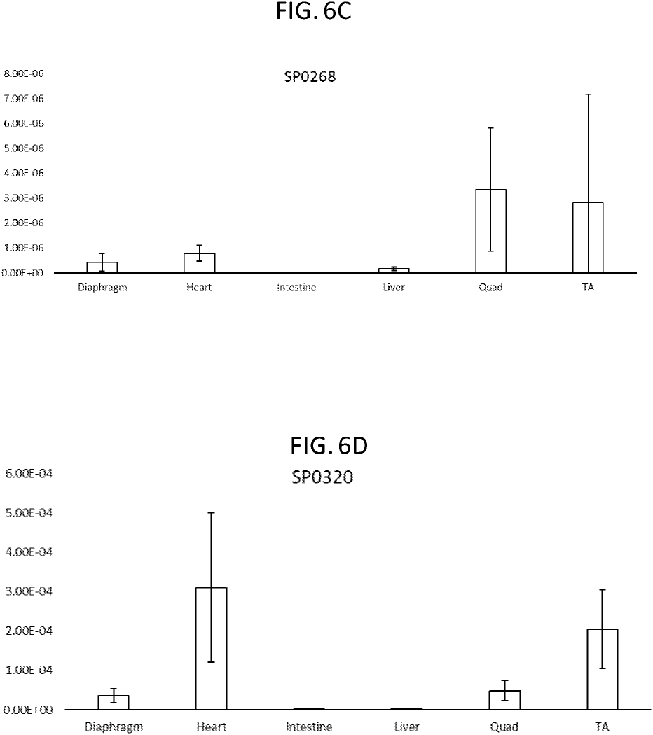

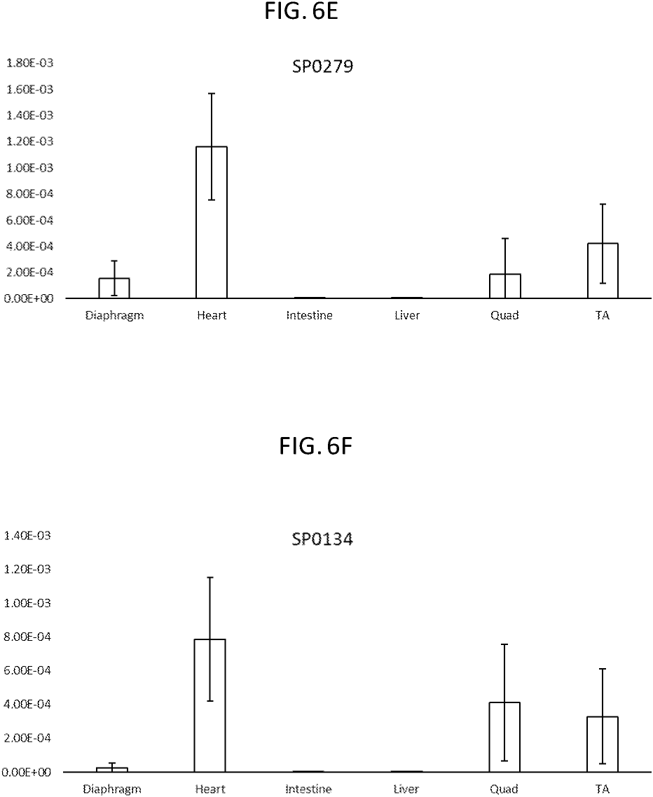

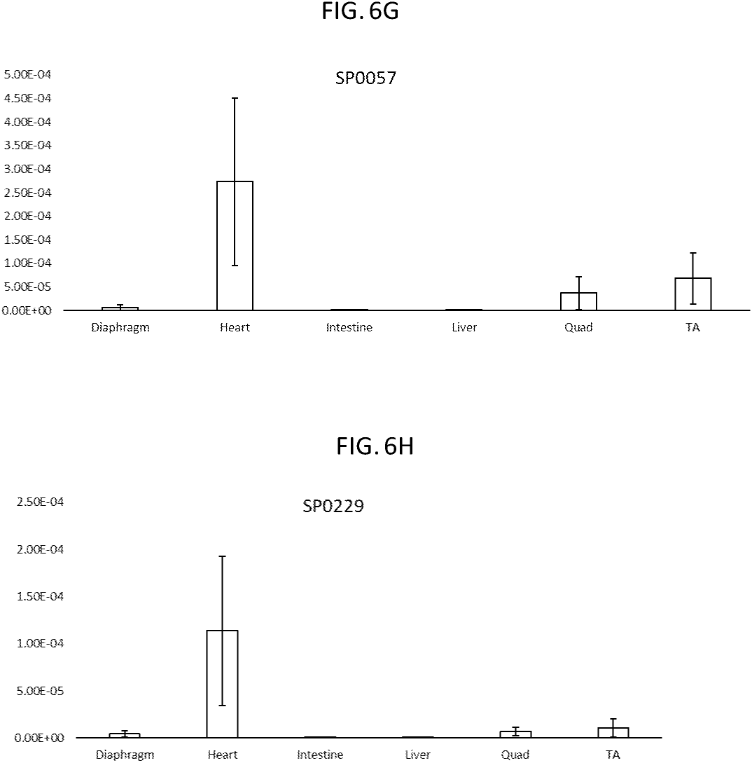

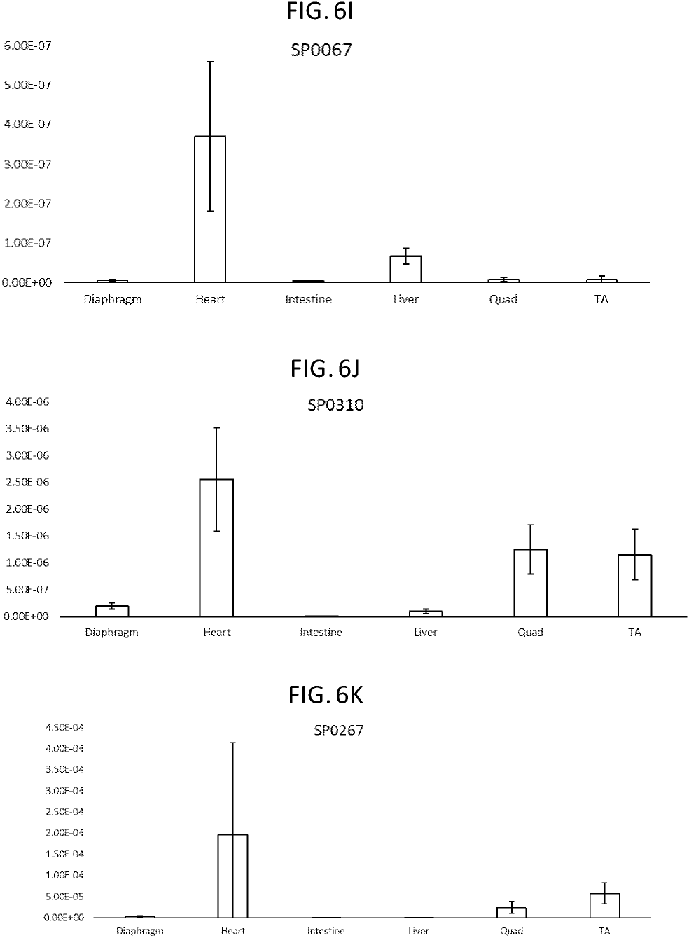

The x axis in FIGS. 5A-5F is in logarithmic scale. In order to plot the data in a logarithmic scale (log₁₀), the normalised RLU values were multiplied by 10⁹ before the conversion to logarithmic scale. The x axis represents log₁₀ of normalised RLU values times 10⁹. The x axis in FIG. 1, FIG. 2, and FIGS. 6A-6F represents RLU values (pg/ml).

The synthetic promoters tested in vivo were much more active in the heart, diaphragm, quadriceps and tibialis anterior than in the liver and intestine as shown in FIGS. 6A-6F.

Some promoters such as SP0270 and SP0268 (FIGS. 6B and 6C) were more active in skeletal muscle (diaphragm and tibialis anterior) than cardiac muscle (heart). Other promoters such as SP0057, SP0229, and SP0067 (FIGS. 6G, 6H, and FIG. 6I) were more active in the cardiac muscle (heart) than the skeletal muscle (diaphragm and tibialis anterior).

FIG. 1 and FIG. 6I show that cardiac muscle-specific promoter SP0067 is not active in skeletal muscle (diaphragm, quadriceps, tibialis anterior, intestine) but is active in the heart. SP0067 is less active in the heart muscle than control promoters CBA and CK8 but unlike these generic control promoters, it is highly specific for cardiac muscle compared to skeletal muscle. SP0067 also has some activity in liver.

### Example 3

### Assessment of Cardiac-Specific Promoters in Vivo:

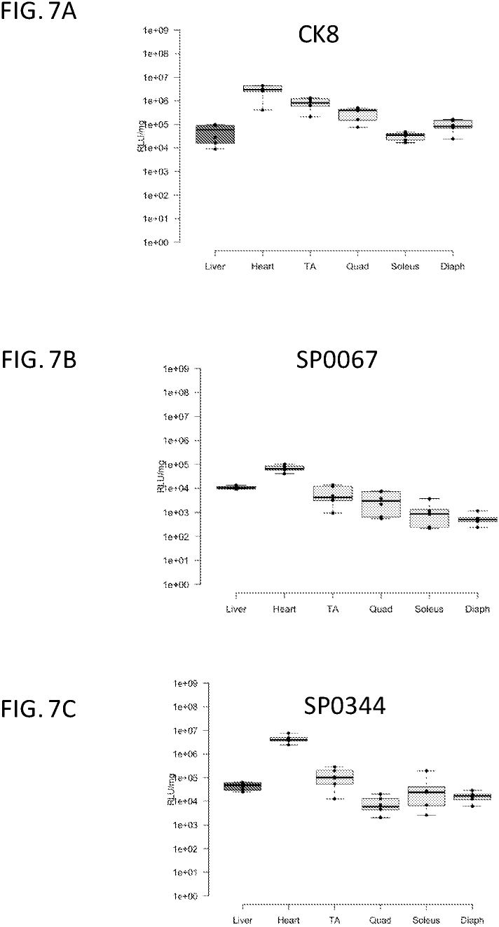

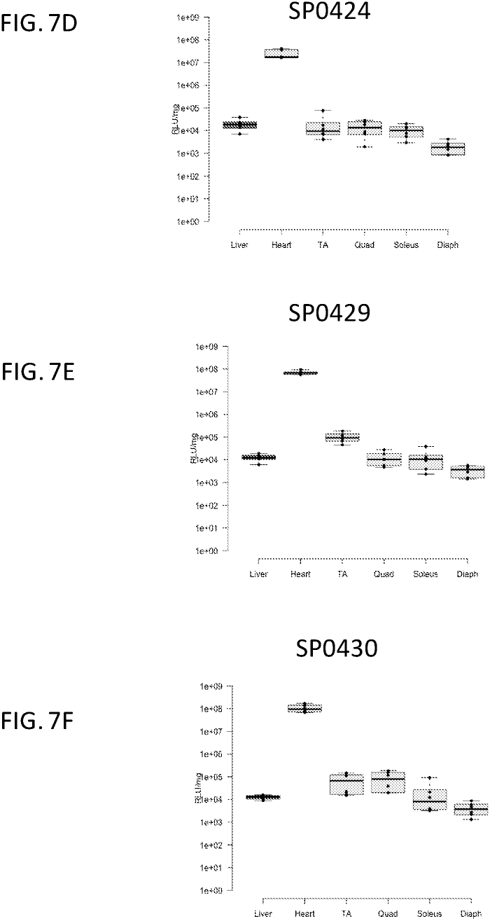

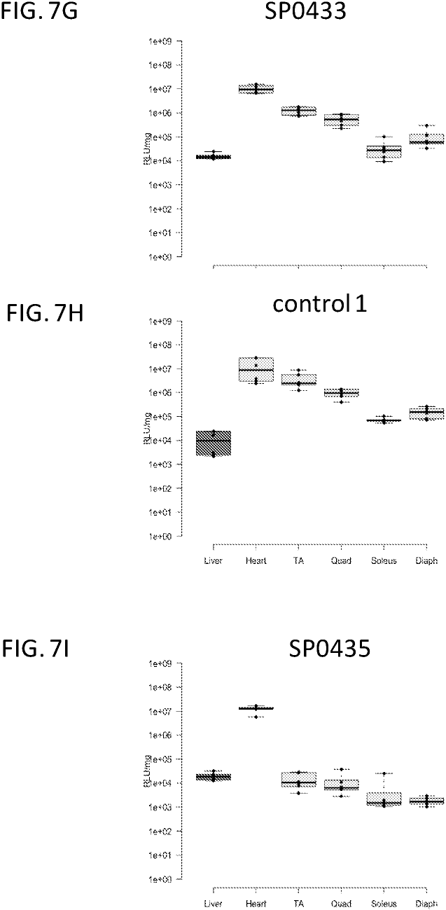

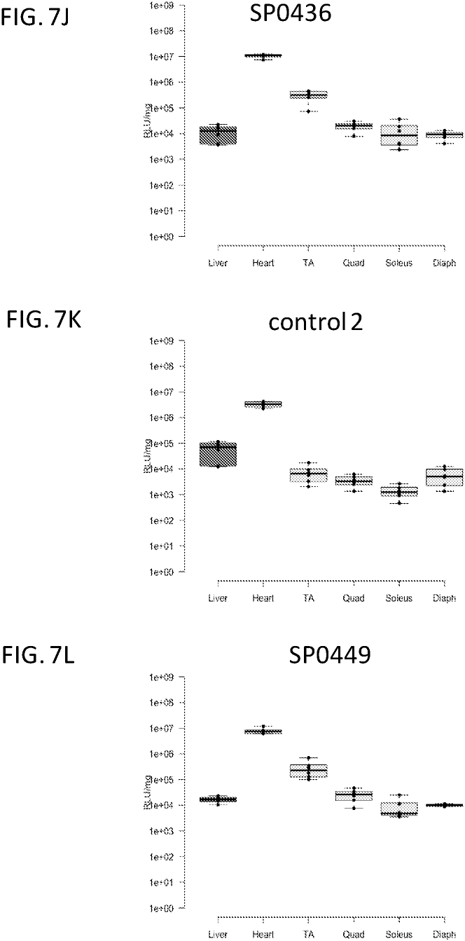

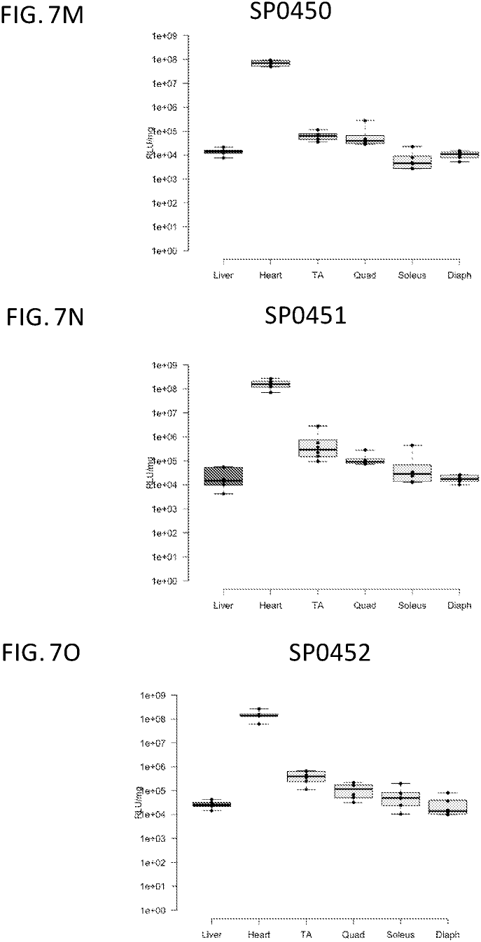

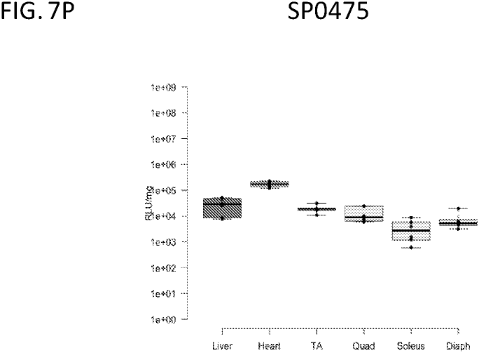

Expression cassettes comprising each of cardiac-specific promoters SP0067, SP0451, SP0452, SP0430, SP0450, SP0429, SP0424, SP0435, SP0436, SP0433, SP0449, SP0344, SP0475, or the control promoters (CK8 — as a muscle promoter control, or cardiac specific promoters control 1 (SEQ ID NO: 288) and control 2 (SEQ ID NO: 289)) promoters as cardiac-specific positive control) in driving the luciferase gene were created and AAV2/9 comprising these expression cassettes were produced as disclosed in Example 2. Cardiac specific control promoters 1 and 2 have been previously described in Bezzerides, et al. “Gene therapy for catecholaminergic polymorphic ventricular tachycardia by inhibition of Ca2+/calmodulin-dependent kinase II.” Circulation 140.5 (2019): 405-419. The rAAV were purified through iodixanol gradient. Mice were administered the rAAV as disclosed in Example 2, and sacrificed 6 weeks after, and the diaphragm (skeletal muscle), heart (cardiac muscle), liver (specificity control tissue), quadriceps (skeletal muscle), solus (slow-twitch muscle), and tibialis anterior (skeletal muscle) were collected, and divided into 3 parts. Samples were snap-frozen in liquid nitrogen, immediately after dissection, and stored at -80° C. The readouts for Diaphragm, Heart, Liver, Quadriceps, and Tibialis anterior were created by protein quantification (using BCA Pierce protein assay kit; Promega 23225) and Luciferase quantification (using ONE-Glo Luciferase Assay System; Promega E6120). RLU values were calculated as RLU/mg of extracted protein (FIGS. 7A-7M).

The synthetic promoters tested in vivo were much more active in the heart, diaphragm, quadriceps and tibialis anterior than in the liver as shown in FIGS. 7A-7M.

Some cardiac-specific promoters such as SP0424 (FIG. 7C), SP0429 (FIG. 7E), SP0430 (FIG. 7F), SP0435 (FIG. 7I), SP0450 (FIG. 7M), SP0451 (FIG. 7N) and SP0452 (FIG. 7O) all showed specific expression in the heart, with lower expression levels in other muscle tissues. The promoters show significantly low experession in liver that makes the use of these rAAV comprising these promoters as having liver detargetting effect. Furthermore, these rAAVs, with any AAV serotype except AAV2i8 or, BNP116, comprising these cardiac specific promoters with liver detargetting effect can be used for repeat administration, where, AAV2i8 is used in one administration and rAAVs with any AAV serotype except AAV2i8 or, BNP116 comprising these cardiac specific promoters, can be used in the other administration.

The control 1 (FIG. 7H) and control 2 (FIG. 7K) cardiac specific promoters were previously disclosed in Bezzerides, et al. “Gene therapy for catecholaminergic polymorphic ventricular tachycardia by inhibition of Ca2+/calmodulin-dependent kinase II.” Circulation 140.5 (2019): 405-419 and used as controls in this study along with the CK8 control.

### Example 4

rAA V vector manufacturing: Described herein in is a method of manufacturing rAAV viral vectors from Pro10/HEK293 cells that have been engineered to stably express the I-1 gene.

The stable cell line, Pro10/HEK293, as described in U.S. Pat. No. 9,441,206, is ideal for scalable production of AAV vectors. This cell line can be contacted with an expression vector used to express the I-1 gene operatively linked to cardiac-specific promoter SP0067 (SEQ ID NO: 3). Clonal populations having I-1 expression integrated into their genome are selected using methods well known in the art. Expression of the I-1 gene is confirmed via western-blotting.

Pro10/HEK293 cells stably expressing I-1 are transfected with a Packaging plasmid encoding Rep2 and serotype-specific Cap2: AAV-Rep/Cap, and the Ad-Helper plasmid (XX680: encoding adenoviral helper sequences).

Transfection. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before so transfection. The wave bioreactor cell culture is then transfected. Cell culture is harvested from the wave bio-reactor bag at least 48 hours post-transfection.

Titer: AAV titers are calculated after DNase digestion using qPCR against a standard curve (AAV ITR specific) and primers specific to I-1 gene.

Harvesting Suspension Cells from Shaker Flasks and 60 Wave Bioreactor Bags. 48 hours post-transfection, cell cultures are collected into 500 mL polypropylene conical tubes (Coming) either by pouring from shaker flasks or pumping from wave bioreactor bags. The cell culture is then centrifuged at 655xg for 10 min using a Sorvall RC3C plus centrifuge and H6000A rotor. The supernatant is discarded, and the cells are resuspended in 1xPBS, transferred to a 50 mL conical tube, and centrifuged at 655xg for 10 mM. At this point, the pellet can either be stored in NLT-60° C. or continued through purification.

Titering rAAV from Cell Lysate Using qPCR. 10 mL of cell culture is removed and centrifuged at 655xg for 10 min using a Sorvall RC3C plus centrifuge and H6000A rotor. The supernatant is decanted from the cell pellet. The cell pellet is then resuspended in 5 mL of DNase buffer (5 mM CaC12, 5 mM MgC12, 50 mM Tris-HC1 pH 8.0) followed by sonication to lyse the cells efficiently. 300 uL is then removed and placed into a 1.5 mL microfuge tube. 140 units of DNase I is then added to each sample and incubated at 37° C. for 1 hour. To determine the effectiveness of the DNase digestion, 4-5 mg of I-1 plasmid is spiked into a non-transfected cell lysate with is and without the addition of DNase. 50 µL of EDTA/Sarkosyl solution (6.3% sarkosyl, 62.5 mM EDTA pH 8.0) is added to each tube and incubated at 70° C. for 20 minutes. 50 pL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers 2 s designed to bind to a homologous sequence on the backbones of plasmids XX680, pXR2 and I-1. The second qPCR reaction is set up using a set of primers to bind and amplify a region within the I-1 gene. qPCR is conducted using Sybr green reagents and Light cycler 480 from 30 Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400xg using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc.), which are incorporated herein by reference in their entireties.

### Example 5

### Efficacy of rAAV Expressing I-1c to Treat Heart Failure in Humans:

Described herein in Example 5 is a method of administrating a rAAV viral vector expressing the I-1c gene to human patients for the treatment of heart failure according to the methods disclosed herein. Example 5 shows results of human patients with heart failure (non-ischemic cardiomyopathy) administered AAV2i8.sc-CMV.I1c, which is produced from the plasmid of SEQ ID NO: 441 (AAV 2i8 sc in this example and other examples, refers to AAv2i8 having self complementary genome). The AAV vector AAV2i8.sc-CMV.I1c used in Example 5 used solely an exemplary rAAV, and any rAAV disclosed herein can be used in the methods to treat heart failure as disclosed herein. For example, one of ordinary skill in the art can readily substitute the CMV promoter in the AAV2i8.sc-CMV.I1c used in Example 5 for a cardiac-specific promoter, e.g, any cardiac specific promoter, or muscle specific promoter active in cardiac muscle disclosed in Tables 2A or 5A, or shown in FIGS. 7B-7O. Similarly, the AAV2i8.sc-CMV.I1c used in Example 5 can be readily substituted for a rAAV comprising a codon-optimized nucleic acid sequence encoding I-1c, such as, but not limited to the nucleic acid sequences of SEQ ID NO: 385-412. Similarly, the AAV2i8.sc-CMV.I1c used in Example 5 can be readily substituted for a rAAV comprising a nucleic acid comprising codon optimized nucleic acid sequence encoding I-1c, such as, but not limited to the nucleic acid sequences of SEQ ID NO: 385-412; and the nucleic acid further comprising a poly A e.g HGH poly A or SV40 poly A and RNA polymerase II transcriptional pause signal from alpha 2 globin gene. Similarly, the AAV2i8.sc-CMV.I1c used in Example 5 can be readily substituted for a rAAV comprising a nucleic acid sequence of SEQ ID NO: 413-440, or modified version thereof. rAAV lacking bacterial sequence and comprising the AAV2i8.sc-CMV.I1c used in Example 5 can be manufactured from any of linear duplex DNA of SEQ ID NO: 357-384.

7 Patients with symptomatic congestive heart failure received infusions of AAV2i8.sc-CMV.I1c, where the rAAV vector is AAV2i8, and which expresses the I-1c protein (amino acids 1-65 with the T35D modification) under the CMV promoter. A single intracoronary infusion of AAV2i8.sc-CMV.I1c was administered to 7 patients with NYHA Class III heart failure, where administration occurred as single administration of a total dose selected from (i) 3 × 10¹³ vg (n=3) (ii) 1 × 10¹⁴ vg (n=4). All the patients in the study had non-ischemic cardiomyopathy. The total dose was administered to patients in a series of 5 sub-doses, each sub-dose administered from a separate syringe, and where the 5 sub-doses were administered to the subject over a period of about 25-30 minutes. In such instances, the total rAAV dose is diluted in 50 ml saline and administered as 5 sub-doses in 5 syringes, with 10 ml volume in each syringe. The administration is performed over a time period of about 5 minutes for each of the five syringes, one syringe after another, with frequent stops checking the position of catheters in each coronary artery using contrast injections. All patients were followed until 12 months post treatment intervention, and then undergo long-term follow-up via semi-structured telephone questionnaires every 6 months for an additional 24 months (+/- 30 days). The patients were coadministered with vasodilator e.g. nitroglycerin about 25 minutes prior to rAAV administration.

Before administration (i.e. at screening), and after 4-weeks (+/- 3 days), 6 months and 12-months post-administration, the patients were assessed using a variety of physiological assessments and clinical parameters. For example, echocardiographic assessments of LVEF, LVEVD, LVEDVI, VLESV, LVEVI, SpI and GLS and degree of mitral regurgitation was assessed, as well as serum NT-proBNP level, as well as secondary effects assessed to determine efficacy of the administration of the AAV2i8.sc-CMV.I1c rAAV vector. Secondary effects include, e.g., but are not limited to (i) Peak VO₂ assessed by cardiopulmonary exercise testing, (ii) 6-minute walk test (6MWT), (iii) New York Heart Association (NYHA) Classification, (iv), total number of days alive out-of-hospital (as well as total days out-of-hospital as a% of total days alive post study intervention) (v), quality of Life at 6 and 12 months compared to baseline, (vi) Health related quality of life as assessed by Minnesota Living with Heart Failure Questionnaire (MLWHFQ). Also assessed at 12-months and long-term follow-up period (until month 36 post-intervention) was patient, need for cardiac transplantation, or left ventricular assist device (LVAD) implantation. Each of these clinical parameters are discussed below:

NYHA: NYHA class is a clinician-assessed measure of patients’ functional status, and is widely used in clinical trials due to its simplicity and prognostic value.

LVEF: LVEF is indicative of cardiac systolic function and measures as a percentage the amount of blood pumped out of the left ventricle with each contraction. Reduction in EF is associated with cardiac remodeling. Treatment-induced changes in LVEF may be predictive of an effect on mortality. Each patient had an improvement in LVEF from baseline to Month 12. The absolute percent increase in patient 1 was +10.5%, in patient 2, +22%, and in patient 3, +6%. In some patients, the improvement in LVEF was associated with decreases in both left ventricular end systolic volumes and left ventricular end-diastolic volumes. In addition, it was noted that from the time of screening to month 12, all patients had improvements in left ventricular wall motion abnormalities.

6MW Test: The 6MWT is a standard means for assessing submaximal exercise capacity, and is defined as the distance in meters walked in 6 minutes. 6MWT is considered a surrogate for quality of life, and performance correlates strongly with subsequent clinical outcomes.

VO2Max: Cardiopulmonary testing and measurement of Peak VO2 measure during maximal exercise have been used for assessment of aerobic capacity, establishment of prognosis in heart failure patients, and evaluation of benefits of various forms of intervention. Most recently, EXPLORER-HCM trial evaluated the effectiveness of Mavacamten in patients with obstructive cardiomyopathies using the primary composite functional end point at week 30 compared to baseline defined as either (1) an increase in peak oxygen consumption ≥1.5 mL/kg/min and reduction of at least one New York Heart Association class; or (2) an improvement of ≥3.0 mL/kg/min in peak oxygen consumption with no worsening of New York Heart Association class.

MLWHFQ: Quality of life is most often assessed with well-validated questionnaires such as the Minnesota Living with Heart Failure Questionnaire (MLWHFQ), which have been shown to be more reproducible than clinician-assessed symptoms.

BNP: Biomarkers such as brain natriuretic peptides (BNPs) and NTpro-BNP have been used as inclusion criteria for patients with heart failure and to track the effects of treatments in HF patients. Some Patients had a decrease in BNP levels from baseline to Month 12 (-38 and -60), indicative of improvements.

Primary outcome measures were assessed, which include a change in Peak VO₂ and LVEF levels. To determine the Peak VO₂ change, the Peak VO₂ measured at 6- and 12-months post-administration as compared to the Peak VO₂ before, or at the time of administration (i.e., as compared to baseline), as determined by Cardiopulmonary exercise testing using a modified Bruce protocol. Other primary outcome measures assessed were a change in echocardiographic assessment in Left Ventricular Ejection Fraction (LVEF) measured at the time of administration or screening, 18-24 hours post-administration, 4-weeks, 6 months and 12 months post-administration as compared to the LVEF measured before or at the time of administration (e.g., baseline LVEF level).

Secondary outcome measures were also assessed, which include a change in results from the 6MWT (6-minute walk test), measured at 6- and 12-months post administration as compared to the results from the 6MWT measured at, or before administration (i.e., as compared to the baseline). Secondary outcome measures of the analysis of % predicted in heart failure subjects as compared to normal subjects was also determined.

TABLE 19A show the result of clinical improvement using the NYHA classification of patients receiving 3 × 10¹³ vg rAAV expressing I-1c (n=3), where NYHA classification system was assessed at 1-month, 2-months, 3-months, 6-months, 9-months and 12-months post administration.

Dose: 3 × 10¹³ vg/patient

| Patient No: | 2 | | 3 | | 5 | |
|---|---|---|---|---|---|---|
| | NYHA | EF (%) | NYHA | EF (%) | NYHA | EF (%) |
| Screening | III | 35^{∗} | III | 27 | III | 29 |
| 1 month | | | II | 40 | | 25 |
| 3 months | | 39 | | | II | |
| 6 months | II | 41 | I | 41 | II | 41 |
| 9 months | | | | | | |
| 12 months | II | 39 | I | 35 | | |

TABLE 19B show the result of clinical improvement using the NYHA classification of patients receiving 1 × 10¹⁴ vg rAAV expressing I-1c (n=3), where NYHA classification system was assessed at 1-month, 2-months, 3-months and 6-months post administration.

Dose: 1 × 10¹⁴ vg/patient

| Patient No: | 11 | | 9 | | 13 | | 12 | |
|---|---|---|---|---|---|---|---|---|
| | NYHA | EF (%) | NYHA | EF (%) | NYHA | EF (%) | NYHA | EF (%) |
| Screening | III | 14 | III | 18 | III | 28 | III | 22.5 |
| 1 month | | 23 | | 19 | | 31 | | |
| 3 months | II | 26 | | | | | | |
| 6 months | II | 15 | | | | | | |
| 12 months | | | | | | | | |

The results (see Table 19A) showed improved classification of patients (receiving 3×10¹³ vg rAAV) of at least one class of the NYHA classification system, and in some instances, subject improve two classes of the NYHA classification within 6 months. For instance, patient No. 2 improved from a class III to a class II within 6 months, and patient No. 3 improved from a class III to a class I within 6 months, demonstrating a suprising and significant improvement in two levels in the NYHA classification system within 6-months of administration. Stated another way, patient No. 3 had a 2 class decrease in the NYHA classification system in 13 months after administration of the rAAV encoding the I-1c. Similar to the 3×10¹³ dose, patients receiving 1×10¹⁴ vg rAAV (Table 19B) also showed improvement of NYHA classification, e.g patient 11 improved by one class as shown by NYHA classification value at 3 months and at 6 months.

The Ejection Fraction (%EF) was also assessed at the time of screening and at multiple times post administration (e.g., 1-month, 2-months, 3-months, 6-months, 9-months and 12-months post administration) and the results shown in Tables 19A-19B. There was a 5% or greater than 5% increase in EF in most treated patients, e.g., patient Nos. 3 and 5 had%EF increased upto 41% from 27% at baseline and upto 41% from 29% at baseline in 6 months respectively after administration of 3×10¹³ vg rAAV expressing I-1c.

Summary result of other parameters:

In 6 minute walk test (6MWT), all patients increased 6 minute walk distance (6MWD) by at least 50 meters within 12 months and some patients increased by 50 meters within 6 months. With respect to MWHF questionnaire, all patients showed a decrease of more than 10 scores in 12 months. All patients increased pVO2max or, MVO2 by 1.5 ml/kg/min or, more in 12 months

Clinically meaningful changes for each functional test is shown in Table 19C. Administration of the AAV2i8.sc-CMV.I1c rAAV to patients was shown to achieve one or more endpoints in Table 19C.

| | |
|---|---|
| PARAMETER | CHANGE FROM BASELINE |
| Left Ventricular Ejection Fraction | ≥5% Increase |
| Left Ventricular End Systolic Volume | >20 mL or 10% Decrease |
| New York Heart Association Class | 1 Class Decrease |
| Quality of Life (Minnesota Living With Heart Failure Questionaire) | 10-Point Decrease |
| 6 Minute Walk Test | 50-Meter Increase |
| BNP (pg/mL) | 40% Decrease |
| NT-pro-BNP (pg/mL) | 35% Decrease |
| MVO 2 | 1.5 mL/kg/min Increase |

Accordingly, the results show improved clinical parameters (such as decreased serum NT-pro BNP, increased peak VO₂ (data not shown), increased LVEF etc.) in patients administered AAV2i8.sc-CMV.I1c in a single administration of a total dose selected from (i) 3 × 10¹³ vg (n=3) (ii) 1 × 10¹⁴ vg (n=4), as compared to the clinical parameters (e.g., change in serum NT-pro BNP, change in peak VO2, change LVEF) measured in patients prior to administration.

In a pig study (data not shown) enhanced transduction of rAAV in the heart of pigs is observed, when the total dose of AAV2i8.sc-CMV.I1c rAAV vector is administered with 5 syringes over a period of about 25 minutes wherein each syringe has 10 ml. saline, as compared to, when the same total dose of AAV2i8.sc-CMV.I1c rAAV vector is administered with two syringes over a time period of 10 minutes, wherein each syringe has 10 ml. saline.

Based on the significant improvement in HF clinical parameters of non-ischemic cardiomyopathy patients in this Example as detected at 6- or 12 months after administration of the AAV2i8.sc-CMV.I1c, where the patients showed significant improvement in clinical parameters, such as improved NYHA class, increased%EF, increased walk distance on 6MWT, increased peak VO₂ (pVO2 or, MVO2), increased LVEF (or, EF), improved score of MWHFQ (or, KCCQ) etc., it is expected that patients with non-ischemic cardiomyopathies from a range of causes or diseases will also have similar improvement in the HR clinical parameters at least 12-months post administration, including patients with non-ischemic cardiomyopathy due to infections resulting in myocardial inflammation (such as infections by various viruses, including possibly, patients previously infected with SARS-Cov2, or subjects who have had COVID-19 infection, bacterial infections and other parasites), noninfectious inflammations (such as those due to autoimmune diseases, peripartum cardiomyopathy, hypersensitivity reactions or transplantation rejections), metabolic disturbances causing myocarditis (including nutritional, endocrinologic and electrolyte abnormalities) and exposure to toxic agents causing myocarditis (including alcohol, as well as certain chemotherapeutic drugs and catecholamines). Thus Example 5 demonstrated the methods disclosed herein are useful for the treatment of subject with HF where the cause of disease remains unknown, and thus is useful to treat patients with “idiopathic dilated cardiomyopathy” (or “IDCM”).

### Example 6

In vivo treatment of human subjects with non-ischemic inherited cardiomyopathies.

Described in Example 5 is the treatment of subjects with heart failure due to non-ischemic cardiomyopathy. Example 6 herein describes a method of administrating a rAAV viral vector expressing the I-1c gene to human patients for the treatment of inherited cardiomyopathies causes and/or, cardiomyopathies associated with genetic mutation, according to the methods disclosed herein. Example 6 discusses administration of AAV2i8.sc-CMV.I1c as an exemplary rAAV, which is produced from the plasmid of SEQ ID NO: 441. The rAAV vector AAV2i8.sc-CMV.I1c discussed in Example 6 is an exemplary rAAV, and any rAAV disclosed herein can be used in the methods to treat heart failure from a variety of non-ischemic cardiomyopathies due to a variety of different causes, as disclosed herein. For example, one of ordinary skill in the art can readily substitute the CMV promoter in the AAV2i8.sc-CMV.I1c discussed in Example 6 for a cardiac-specific promoter, e.g, any cardiac specific promoter disclosed in Tables 2A or 5A or shown in FIGS. 7B-7O. Similarly, the AAV2i8.sc-CMV.I1c discussed in Example can be readily substituted for a rAAV comprising a codon-optimized nucleic acid sequence encoding I-1c, such as, but not limited to the nucleic acid sequences of SEQ ID NO: 385-412. Similarly, the AAV2i8.sc-CMV.I1c discussed in Example 6 can be readily substituted for a rAAV comprising a nucleic acid sequence of SEQ ID NO: 413-440, or modified version thereof. rAAV lacking bacterial sequence and comprising the AAV2i8.sc-CMV.I1c used in Example 6 can be manufactured from any of linear duplex DNA of SEQ ID NO: 357-384 and be used to treat variety of non-ischemic cardiomyopathies due to a variety of different causes including treating cardiomyopathy with patients having phospholamban mutation as discussed in this example.

Pathogenic mutations in the phospholamban (PLN) give rise to inherited cardiomyopathies due to its role in calcium homeostasis. Several PLN mutations have been identified, with the R14del mutation being the most prevalent cardiomyopathy-related mutation in the Netherlands. The R14del mutation in the PLN gene is present in patients diagnosed with arrhythmogenic cardiomyopathy as well as dilated cardiomyopathy. Patients with the R14del mutation are characterised by older age at onset, low-voltage electrocardiograms and a high frequency of ventricular arrhythmias. Additionally, these patients have a poor prognosis often with left ventricular dysfunction and early-onset heart failure (Hof et al., Prevalence and cardiac phenotype of patients with a phospholamban mutation. Neth Heart J. 2019 Feb;27(2):64-69; HofIE et al., Prevalence and cardiac phenotype of patients with a phospholamban mutation. Neth Heart J. 2019 Feb;27(2):64-69).

Human patients identified to have the R14del mutation in the PLN gene can be administered the AAV2i8.sc-CMV.I1c as disclosed herein. For example, patients from the Netherlands with arrhythmogenic cardiomyopathy and/or dilated cardiomyopathy and/or patients with the R14del mutation in the PLN gene can receive infusions of AAV2i8.sc-CMV.I1c, where the rAAV vector is AAV2i8, and which expresses the I-1c protein (amino acids 1-65 with the T35D modification) under the CMV promoter. A single intracoronary infusion of AAV2i8.sc-CMV.I1c will be administered to human patients with at least a NYHA Class III heart failure, where administration can occur as single administration of a total dose selected from any of: 3 × 10¹³ vg, 1 × 10¹⁴ vg, 3 × 10¹⁴ vg, 1 × 10¹⁵ vg, or 1 × 10¹⁵ vg. All the patients in the study will have been identified to have the R14del mutation in the PLN gene and have non-ischemic cardiomyopathy. In some patients, the total dose will be administered in a series of 5 sub-doses, each sub-dose administered from a separate syringe, and where the 5 sub-doses are administered to the subject over a period of about 25-30 minutes. In such instances, the total rAAV dose is diluted in 50 ml saline and administered as 5 sub-doses in 5 syringes, with 10 ml volume in each syringe. The administration will be performed over a time period of about 5 minutes for each of the five syringes, one syringe after another, with frequent stops checking the position of catheters in each coronary artery using contrast injections. All patients will be followed until 12 months post treatment intervention, and then undergo long-term follow-up via semi-structured telephone questionnaires every 6 months for an additional 24 months (+/- 30 days).

Before administration, and after 4-weeks (+/- 3 days), 6 months and 12-months post-administration, the patients will be assessed using a variety of physiological assessments. For example, echocardiographic assessments of LVEF, LVEVD, LVEDVI, VLESV, LVEVI, SpI and GLS and degree of mitral regurgitation can be assessed, as well as serum NT-proBNP levels, as well as secondary effects assessed to determine efficacy of the administration of the AAV2i8.sc-CMV.I1c rAAV vector. Secondary effects include, e.g., but are not limited to (i) Peak VO₂ assessed by cardiopulmonary exercise testing, (ii) 6-minute walk test (6MWT), (iii) New York Heart Association (NYHA) Classification, (iv), total number of days alive out-of-hospital (as well as total days out-of-hospital as a% of total days alive post study intervention) (v), Quality of Life at 6 and 12 months compared to baseline, (vi) Health related quality of life as assessed by Minnesota Living with Heart Failure Questionnaire (MLWHFQ). Patients can also be assessed at 12-months and long-term follow-up period (until month 36 post-intervention), or assessed for a need for cardiac transplantation, or left ventricular assist device (LVAD) implantation.

Primary outcome measures will be assessed, which include a change in Peak VO₂ and LVEF levels. To determine the Peak VO₂ change, the Peak VO₂ will be measured at 6- and 12-months post-administration as compared to the Peak VO₂ before, or at the time of administration (i.e., as compared to baseline), as determined by Cardiopulmonary exercise testing using a modified Bruce protocol. Other primary outcome measures that will be assessed are a change in echocardiographic assessment in Left Ventricular Ejection Fraction (LVEF) as compared to the LVEF measured at the time of administration or screening, or 18-24 hours post-administration, or 4-weeks, 6 months and 12 months post-administration as compared to the LVEF measured before or at the time of administration (e.g., baseline LVEF level).

Secondary outcome measures can also be assessed, which include a change in results from the 6MWT (6-minute walk test), measured at 6- and 12-months post administration as compared to the results from the 6MWT measured at, or before administration (i.e., as compared to the baseline). Secondary outcome measures of the analysis of% predicted in heart failure subjects as compared to normal subjects was also determined.

Based on the significant improvement in HF clinical parameters of non-ischemic cardiomyopathy patients in Example 5 detected at 6- or 12 months after administration of the AAV2i8.sc-CMV.I1c (e.g., improved clinical parameters, such as improved NYHA class, increased%EF, increased walk distance on 6MWT, decreased serum NT-pro BNP, increased peak VO2, increased LVEF, improved MWHF score, etc.), it is expected that patients with non-ischemic inherited cardiomyopathies, including non-ischemic cardiomyopathy due to a R14del mutation in the PLN gene will also have similar improvement in the HR clinical parameters at least 12-months post administration. Thus Example 5 and 6 suggests the methods disclosed herein are useful for the treatment of subject with HF where the cause of disease remains unknown, and thus is useful to treat patients with non-ischemic cardiomyopathy due a genetic disorder with a cardiac manifestation, or a familial cardiomyopathy (such as that associated with progressive muscular dystrophy, myotonic muscular dystrophy, Freidrich’s ataxia, and hereditary dilated cardiomyopathy, and others as disclosed herein).

### Example 7

In vivo treatment of human subjects with ischemic cardiomyopathy.

Described in Example 7 is the treatment of subjects with heart failure due to ischemic cardiomyopathy. Here Example 7 describes a method of administrating a rAAV viral vector expressing the I-1c gene to human patients for the treatment of heart failure from ischemic cardiomyopathy according to the methods disclosed herein. Example 7 discusses administration of AAV2i8.sc-CMV.I1c as an exemplary rAAV, which is produced from the plasmid of SEQ ID NO: 441. The rAAV vector AAV2i8.sc-CMV.I1c discussed in Example 7 is an exemplary rAAV, and any rAAV disclosed herein can be used in the methods to treat heart failure from a variety of non-ischemic cardiomyopathies due to a variety of different causes, as disclosed herein. For example, one of ordinary skill in the art can readily substitute the CMV promoter in the AAV2i8.sc-CMV.I1c discussed in Example 7 for a cardiac-specific promoter, e.g, any cardiac specific promoter disclosed in Tables 2A or 5A or shown in FIGS. 7B-7O. Similarly, the AAV2i8.sc-CMV.I1c discussed in Example can be readily substituted for a rAAV comprising a codon-optimized nucleic acid sequence encoding I-1c, such as, but not limited to the nucleic acid sequences of SEQ ID NO: 385-412. Similarly, the AAV2i8.sc-CMV.I1c discussed in Example 7 can be readily substituted for a rAAV comprising a nucleic acid sequence of SEQ ID NO: 413-440, or modified version thereof. rAAV lacking bacterial sequence and comprising the AAV2i8.sc-CMV.I1c used in Example 5 can be manufactured from any of linear duplex DNA of SEQ ID NO: 357-384 and be used to treat ischemic cardiomyopathy as discussed in this example.

All patients who were administered the AAV2i8.sc-CMV.I1c as disclosed herein had improvements in left ventricular wall motion abnormalities (as detected by an improvement in LVEF). In particular, one human patient who was administered the AAV2i8.sc-CMV.I1c as disclosed herein had heart failure due to a wall motion left ventricle heart abnormality. Central echocardiogram readings, including echocardiographic assessments of LVEF, LVEVD, LVEDVI, VLESV, LVEVI, SpI and GLS and degree of mitral regurgitation was assessed were taken before administration and at 1-month, 2-months, 3-months, 6-months, 9-months and 12-months post administration. There was a significant improvement in the echocardiogram readings, and in particular, a significant improvement in LVEF.

Based on the significant improvement in LVEF in all patients in Example 5, and a significant improvement in LVEF of one patient with left ventricular wall motion abnormalities, as detected at 6- or 12 months after administration of the AAV2i8.sc-CMV.I1c, it is expected that patients with ischemic cardiomyopathies will also have similar improvement in LVEF and other HR clinical parameters at least 12-months post administration. Thus Example 7 suggests the methods disclosed herein are useful for the treatment of subject with ischemic cardiomyopathy, and thus is useful to treat patients with ischemic cardiomyopathy, including patients with ischemic cardiomyopathy prior to the manifestation of heart failure symptoms or CHF symptoms. Accordingly, it is expected that patients with ischemic cardiomyopathies will also have similar improvement in the HR clinical parameters at least 12-months post administration, including patients with ischemic cardiomyopathy due to myocardial infarction (MI), infarction, tissue ischemia, cardiac ischemia, atherosclerosis or coronary artery disease (CAD).

As such, patients with ischemic cardiomyopathy can receive infusions of AAV2i8.sc-CMV.I1c, where the rAAV vector is AAV2i8, and which expresses the I-1c protein (amino acids 1-65 with the T35D modification) under the CMV promoter. A single intracoronary infusion of AAV2i8.sc-CMV.I1c will be administered to human patients with at least a NYHA Class III heart failure, where administration can occur as single administration of a total dose selected from any of: 3 × 10¹³ vg, 1 × 10¹⁴ vg, 3 × 10¹⁴ vg, 1 × 10¹⁵ vg, or 1 × 10¹⁵ vg. All the patients in the study will have been identified to have ischemic cardiomyopathy. In some patients, the total dose will be administered in a series of 5 sub-doses, each sub-dose administered from a separate syringe, and where the 5 sub-doses are administered to the subject over a period of about 25-30 minutes. In such instances, the total rAAV dose is diluted in 50 ml saline and administered as 5 sub-doses in 5 syringes, with 10 ml volume in each syringe. The administration will be performed over a time period of about 5 minutes for each of the five syringes, one syringe after another, with frequent stops checking the position of catheters in each coronary artery using contrast injections. All patients will be followed until 12 months post treatment intervention, and then undergo long-term follow-up via semi-structured telephone questionnaires every 6 months for an additional 24 months (+/- 30 days). In some instances, the subject can be administered a larger dose of rAAV as compared to the dose of rAAV administered to a subject identified to have non-ischemic cardiomyopathy. In some instances, the administration of the rAAV to a subject with ischemic cardiomyopathy will be by intracoronary injection, or a direct injection into the heart muscle tissue, including but not limited to the muscle of the left ventricle and/or the location of the myocardial infarct (MI). In some instances, the subject can also be administered an additional therapeutic agent to increase blood flow to the infarct region.

Before administration, and after 4-weeks (+/- 3 days), 6 months and 12-months post-administration, the patients will be assessed using a variety of physiological assessments. For example, echocardiographic assessments of LVEF, LVEVD, LVEDVI, VLESV, LVEVI, SpI and GLS and degree of mitral regurgitation can be assessed, as well as serum NT-proBNP levels, as well as secondary effects assessed to determine efficacy of the administration of the AAV2i8.sc-CMV.I1c rAAV vector. Secondary effects include, e.g., but are not limited to (i) Peak VO₂ assessed by cardiopulmonary exercise testing, (ii) 6-minute walk test (6MWT), (iii) New York Heart Association (NYHA) Classification, (iv), total number of days alive out-of-hospital (as well as total days out-of-hospital as a% of total days alive post study intervention) (v), Quality of Life at 6 and 12 months compared to baseline, (vi) Health related quality of life as assessed by Minnesota Living with Heart Failure Questionnaire (MLWHFQ). Patients can also be assessed at 12-months and long-term follow-up period (until month 36 post-intervention), or assessed for a need for cardiac transplantation, or left ventricular assist device (LVAD) implantation.

Primary outcome measures will be assessed, which include a change in Peak VO₂ and LVEF levels. To determine the Peak VO₂ change, the Peak VO₂ will be measured at 6- and 12-months post-administration as compared to the Peak VO₂ before, or at the time of administration (i.e., as compared to baseline), as determined by Cardiopulmonary exercise testing using a modified Bruce protocol. Other primary outcome measures that will be assessed are a change in echocardiographic assessment in Left Ventricular Ejection Fraction (LVEF) as compared to the LVEF measured at the time of administration or screening, or 18-24 hours post-administration, or 4-weeks, 6 months and 12 months post-administration as compared to the LVEF measured before or at the time of administration (e.g., baseline LVEF level).

Secondary outcome measures can also be assessed, which include a change in results from the 6MWT (6-minute walk test), measured at 6- and 12-months post administration as compared to the results from the 6MWT measured at, or before administration (i.e., as compared to the baseline). Secondary outcome measures of the analysis of% predicted in heart failure subjects as compared to normal subjects was also determined.

Based on the improvement of in Left Ventricular Ejection Fraction (LVEF) a determined by echocardiographic assessment after administration of the AAV2i8.sc-CMV.I1c in the patient that had heart failure due to a low motion left ventricle heart abnormality, it is expected that patients with ischemic cardiomyopathy will also have in improved Left Ventricular Ejection Fraction (LVEF) at least 12-months post administration.

### References

All references cited in the specification and the Examples are incorporated in their entirety by reference.

CARR, et al. “Type 1 phosphatase, a negative regulator of cardiac function.” Molecular and cellular biology 22.12 (2002): 4124-4135

DOKAINISH “Left ventricular diastolic function and dysfunction: Central role of echocardiography.” Global Cardiology Science & Practice, 3: 1-12 (2015)

GYÖNGYÖSI et al. “Porcine model of progressive cardiac hypertrophy and fibrosis with secondary postcapillary pulmonary hypertension.” Journal of Translation Medicine, 15(202): 1-15 (2017)

HIGGINS “Natriuretic peptide measurement in heart failure.” acutecaretesting.org: 1-7 (2017)

HOF et al., Prevalence and cardiac phenotype of patients with a phospholamban mutation. Neth Heart J. 2019 Feb;27(2):64-69.

HOF et al., Prevalence and cardiac phenotype of patients with a phospholamban mutation. Neth Heart J. 2019 Feb;27(2):64-69; HofIE et al., Prevalence and cardiac phenotype of patients with a phospholamban mutation. Neth Heart J. 2019 Feb;27(2):64-69.

ISHIKAWA et al. “Cardiac I-1c Overexpression With Reengineered AAV Improves Cardiac Function in Swine Ischemic Heart Failure.” The American Society of Gene & Cell Therapy, 22(12): 2038-2045, (2014).

ISHIKAWA et al. “Gene Transfer for Ischemic Heart Failure in a Preclinical Model.” Journal of Visualized Experiments, 51: 1-3 (2011).

KONERMANN et al. “Changes of the Left Ventricle after Myocardial Infarction-Estimation with Cine Magnetic Resonance Imagine during the First Six Months.” Clinical Cardiology, 20: 201-212 (1997).

PATHAK, et al. “Enhancement of cardiac function and suppression of heart failure progression by inhibition of protein phosphatase 1.” Circulation research 96.7 (2005): 756-766.

SWANK et al. “Modest Increase in Peak VO₂ Is Related to Better Clinical Outcomes in Chronic Heart Failure Patients: Results from Heart Failure and a Controlled Trial to Investigate Outcomes of Exercise Training.” American Heart Association, 579-585 (2012).

TAYLOR et al. “Diagnostic accuracy of point-of-care natriuretic peptide testing for chronic heart failure in ambulatory care: systematic review and meta-analysis.” Nuffield Department of Primary Care Health Sciences at University of Oxford: 1-14 (2018)

WATANABE et al. “Protein Phosphatase Inhibitor-1 Gene Therapy in a Swine Model of Nonischemic Heart Failure.” Journal of the American College of Cardiology, 70(14): 1744-1756 (2017).

1. A method of treating a patient having a heart failure, comprising:
administering into heart cells of the patient having a classification of congestive heart failure (CHF), at least one total dose of a rAAV vector comprising a nucleic acid sequence encoding a phosphatase inhibitor (I-1) protein that inhibits phosphatase activity,
wherein, at least one dose of the rAAV is selected from a total dose-range of about 10¹³ vg to about 10¹⁵ vg, and
wherein, at least twelve months post-administration there is an improvement in the classification of congestive heart failure.

2. The method of claim 1, wherein the classification is based upon a classification system used by the American Heart Association (AHA), the American College of Cardiology (ACC), Minnesota LIVING WITH HEART FAILURE® Questionnaire (MLHFQ), Kansas City Cardiomyopathy questionnaire (KCCQ), or the 2016 European Society of Cardiology guidelines (ESCG), the Japanese heart failure Society (JHFS) guidelines, The Japanese Circulation Society (JCS) Guidelines, or, the New York Heart Association (NYHA).

3. The method of claim 1 or 2, wherein there is an improvement in the classification of at least one level 12 months after administration of the rAAV.

4. The method of claim 1 or 2, wherein there is an improvement in the classification of at least one level within six months after administration of the rAAV.

5. The method of claim 1, wherein twelve months post-administration there is an improvement of at least 2 levels in the Classification.

6. The method of any of claims 1-5, wherein the classification system is NYHA and the level of classification is selected from the group consisting of: Class I, Class II, Class III, and Class IV.

7. The method of any of claims 1-5, wherein the classification system is the American College of Cardiology/American Heart Association (ACC/AHA) complementary staging system and the level of classification is selected from the group consisting of: Stages A, Stage B, Stage C, Stage D.

8. The method of any of claims 1-5, wherein the classification system is KCCQ and the level of classification is a KQQC overall summary score range selected from the group consisting of: KCCQ fair to excellent scores of 50 to 100, very poor to fair scores of 0 to 49, good to excellent scores of 75 to 100, and very poor to good scores of 0 to 74.

9. A method of treating a patient having cardiomyopathy, comprising:
administering into heart cells of the patient at least one total dose of a rAAV vector comprising a nucleic acid sequence encoding a phosphatase inhibitor (I-1) protein that inhibits phosphatase activity,
wherein, at least one dose of the rAAV is selected from a total dose-range of about 10¹³ vg to about 10¹⁵ vg, and
wherein, at least twelve months post-administration there is an improvement in the at least one parameter from a baseline level in the patient, where the at least one parameter is selected from the group consisting essentially of:
a. ejection fraction (EF),
b. end systolic volume (ESV)
c. cardiac contractility, selected from ejection fraction (EF) and fractional shortening (FS)
d. cardiac volumes selected from any of: end diastolic volume (DV) and end systolic volume (ESV),
e. functional criteria, selected from any of: a 6-minute walk test (6MWT), exercise and VO2max;
f. BNP level,
g. Pro-BNP level
h. biomarker level, wherein the biomarker level is selected from the group of: troponin, serum creatinine, cystatin-C, or hepatic transaminases,
i. Patient-reported outcomes (PROs), such as reduced symptoms, health-related quality of life (HRQOL), or patient perceived health status, and
j. decrease in any of: mortality risk due to heart failure, reduced hospitalization due to heart failure symptoms, or therapeutic intervention for treatment of heart failure.

10. The method of claim 9, wherein there is an improvement of at least 2 parameters at least 12 months after administration.

11. The method of claim 10, wherein there is an improvement of at least 3 parameters at least 12 months after administration.

12. The method of claim 11, wherein there is an improvement of at least 4 parameters at least 12 months after administration.

13. The method of claim 12, wherein there is an improvement of at least 5 parameters at least 12 months after administration.

14. The method of claim 9, wherein the improvement is selected from any of:
a. at least a 5% or more increase in ejection fraction from baseline,
b. at least a 10% decrease, or at least a 20 ml decrease in end systolic volume from baseline,
c. at least a 50-meter increase in 6-minute walk test from baseline,
d. at least a 40% decrease in BNP levels (pg/ml) in the blood from baseline,
e. at least a 35% decrease in pro-BNP levels (pg/ml) in the blood from baseline,
f. at least a 10% reduction in a biomarker selected from: troponin, serum creatinine, cystatin-C, or hepatic transaminases from a baseline level of the same biomarker,
g. at least a 1.5 ml/kg/min increase in myocardial oxygen consumption (MVO2) from baseline,or
h. a discharge from hospital due to improved HF symptoms, or a reduced intervention selected from a decrease in the use of any of: inotropes, vasodilators, diuretcis due to improved HF symptoms in the subject.

15. The method of claim 1 or 14, wherein the rAAV vector further comprises CMV promoter or a synthetic promoter, operatively linked to the phosphatase inhibitor protein.

16. The method of any of claims 1-15, wherein the total dose is administered as any of the following administration methods:
a. over a period of time of about 20 minutes to about 30 minutes,
b. administered in a series of sub-doses, wherein each sub-dose is administered over a period of time of about 1 minute to about 5 minutes,
c. administered in a series of five sub-doses, each sub-dose is administered over a period of time of about 1 minute to about 5 minutes, and wherein the five sub-doses are administered over a period of about 20 minutes to about 30 minutes.

17. The method of any of claims 1-16, wherein there rAAV vector comprises a capsid that detargets the liver.

18. The method of any of claims 1-17, wherein the rAAV is selected from the group consisting of AAV1, AAV2, AAV6, AAV8, AAV9, AAV2i8, rh10, AAV2.5 and AAV2G9.

19. The method of any of claims 1-18, wherein the rAAV vector is AAV2i8.

20. The method of any of claim 1-19, wherein at least one total dose of the rAAV is 10¹³ vg, 3X10¹³ vg, 10¹⁴ vg, 3X10¹⁴ vg, or, 10¹⁵ vg.

21. The method of any of claims 1-20, wherein the phosphatase inhibitor (I-1) protein is a constitutively active protein (I-1c).

22. The method of claim 21, wherein the s I-1c is selected from any of:
a. a polypeptide comprises at least amino acid residues 1-54 of SEQ ID NO: 1, wherein SEQ ID NO: 1 is truncated at the C-terminus at amino acid 70, 67, 66, 65, or 61, or 54, and where the there is an aspartic acid at position 35 (T35D),
b. a polypeptide comprising amino acids 1-54 of SEQ ID NO:1 or a functional fragment thereof, wherein the functional fragment has at least 85% sequence identity to amino acid residues 1-54 of SEQ ID NO: 1, or truncated at the C-terminus at amino acid 70, 67, 66, 65, or 61, or 54, and where the there is an aspartic acid at position 35 (T35D).

23. The method of any of claims 1-22, wherein the rAAV genome comprises nucleic acid sequence selected from the group consisting of: SEQ ID NO: 413-441.

24. The method of claim 21, wherein the nucleic acid sequence encoding the I-1 polypeptide is selected from
a. a nucleic acid sequence encoding a polypeptide comprising amino acids 1-65 of SEQ ID NO: 1, wherein threonine (T) at position 35 of SEQ ID NO: 1 is replaced with an amino acid that is not T,
b. a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q),
c. a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

25. The method of any of claims 1-24, wherein the nucleic acid sequence encoding the I-1 protein is a codon optimized nucleic acid sequence.

26. The method of any of claims 1-25, wherein the nucleic acid sequence encoding the I-1 protein is selected from any of SEQ ID NO: 385-412, or a nucleic acid sequence having at least 80% sequence identity to SEQ ID NO: 385-412.

27. The method of any of claims 1-26, wherein the subject with cardiomyopathy has non-ischemic heart failure and/or non-ischemic cardiomyopathy.

28. The method of any of claims 1-27, wherein the subject with cardiomyopathy has a congenital cardiomyopathy or a genetic disorder with a cardiac manifestation.

29. The method of claim 29, wherein the subject with a congenital cardiomyopathy or a genetic disorder with a cardiac manifestation has a disease or disorder selected from the group consisting of:
Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

30. The method of any of claims 1-29, wherein the subject with cardiomyopathy has an ischemic cardiomyopathy.

31. The method of any of claims 1-30, wherein the subject with cardiomyopathy has heart failure.

32. The method of claim 31, wherein the subject with heart failure has a classification that is equivalent to class III or above in the New York Heart Association (NYHA) classification system.

33. The method of any of claims 30-32, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

34. The method of any of claims 1-33, wherein the subject with cardiomyopathy has reduced ejection fraction (rEF or HFrEF), or, preserved ejection fraction (HFpEF).

35. The method of any of claims 1-34, wherein at least twelve months post-administration of the rAAV there is an improvement of at least one class in a classification of heart failure from a baseline level, wherein the classification of heart failure is assessed by at least one of the following:
a. a classification system used by the American Heart Association (AHA), the American College of Cardiology (ACC), the 2016 European Society of Cardiology guidelines (ESCG), the Japanese heart failure Society (JHFS) guidelines, The Japanese Circulation Society (JCS) Guidelines, or, the New York Heart Association (NYHA); or equivalent thereof, or
b. a health-related quality of life (HRQL) questionnaire selected from the group consisting from any of: Minnesota LIVING WITH HEART FAILURE® Questionnaire (MLHFQ), or Kansas City Cardiomyopathy questionnaire (KCCQ), Chronic Heart Failure Questionnaire (CHFQ), Quality of Life Questionnaire for Severe Heart Failure (QLQ-SHF), Left Ventricular Dysfunction (LVD-36) questionnaire, and the Left Ventricular Disease Questionnaire (LVDQ).

36. The method of claim 35, wherein there is an improvement in the classification of at least one level within six months after administration of the rAAV.

37. The method of claim 35, wherein there is an improvement in the classification of at least two levels within twelve months after administration of the rAAV.

38. The method of claim 35, wherein there is an improvement of at least a 10 point decrease in quality of life MLWHFQ or KCCQ from the baseline level.

39. The method of any of claims 1-38, wherein the subject is administered a vasodilator concurrent with and/or, before, and/or, after the administration of the at least one total dose of a rAAV vector.

40. The method of any of claims 1-39, wherein the subject is administered an immune modulator concurrent with, or before, or after the administration of the at least one total dose of a rAAV vector.

41. A pharmaceutical composition comprising an AAV vector that comprises a codon optimized I-Ic nucleic sequence selected from any of SEQ ID NO: 385-412, or nucleic acid sequence having at least 80% sequence identity to SEQ ID NOS: 385-412.

42. The pharmaceutical composition of claim 41, wherein the codon optimized nucleic acid sequence is operably linked to a CMV promoter or a synthetic promoter.

43. The pharmaceutical composition of claim 41, comprising a nucleic acid sequence selected from the group consisting of: SEQ ID NO: 41-42, or a nucleic acid sequence having at least 80% sequence identity to SEQ ID NOS: 385-412.

44. The pharmaceutical composition of any of claims 41-43, for the use in a method according to any of claims 1-40.

45. An adeno-associated virus (AAV) vector comprising a nucleic acid sequence encoding a Phosphatase inhibitor (I-1) polypeptide operably linked to a promoter selected from any of:
a cardiac-specific promoter selected from Table 2A or a variant thereof,
a muscle-specific promoter active in cardiac and skeletal muscle, or a variant thereof, or
any promoter when a cardiac tissue specific enhancer is present.

46. The AAV vector of claim 45, wherein muscle-specific promoter active in cardiac and skeletal muscle is selected from Table 5A or Table 13A or a variant thereof.

47. The AAV vector of any of claims 45-46, wherein the AAV is selected from the group consisting of adeno-associated virus-1 (AAV1), AAV2, AAV3, AAV4, AAV5, AAV6, AAV7, AAV8, AAV9 and AAV2i8.

48. The AAV vector of any of claims 45-47, wherein the AAV comprises a capsid that detargets the liver.

49. The AAV vector of any of claim 45-48, wherein the AAV is AAV2i8.

50. The AAV vector of any of claims 45-49, wherein the phosphatase inhibitor (I-1) polypeptide is a constitutively active protein (I-1c).

51. The AAV vector of any of claims 45-50, wherein the I-1c is selected from any of:
a. a polypeptide comprises at least amino acid residues 1-65 of SEQ ID NO: 1 or a functional equivalent thereof;
b. a polypeptide comprising at least amino acids 1-54 of SEQ ID NO: 1, wherein the polypeptide is truncated at a C-terminus at amino acid selected from residue 70, 67, 66, 65, or 61, or 54, and where the there is an aspartic acid at position 35 (T35D),
c. a polypeptide comprising amino acids 1-65 of SEQ ID NO:1 or a functional equivalent thereof having at least 85% sequence identity to amino acid residues 1-65 of SEQ ID NO: 1, or,
d. a polypeptide selected from any of: SEQ ID NOS: 507 or 527-532 or a functional equivalent thereof having at least 85% sequence identity to amino acid residues of any of SEQ ID NOS: 507 or 527-532.

52. The AAV vector of any of claims 45-51, wherein the nucleic acid sequence encoding a I-1 polypeptide is selected from:
a. a nucleic acid sequence encoding a polypeptide comprising amino acids 1-65 of SEQ ID NO: 1, wherein threonine (T) at position 35 of SEQ ID NO: 1 is replaced with an amino acid that is not T,
b. a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q),
c. a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

53. The AAV vector of any of claims 45-52, wherein the polypeptide is selected from: amino acids 1-54 of SEQ ID NO: 1, amino acids 1-61 of SEQ ID NO: 1, amino acids 1-65 of SEQ ID NO: 1, amino acids 1-66 of SEQ ID NO: 1, amino acids 1-67 of SEQ ID NO: amino acids 1 or 1-77 of SEQ ID NO: 2, or a functional variant thereof, wherein the threonine at position 35 of SEQ ID NO: 1 is replaced with an aspartate acid (T35D), or a conservative amino acid of aspartate.

54. The AAV vector of any of claims 45-50, wherein the nucleic acid sequence encoding the I-1 polypeptide is a codon optimized nucleic acid sequence.

55. The AAV vector of any of claims 45-54, wherein the codon optimized nucleic acid sequence has reduced CpG content or reduced CpG islands as compared to the wild-type reference sequence of a SEQ ID NO: 1, or a fragment thereof.

56. The AAV vector of any of claims 45-54, wherein the nucleic acid sequence encoding the I-1 polypeptide is a codon optimized nucleic acid sequence selected from any of: SEQ ID NO: 385-412 or a nucleic acid sequence at least 80% sequence identity to SEQ ID NO: 385-412.

57. The AAV vector of any of claim 45-56, further comprising at least one ITR located 5′ of the nucleic acid sequence encoding a Phosphatase inhibitor (I-1) polypeptide operably linked to the cardiac-specific promoter or muscle-specific promoter.

58. The AAV vector of any of claim 45-57, further comprising at least two ITRs flanking the nucleic acid sequence encoding a Phosphatase inhibitor (I-1) polypeptide operably linked to the cardiac-specific promoter or muscle-specific promoter.

59. The AAV vector of any of claim 45-58, wherein the ITR sequences are selected from any one or more of: SEQ ID NO: 70-78, or a nucleic acid having at least 85% sequence identity to SEQ ID NO: 70-78.

60. The AAV vector of any of claims 45-59, further comprising a reverse poly A sequence or double stranded RNA termination element, wherein the reverse polyA sequence or double stranded termination element are located 3′ of the nucleic acid sequence encoding a Phosphatase inhibitor (I-1) polypeptide.

61. The AAV vector of claim 60, wherein the reverse poly A sequence, or double stranded RNA termination element is located between 3′ of the nucleic acid sequence encoding a Phosphatase inhibitor (I-1) polypeptide and 5′ of the right ITR.

62. The AAV vector of any of claims 45-61, wherein the nucleic acid sequence can further comprise a nucleic acid sequence encoding at least one immune modulator.

63. The AAV vector of any of claims 45-61, present in a composition or solution, further comprising an immune modulator.

64. The AAV vector of any of claims 45-63, further comprising a polyA sequence selected from any of SV40 polyA (SEQ ID NO: 334), HGH poly A (SEQ ID NO: 66), SEQ ID NO: 284-287, SEQ ID NO 331-335, wherein the polyA sequence is located 3′ of the nucleic acid sequence encoding a Phosphatase inhibitor (I-1) polypeptide.

65. A pharmaceutical composition comprising:
(i) adeno-associated virus (AAV) vector comprising a nucleic acid sequence encoding a Phosphatase inhibitor (I-1) polypeptide operably linked to any one of:
a. a cardiac-specific promoter selected from Table 2A or a variant thereof,
b. a muscle-specific promoter active in cardiac and skeletal muscle, or
c. any promoter when a cardiac tissue specific enhancer is present, or a variant thereof; and
(ii) a pharmaceutically acceptable carrier.

66. The pharmaceutical composition of claim 65, wherein muscle-specific promoter active in cardiac and skeletal muscle is selected from Table 5A or Table 13A or a variant thereof.

67. The pharmaceutical composition of claims 65-66, wherein the AAV is selected from the group consisting of adeno-associated virus-1 (AAV1), AAV2, AAV3, AAV4, AAV5, AAV6, AAV7, AAV8, AAV9 and AAV2i8.

68. The pharmaceutical composition of claims 65-67, wherein the AAV comprises a capsid that detargets the liver.

69. The pharmaceutical composition of any of claims 65-68, wherein the AAV is AAV2i8.

70. The pharmaceutical composition of any of claims 65-69, wherein the AAV comprises a nucleic acid selected from the group consisting of SEQ ID NO: 413-440, or a nucleic acid sequence at least 80% sequence identity to a sequence selected from SEQ ID NO: 413-440, wherein the nucleic acids seet forth in SEQ ID NO: 413-440 comprise a CMV promoter of SEQ ID NO: 330, wherein the CMV promoter of SEQ ID NO: 330 is replaced by any of:
a. a cardiac-specific promoter selected from Table 2A or a variant thereof,
b. a muscle-specific promoter active in cardiac and skeletal muscle, or
c any promoter when a cardiac tissue specific enhancer is present, or a variant thereof.

71. The pharmaceutical composition of any of claims 64-69, further comprises a vasodilator.

72. The pharmaceutical composition of any of claims 64-69, further comprises an immune modulator.

73. The pharmaceutical composition of claim 65, wherein the phosphatase inhibitor (I-1) polypeptide is a constitutively active protein (I-1c).

74. The pharmaceutical composition of claim 73, wherein the 1-1c is selected from any of:
a. a polypeptide comprises at least amino acid residues 1-65 of SEQ ID NO: 1 or a functional equivalent thereof;
b. a polypeptide comprising at least amino acids 1-54 of SEQ ID NO: 1, wherein the polypeptide is truncated at the C-terminus at amino acid 70, 67, 66, 65, or 61, or 54, and where the there is an aspartic acid at position 35 (T35D),
c. a polypeptide comprising amino acids 1-65 of SEQ ID NO:1 or a functional equivalent thereof having at least 85% sequence identity to amino acid residues 1-65 of SEQ ID NO: 1, or
d. a polypeptide selected from any of: SEQ ID NOS: 507 or 527-532 or a functional equivalent thereof having at least 85% sequence identity to amino acid residues of any of SEQ ID NOS: 507 or 527-532.

75. The pharmaceutical composition of any of claims 65-74, wherein the nucleic acid sequence encoding a I-1 polypeptide is selected from:
a. a nucleic acid sequence encoding a polypeptide comprising amino acids 1-65 of SEQ ID NO: 1, wherein threonine (T) at position 35 of SEQ ID NO: 1 is replaced with an amino acid that is not T,
b. a nucleic acid sequence encoding a polypeptide comprising amino acids 1-65 of SEQ ID NO: 1, wherein threonine (T) at position 35 of SEQ ID NO: 1 is replaced with an acid amino acid selected from any of: aspartic acid (D), glutamic acid (E), asparagine (N), glutamine (Q),
c. a nucleic acid sequence encoding a polypeptide comprising amino acids 1-65 of SEQ ID NO: 1, wherein threonine (T) at position 35 of SEQ ID NO: 1 is replaced with aspartic acid (D), or a conservative amino acid of aspartic acid.

76. The pharmaceutical composition of any of claims 65-745, wherein the nucleic acid sequence encoding the I-1 protein is a codon optimized nucleic acid sequence.

77. The pharmaceutical composition of claim 76, wherein the codon optimized nucleic acid sequence has reduced CpG content as compared to a reference wild type sequence.

78. The pharmaceutical composition of any of claims 65-77, wherein the codon optimized nucleic acid sequence encoding the I-1 polypeptide is selected from any of SEQ ID NO: 385-412, or a nucleic acid sequence having at least 80% sequence identity to a sequence selected from any of SEQ ID NOS: 385-412.

79. Use of an AAV vector according to any one of claims 45-64, for the manufacturer of a pharmaceutical composition for the treatment of a subject having cardiomyopathy.

80. The use of the AAV vector of claim 79, wherein the subject with cardiomyopathy has non-ischemic heart failure and/or non-ischemic cardiomyopathy.

81. The use of the AAV vector of claim 79, wherein the subject with cardiomyopathy has a congenital cardiomyopathy or a genetic disorder with a cardiac manifestation.

82. The use of the AAV vector of claim 80, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

83. The use of the AAV vector of claim 79, wherein the subject with cardiomyopathy has an ischemic cardiomyopathy.

84. The use of the AAV vector of claim 79, wherein the subject with cardiomyopathy has heart failure.

85. The use of the AAV vector of claim 84, wherein the subject with heart failure has a classification of heart failure based upon a classification system used by the American Heart Association (AHA), the American College of Cardiology (ACC) or the New York Heart Association (NYHA).

86. The use of the AAV vector of claim 85, wherein the subject with heart failure has a classification of a class III or above class III in the New York Heart Association (NYHA) classification system.

87. Use of an AAV vector according to any one of claims 45-64, for the manufacturer of a pharmaceutical composition for the treatment of a subject having a condition or disease associated with heart failure.

88. The use of claim 87, wherein the subject has a classification of congestive heart failure (CHF).

89. The use of claim 87, wherein the classification is based upon a classification system used by the American Heart Association (AHA), the American College of Cardiology (ACC) or the New York Heart Association (NYHA).

90. The use of claim 87, wherein the subject has non-ischemic heart failure or non-ischemic cardiomyopathy.

91. The use of claim 87, wherein the subject has ischemic heart failure or ischemic cardiomyopathy.

92. The use of any of claims 79 or 87, wherein the subject has reduced ejection fraction (rEF or HFrEF).

93. A cell comprising the AAV vector of any of claims 45-64.

94. The cell of claim 93, wherein the cell is a cardiac cell or muscle cells.

95. The cell of any of claims 93-94, wherein the cell is in cell culture or a cell present in a subject.

96. An AAV vector according to claims 45-64, a pharmaceutical formulation of any of claims 65-78, or a cell according any of claims 93-95 for use in the treatment of a subject having cardiomyopathy.

97. The AAV vector of claim 96, wherein the subject with cardiomyopathy has non-ischemic heart failure and/or non-ischemic cardiomyopathy.

98. The AAV vector of claim 96, wherein the subject with cardiomyopathy has a congenital cardiomyopathy or a genetic disorder with a cardiac manifestation.

99. The AAV vector of claim 98, wherein the subject with a congenital cardiomyopathy or a genetic disorder with a cardiac manifestation has a disease or disorder selected from the group consisting of:
Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

100. The AAV vector of claim 96, wherein the subject with cardiomyopathy has an ischemic cardiomyopathy.

101. The AAV vector of claim 96, wherein the subject with cardiomyopathy has heart failure.

102. The AAV vector of claim 101, wherein the subject with heart failure has a classification of heart failure based upon a classification system used by the American Heart Association (AHA), the American College of Cardiology (ACC) or the New York Heart Association (NYHA).

103. The AAV vector of claim 102, wherein the subject with heart failure has a classification of a class III or above class III in the New York Heart Association (NYHA) classification system.

104. An AAV vector according to claims 45-64, a pharmaceutical formulation of any of claims 65-78, or a cell according any of claims 93-95 for use in the treatment of a patient having heart failure.

105. The AAV vector of claim 104, wherein the subject has a classification of congestive heart failure (CHF).

106. The AAV vector of claim 105, wherein the classification is based upon a classification system used by the American Heart Association (AHA), the American College of Cardiology (ACC) or the New York Heart Association (NYHA).

107. The AAV vector of claim 104, wherein the subject has non-ischemic heart failure or non-ischemic cardiomyopathy.

108. The AAV vector of claim 104, wherein the subject has ischemic heart failure or ischemic cardiomyopathy.

109. The AAV vector of claim 96 or 104, wherein the subject has reduced ejection fraction (rEF or HFrEF).

110. The AAV vector of claim 104, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

111. The AAV vector of claim 104, wherein the subject has one or more of:
a. non-ischemic heart failure;
b. non-ischemic cardiomyopathy
c. a classification of congestive heart failure (CHF) is based upon a classification system used by the American Heart Association (AH), the American College of Cardiology (ACC) or the New York Heart Association (NYHA); or
d. a reduced ejection fraction (rEFor HFrEF).

112. A method of expressing a phosphatase inhibitor (I-1) polypeptide in a subject with cardiomyopathy, the method comprising introducing into the subject with cardiomyopathy, at least one dose of the AAV vector according to any of claims 45-63,
wherein the subject with cardiomyopathy has a classification of heart failure,
wherein the at least one dose of the rAAV is selected from a total dose-range of about 10¹³ vg to about 10¹⁵ vg, and
wherein at least twelve months post-administration there is an improvement in the classification of heart failure.

113. The method of claim 112, wherein the classification of heart failure is based upon a classification system used by the American Heart Association (AH), the American College of Cardiology (ACC) or the New York Heart Association (NYHA).

114. The method of claim 112, wherein there is an improvement of classification of at least one level 12 months after administration of the rAAV.

115. The method of claim 112, wherein there is an improvement of classification of at least one level within six months after administration of the rAAV.

116. The method of claim 112, wherein twelve months post-administration there is an improvement of at least 2 levels in the classifications in any one or more of:
the American Heart Association (AH),
the American College of Cardiology (ACC), or
the New York Heart Association (NYHA).

117. The method of any of claims 112-116, further comprising administering an immune modulator concurrent with, or before, or after the administration of the at least one total dose of a rAAV vector.

118. The method of any of claims 112-116, further comprising administering a vasodilator concurrent with, and/or before, and/or after the administration of the at least one total dose of a rAAV vector.

119. The method of any of claims 112-118, wherein the subject has non-ischemic heart failure or non-ischemic cardiomyopathy.

120. The method of any of claims 112-119, wherein the subject with non-ischemic heart failure or non-ischemic cardiomyopathy is has a congenital cardiomyopathy or a genetic disorder with a cardiac manifestation.

121. The method of claim 120, wherein the subject with a congenital cardiomyopathy or a genetic disorder with a cardiac manifestation has a disease or disorder selected from the group consisting of: Arrhythmogenic right ventricular cardiomyopathy, Atrial myxoma, familial, Atrial septal defect ostium primum, Atrial septal defect sinus venosus, Barth syndrome, muscular dystrophy, Buerger disease, Cardioencephalomyopathy, Chromosome 1p36 deletion syndrome, Congenital generalized lipodystrophy type 4, Congenital heart block, Dilated cardiomyopathy, Duchenne muscular dystrophy (DMD), Fabry disease, Familial atrial fibrillation, Familial dilated cardiomyopathy, Familial hypertrophic cardiomyopathy, Familial progressive cardiac conduction defect, Familial thoracic aortic aneurysm and aortic dissection, Fibromuscular dysplasia, Friedreich ataxia, Gaucher disease, Glycogen storage disease (types 2, 3 or 4), His bundle tachycardia, Hurler syndrome, Hypoplastic left heart syndrome, Infantile histiocytoid cardiomyopathy, Intracranial arteriovenous malformation, Isobutyryl-CoA dehydrogenase deficiency, Kallikrein hypertension, Kawasaki disease, Kearns-Sayre syndrome, Left ventricular noncompaction, Limb-girdle muscular dystrophy (types 1B, 2E, 2F, 2M, 2C, 2D), Limited systemic sclerosis, Long QT syndrome 1, Lymphedema and cerebral arteriovenous anomaly, Lymphocytic vasculitis, Microcephaly-cardiomyopathy; Mitochondrial encephalomyopathy lactic acidosis and stroke-like episodes, Mitochondrial trifunctional protein deficiency, Myotonic dystrophy type 1, Neonatal stroke, Noonan syndromes 1 -, 2-, 3-, 4-, 5- and 6, Peripartum cardiomyopathy, Peters plus syndrome, PGM1-CDG, PHACE syndrome, Phospholamban Arg 14 Deletion, Postural orthostatic tachycardia syndrome, Primary carnitine deficiency, Progressive familial heart block (types 1A, 1B and 2), Pseudohypoaldosteronism type 2, Pulmonary arterial hypertension, Pulmonary atresia with intact ventricular septum, Pulmonary atresia with ventricular septal defect, Pulmonary valve stenosis, Pulmonary vein stenosis, Pulmonic stenosis, Renoprival hypertension, Retinal arterial macroaneurysm with supravalvular pulmonic stenosis, Right ventricle hypoplasia, Sarcoidosis, Sengers syndrome, Situs inversus, Sudden Arrhythmia Death Syndrome, Supravalvular aortic stenosis, Swyer syndrome, TANGO2-Related Metabolic Encephalopathy and Arrhythmias, TARP syndrome, Tetralogy of Fallot, Timothy syndrome, Tricuspid atresia, Vici syndrome, VLCAD deficiency and Williams syndrome.

122. The method of any of claims 111-117, wherein the subject with heart failure is an ischemic cardiomyopathy.

123. The method of any of claims 111-120, wherein the subject with heart failure has a cardiovascular disease or heart disease is selected from any of: congestive heart failure (CHF), left ventricular remodeling, peripheral arterial occlusive disease (PAOD), dilated cardiomyopathy (DCM) including idiopathic dilated cardiomyopathy (IDCM), coronary artery disease, ischemia, arrhythmia, myocardial infarction (MI), abnormal heart contractility, acute (decompensated) heart failure (AHF), abnormal Ca2+ metabolism, myocardial ischemia, atherosclerosis, cardiomyopathy, idiopathic cardiomyopathy, genetic disorder induced cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease and pulmonary heart hypertension.

124. The method of any of claims 111-122, wherein the subject has reduced ejaculation fraction (rEFor HFrEF).

125. The method of any of claims 111-123, wherein the heart failure comprises ischemia, arrhythmia, myocardial infarction, abnormal heart contractibility, or abnormal Ca2+ metabolism.

126. The method of any of claims 111-124, wherein the administration is into the lumen of the coronary artery of the heart of the patient.

127. The method of any of claims 111-125, wherein the at least one dose is a total dose-range of about 10¹³ vg to about 10¹⁵ vg., administered in one dose or 2 to 5 sub-doses.

128. The method of any of claims 111-126, wherein the total dose is administered as any of the following administration methods:
a. over a period of time of about 20 minutes to about 30 minutes,
b. administered in a series of sub-doses, wherein each sub-dose is administered over a period of time of about 1 minute to about 5 minutes,
c. administered in a series of five sub-doses, each sub-dose is administered over a period of time of about1 minute to about 5 minutes, and wherein the five sub-doses are administered over a period of about 20 minutes to about 30 minutes.
